(19) United States
(12) Patent Application Publication
(10) Pub. No.: US 2025/0197388 A1
(43) Pub. Date: Jun. 19, 2025

(54) COMPOUNDS, PHARMACEUTICAL COMPOSITIONS AND METHODS FOR TREATING INFLAMMATORY BOWEL DISEASE

(71) Applicant: FLAGSHIP PIONEERING INNOVATIONS VI, LLC, Cambridge, MA (US)

(72) Inventors: Pranatchareeya CHANKHAMJON, Cambridge, MA (US); Kevin D. KREUTTER, Cambridge, MA (US); Jason PARK, Cambridge, MA (US); Zachary Lawrence REITZ, Cambridge, MA (US); Francesco G. SALITURO, Cambridge, MA (US); Sabrina Yusang YANG, Cambridge, MA (US); Nir GRABIE, Cambridge, MA (US); David Richard JACKSON, Cambridge, MA (US); Hsin-yu LEE, Cambridge, MA (US)

(21) Appl. No.: 18/850,640

(22) PCT Filed: Mar. 29, 2023
(86) PCT No.: PCT/US2023/016739

§ 371 Date: Sep. 25, 2024

Related U.S. Application Data
(60) Provisional application No. 63/324,801, filed on Mar. 29, 2022.
(60) Provisional application No. 63/324,934, filed on Mar. 29, 2022.

(51) Int. Cl. C07D 417/14; A61K 31/427; A61K 31/4439; C07D 277/10

(52) U.S. Cl. CPC C07D 417/14; A61K 31/427; A61K 31/4439; C07D 277/10

(57) ABSTRACT

The disclosure provides pharmaceutical compositions comprising a therapeutically effective amount of compound (A), compound (B), compound (C), compound (D), compound (E), compound (F), compound (G), compound (H), compound (J), compound (K), compound (L), compound (M), compound (N), compound (O), compound (P), or compound (Q) or a pharmaceutically acceptable salt thereof, and a pharmaceutically acceptable excipient: Also provided are dosage units comprising one or more of compound (A), compound (B), compound (C), compound (D), compound (E), compound (F), compound (G), compound (H), compound (J), compound (K), compound (L), compound (M), compound (N), compound (O), compound (P), or compound (Q) or the pharmaceutical compositions described herein, methods of treating an inflammatory bowel disease in a subject in need thereof, or methods of modulating an inflammatory bowel disease marker in a subject in need thereof.

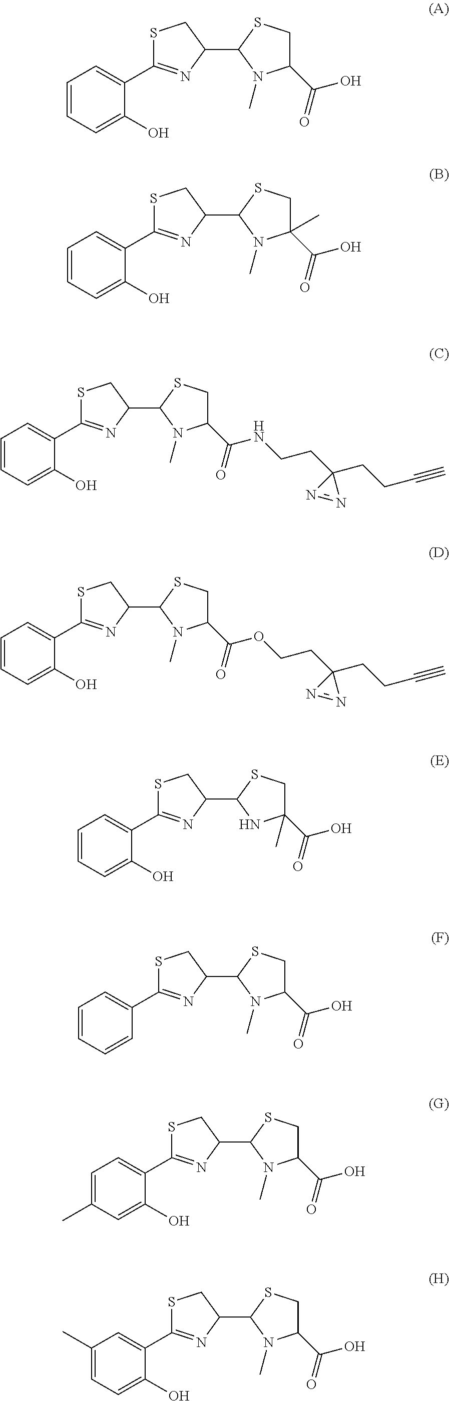

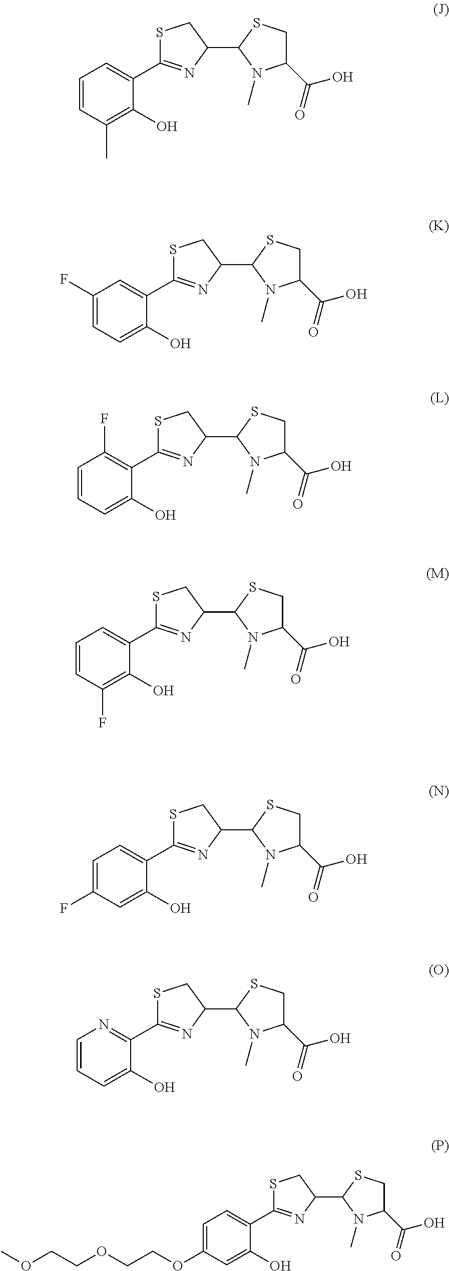

### CROSS-REFERENCE TO RELATED APPLICATIONS

This application claims the benefit of the priorities of US Provisional Patent Application Nos. 63/324,741, filed Mar. 29, 2022; 63/324,801, filed Mar. 29, 2022; 63/324,897, filed Mar. 29, 2022; and 63/324,934, filed Mar. 29, 2022, the disclosures of which are incorporated by reference herein.

### TECHNICAL FIELD

The disclosure relates to compounds, pharmaceutical compositions and methods for treating inflammatory bowel disease.

### BACKGROUND

Inflammatory bowel disease (IBD) typically refers to two conditions: Crohn's disease and ulcerative colitis. Crohn's disease is characterized by inflammation of the digestive tract lining and may often spread deep into affected tissues. Ulcerative colitis is a condition causing long-lasting inflammation and sores (ulcers) in the innermost lining of the large intestine (colon) and rectum. Typical symptoms of ulcerative colitis and Crohn's disease include severe diarrhea, abdominal pain, fatigue, and weight loss. Without appropriate management, inflammatory bowel disease can progress and increase the subject's risk of colon cancer, primary sclerosing cholangitis, and blood clots. Complications of Crohn's disease include, e.g., bowel obstruction, malnutrition, ulcers, fistulas, and anal fissures. Complications of ulcerative colitis include, e.g., toxic megacolon, colon perforation, and severe dehydration. While various approaches for inflammatory bowel disease management have been developed, these approaches are typically systemic and rely on inflammation suppression rather than the treatment of the underlying condition.

There is a need for small molecule-based therapeutic approaches to the treatment of inflammatory bowel disease.

### SUMMARY

In some aspects, the disclosure provides compounds that are compound A, or compound G or a pharmaceutically acceptable salt thereof:

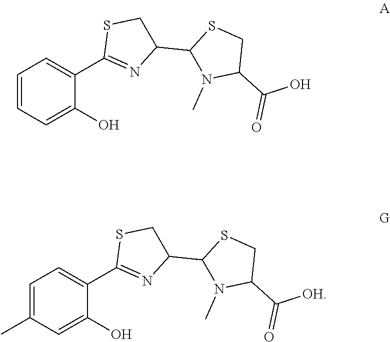 [SMILES: CN1C(C(=O)O)CSC1C1CSC(c2ccccc2O)=N1 ; Cc1ccc(C2=NC(C3SCC(C(=O)O)N3C)CS2)c(O)c1]

In other aspects, the disclosure provides compounds that are compound A, compound B, compound C, compound D, compound E, compound F, or compound G or a pharmaceutically acceptable salt thereof:

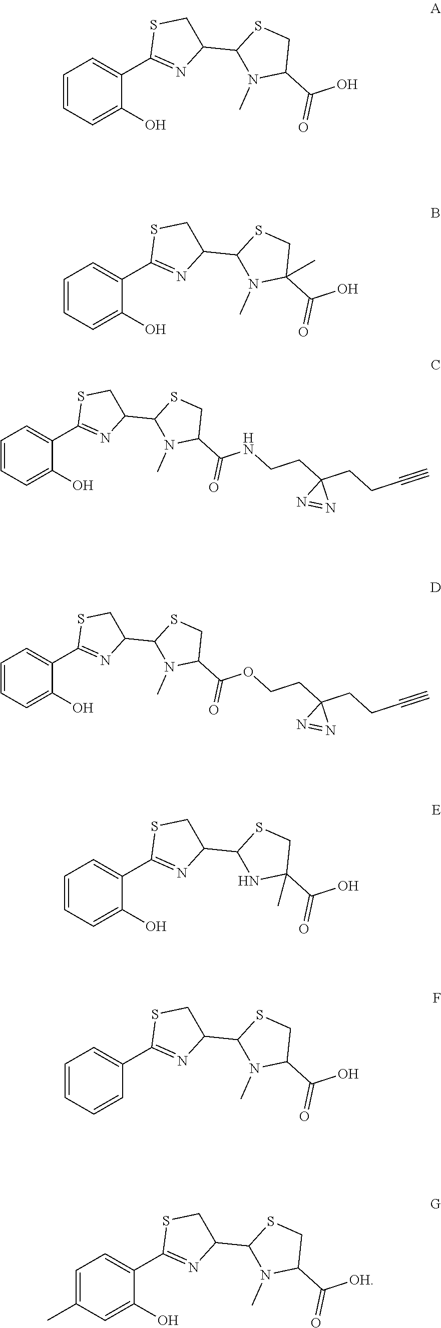

In yet other aspects, the disclosure provides compounds that are compound H, compound J, compound K, compound L, compound M, compound N, compound O, compound P, or a pharmaceutically acceptable salt thereof.

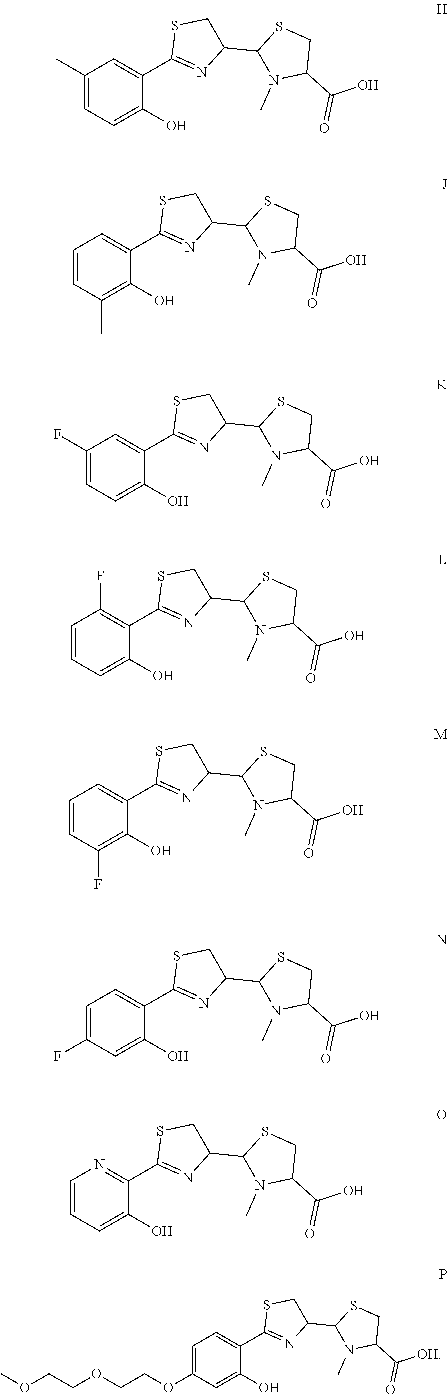

In further aspects, the disclosure provides pharmaceutical compositions comprising one or more compounds described herein and a pharmaceutically acceptable excipient.

In other aspects, the disclosure provides dosage units comprising one or more compound described herein the pharmaceutical composition described herein.

In other embodiments, the disclosure provides methods of treating inflammatory bowel disease in a subject in need thereof, comprising administering one or more pharmaceutical compositions or dosage units described herein to the subject.

In further embodiments, the disclosure provides methods of modulating an inflammatory bowel disease marker in a subject in need thereof, comprising administering a therapeutically effective amount of one or more pharmaceutical composition or dosage unit described herein to the subject.

Other aspects and embodiments of the invention will be readily apparent from the following detailed description of the invention.

### BRIEF DESCRIPTION OF THE DRAWINGS

FIG. 1 is a bar graph showing the dectin-1b activity for compound A-3. In this figure, Syki is the Syk inhibitor IV (BAY 61-3606) and OD650 nM values were adjusted to cell numbers.

FIG. 2 are Luminex assay bar graphs for compound A-3.

FIG. 3 is a graph showing the dose response for a combination of compounds A-2 and A-3.

FIG. 4 is a bar graph showing the dectin-1b activity for compounds A1, B1, and E1.

FIG. 5 are Luminex assay bar graphs for compounds A1 and B1 at concentrations of 0.125, 0.25, 0.5, and 1 μM and show suppression of zymosan-induced IL-12p40 expression in monocyte-derived dendritic cells.

FIGS. 6A, 6B, and 6C are Luminex assay bar graphs for compounds A1, A2, and A3 at concentrations of 15.6 nM, 62.5 nM, 250 nM, and 1 μM for 3 donors and show inhibition of Zymosan-induced production of the pro-inflammatory cytokine IL-12p40 in monocyte-derived dendritic cells.

FIGS. 7A, 7B, and 7C are IC₅₀ curves for compounds A1, A2, and A3, respectively, and show inhibition of Zymosan-induced production of the pro-inflammatory cytokine IL-12p40 in monocyte-derived dendritic cells.

FIGS. 8A and 8B are bar graphs for compounds I-100 and A6, respectively, and show inhibition of LPS-induced IRF reporter activity.

FIGS. 9A-9H are bar graphs for compounds I-100 and A6 and show inhibition of pro-inflammatory cytokines in LPS-stimulated monocyte-derived dendritic cells.

FIGS. 10A-10H are bar graphs for compounds I-100 and A6 and show inhibition of pro-inflammatory cytokines in R848-stimulated monocyte-derived dendritic cell.

FIGS. 11A and 11B are Luminex assay bar graphs for compounds A3 and C3 at concentrations of 0.5 μM.

FIG. 12 is a bar graph of the inhibition of BGP-induced Dectin-1b signaling in HEK cells for compounds A6, C2, and C3 at concentrations of 1, 10, and 30 μM.

### DETAILED DESCRIPTION OF ILLUSTRATIVE EMBODIMENTS

In the disclosure, the singular forms “a”, “an” and “the” include the plural reference, and reference to a particular numerical value includes at least that particular value, unless the context indicates otherwise. For example, reference to “a material” is a reference to at least one of such materials and equivalents thereof known to those skilled in the art, and so forth.

When a value is expressed as an approximation by use of the descriptor “about” or “approximately” it will be understood that the particular value forms another embodiment. In general, use of the term “about” or “approximately” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “approximately.” In other cases, the gradations used in a series of values may be used to determine the intended range available to the term “about” or “approximately” for each value. Where present, all ranges are inclusive and combinable. That is, references to values stated in ranges include every value within that range.

When a list is presented, unless stated otherwise, it is to be understood that each individual element and every combination is to be interpreted as separate embodiments. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude an optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

The compounds disclosed herein may exist in one or more particular chiral forms, such as R- and S-forms. In some embodiments, the compounds are enantiomers. In other embodiments, the compounds are diastereomers. In certain embodiments, one, two, three, or four carbon atoms is in the (R)-configuration. In other embodiments, one, two, three, or four carbon atoms is in the (S)-configuration. The compounds described herein may also be enriched for an isomer of the compound having a selected stereochemistry, e.g., R or S. In some embodiments, the compounds have an enantiomeric excess (ee %) of at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99%. In other embodiments, the compounds are racemic, i.e., an ee of 0%. In further embodiments, the compounds are a single enantiomer, i.e., an ee of 100%.

The terms “subject” and “patient” are used interchangeably and include, without limitation, mammals. In some embodiments, the patient or subject is a human. In other embodiments, the patient or subject is a veterinary or farm animal, a domestic animal or pet, or animal used for clinical research.

“Treating” or variations thereof refers ameliorating or reducing the development of a disease or disorder, i.e., delaying the onset of the disease. In certain embodiments, “treating” refers to ameliorating or reducing at least one physical parameter of the disease or disorder. In other embodiments“treating” is directed to improving the disease or disorder. In further embodiments, “treating” is directed to the cause of the disease or disorder. In yet other embodiments, “treating” is directed to relieving symptoms of the disease or disorder. In still further embodiments, “treating” is directed to treating the disease or disorder as a supplement another therapy.

The term “halo” represents chlorine, fluorine, bromine, or iodine.

The term “alkyl,” as used herein refers to a straight- or branched-chain alkyl groups. In some embodiments, the alkyl group has from 1 to 20 carbons atoms, i.e., C₁₋₂₀alkyl. In other embodiments, the alkyl group has from 1 to 6 carbon atoms, i.e., C₁₋₆alkyl.

Examples of alkyls include methyl (C₁alkyl) ethyl (C₂alkyl), n-propyl (C₃alkyl), isopropyl (C₃alkyl), among others. In some embodiments, the alkyl is methyl. In other embodiments, the alkyl is ethyl. In further embodiments, the alkyl is n-propyl or isopropyl. In still further embodiments, the alkyl is n-butyl, i-butyl, s-butyl, or t-butyl. In other embodiments, the alkyl is pentyl. In further embodiments, the alkyl is hexyl. The alkyl is optionally substituted with one, two, or three substituents selected from halo, OH, OC₁₋₆alkyl, CN, NH₂, NH(C₁₋₆alkyl), NH(C₁₋₆alkyl)₂, C₃₋₈cycloalkyl, heterocyclyl, or aryl.

The term “alkoxy,” as used herein refers to O—C₁₋₆alkyl, wherein alkyl is defined herein and the point of attachment is through the oxygen atom of the alkoxy. In some embodiments, the alkoxy group has from 1 to 20 carbons atoms, i.e., C₁₋₂₀alkoxy. In other embodiments, the alkoxy has from 1 to 6 carbon atoms, i.e., C₁₋₆alkoxy. Examples of alkoxys include methoxy (C₁alkoxy) ethoxy (C₂alkoxy), n-propoxy (C₃alkoxy), isopropoxy (C₃alkoxy), among others. In some embodiments, the alkoxy is methoxy. In other embodiments, the alkoxy is ethoxy. In further embodiments, the alkoxy is n-propoxy or isopropoxy. In still further embodiments, the alkoxy is n-butoxy, i-butoxy, s-butoxy, or t-butoxy. In other embodiments, the alkoxy is pentoxy. In further embodiments, the alkoxy is hexoxy. The alkoxy is optionally substituted with one, two, or three substituents selected from halo, OH, OC₁₋₆alkyl, CN, NH₂, NH(C₁₋₆alkyl), NH(C₁₋₆alkyl)₂, C₃₋₈cycloalkyl, heterocyclyl, or aryl.

The term “alkylene” or “-alk-” as used herein refer to a straight- or branched-chain alkyl groups that links two groups, i.e., an internal group. In some embodiments, the alkylene group has from 1 to 6 carbons atoms, i.e., C₁₋₆alkyl. Examples of alkylene include C₁alkyl (—CH₂—), C₂alkyl (—CH₂CH₂— or CH(CH₃)—), C₃alkyl, C₄alkyl, C₅alkyl, or C₆alkyl. The alkylene is optionally substituted with one, two, or three substituents selected from halo, OH, OC₁₋₆alkyl, CN, NH₂, NH(C₁₋₆alkyl), NH(C₁₋₆alkyl)₂, C₃₋₈cycloalkyl, heterocyclyl, or aryl.

As used herein, “alkenyl” refers to a straight or branched chain hydrocarbon radical having 2 to 20 carbon atoms that has at least one point of unsaturation that is a double bond. In other embodiments, the alkenyl group has from 2 to 6 carbon atoms, i.e., C₂₋₆alkenyl. Examples of alkenyls include ethenyl (C₂alkenyl), propenyl (C₃alkenyl), among others. In some embodiments, the alkenyl is —CH═CH₂. In some embodiments, the alkenyl is a straight chain hydrocarbon. In other embodiments, the alkenyl is a branched chain hydrocarbon. In some embodiments, the alkenyl has one double bond. In other embodiments, the alkenyl has two or more double bonds. The alkenyl group may be substituted with one or more group as described herein. For example, an alkenyl is optionally substituted with one, two, or three substituents selected from halo, OH, OC₁₋₆alkyl, CN, NH₂, NH(C₁₋₆alkyl), NH(C₁₋₆alkyl)₂, C₃₋₈cycloalkyl, heterocyclyl, or aryl.

As used herein, “alkynyl” refers to a straight or branched chain hydrocarbon radical having 2 to 20 carbon atoms that has at least one point of unsaturation that is a triple bond. In other embodiments, the alkynyl group has from 2 to 6 carbon atoms, i.e., C₂₋₆alkynyl. Examples of alkynyls include ethynyl (C₂alkynyl), propynyl (C₃alkynyl), among others. In some embodiments, the alkynyl is —C≡C. In some embodiments, the alkynyl is a straight chain hydrocarbon. In other embodiments, the alkynyl is a branched chain hydrocarbon. In some embodiments, the alkynyl has one triple bond. In other embodiments, the alkynyl has two or more triple bonds. The alkynyl may be substituted with one or more group as described herein. For example, an alkenyl is optionally substituted with one, two, or three substituents selected from halo, OH, OC₁₋₆alkyl, CN, NH₂, NH(C₁₋₆alkyl), NH(C₁₋₆alkyl)₂, C₃₋₈cycloalkyl, heterocyclyl, or aryl.

“Cycloalkyl” as used herein refers to a monocyclic, non-aromatic hydrocarbon group. In some embodiments, the cycloalkyl group has from 3 to 8 carbon atoms (“C₃₋₈”). In some embodiments, the cycloalkyl has from 3 to 6 carbon atoms. Examples of cycloalkyl groups include, e.g., cyclopropyl (C₃), cyclobutyl (C₄), cyclopentyl (C₅), 1-methylcyclopropyl (C₄), or 2-methylcyclopentyl (C₄), among others. A cycloalkyl is optionally substituted with one, two, or three substituents selected from halo, OH, OC₁₋₆alkyl, CN, NH₂, NH(C₁₋₆alkyl), NH(C₁₋₆alkyl)₂, C₃₋₈cycloalkyl, heterocyclyl, or aryl.

The term “heterocyclyl” refers to a stable 3- to 14-membered non-aromatic ring radical that comprises two to twelve carbon atoms and from 1 to 6 heteroatoms selected from nitrogen, oxygen and sulfur. In some embodiments, the heterocyclyl contains 3 ring atoms, 4 ring atoms, etc., up to and including 14 ring atoms. The heterocyclyl is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is unsaturated, partially saturated, or fully saturated. The heterocyclyl may be attached to the rest of the molecule through any atom of the ring(s). “Heterocyclyl” also includes bicyclic ring systems wherein one non-aromatic ring contains at least 2 carbon atoms in addition to 1-3 heteroatoms independently selected from oxygen, sulfur, and nitrogen, as well as combinations comprising at least one of the foregoing heteroatoms; and the other ring optionally contains 1-3 heteroatoms independently selected from oxygen, sulfur, and nitrogen and is not aromatic. In some embodiments, the heterocyclyl is indolyl. Examples of heterocyclyl include, but are not limited to, azepanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl, azepinyl, acridinyl, benzimidazolyl, benzindolyl, 1,3-benzodioxolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a, 7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). A heterocyclyl is optionally substituted with one, two, or three substituents selected from halo, OH, OC₁₋₆alkyl, CN, NH₂, NH(C₁₋₆alkyl), NH(C₁₋₆alkyl)₂, C₃₋₈cycloalkyl, heterocyclyl, or aryl.

The term “aryl” refers to carbocyclic aromatic groups having from 6 to 10 carbon atoms (“C₆₋₁₀”) such as phenyl, naphthyl, and the like. An aryl is optionally substituted with one, two, or three substituents selected from halo, OH, OC₁₋₆alkyl, CN, NH₂, NH(C₁₋₆alkyl), NH(C₁₋₆alkyl)₂, C₃₋₈cycloalkyl, heterocyclyl, or aryl.

### The Compounds

The disclosure provides compounds of Formula I:

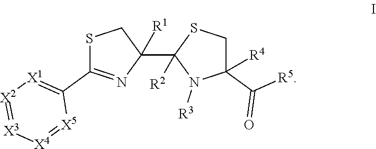

According to the disclosure, R¹, R², R³, and R⁴ are, independently, H or optionally substituted C₁₋₆alkyl. In some embodiments, R¹, R², R³, and R⁴ are, independently, H. In other embodiments, R¹, R², R³, and R⁴ are, independently, optionally substituted C₁₋₆ alkyl. In further embodiments, R¹, R², R³, and R⁴ are, independently, methyl, ethyl, propyl, butyl, pentyl, or hexyl. In yet other embodiments, R¹, R², R³, and R⁴ are, independently, methyl. In still further embodiments R¹ is methyl. In other embodiments, R² is methyl. In further embodiments, R³ is methyl. In yet other embodiments, R⁴ is methyl. In still further embodiments, R¹ is H. In other embodiments, R² is H. In further embodiments, R³ is H. In still other embodiments, R⁴ is H.

According to the disclosure, R⁵ is OH, optionally substituted C₁₋₆alkoxy, —N(R^{A})(optionally substituted C₁₋₆alkyl), or —X(R^{A})—R¹³—CH(X⁶)(X⁷)R¹¹R¹². In some embodiments, R⁵ is OH. In other embodiments, R⁵ is optionally substituted C₁₋₆alkoxy. In further embodiments, R⁵ is optionally substituted C₅₋₆alkyl. In yet other embodiments, R⁵ is optionally substituted C₅alkyl. In still further embodiments, R⁵ is optionally substituted C₆alkyl. In other embodiments, R⁵ is —N(R^{A})(optionally substituted C₁₋₆alkyl). In further embodiments, R⁵ is —N(R^{A})(optionally substituted C₅₋₆alkyl). In yet other embodiments, R⁵ is N(R^{A})(optionally substituted C5alkyl). In still further embodiments, R⁵ is —N(R^{A})(optionally substituted C6alkyl). In other embodiments, R⁵ is —X(R^{A})—R13-CH(X⁶)(X⁷)R¹¹R¹². In further embodiments, R⁵ is

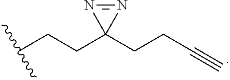 [SMILES: *CCC1(CCC#C)N=N1]

According to the disclosure, R¹⁵ is —C₁₋₆alk-. In some embodiments, R¹⁵ is —C₁alk-. In other embodiments, R¹⁵ is —C₂alk-. In further embodiments, R¹⁵ is —C₃alk-. In yet other embodiments, R¹⁵ is —C₄alk-. In still further embodiments, R¹⁵ is —C₅alk-. In other embodiments, R¹⁵ is —C₆alk-. In further embodiments, R¹⁵ is —CH₂—, —CH₂CH₂—, —CH₂CH₂CH₂—, or —CH₂CH₂CH₂CH₂—.

According to the disclosure, R^{A} is H or optionally substituted C₁₋₆alkyl. In some embodiments, R^{A} is H. In other embodiments, R^{A} is optionally substituted C₁₋₆alkyl such as methyl, ethyl, propyl, butyl, pentyl, or hexyl. In further embodiments, R^{A} is methyl.

According to the disclosure, X is O or NH. In some embodiments, X is O. In other embodiments, X is NH.

According to the disclosure, X¹ is CR⁶ or N. In some embodiments, X¹ is CR⁶. In other embodiments, X¹ is N.

According to the disclosure, X² is CR⁷ or N. In some embodiments, X² is CR⁷. In other embodiments, X² is N.

According to the disclosure, X³ is CR⁸ or N. In some embodiments, X³ is CR⁸. In other embodiments, X³ is N.

According to the disclosure, X⁴ is CR⁹ or N. In some embodiments, X⁴ is CR⁹. In other embodiments, X⁴ is N.

According to the disclosure, X⁵ is CR¹⁰ or N. In some embodiments, X⁵ is CR¹⁰. In other embodiments, X⁵ is N.

In certain embodiments, only one of X¹—X⁵ is N.

According to the disclosure, X⁶ and X⁷ are, independently, H or optionally substituted C₁₋₆alkyl, or both are N and join to form a diazirinyl ring. In some embodiments, X⁶ and X⁷ are, independently, H. In other embodiments, X⁶ is H. In further embodiments, X⁷ is H. In yet other embodiments, X⁶ and X⁷ are, independently, optionally substituted C₁₋₆alkyl. In still further embodiments, X⁶ and X⁷ are, independently, such as methyl, ethyl, propyl, butyl, pentyl, or hexyl. In other embodiments, X⁶ and X⁷ are, independently, methyl. In further embodiments, X⁶ and X⁷ are N and join to form an optionally substituted diazirinyl ring.

According to the disclosure, R⁶, R⁷, R⁸, R⁹, and R¹⁰ are, independently, H, OH, halo, optionally substituted C₁₋₆alkyl, optionally substituted C₁₋₆alkoxy, or —(OC₁₋₆alk)ₙ-C₁₋₆alkyl. In some embodiments, R⁶, R⁷, R⁸, R⁹, and R¹⁰ are, independently, H. In other embodiments, R⁶, R⁷, R⁸, R⁹, and R¹⁰ are, independently, OH. In further embodiments, R⁶, R⁷, R⁸, R⁹, and R¹⁰ are, independently, halo such as F, Cl, or Br. In still other embodiments, R⁶, R⁷, R⁸, R⁹, and R¹⁰ are, independently, optionally substituted C₁₋₆alkyl. In yet further embodiments, R⁶, R⁷, R⁸, R⁹, and R¹⁰ are, independently, methyl, ethyl, propyl, butyl, pentyl, or hexyl. In other embodiments, one of R⁶, R⁷, R⁸, R⁹, and R¹⁰ are, independently, methyl. In further embodiments, R⁶, R⁷, R⁸, R⁹, and R¹⁰ are, independently, optionally substituted C₁₋₆alkoxy. In yet other embodiments, R⁶, R⁷, R⁸, R⁹, and R¹⁰ are, independently, methoxy, ethoxy, propoxy, butoxy, pentoxy, or hexoxy. In still further embodiments, one of R⁶, R⁷, R⁸, R⁹, and R¹⁰ are, independently, methoxy. In other embodiments, R⁶, R⁷, R⁸, R⁹, and R¹⁰ are, independently, —(OC₁₋₆alk)ₙ-C₁₋₆alkyl. In further embodiments, one of R⁶, R⁷, R⁸, R⁹, and R¹⁰ are, independently, OCH₂CH₂OCH₂CH₂OCH₃.

According to the disclosure, R¹¹ and R¹² are, independently, optionally substituted C₁₋₆alkyl, optionally substituted C₂₋₆alkenyl, optionally substituted C₂₋₆alkynyl, optionally substituted C₁₋₆alkoxy, OH, NH₂, or N(R¹³)(R¹⁴). In some embodiments, R¹¹ and R¹² are, independently, optionally substituted C₁₋₆alkyl. In other embodiments, R¹¹ and R¹² are, independently methyl, ethyl, propyl, butyl, pentyl, or hexyl. In further embodiments, R¹¹ and R¹² are, independently, optionally substituted C₂₋₆alkenyl. In yet other embodiments, R¹¹ and R¹² are, independently, ethenyl, propenyl, butenyl, pentenyl, or hexenyl. In still further embodiments, R¹¹ and R¹² are, independently, optionally substituted C₂₋₆alkynyl. In other embodiments, R¹¹ and R¹² are, independently, ethynyl, propynyl, butynyl, pentynyl, or hexynyl. In further embodiments, R¹¹ and R¹² are, independently, optionally substituted C₁₋₆ alkoxy. In still other embodiments, R¹¹ and R¹² are, independently, methoxy, ethoxy, propoxy, butoxy, pentoxy, or hexoxy. In yet further embodiments, R¹¹ and R¹² are, independently, OH. In other embodiments, R¹¹ and R¹² are, independently, NH₂. In further embodiments, R¹¹ and R¹² are, independently, N(R¹³)(R¹⁴). In still further embodiments, R¹¹ and R¹² are, independently, NHCH₃. In other embodiments, R¹¹ and R¹² are, independently, N(CH₃)₂.

According to the disclosure, R¹³ and R¹⁴ are, independently, H or C₁₋₆alkyl. In some embodiments, R¹³ and R¹⁴ are, independently, H. In other embodiments, R¹³ and R¹⁴ are, independently, C₁₋₆alkyl. In further embodiments, R¹³ and R¹⁴ are, independently, methyl, ethyl, propyl, butyl, pentyl, or hexyl.

According to the disclosure, n is 2-8. In some embodiments, n is 2. In other embodiments, n is 3. In further embodiments, n is 4. In yet other embodiments, n is 5. In still further embodiments, n is 6. In other embodiments, n is 7. In further embodiments, n is 8.

The disclosure also provides compound A, compound B, compound C, compound D, compound E, compound F, compound G, compound H, compound J, compound K, compound L, compound M, compound N, compound O, compound P, or compound Q:

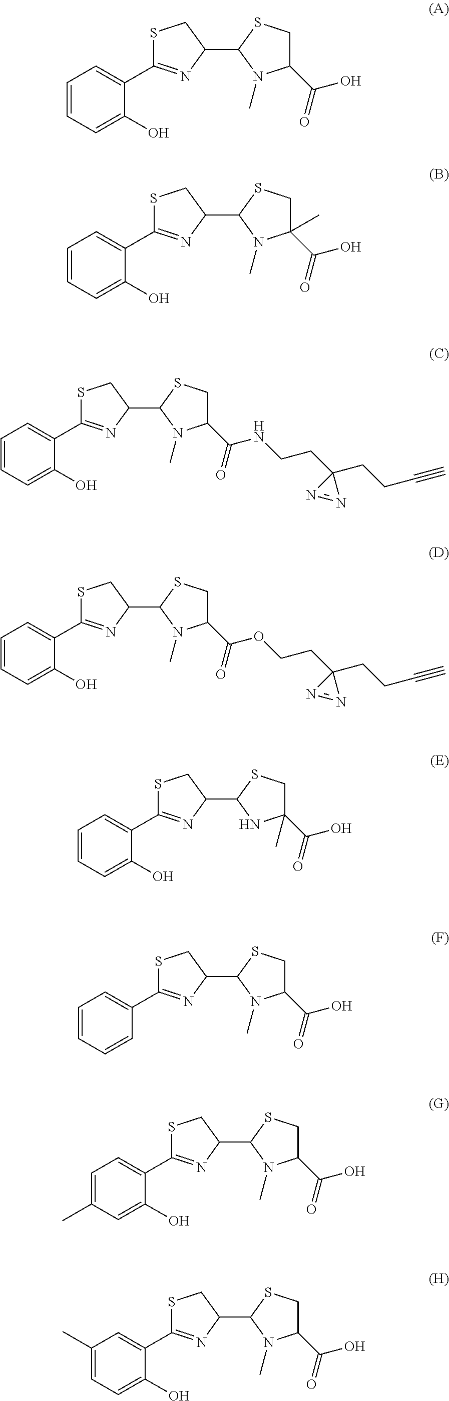

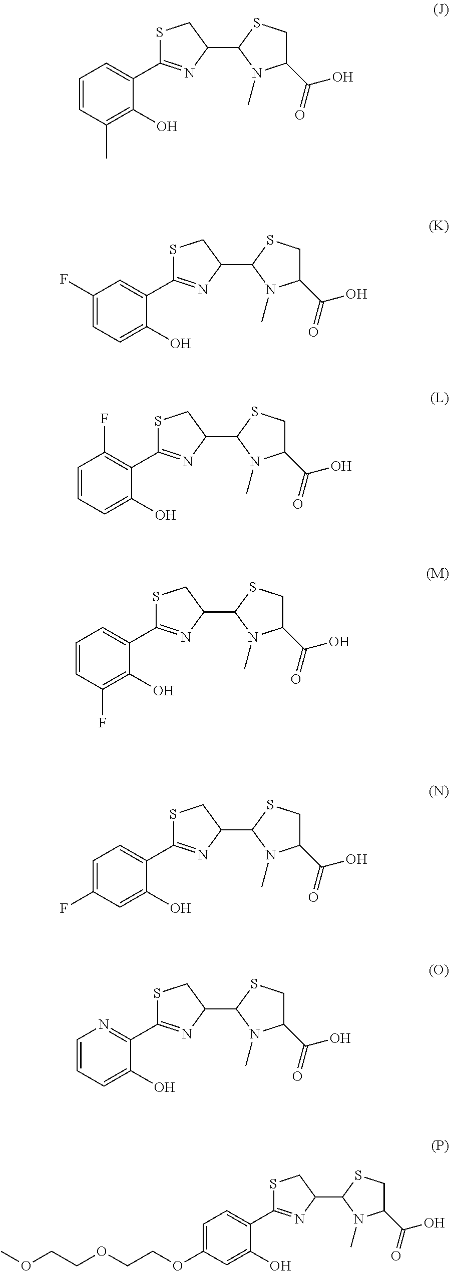

or a pharmaceutically acceptable salt thereof.

In some embodiments, the disclosure provides compound A, or a pharmaceutically acceptable salt thereof:

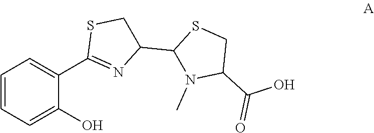 [SMILES: CN1C(C(=O)O)CSC1C1CSC(c2ccccc2O)=N1]

or a pharmaceutically acceptable salt thereof. In certain aspects, compound A is

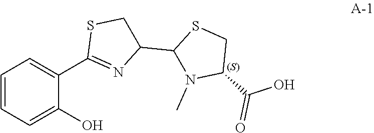

or a pharmaceutically acceptable salt thereof. In other aspects, compound A is

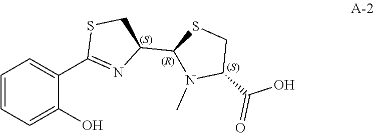

or a pharmaceutically acceptable salt thereof. In further aspects, compound A is

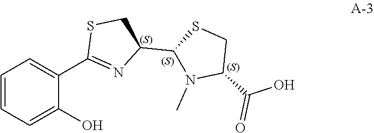

or a pharmaceutically acceptable salt thereof. In yet other aspects, compound A is

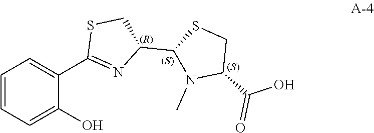

or a pharmaceutically acceptable salt thereof. In still further aspects, compound A is

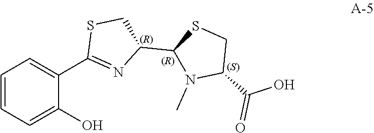

or a pharmaceutically acceptable salt thereof.

In other embodiments, the disclosure provides compound B, or a pharmaceutically acceptable salt thereof:

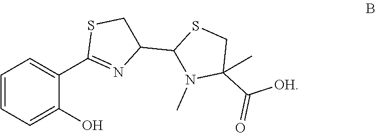 [SMILES: CN1C(C2CSC(c3ccccc3O)=N2)SCC1(C)C(=O)O]

In certain aspects, compound B is

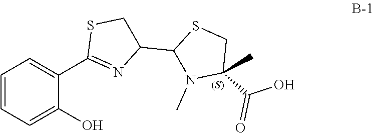

or a pharmaceutically acceptable salt thereof. In other aspects, compound B is

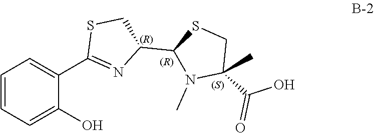

or a pharmaceutically acceptable salt thereof. In further aspects, compound B is

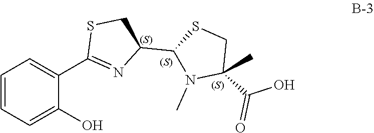

or a pharmaceutically acceptable salt thereof. In yet other aspects, compound B is

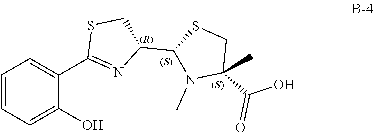

or a pharmaceutically acceptable salt thereof. In still further aspects, compound B is

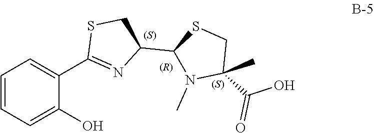

or a pharmaceutically acceptable salt thereof.

In further embodiments, the disclosure provides compound C, or a pharmaceutically acceptable salt thereof:

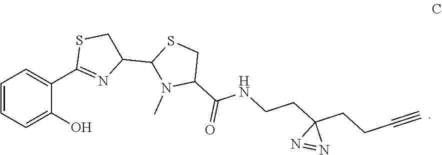 [SMILES: C#CCCC1(CCNC(=O)C2CSC(C3CSC(c4ccccc4O)=N3)N2C)N=N1]

In certain aspects, the compound C

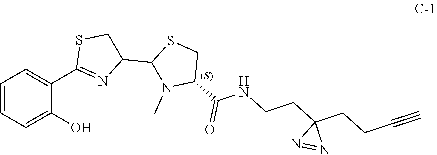

or a pharmaceutically acceptable salt thereof. In other aspects, compound C is

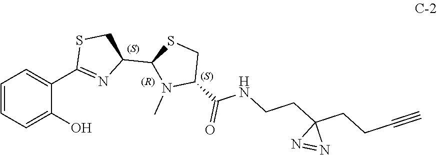

or a pharmaceutically acceptable salt thereof. In further aspects, compound C is

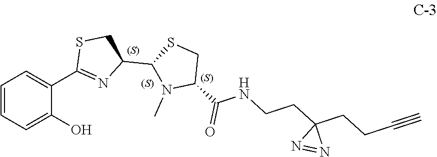

or a pharmaceutically acceptable salt thereof. In yet other aspects, compound C is

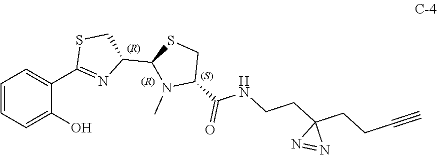

or a pharmaceutically acceptable salt thereof. In still further aspects, compound C is

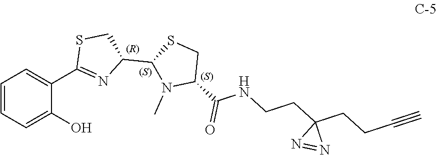

or a pharmaceutically acceptable salt thereof. In other aspects, compound C is

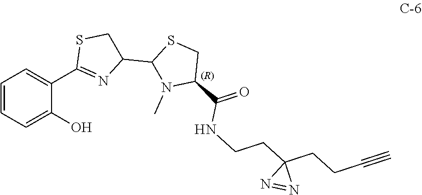

or a pharmaceutically acceptable salt thereof. In further aspects, compound C is

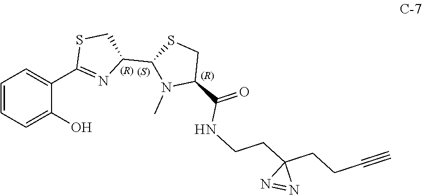

or a pharmaceutically acceptable salt thereof. In other aspects, compound C is

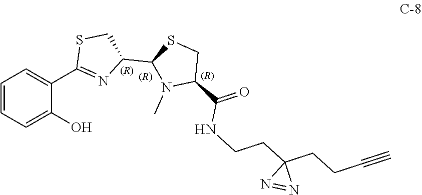

or a pharmaceutically acceptable salt thereof. In yet other aspects, compound C is

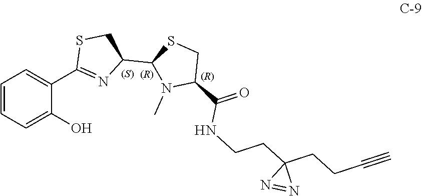

or a pharmaceutically acceptable salt thereof. In yet other aspects, compound C is

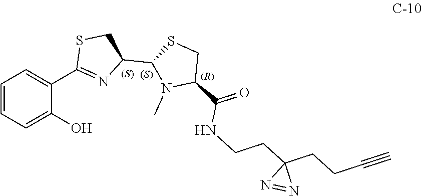

or a pharmaceutically acceptable salt thereof.

In yet other embodiments, the disclosure provides compound D or a pharmaceutically acceptable salt thereof:

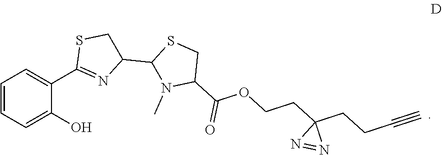 [SMILES: C#CCCC1(CCOC(=O)C2CSC(C3CSC(c4ccccc4O)=N3)N2C)N=N1]

In certain aspects, compound D is

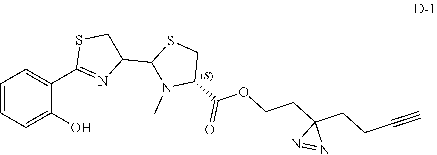

or a pharmaceutically acceptable salt thereof. In other aspects, compound D is

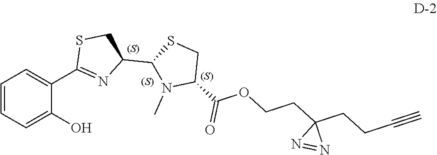

or a pharmaceutically acceptable salt thereof. In further aspects, compound D is

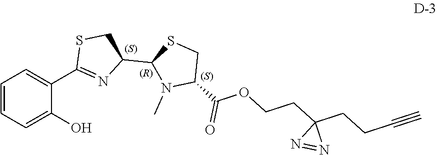

or a pharmaceutically acceptable salt thereof. In yet other aspects, compound D is

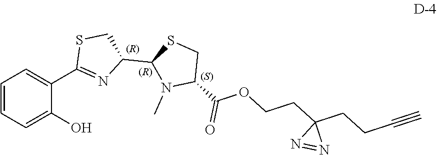

or a pharmaceutically acceptable salt thereof. In still further aspects, compound D is

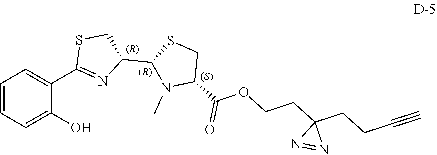

or a pharmaceutically acceptable salt thereof.

In other embodiments, the disclosure provides compound E, or a pharmaceutically acceptable salt thereof:

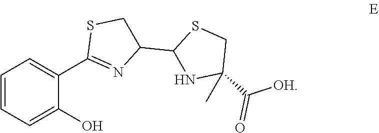 [SMILES: C[C@]1(C(=O)O)CSC(C2CSC(c3ccccc3O)=N2)N1]

In some aspects, compound E is

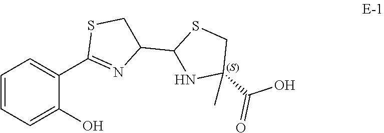

or a pharmaceutically acceptable salt thereof. In further aspects, compound E is

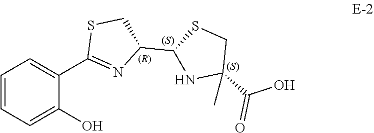

or a pharmaceutically acceptable salt thereof. In other aspects, compound E is

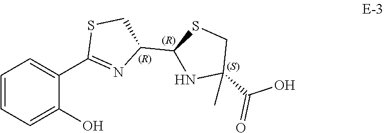

or a pharmaceutically acceptable salt thereof. In still further aspects, compound E is

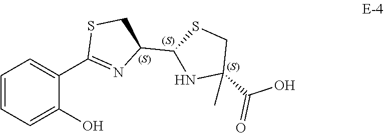

or a pharmaceutically acceptable salt thereof. In yet other aspects, compound E is

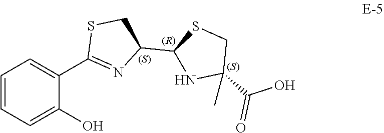

or a pharmaceutically acceptable salt thereof.

In further embodiments, the disclosure provides compound F or a pharmaceutically acceptable salt thereof:

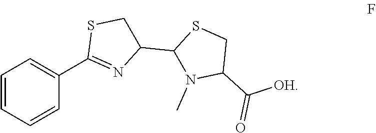 [SMILES: CN1C(C(=O)O)CSC1C1CSC(c2ccccc2)=N1]

In certain aspects, compound F is

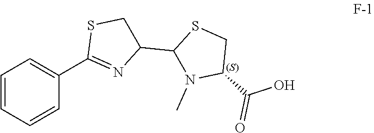

or a pharmaceutically acceptable salt thereof. In other aspects, compound F is

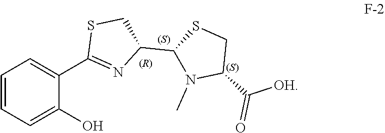

In further aspects, compound F is

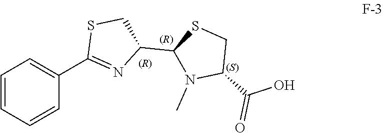

or a pharmaceutically acceptable salt thereof. In yet other aspects, compound F is

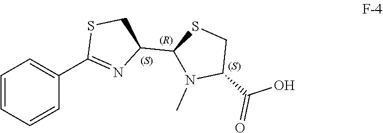

or a pharmaceutically acceptable salt thereof. In still further aspects, compound F is

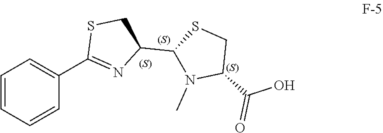

or a pharmaceutically acceptable salt thereof.

In yet other embodiments, the disclosure provides compound G or a pharmaceutically acceptable salt thereof:

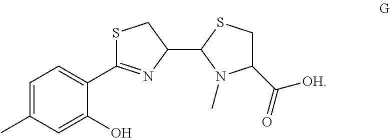 [SMILES: Cc1ccc(C2=NC(C3SCC(C(=O)O)N3C)CS2)c(O)c1]

In certain aspects, compound G is

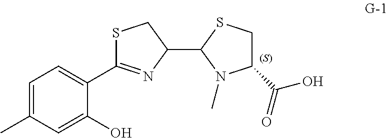

or a pharmaceutically acceptable salt thereof. In other aspects, compound G is

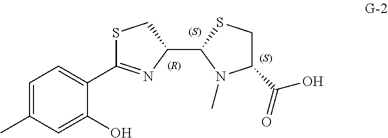

or a pharmaceutically acceptable salt thereof. In further aspects, compound G is

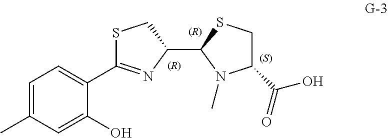

or a pharmaceutically acceptable salt thereof. In yet other aspects, compound G is

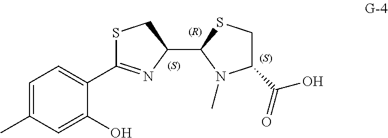

or a pharmaceutically acceptable salt thereof. In still further aspects, compound G is

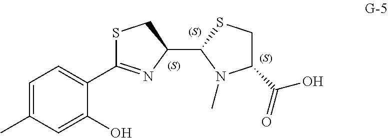

or a pharmaceutically acceptable salt thereof.

In still further embodiments, the disclosure provides a compound that is

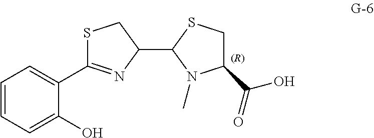

or a pharmaceutically acceptable salt thereof. In certain aspects, the compound is

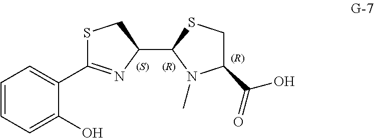

or a pharmaceutically acceptable salt thereof. In other aspects, the compound is

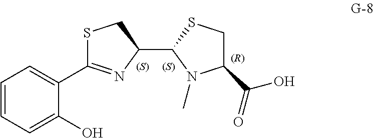

or a pharmaceutically acceptable salt thereof. In further aspects, the compound is

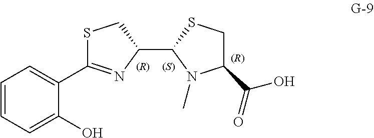

or a pharmaceutically acceptable salt thereof. In yet other aspects, the compound is

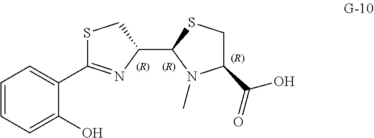

or a pharmaceutically acceptable salt thereof.

In further embodiments, the compound is

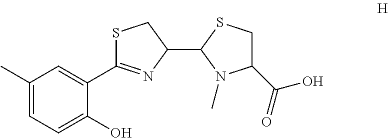 [SMILES: Cc1ccc(O)c(C2=NC(C3SCC(C(=O)O)N3C)CS2)c1]

or a pharmaceutically acceptable salt thereof. In some aspects, compound H is

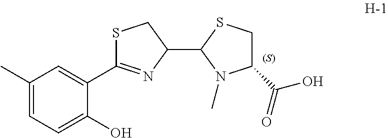

or a pharmaceutically acceptable salt thereof. In certain aspects, compound H is

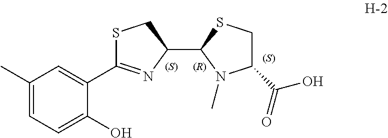

or a pharmaceutically acceptable salt thereof. In other aspects, compound H is

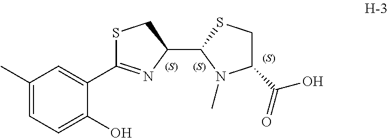

or a pharmaceutically acceptable salt thereof. In further aspects, the compound is

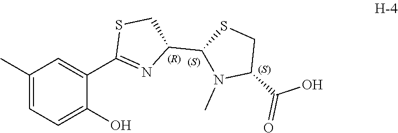

or a pharmaceutically acceptable salt thereof. In yet other aspects, compound H is

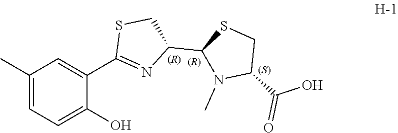

or a pharmaceutically acceptable salt thereof.

In still other embodiments, the compound is

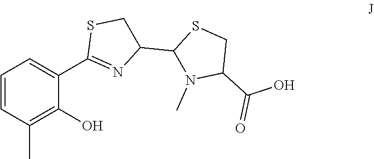 [SMILES: Cc1cccc(C2=NC(C3SCC(C(=O)O)N3C)CS2)c1O]

or a pharmaceutically acceptable salt thereof. In certain aspects, compound J is

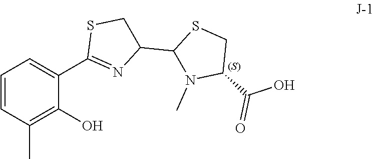

or a pharmaceutically acceptable salt thereof. In other aspects, compound J is

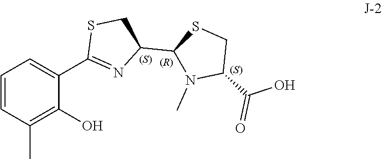

or a pharmaceutically acceptable salt thereof. In further aspects, compound J is

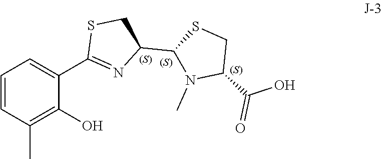

or a pharmaceutically acceptable salt thereof. In other aspects, compound J is

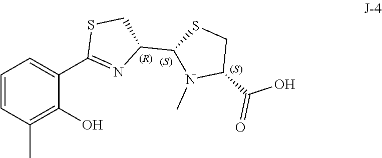

or a pharmaceutically acceptable salt thereof. In yet other aspects, compound J is

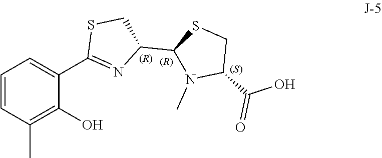

or a pharmaceutically acceptable salt thereof.

In yet further embodiments, the compound is

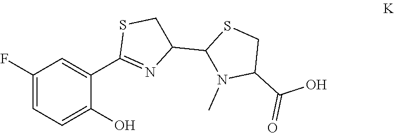 [SMILES: CN1C(C(=O)O)CSC1C1CSC(c2cc(F)ccc2O)=N1]

or a pharmaceutically acceptable salt thereof. In certain aspects, compound K is

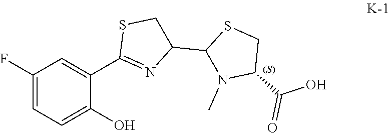

or a pharmaceutically acceptable salt thereof. In some aspects, compound K is

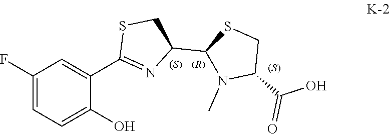

or a pharmaceutically acceptable salt thereof. In other aspects, compound K is

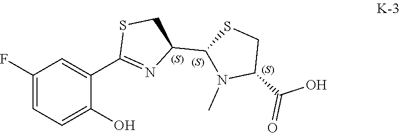

or a pharmaceutically acceptable salt thereof. In further aspects, compound K is

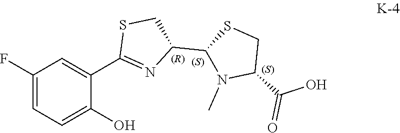

or a pharmaceutically acceptable salt thereof. In yet other aspects, compound K is

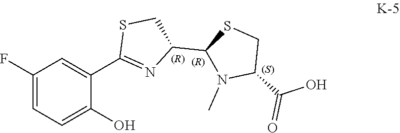

or a pharmaceutically acceptable salt thereof.

In other embodiments, the compound is

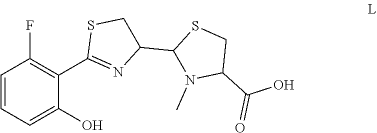 [SMILES: CN1C(C(=O)O)CSC1C1CSC(c2c(O)cccc2F)=N1]

or a pharmaceutically acceptable salt thereof. In certain aspects, compound L is

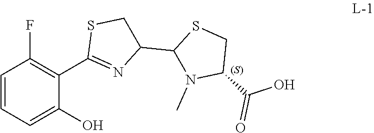

or a pharmaceutically acceptable salt thereof. In further aspects, compound L is

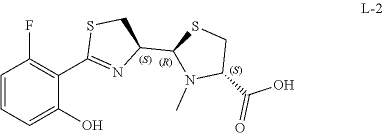

or a pharmaceutically acceptable salt thereof. In yet other aspects, compound L is

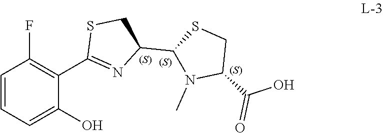

or a pharmaceutically acceptable salt thereof. In still further aspects, compound L is

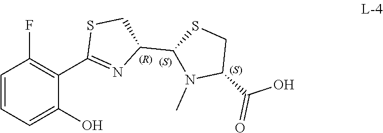

or a pharmaceutically acceptable salt thereof. In other aspects, compound L is

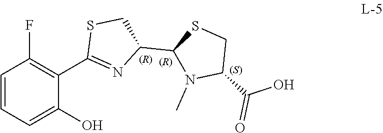

or a pharmaceutically acceptable salt thereof.

In further embodiments, the compound is

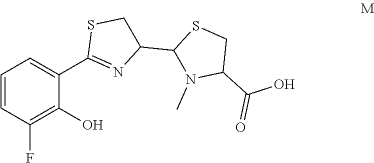 [SMILES: CN1C(C(=O)O)CSC1C1CSC(c2cccc(F)c2O)=N1]

or a pharmaceutically acceptable salt thereof. In certain aspects, compound M is

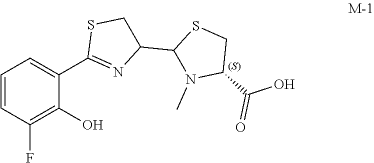

or a pharmaceutically acceptable salt thereof. In other aspects, compound M is

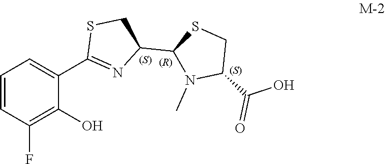

or a pharmaceutically acceptable salt thereof. In further aspects, compound M is

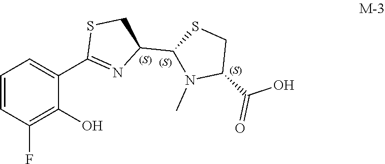

or a pharmaceutically acceptable salt thereof. In yet other aspects, compound M is

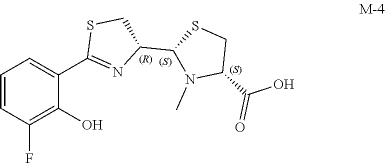

or a pharmaceutically acceptable salt thereof. In still further aspects, compound M is

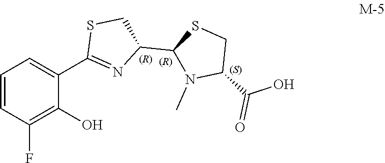

or a pharmaceutically acceptable salt thereof.

In still other embodiments, the compound is

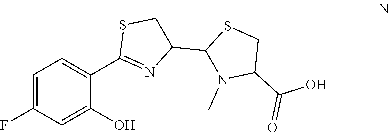 [SMILES: CN1C(C(=O)O)CSC1C1CSC(c2ccc(F)cc2O)=N1]

or a pharmaceutically acceptable salt thereof. In certain aspects, compound N is

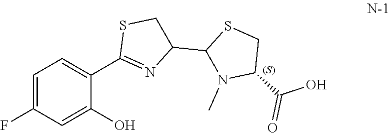

or a pharmaceutically acceptable salt thereof. In other aspects, compound N is

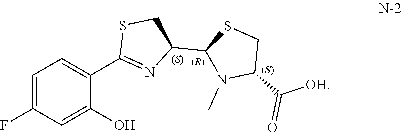

or a pharmaceutically acceptable salt thereof. In further aspects, compound N is

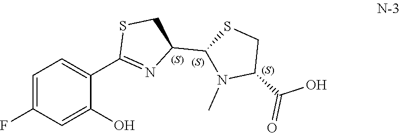

or a pharmaceutically acceptable salt thereof. In yet other aspects, compound N is

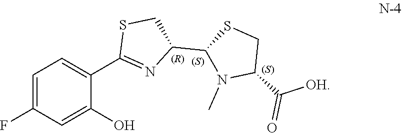

In still further aspects, compound N is

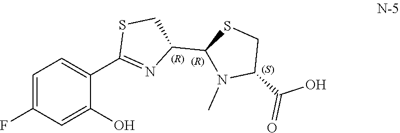

or a pharmaceutically acceptable salt thereof.

In yet further embodiments, the compound is

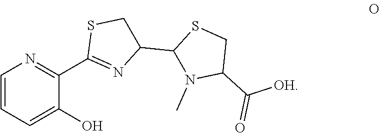 [SMILES: CN1C(C(=O)O)CSC1C1CSC(c2ncccc2O)=N1]

or a pharmaceutically acceptable salt thereof. In certain aspects, compound O is

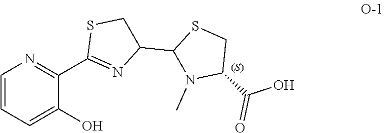

or a pharmaceutically acceptable salt thereof. In other aspects, compound O is

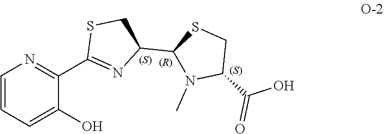

or a pharmaceutically acceptable salt thereof. In further aspects, compound O is

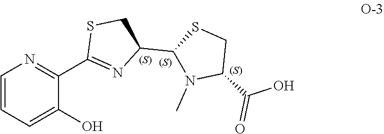

or a pharmaceutically acceptable salt thereof. In yet other aspects, compound O is

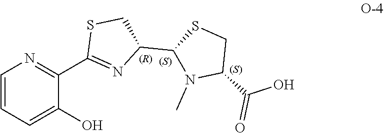

or a pharmaceutically acceptable salt thereof. In still further aspects, compound O is

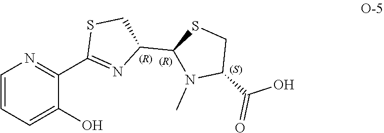

or a pharmaceutically acceptable salt thereof.

In other embodiments, the compound is

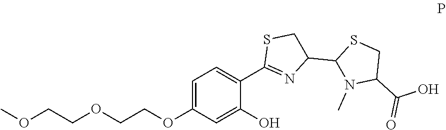 [SMILES: COCCOCCOc1ccc(C2=NC(C3SCC(C(=O)O)N3C)CS2)c(O)c1]

or a pharmaceutically acceptable salt thereof. In certain aspects, the compound is

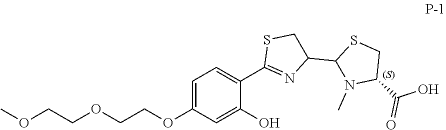

or a pharmaceutically acceptable salt thereof. In other aspects, compound P is

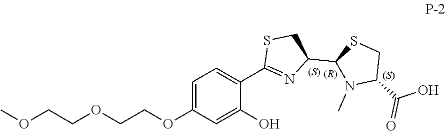

or a pharmaceutically acceptable salt thereof. In further aspects, compound P is

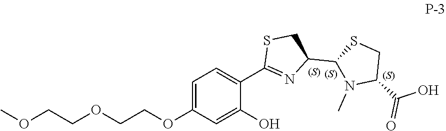

or a pharmaceutically acceptable salt thereof. In still other aspects, compound P is

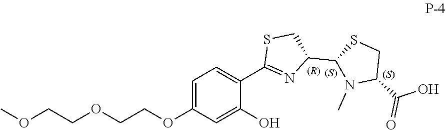

or a pharmaceutically acceptable salt thereof. In yet further aspects, compound P is

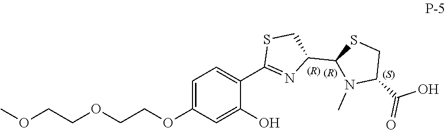

or a pharmaceutically acceptable salt thereof.

The compounds described herein may be synthetically prepared, i.e., not a product found in nature. Alternatively, such compounds are isolated.

The term “pharmaceutically acceptable salt,” as used herein, includes salts that suitable for administration to a subject as defined herein. Pharmaceutically acceptable salts are well known in the art. See, e.g., Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like.

Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

### Pharmaceutical Compositions

Also provided by the disclosure are pharmaceutical compositions comprising one or more compound described herein, i.e., compound A, compound B, compound C, compound D, compound E, compound F, compound G, compound H, compound J, compound K, compound L, compound M, compound N, compound O, compound P, or compound Q. The pharmaceutical compositions may also contain one or more stereoisomers of compound A, compound B, compound C, compound D, compound E, compound F, compound G, compound H, compound J, compound K, compound L, compound M, compound N, compound O, compound P, or compound Q. In some embodiments, the pharmaceutical composition comprises compound A or a pharmaceutically acceptable salt thereof. In certain aspects, the pharmaceutical composition contains

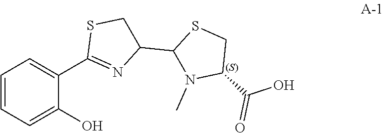

or a pharmaceutically acceptable salt thereof. In other aspects, the pharmaceutical composition contains

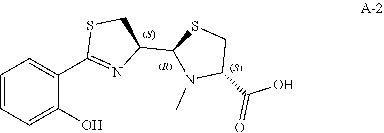

or a pharmaceutically acceptable salt thereof. In further aspects, the pharmaceutical composition contains

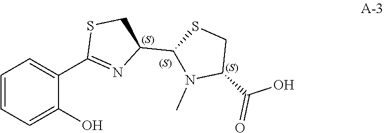

or a pharmaceutically acceptable salt thereof. In yet other aspects, the pharmaceutical composition contains

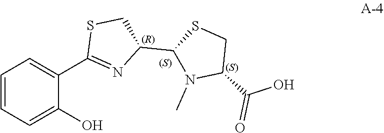

or a pharmaceutically acceptable salt thereof. In still further aspects, the pharmaceutical composition contains

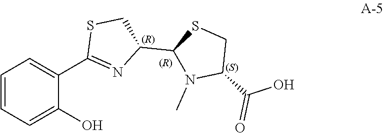

or a pharmaceutically acceptable salt thereof. In some aspects, the pharmaceutical composition one or more of

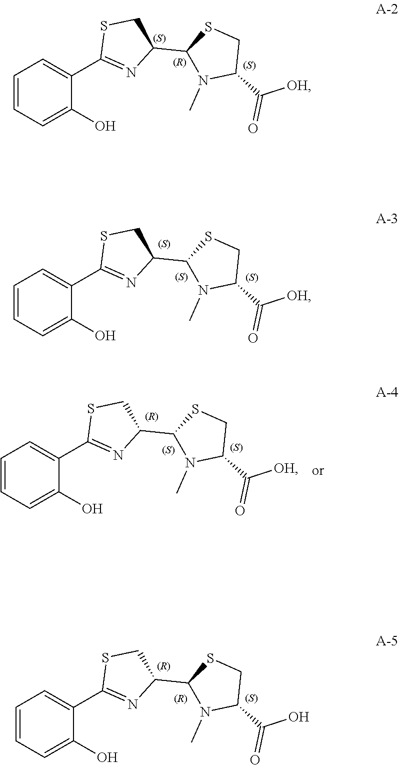

or a pharmaceutically acceptable salt thereof. In other aspects, the pharmaceutical composition contains

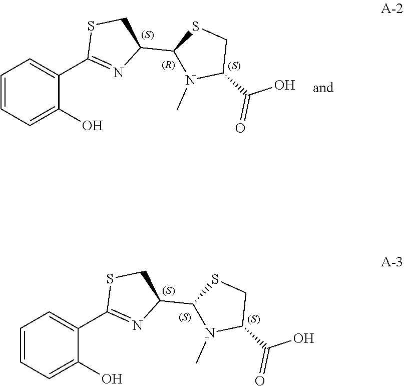

or pharmaceutically acceptable salts thereof. In further aspects, the pharmaceutical composition contains

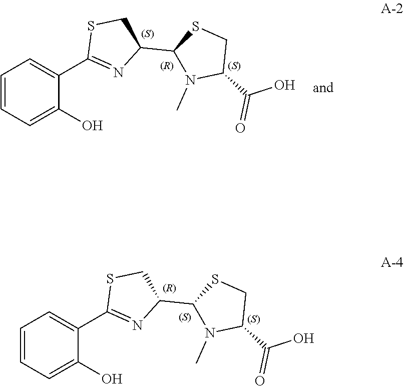

or pharmaceutically acceptable salts thereof. In yet other aspects, the pharmaceutical composition contains

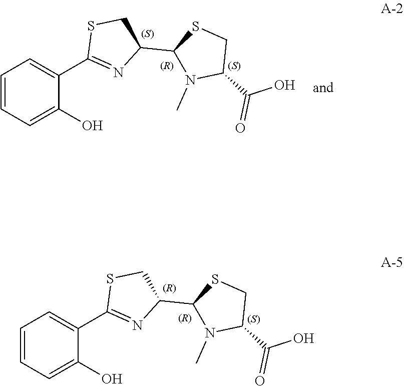

or pharmaceutically acceptable salts thereof. In still further aspects, the pharmaceutical composition contains

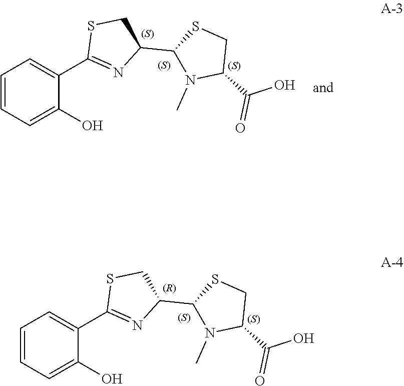

or pharmaceutically acceptable salts thereof. In other aspects, the pharmaceutical composition contains

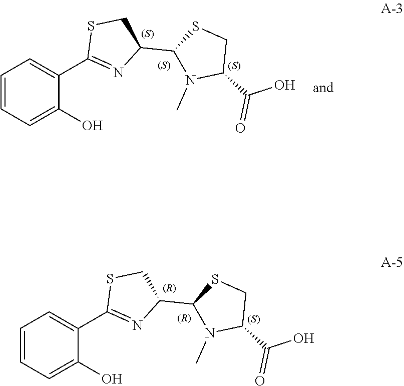

or pharmaceutically acceptable salts thereof. In further aspects, the pharmaceutical composition contains

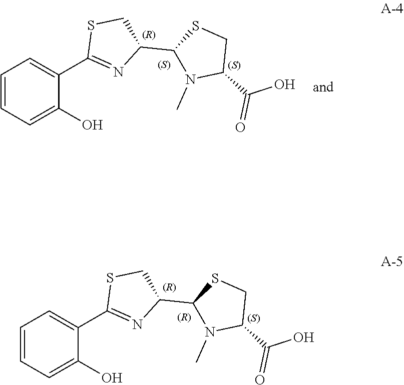

or pharmaceutically acceptable salts thereof.

In other embodiments, the disclosure provides pharmaceutical compositions comprising compound B or a pharmaceutically acceptable salt thereof, and a pharmaceutically acceptable excipient. In certain aspects, the pharmaceutical composition contains

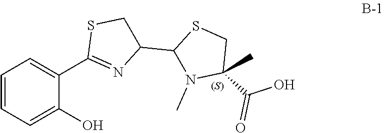

or a pharmaceutically acceptable salt thereof. In other aspects, the pharmaceutical composition contains

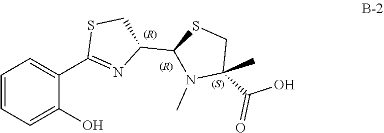

or a pharmaceutically acceptable salt thereof. In further aspects, the pharmaceutical composition contains

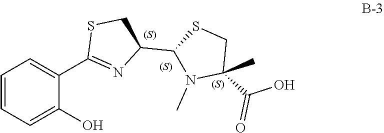

or a pharmaceutically acceptable salt thereof. In yet other aspects, the pharmaceutical composition contains

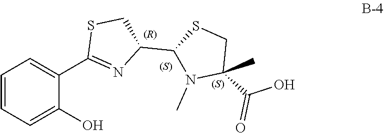

or a pharmaceutically acceptable salt thereof. In still further aspects, the pharmaceutical composition contains

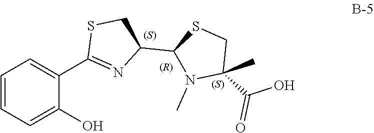

or a pharmaceutically acceptable salt thereof. In some aspects, the pharmaceutical composition contains one or more of

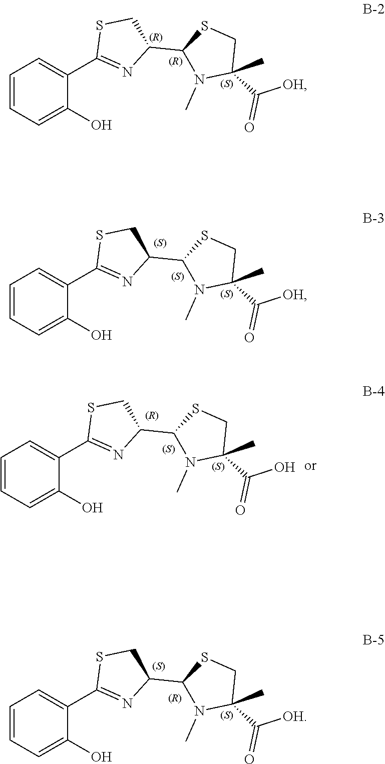

In further aspects, the pharmaceutical composition contains,

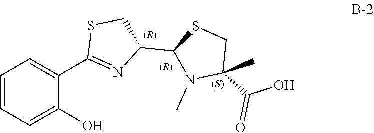

and

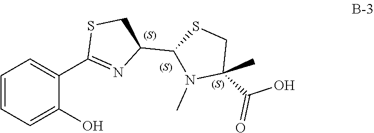

or pharmaceutically acceptable salts thereof. In still further aspects, the pharmaceutical composition contains

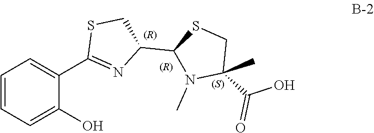

and

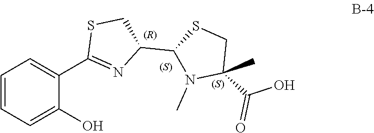

or pharmaceutically acceptable salts thereof. In other aspects, the pharmaceutical composition contains

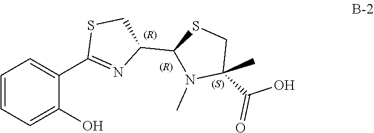

and

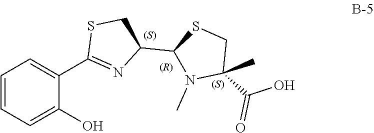

or pharmaceutically acceptable salts thereof. In still further aspects, the pharmaceutical composition contains

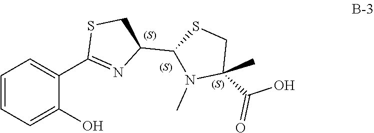

and

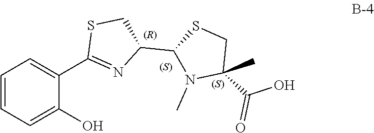

or pharmaceutically acceptable salts thereof. In other aspects, the pharmaceutical composition contains

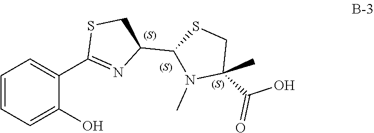

and

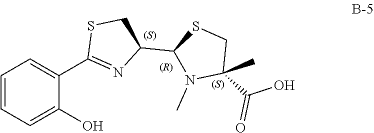

or pharmaceutically acceptable salts thereof. In further aspects, the pharmaceutical composition contains

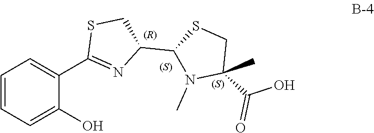

and

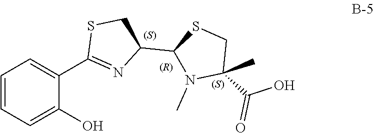

or pharmaceutically acceptable salts thereof.

In further embodiments, the disclosure provides pharmaceutical compositions comprising compound C, or a pharmaceutically acceptable salt thereof, and a pharmaceutically acceptable excipient. In certain aspects, the pharmaceutical composition contains

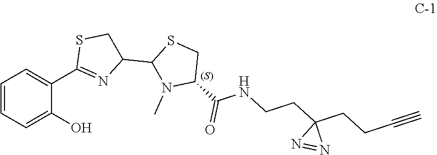

or a pharmaceutically acceptable salt thereof. In other aspects, the pharmaceutical composition contains

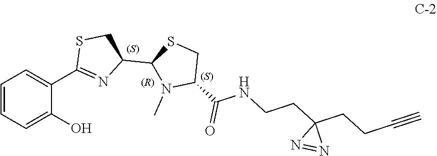

or a pharmaceutically acceptable salt thereof. In further aspects, the pharmaceutical composition comprises

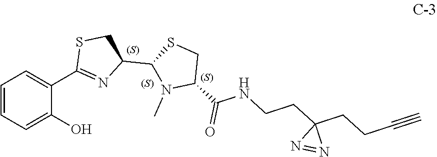

or a pharmaceutically acceptable salt thereof. In yet other aspects, the pharmaceutical composition contains

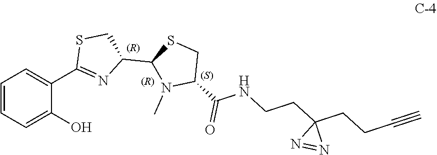

or a pharmaceutically acceptable salt thereof. In still further aspects, the pharmaceutical composition contains

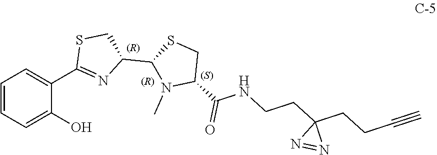

or a pharmaceutically acceptable salt thereof. In some aspects, the pharmaceutical composition contains one or more of:

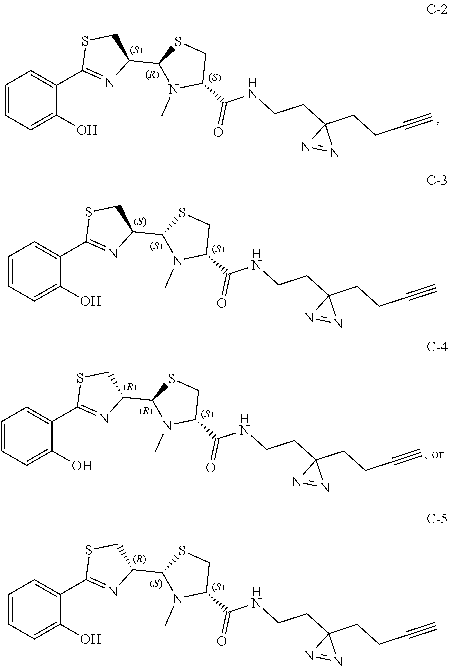

or a pharmaceutically acceptable salt thereof. In further aspects the pharmaceutical composition contains

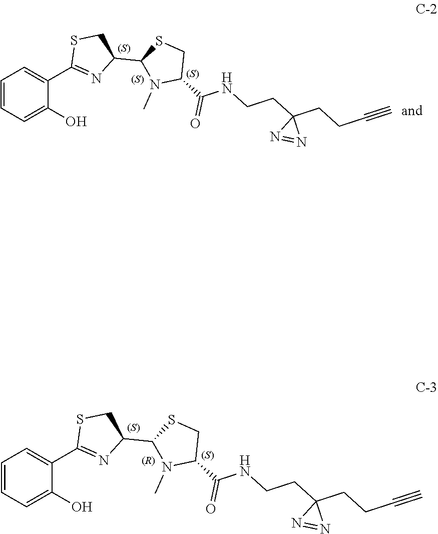

or pharmaceutically acceptable salts thereof. In still further aspects, the pharmaceutical composition contains

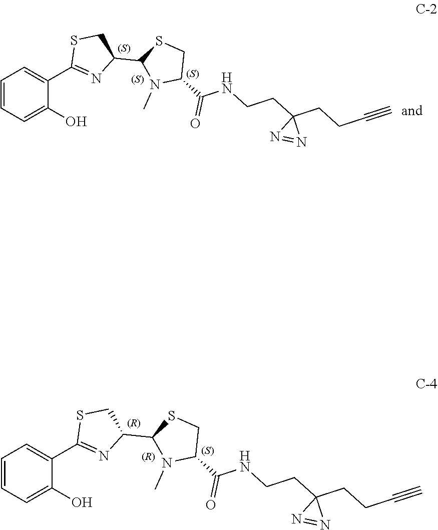

or pharmaceutically acceptable salts thereof. In other aspects, the pharmaceutical composition contains

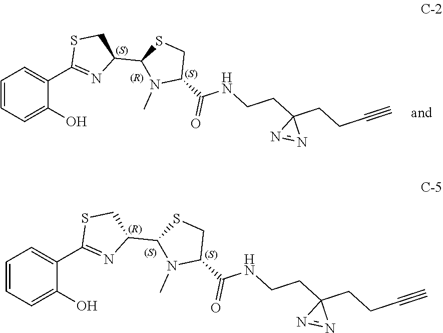

or pharmaceutically acceptable salts thereof. In further aspects, the pharmaceutical compositions contain

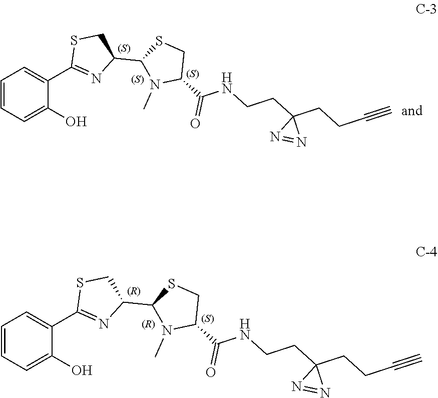

or pharmaceutically acceptable salts thereof. In still other aspects, the pharmaceutical composition contains

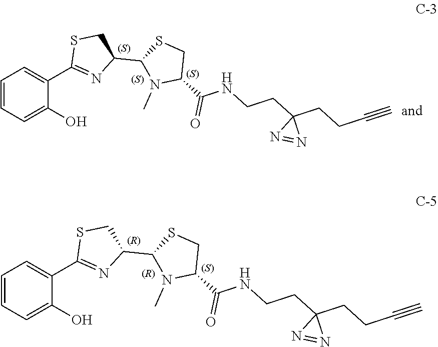

or pharmaceutically acceptable salts thereof. In yet other aspects, the pharmaceutical composition contains

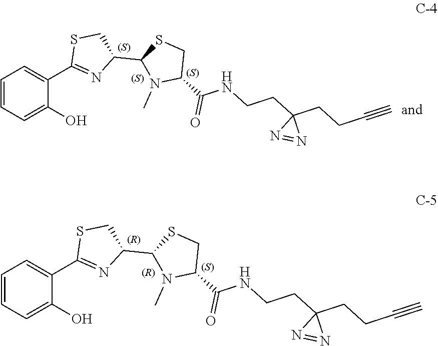

or pharmaceutically acceptable salts thereof.

In yet other embodiments, the disclosure provides pharmaceutical compositions comprising compound D, or a pharmaceutically acceptable salt thereof, and a pharmaceutically acceptable excipient. In further aspects, the pharmaceutical composition contains

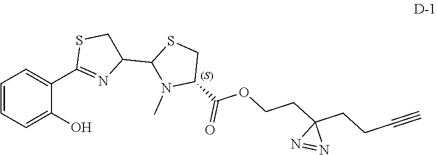

or a pharmaceutically acceptable salt thereof. In other aspects, the pharmaceutical composition contains

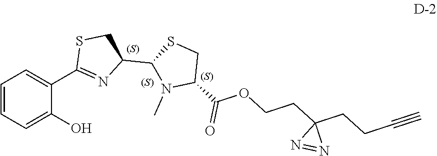

or a pharmaceutically acceptable salt thereof. In further aspects, the pharmaceutical composition contains

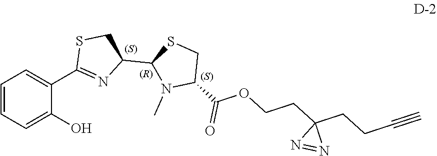

or a pharmaceutically acceptable salt thereof. In yet other aspects, the pharmaceutical composition contains

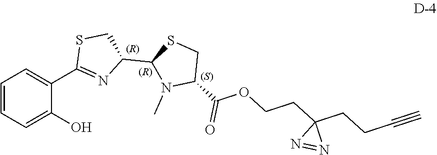

or a pharmaceutically acceptable salt thereof. In still further aspects, the pharmaceutical composition contains

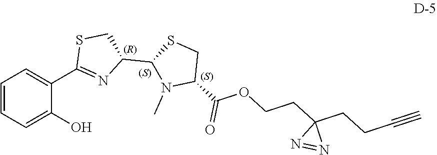

In other aspects, the pharmaceutical composition contains one or more of

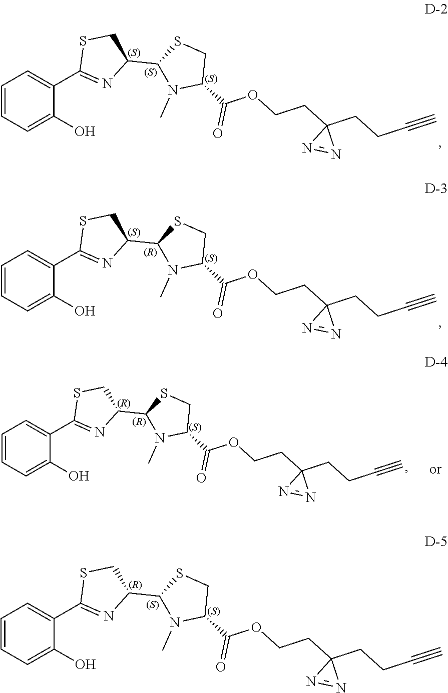

or a pharmaceutically acceptable salt thereof. In other aspects, the pharmaceutical composition contains

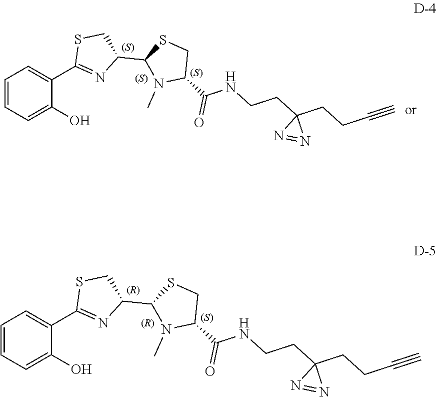

or pharmaceutically acceptable salts thereof. In yet other aspects, the pharmaceutical composition contains

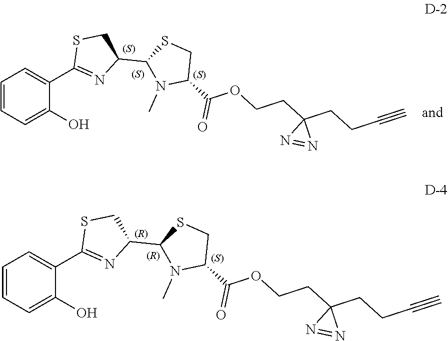

or pharmaceutically acceptable salts thereof. In still further aspects, the pharmaceutical composition contains

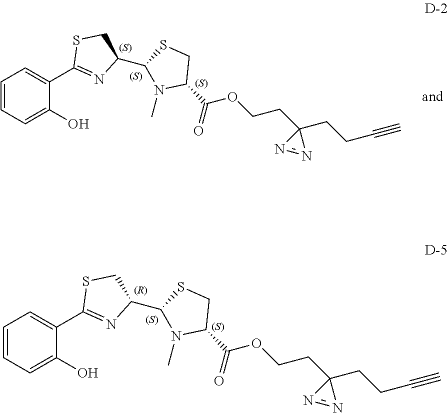

or pharmaceutically acceptable salts thereof. In other aspects, the pharmaceutical composition contains

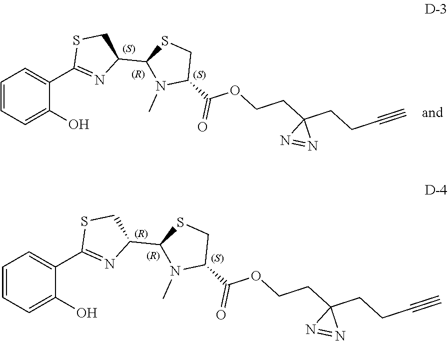

or pharmaceutically acceptable salts thereof. In further aspects, the pharmaceutical composition contains

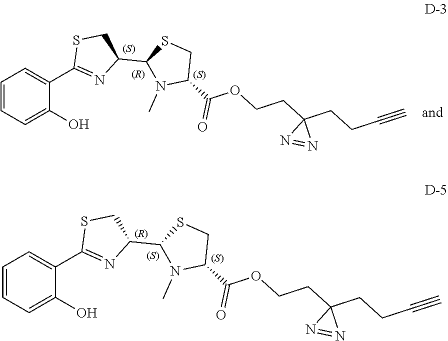

or pharmaceutically acceptable salts thereof. In further aspects, the pharmaceutical composition contains

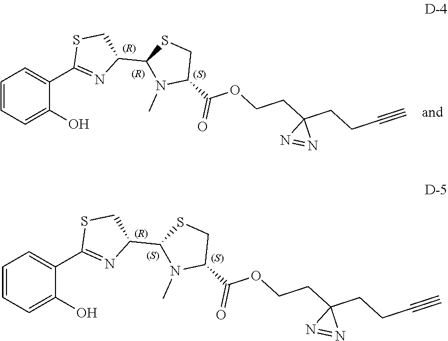

or pharmaceutically acceptable salts thereof.

In further embodiments, the pharmaceutical composition contains compound E or a pharmaceutically acceptable salt thereof. In some aspects, the pharmaceutical composition contains

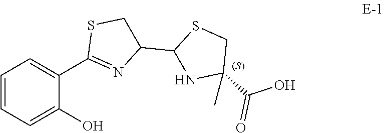

or a pharmaceutically acceptable salt thereof. In further aspects, the pharmaceutical composition contains

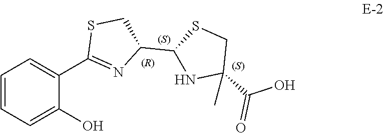

or a pharmaceutically acceptable salt thereof. In other aspects, the pharmaceutical composition contains

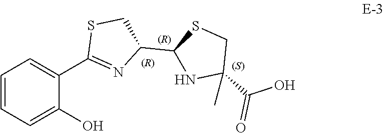

or a pharmaceutically acceptable salt thereof. In still further aspects, the pharmaceutical composition contains

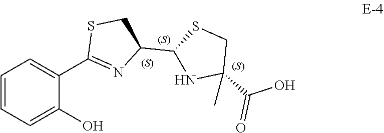

or a pharmaceutically acceptable salt thereof. In yet other aspects, the pharmaceutical composition contains

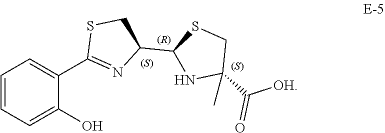

In other aspects, the pharmaceutical composition contains one or more of

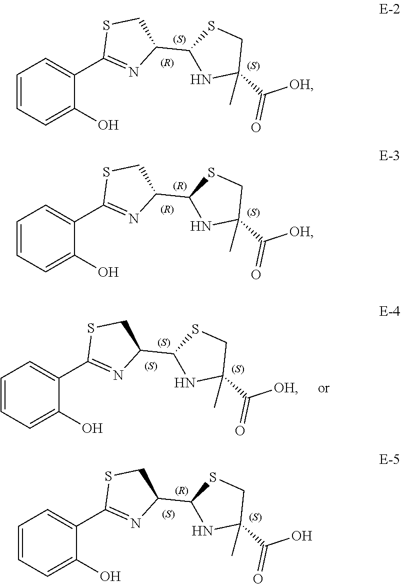

or a pharmaceutically acceptable salt thereof. In still further aspects, the pharmaceutical composition contains

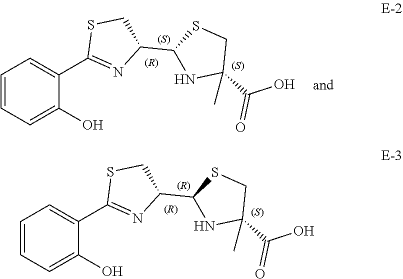

or pharmaceutically acceptable salts thereof. In other aspects, the pharmaceutical composition contains

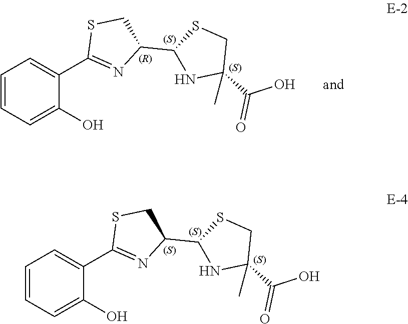

or pharmaceutically acceptable salts thereof. In further aspects, the pharmaceutical composition contains

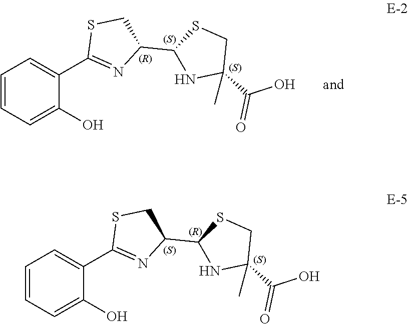

or pharmaceutically acceptable salts thereof. In yet other aspects, the pharmaceutical composition contains

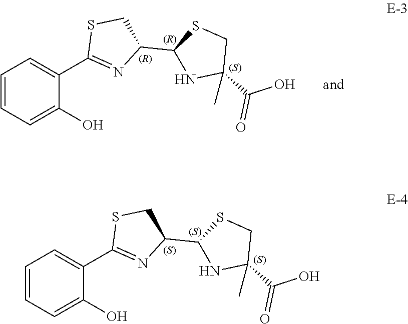

or pharmaceutically acceptable salts thereof. In still further aspects, the pharmaceutical composition contains

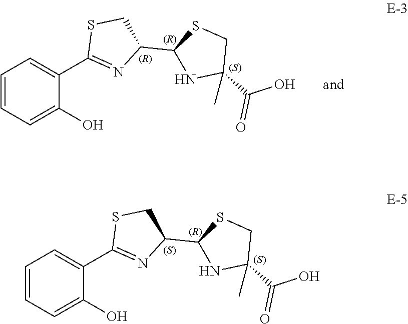

or pharmaceutically acceptable salts thereof. In other aspects, the pharmaceutical composition contains

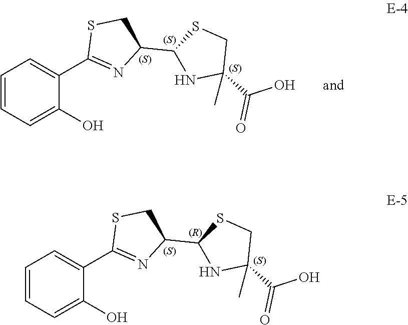

or pharmaceutically acceptable salts thereof.

In yet other embodiments, the pharmaceutical composition contains compound F. In certain aspects, the pharmaceutical composition contains

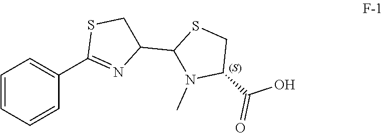

or a pharmaceutically acceptable salt thereof. In other aspects, the pharmaceutical composition contains

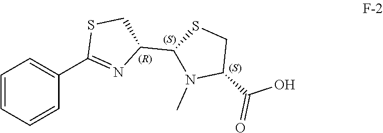

or a pharmaceutically acceptable salt thereof. In further aspects, the pharmaceutical composition contains

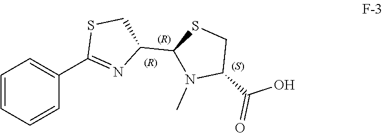

or a pharmaceutically acceptable salt thereof. In yet other aspects, the pharmaceutical composition contains

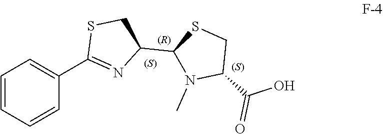

or a pharmaceutically acceptable salt thereof. In still further aspects, the pharmaceutical composition contains

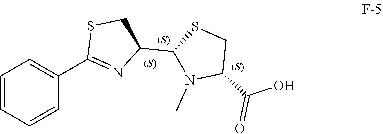

or a pharmaceutically acceptable salt thereof. In further aspects, the pharmaceutical composition contains

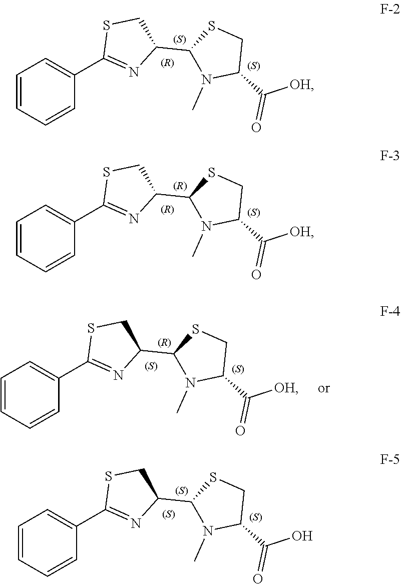

or a pharmaceutically acceptable salt thereof. In other aspects, the pharmaceutical composition contains

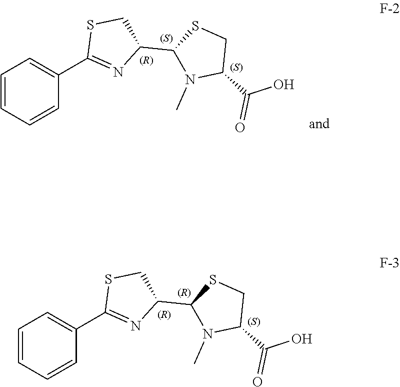

or pharmaceutically acceptable salts thereof. In further aspects, the pharmaceutical composition contains

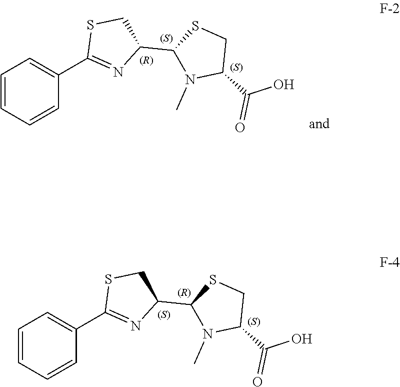

or pharmaceutically acceptable salts thereof. In yet other aspects, the pharmaceutical composition contains

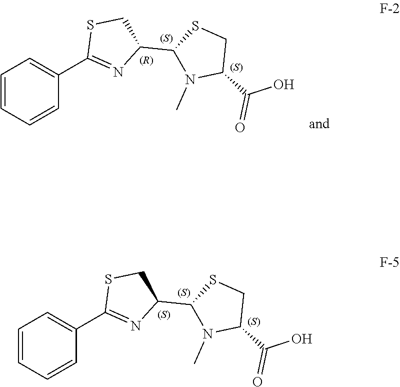

or pharmaceutically acceptable salts thereof. In still further aspects, the pharmaceutical composition contains

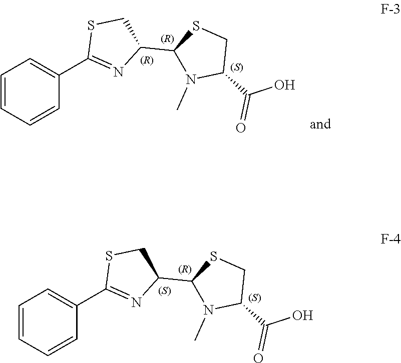

or pharmaceutically acceptable salts thereof. In other aspects, the pharmaceutical composition contains

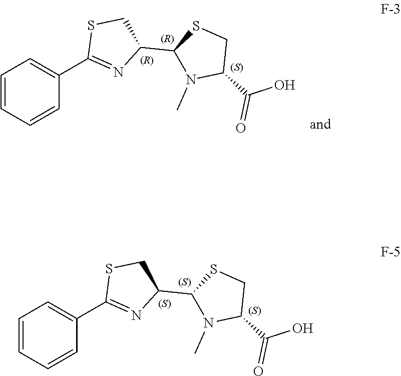

or pharmaceutically acceptable salts thereof. In further aspects, the pharmaceutical composition contains

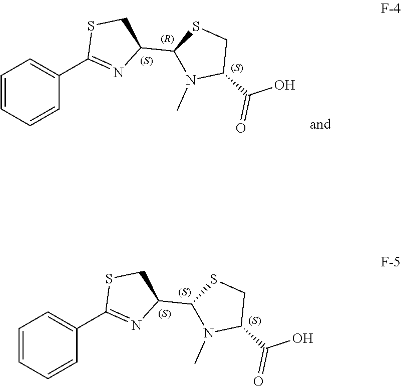

or pharmaceutically acceptable salts thereof.

In still further embodiments, the disclosure provides pharmaceutical compositions containing compound G. In some aspects, the pharmaceutical composition contains

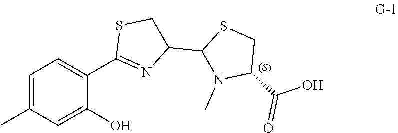

or a pharmaceutically acceptable salt thereof. In other aspects, the pharmaceutical composition contains

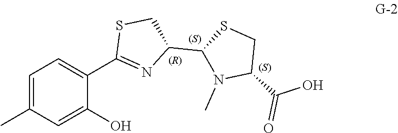

or a pharmaceutically acceptable salt thereof. In further aspects, the pharmaceutical composition contains

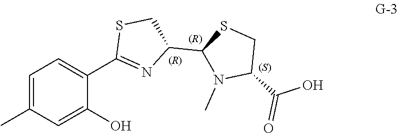

or a pharmaceutically acceptable salt thereof. In yet other aspects, the pharmaceutical composition contains

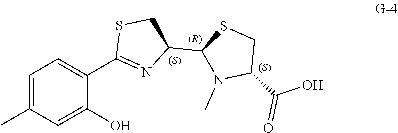

or a pharmaceutically acceptable salt thereof. In still further aspects, the pharmaceutical composition contains

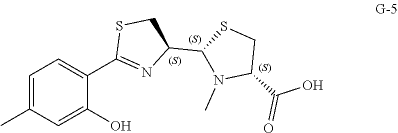

or a pharmaceutically acceptable salt thereof. In further aspects, the pharmaceutical composition contains

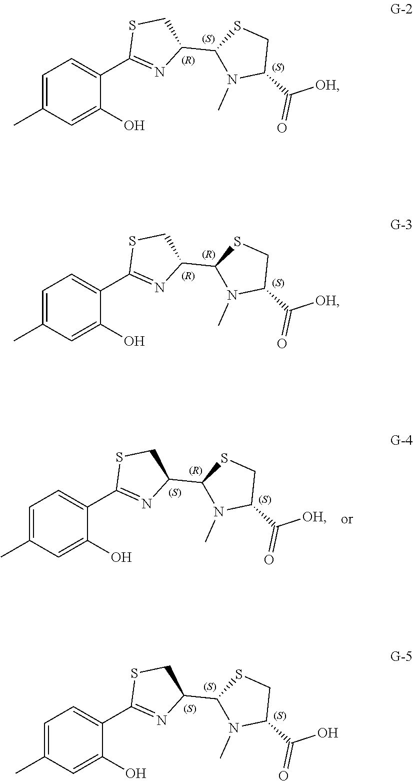

or a pharmaceutically acceptable salt thereof. In other aspects, the pharmaceutical composition contains

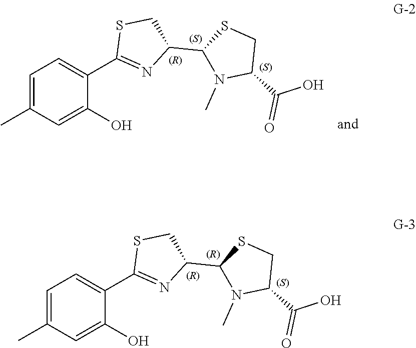

or pharmaceutically acceptable salts thereof. In further aspects, the pharmaceutical composition contains

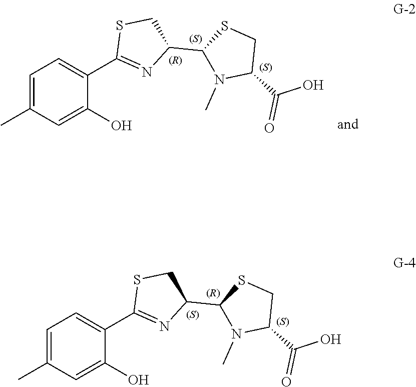

or pharmaceutically acceptable salts thereof. In yet other aspects, the pharmaceutical composition contains

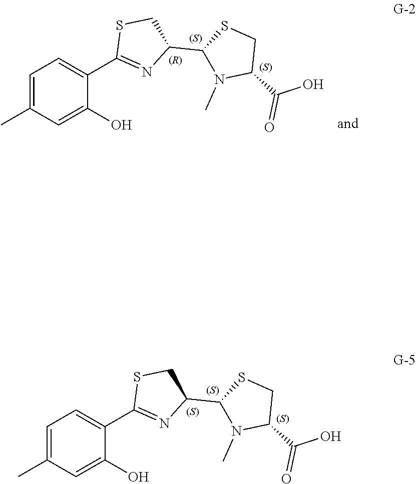

or pharmaceutically acceptable salts thereof. In still further aspects, the pharmaceutical composition contains

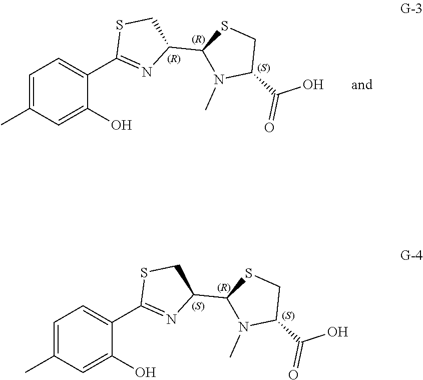

or pharmaceutically acceptable salts thereof. In other aspects, the pharmaceutical composition contains

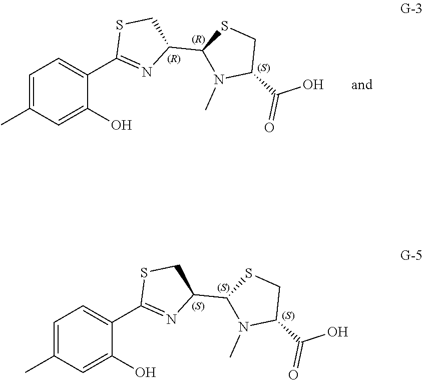

or pharmaceutically acceptable salts thereof. In further aspects, the pharmaceutical composition contains

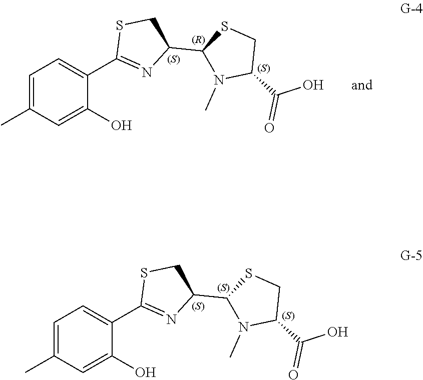

or pharmaceutically acceptable salts thereof.

In other embodiments, the pharmaceutical composition contains

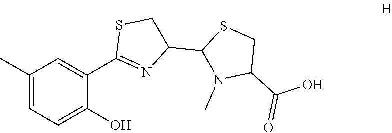 [SMILES: Cc1ccc(O)c(C2=NC(C3SCC(C(=O)O)N3C)CS2)c1]

or a pharmaceutically acceptable salt thereof. In some aspects, the pharmaceutical composition contains

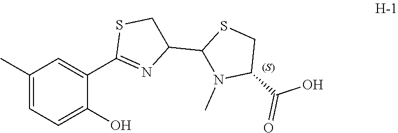

or a pharmaceutically acceptable salt thereof. In certain aspects, the pharmaceutical composition contains

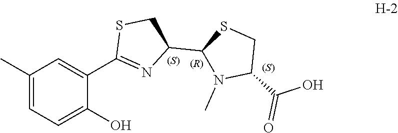

or a pharmaceutically acceptable salt thereof. In other aspects, the pharmaceutical composition contains

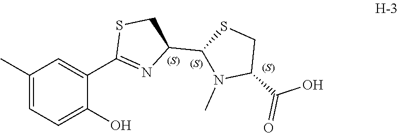

or a pharmaceutically acceptable salt thereof. In further aspects, the pharmaceutical composition contains

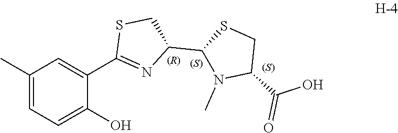

or a pharmaceutically acceptable salt thereof. In yet other aspects, the pharmaceutical composition contains

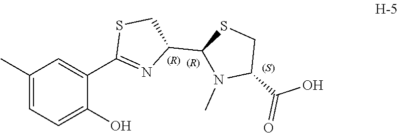

or a pharmaceutically acceptable salt thereof. In still further aspects, the pharmaceutical composition contains one or more of

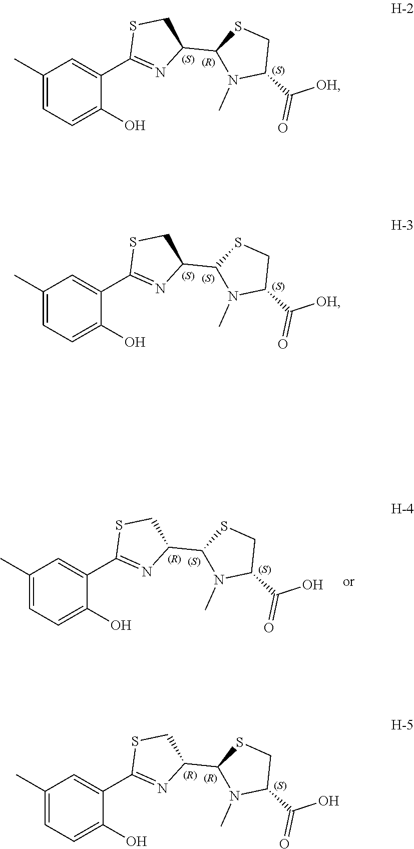

or a pharmaceutically acceptable salt thereof. In other aspects, the pharmaceutical composition contains

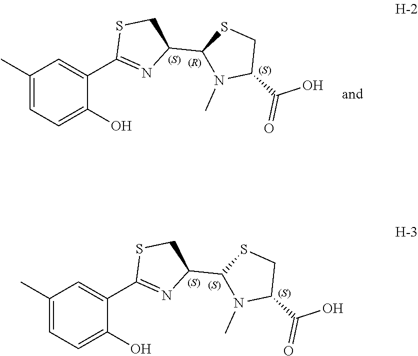

or pharmaceutically acceptable salts thereof. In further aspects, the pharmaceutical composition contains

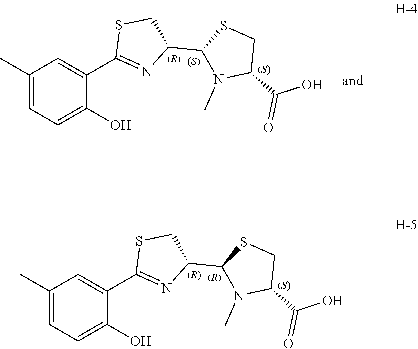

or pharmaceutically acceptable salts thereof. In yet other aspects, the pharmaceutical composition contains

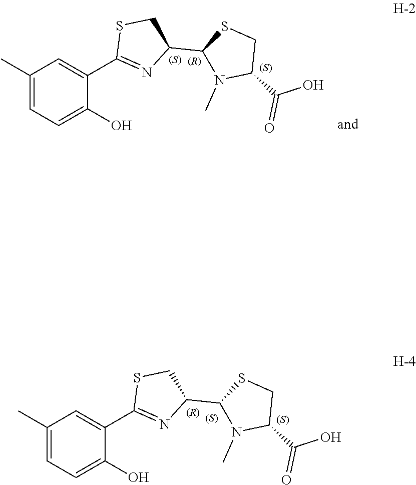

or pharmaceutically acceptable salts thereof. In still further aspects, the pharmaceutical composition contains

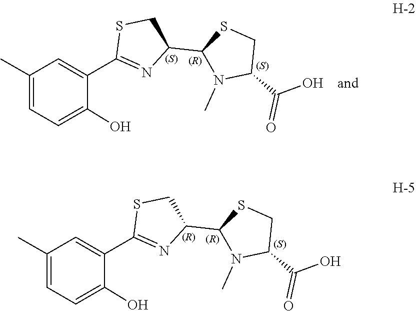

or pharmaceutically acceptable salts thereof. In other aspects, the pharmaceutical composition contains

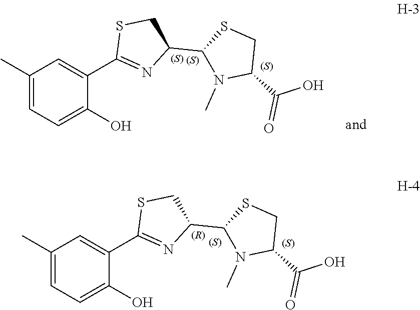

or pharmaceutically acceptable salts thereof. In further aspects, the pharmaceutical composition contains

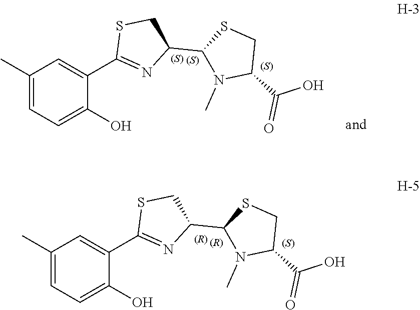

or pharmaceutically acceptable salts thereof.

In further embodiments, the pharmaceutical composition contains

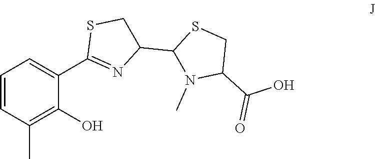 [SMILES: Cc1cccc(C2=NC(C3SCC(C(=O)O)N3C)CS2)c1O]

or a pharmaceutically acceptable salt thereof. In certain aspects, pharmaceutical composition contains

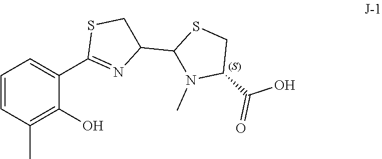

or a pharmaceutically acceptable salt thereof. In other aspects, the pharmaceutical composition contains

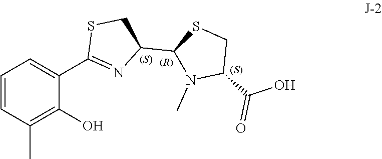

or a pharmaceutically acceptable salt thereof. In further aspects, the pharmaceutical composition contains

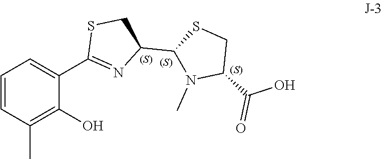

or a pharmaceutically acceptable salt thereof

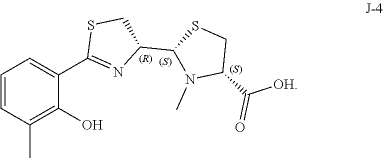

In yet other aspects, the pharmaceutical composition contains

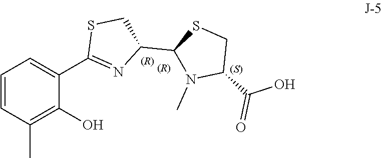

or a pharmaceutically acceptable salt thereof. In still further aspects, the pharmaceutical composition contains one or more of

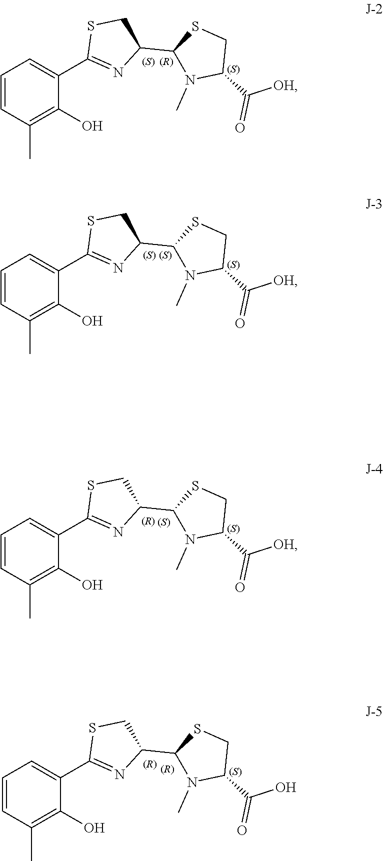

or a pharmaceutically acceptable salt thereof. In other aspects, the pharmaceutical composition contains

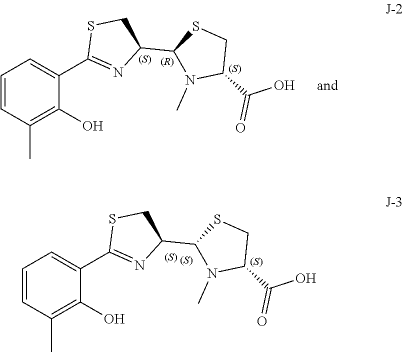

or pharmaceutically acceptable salts thereof. In further aspects, the pharmaceutical composition contains

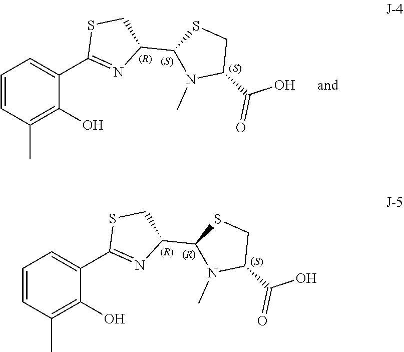

or pharmaceutically acceptable salts thereof. In yet other aspects, the pharmaceutical composition contains

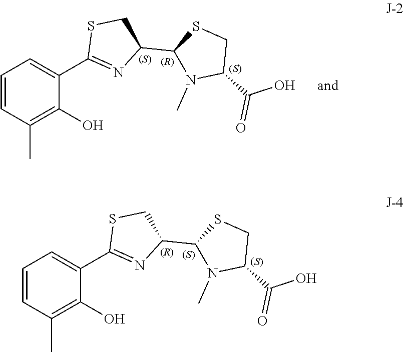

or pharmaceutically acceptable salts thereof. In still further aspects, the pharmaceutical composition contains

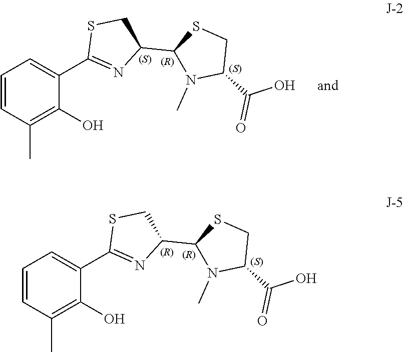

or pharmaceutically acceptable salts thereof. In other aspects, the pharmaceutical composition contains

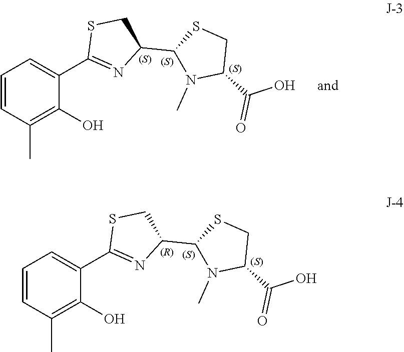

or a pharmaceutically acceptable salt thereof. In further aspects, the pharmaceutical composition contains

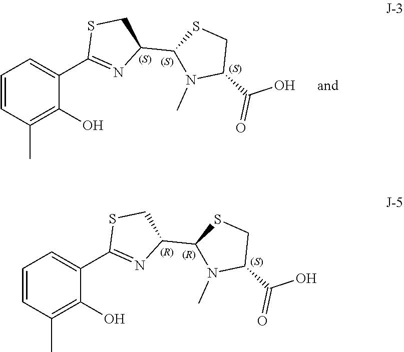

or a pharmaceutically acceptable salt thereof.

In yet further embodiments, pharmaceutical composition contains

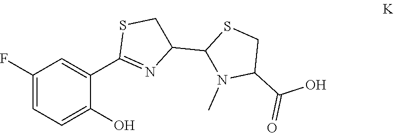 [SMILES: CN1C(C(=O)O)CSC1C1CSC(c2cc(F)ccc2O)=N1]

or a pharmaceutically acceptable salt thereof. In certain aspects, the pharmaceutical composition contains

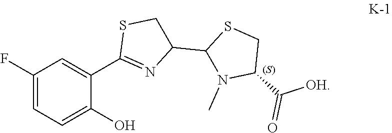

In some aspects, the pharmaceutical composition contains

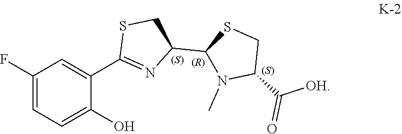

or a pharmaceutically acceptable salt thereof. In other aspects, the pharmaceutical composition contains

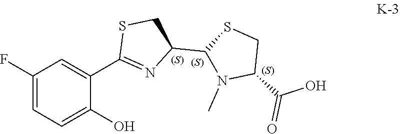

or a pharmaceutically acceptable salt thereof. In further aspects, the pharmaceutical composition contains

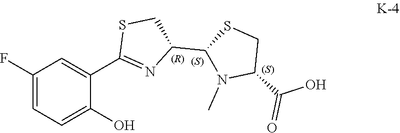

or a pharmaceutically acceptable salt thereof. In yet other aspects, the pharmaceutical composition contains

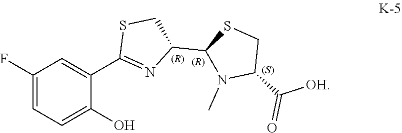

In still further aspects, the pharmaceutical composition contains,

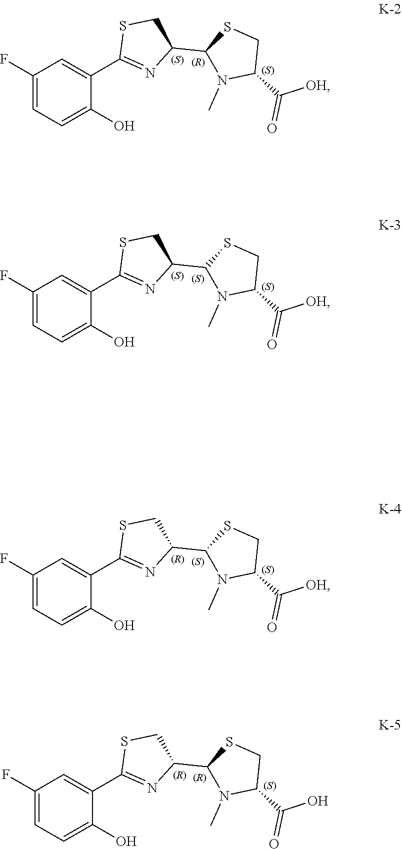

or pharmaceutically acceptable salts thereof. In other aspects, the pharmaceutical composition contains

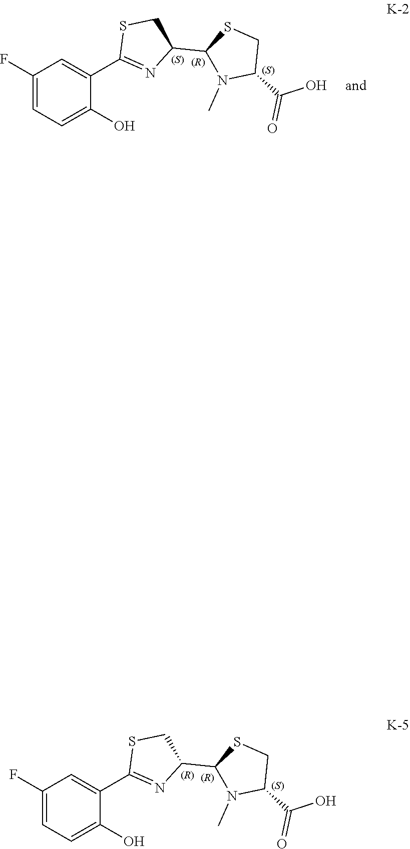

or pharmaceutically acceptable salts thereof. In further aspects, the pharmaceutical composition contains

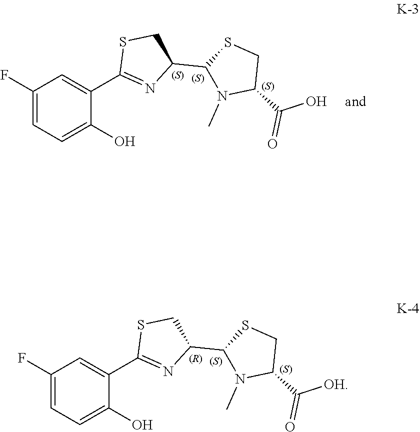

In still other aspects, the pharmaceutical composition contains

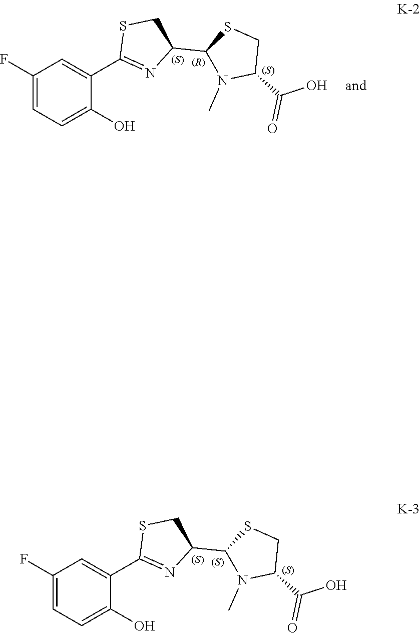

or pharmaceutically acceptable salts thereof. In yet further aspects, the pharmaceutical composition contains

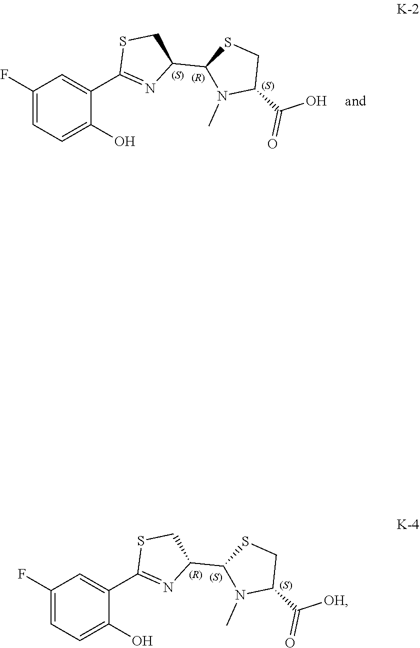

pharmaceutically acceptable salts thereof. In other aspects, the pharmaceutical composition contains

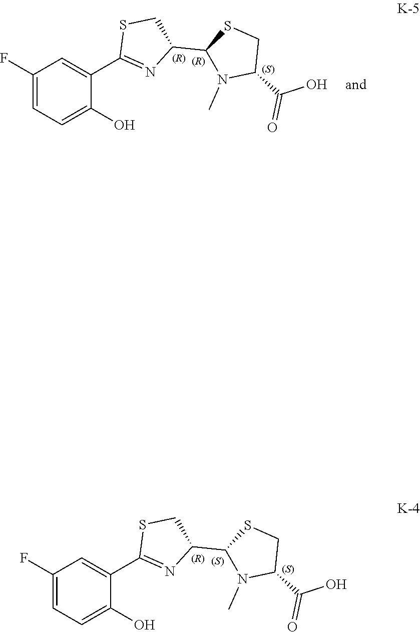

or pharmaceutically acceptable salts thereof. In further aspects, the pharmaceutical composition contains

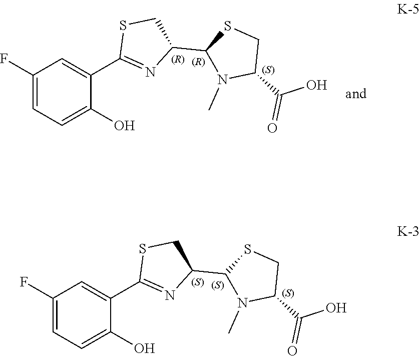

or pharmaceutically acceptable salts thereof.

In other embodiments, the pharmaceutical composition contains

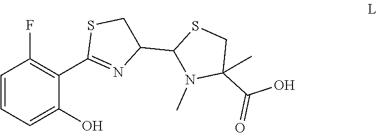 [SMILES: CN1C(C2CSC(c3c(O)cccc3F)=N2)SCC1(C)C(=O)O]

or a pharmaceutically acceptable salt thereof. In certain aspects, the pharmaceutical composition contains

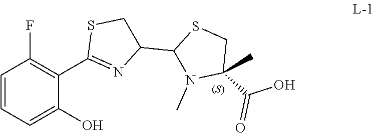

or a pharmaceutically acceptable salt thereof. In further aspects, the pharmaceutical composition contains

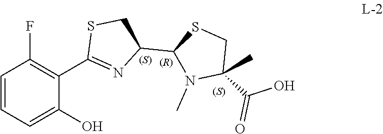

or a pharmaceutically acceptable salt thereof. In yet other aspects, the pharmaceutical composition contains

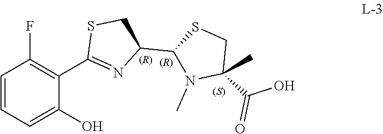

or a pharmaceutically acceptable salt thereof. In still further aspects, the pharmaceutical composition contains

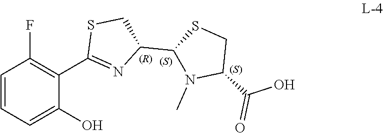

or a pharmaceutically acceptable salt thereof. In other aspects, the pharmaceutical composition contains

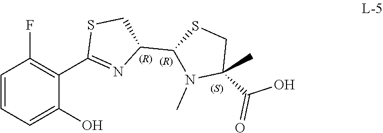

or a pharmaceutically acceptable salt thereof. In still further aspects, the pharmaceutical composition contains one or more of

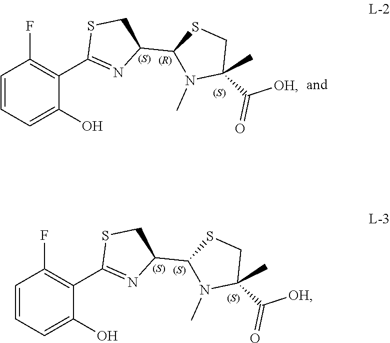

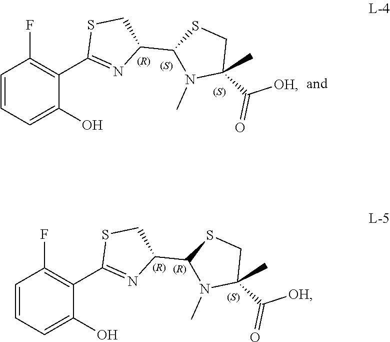

or pharmaceutically acceptable salts thereof. In other aspects, the pharmaceutical composition contains

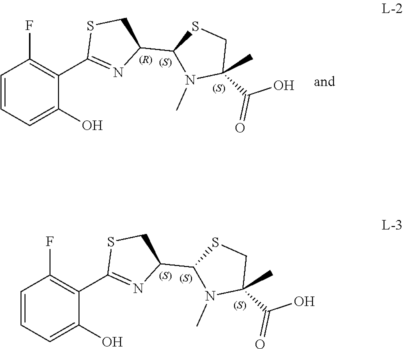

or pharmaceutically acceptable salts thereof. In further aspects, the pharmaceutical composition contains

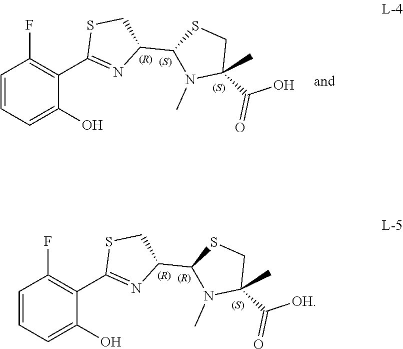

In still other aspects, the pharmaceutical composition contains

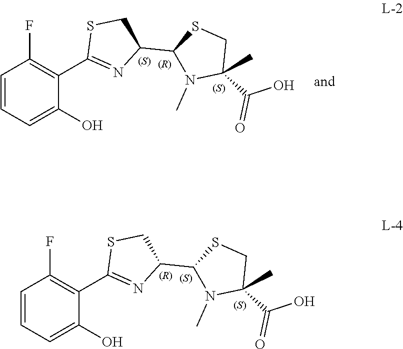

or pharmaceutically acceptable salts thereof. In yet further aspects, the pharmaceutical composition contains

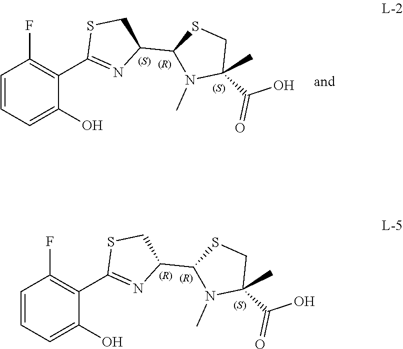

or pharmaceutically acceptable salts thereof. In other aspects, the pharmaceutical composition contains

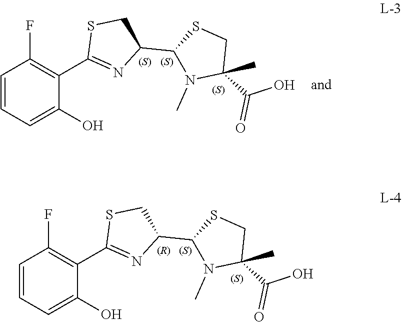

or pharmaceutically acceptable salts thereof. In further aspects, the pharmaceutical composition contains

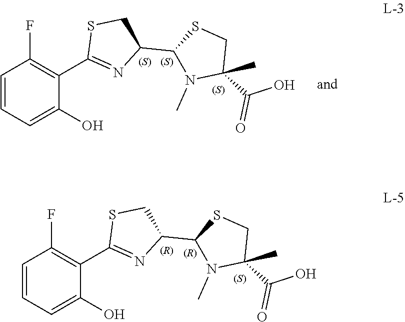

or pharmaceutically acceptable salts thereof.

In further embodiments, the pharmaceutical composition contains

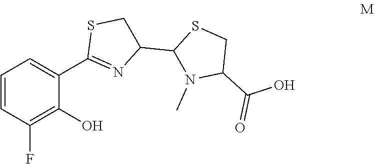 [SMILES: CN1C(C(=O)O)CSC1C1CSC(c2cccc(F)c2O)=N1]

or a pharmaceutically acceptable salt thereof. In certain aspects, the pharmaceutical composition contains

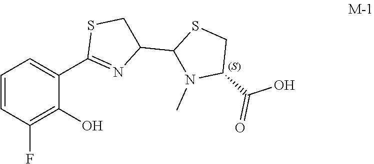

or a pharmaceutically acceptable salt thereof. In other aspects, the pharmaceutical composition contains

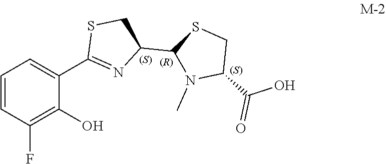

or a pharmaceutically acceptable salt thereof. In further aspects, the pharmaceutical composition contains

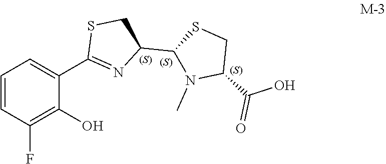

or a pharmaceutically acceptable salt thereof. In yet other aspects, the pharmaceutical composition contains

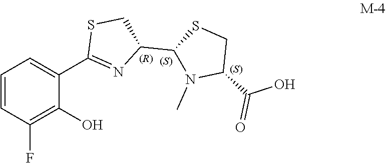

or a pharmaceutically acceptable salt thereof. In still further aspects, the pharmaceutical composition contains

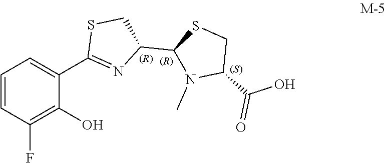

or a pharmaceutically acceptable salt thereof. In still further aspects, the pharmaceutical composition contains one or more of

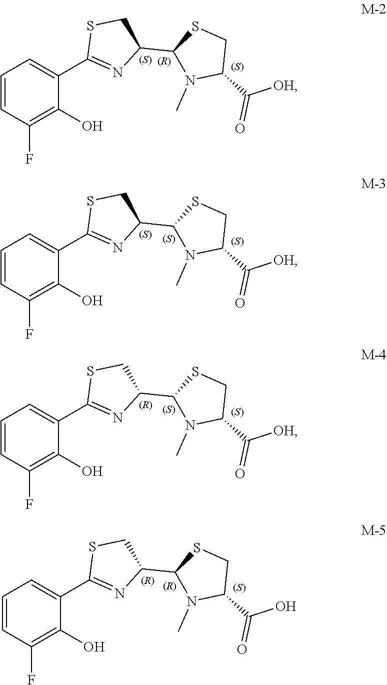

or pharmaceutically acceptable salts thereof. In other aspects, the pharmaceutical composition contains

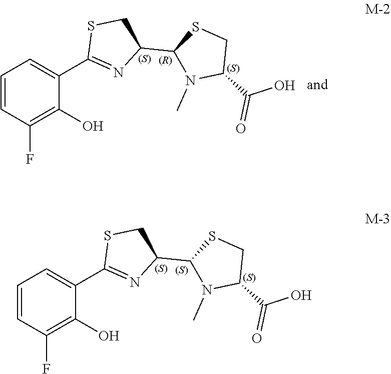

or pharmaceutically acceptable salts thereof. In further aspects, the pharmaceutical composition contains

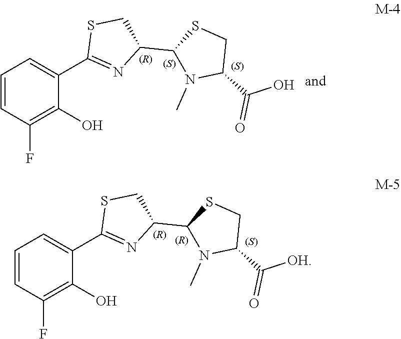

In still other aspects, the pharmaceutical composition contains

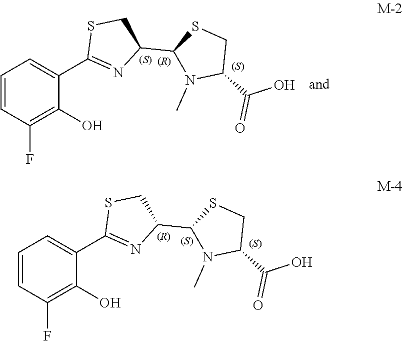

or pharmaceutically acceptable salts thereof. In yet further aspects, the pharmaceutical composition contains

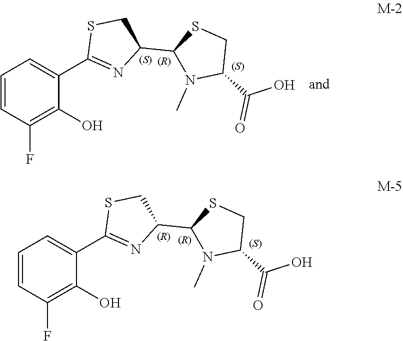

or pharmaceutically acceptable salts thereof. In other aspects, the pharmaceutical composition contains

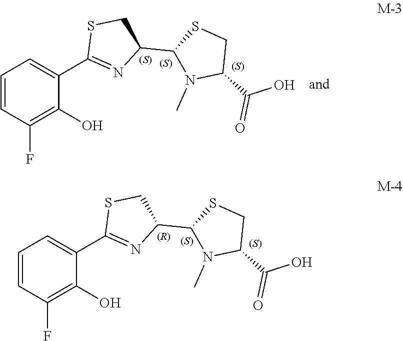

or pharmaceutically acceptable salts thereof. In further aspects, the pharmaceutical composition contains

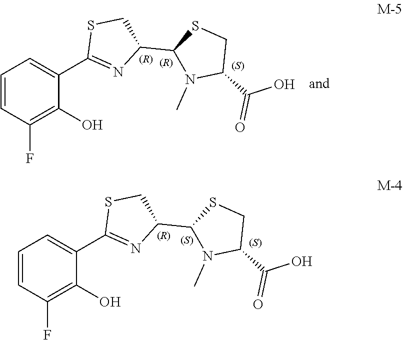

or pharmaceutically acceptable salts thereof.

In still other embodiments, the pharmaceutical composition contains

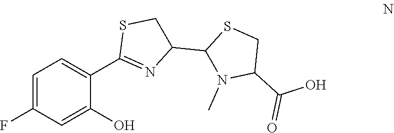 [SMILES: CN1C(C(=O)O)CSC1C1CSC(c2ccc(F)cc2O)=N1]

or a pharmaceutically acceptable salt thereof. In certain aspects, the pharmaceutical composition contains

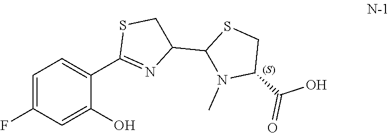

or a pharmaceutically acceptable salt thereof. In other aspects, the pharmaceutical composition contains

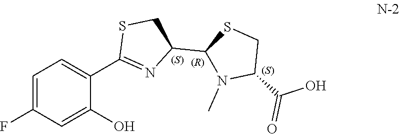

or a pharmaceutically acceptable salt thereof. In further aspects, the pharmaceutical composition contains

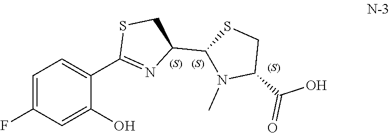

or a pharmaceutically acceptable salt thereof. In yet other aspects, the pharmaceutical composition contains

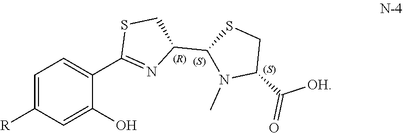

In still further aspects, the pharmaceutical composition contains

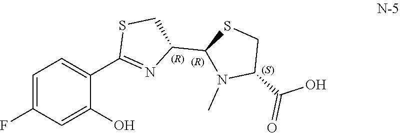

or a pharmaceutically acceptable salt thereof. In still further aspects, the pharmaceutical composition contains one or more of

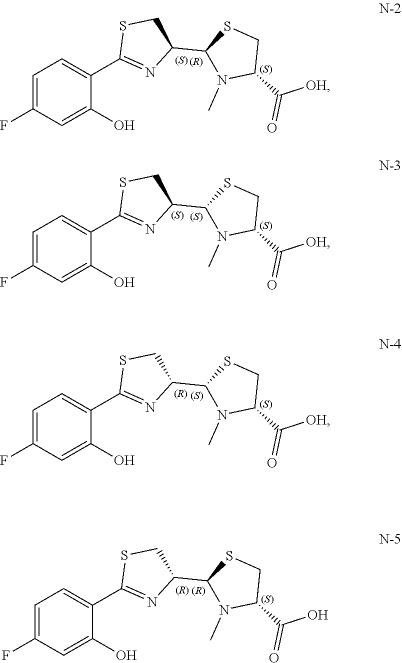

or pharmaceutically acceptable salts thereof. In other aspects, the pharmaceutical composition contains

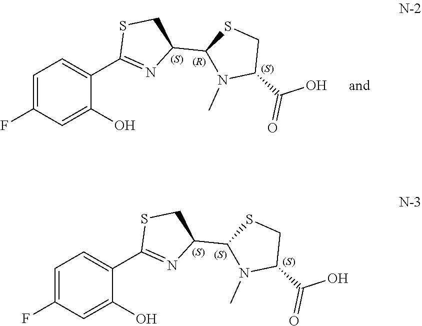

or pharmaceutically acceptable salts thereof. In further aspects, the pharmaceutical composition contains

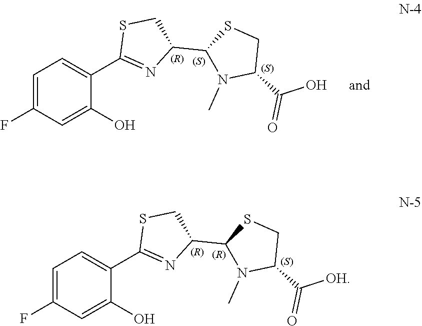

In still other aspects, the pharmaceutical composition contains

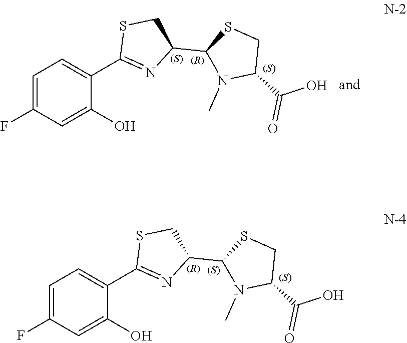

or pharmaceutically acceptable salts thereof. In yet further aspects, the pharmaceutical composition contains

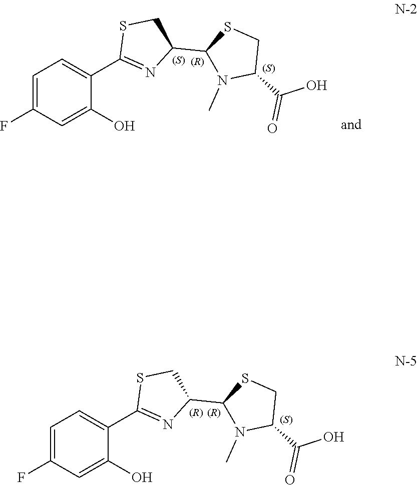

or pharmaceutically acceptable salts thereof. In other aspects, the pharmaceutical composition contains

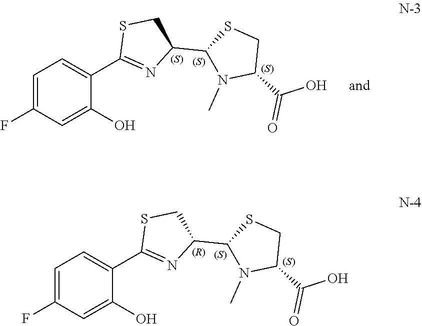

or pharmaceutically acceptable salts thereof. In further aspects, the pharmaceutical composition contains

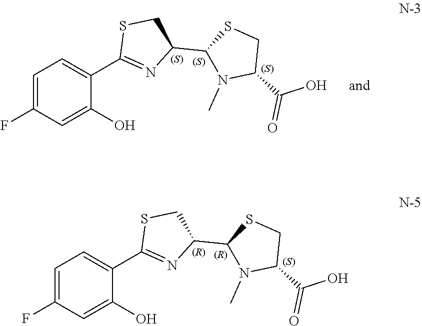

or pharmaceutically acceptable salts thereof.

In yet further embodiments, the pharmaceutical composition contains

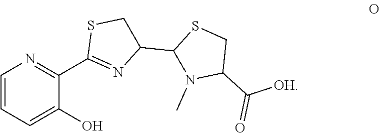 [SMILES: CN1C(C(=O)O)CSC1C1CSC(c2ncccc2O)=N1]

or a pharmaceutically acceptable salt thereof. In certain aspects, the pharmaceutical composition contains

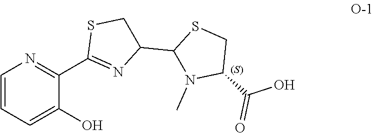

or a pharmaceutically acceptable salt thereof. In other aspects, the pharmaceutical composition contains

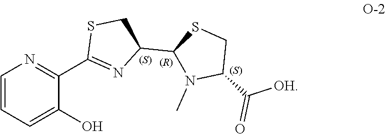

or a pharmaceutically acceptable salt thereof. In further aspects, the pharmaceutical composition contains

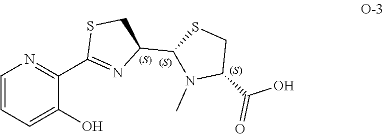

or a pharmaceutically acceptable salt thereof. In yet other aspects, the pharmaceutical composition contains

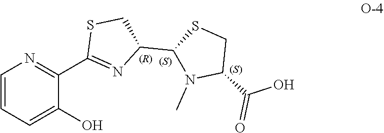

or a pharmaceutically acceptable salt thereof. In still further aspects, the pharmaceutical composition contains

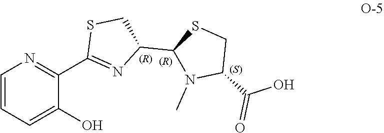

or a pharmaceutically acceptable salt thereof. In still further aspects, the pharmaceutical composition contains one or more of

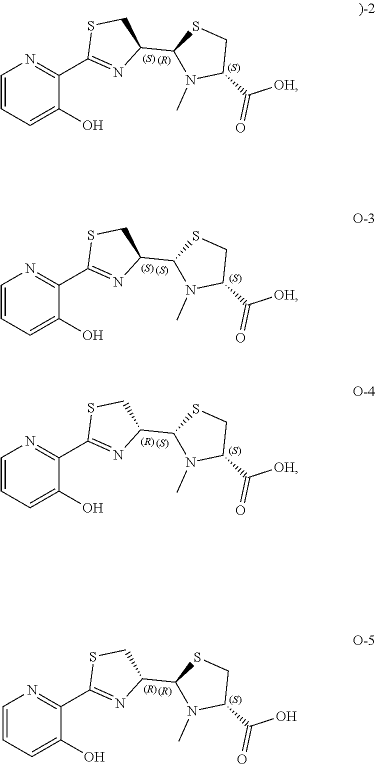

or pharmaceutically acceptable salts thereof. In other aspects, the pharmaceutical composition contains

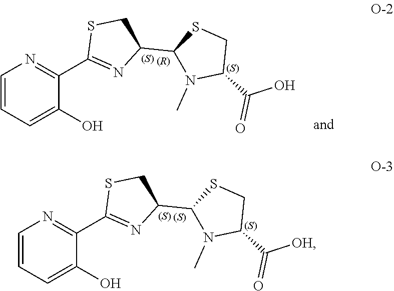

or pharmaceutically acceptable salts thereof. In further aspects, the pharmaceutical composition contains

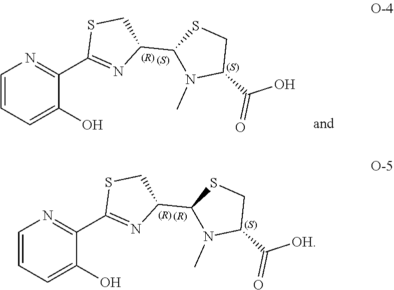

In still other aspects, the pharmaceutical composition contains

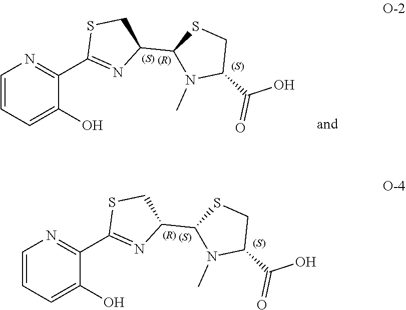

or pharmaceutically acceptable salts thereof. In yet further aspects, the pharmaceutical composition contains

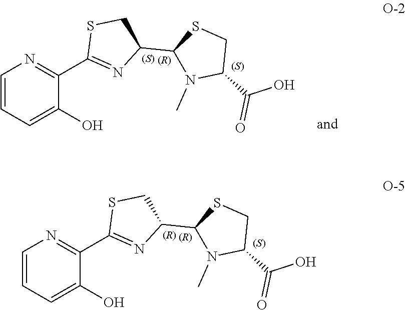

or pharmaceutically acceptable salts thereof. In other aspects, the pharmaceutical composition contains

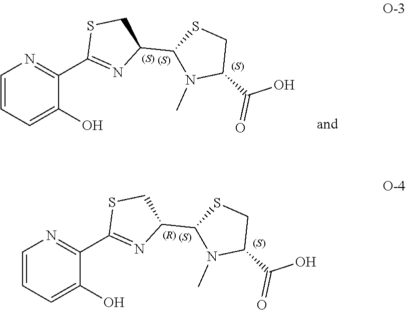

or pharmaceutically acceptable salts thereof. In further aspects, the pharmaceutical composition contains

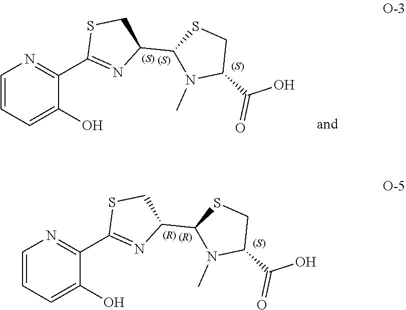

or pharmaceutically acceptable salts thereof.

In other embodiments, the pharmaceutical composition contains

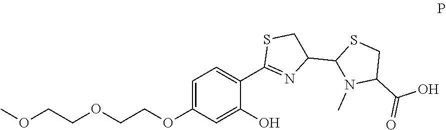 [SMILES: COCCOCCOc1ccc(C2=NC(C3SCC(C(=O)O)N3C)CS2)c(O)c1]

or a pharmaceutically acceptable salt thereof. In certain aspects, the pharmaceutical composition contains

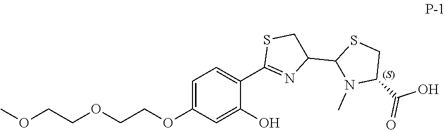

or a pharmaceutically acceptable salt thereof. In other aspects, the pharmaceutical composition contains

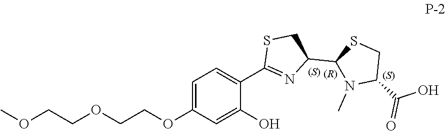

or a pharmaceutically acceptable salt thereof. In further aspects, the pharmaceutical composition contains

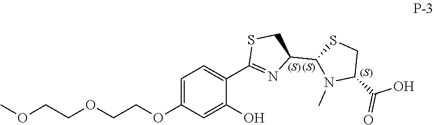

or a pharmaceutically acceptable salt thereof. In still other aspects, the pharmaceutical composition contains

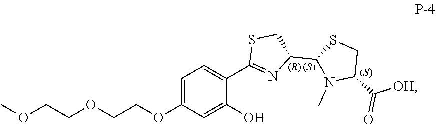

or a pharmaceutically acceptable salt thereof. In yet further aspects, the pharmaceutical composition contains

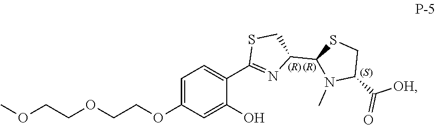

or a pharmaceutically acceptable salt thereof. In still further aspects, the pharmaceutical composition contains one or more of

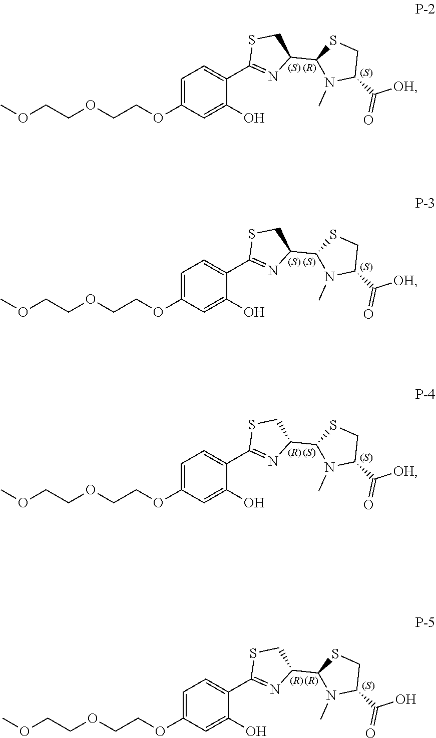

or pharmaceutically acceptable salts thereof. In other aspects, the pharmaceutical composition contains

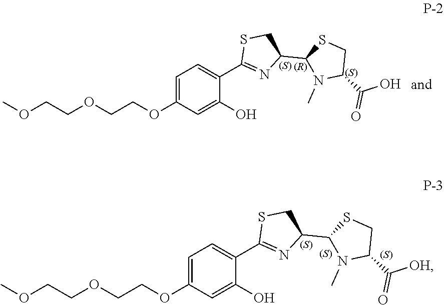

or pharmaceutically acceptable salts thereof. In further aspects, the pharmaceutical composition contains

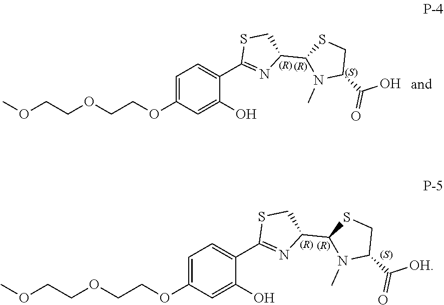

In still other aspects, the pharmaceutical composition contains

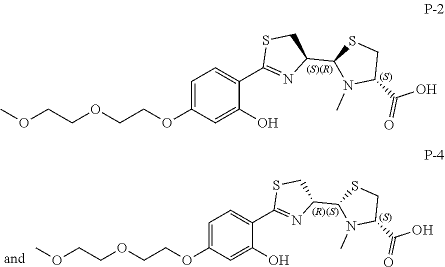

or pharmaceutically acceptable salts thereof. In yet further aspects, the pharmaceutical composition contains

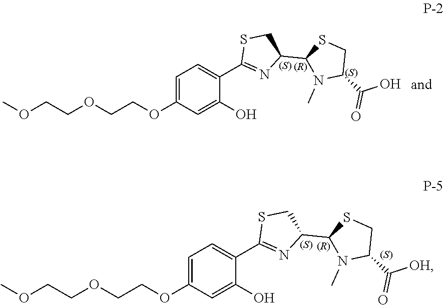

or pharmaceutically acceptable salts thereof. In other aspects, the pharmaceutical composition contains

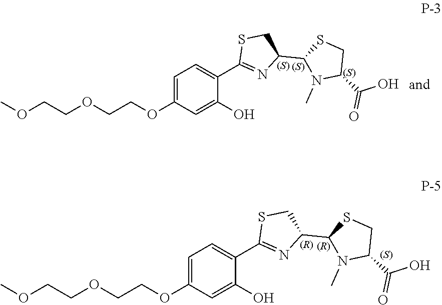

or pharmaceutically acceptable salts thereof. In further aspects, the pharmaceutical composition contains

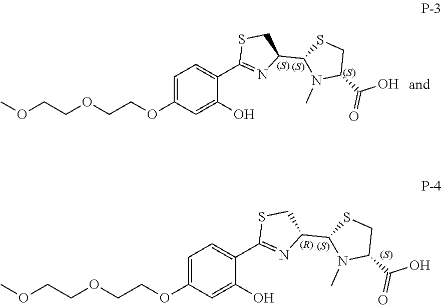

or pharmaceutically acceptable salts thereof.

When the pharmaceutical compositions contain two or more stereoisomers of compound A, the stereoisomers may be present in equal ratios or in varying ratios. In certain embodiments, the compositions contain an excess of the (S,S,S) ratio of one of compound A, compound B, compound C, compound D, compound E, compound F, compound G, compound H, compound J, compound K, compound L, compound M, compound N, compound O, compound P, or compound Q. In other embodiments, the compositions contain an excess of the (R,S,S) ratio of one of compound A, compound B, compound C, compound D, compound E, compound F, compound G, compound H, compound J, compound K, compound L, compound M, compound N, compound O, compound P, or compound Q.

In further embodiments, the compositions contain an excess of the (S,R,S) ratio of one of compound A, compound B, compound C, compound D, compound E, compound F, or compound G. In yet other embodiments, the compositions contain an excess of the (R,R,S) ratio of one of compound A, compound B, compound C, compound D, compound E, compound F, compound G, compound H, compound J, compound K, compound L, compound M, compound N, compound O, compound P, or compound Q.

In certain aspects, the pharmaceutical compositions contain a higher ratio of

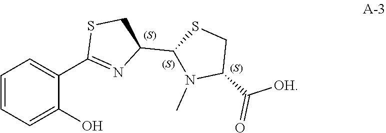

In other embodiments, the pharmaceutical compositions contain

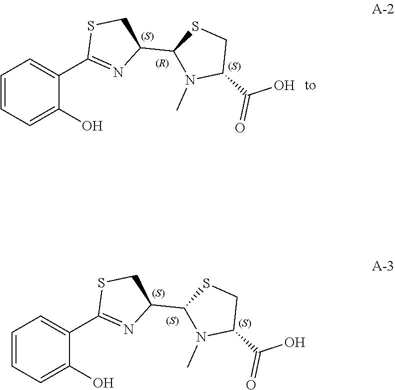

at a ratio of about 10:about 90, or about 50:about 50, or about 90:about 10. In further embodiments, the molar ratio of A-2 to A-3 is about 25 to about 55:about 45 to about 75. In other embodiments, the molar ratio of A-2 to A-3 is about 40 to about 60. In further embodiments, the molar ratio of A-2 to A-3 is about 25:about 75. In yet other embodiments, the molar ratio of A-2 to A-3 is about 30:about 70. In still further embodiments, the molar ratio of A-2 to A-3 is about 35:about 65.

In further embodiments, the pharmaceutical composition contains

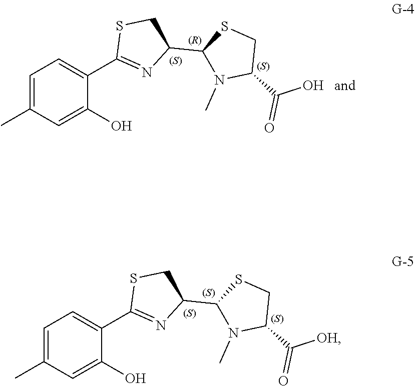

or pharmaceutically acceptable salts thereof, in a molar ratio of about 10:about 90, or about 50:about 50, or about 90:about 10. In further embodiments, the molar ratio of G-4 to G-5 is about 25 to about 55:about 45 to about 75. In other embodiments, the molar ratio of G-4 to G-5 is about 40:about 60. In further embodiments, the molar ratio of G-4 to G-5 is about 25:about 75. In yet other embodiments, the molar ratio of G-4 to G-5 is about 30:about 70. In still further embodiments, the molar ratio of G-4 to G-5 is about 35:about 65.

In further embodiments, the pharmaceutical composition contains

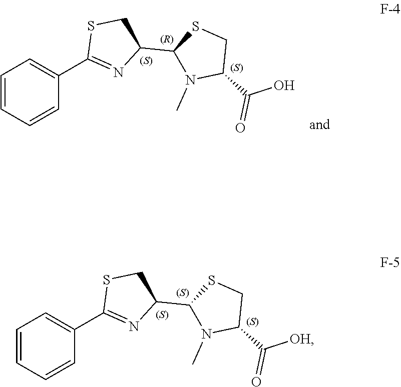

or pharmaceutically acceptable salts thereof, in a molar ratio of about 10:about 90, about 50:about 50, or about 90 to about 10. In further embodiments, the molar ratio of F-4 to F-5 is about 25 to about 55:about 45 to about 75. In other embodiments, the molar ratio of F-4 to F-5 is about 40:about 60. In further embodiments, the molar ratio of F-4 to F-5 is about 25:about 75. In yet other embodiments, the molar ratio of F-4 to F-5 is about 30:about 70. In still further embodiments, the molar ratio of F-4 to F-5 is about 35:about 65.

In yet other embodiments, the pharmaceutical composition contains

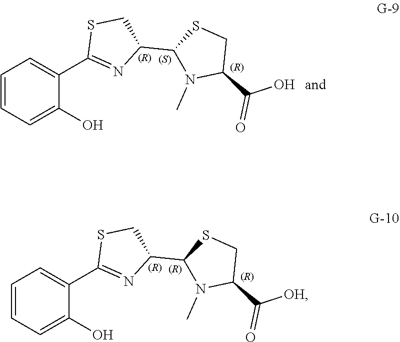

or pharmaceutically acceptable salts thereof, in a molar ratio of about 10:about 90, or about 50:about 50, or about 90:about 10. In further embodiments, the molar ratio of G-9 to G-10 is about 25 to about 55:about 45 to about 75. In other embodiments, the molar ratio of G-9 to G-10 is about 40:about 60. In further embodiments, the molar ratio of G-9 to G-10 is about 25:about 75. In yet other embodiments, the molar ratio of G-9 to G-10 is about 30:about 70. In still further embodiments, the molar ratio of G-9 to G-10 is about 35:about 65.

In other embodiments, the pharmaceutical composition contains

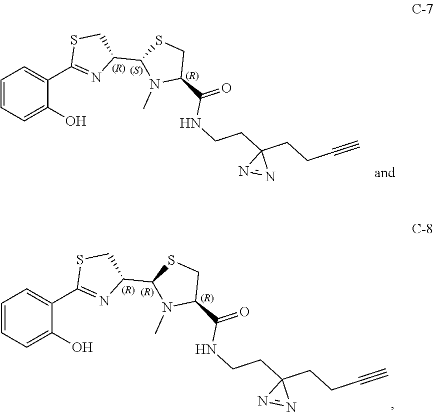

or pharmaceutically acceptable salts thereof, in a molar ratio of about 25 to about 55:about 45 to about 75. In other embodiments, the molar ratio of C-7 to C-8 is about 10:about 90, or about 50:about 50, or about 90:about 10. In further embodiments, the molar ratio of C-7 to C-8 is about 40:about 60. In further embodiments, the molar ratio of to is about 25:about 75. In yet other embodiments, the molar ratio of C-7 to C-8 is about 30:about 70. In still further embodiments, the molar ratio of C-7 to C-8 is about 35:about 65.

In further embodiments, the pharmaceutical composition contains

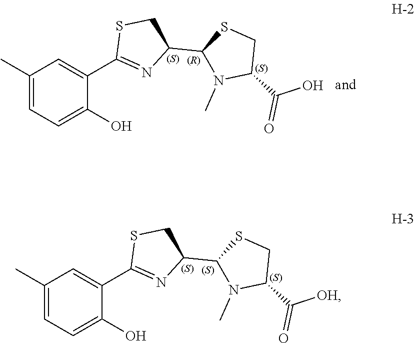

or pharmaceutically acceptable salts thereof, in a molar ratio of about 10:about 90, or about 50:about 50, or about 90:about 10. In further embodiments, the molar ratio of H-2 to H-3 is about 25 to about 55:about 45 to about 75. In other embodiments, the molar ratio of H-2 to H-3 is about 40:about 60. In further embodiments, the molar ratio of H-2 to H-3 is about 25:about 75. In yet other embodiments, the molar ratio of H-2 to H-3 is about 30:about 70. In still further embodiments, the molar ratio of H-2 to H-3 is about 35:about 65.

In still other embodiments, the pharmaceutical composition contains

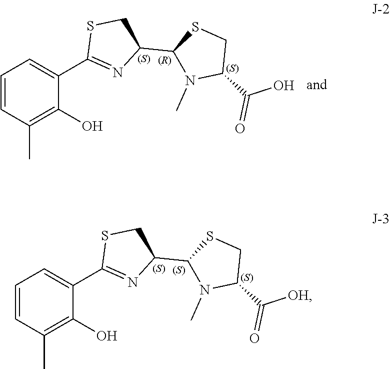

or pharmaceutically acceptable salts thereof, in a molar ratio of about 10:about 90, or about 50:about 50, or about 90:about 10. In further embodiments, the molar ratio of J-2 to J-3 is about 25 to about 55:about 45 to about 75. In other embodiments, the molar ratio of J-2 to J-3 is about 40:about 60. In further embodiments, the molar ratio of J-2 to J-3 is about 25:about 75. In yet other embodiments, the molar ratio of J-2 to J-3 is about 30:about 70. In still further embodiments, the molar ratio of J-2 to J-3 is about 35:about 65.

In yet further embodiments, the pharmaceutical composition contains

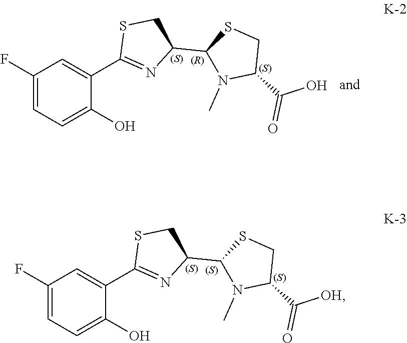

or pharmaceutically acceptable salts thereof, in a molar ratio of about 10:about 90, or about 50:about 50, or about 90:about 10. In further embodiments, the molar ratio of K-2 to K-3 is about 25 to about 55:about 45 to about 75. In other embodiments, the molar ratio of K-2 to K-3 is about 40:about 60. In further embodiments, the molar ratio of K-2 to K-3 is about 25:about 75. In yet other embodiments, the molar ratio of K-2 to K-3 is about 30:about 70. In still further embodiments, the molar ratio of K-2 to K-3 is about 35:about 65.

In other embodiments, the pharmaceutical composition contains

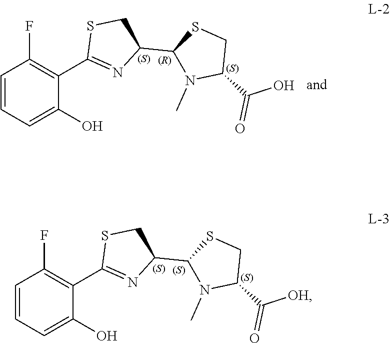

or pharmaceutically acceptable salts thereof, in a molar ratio of about 10:about 90, or about 50:about 50, or about 90:about 10. In further embodiments, the molar ratio of L2 to L-3 is about 25 to about 55:about 45 to about 75. In other embodiments, the molar ratio of L-2 to L-3 is about 40:about 60. In further embodiments, the molar ratio of L-2 to L-3 is about 25:about 75. In yet other embodiments, the molar ratio of L-2 to L-3 is about 30:about 70. In still further embodiments, the molar ratio of L-2 to L-3 is about 35:about 65.

In further embodiments, the pharmaceutical composition contains

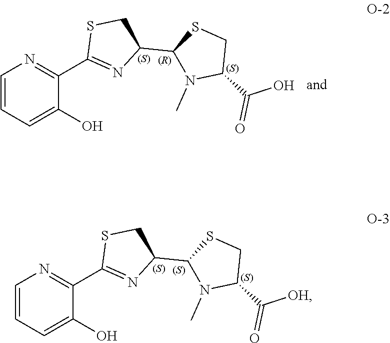

or pharmaceutically acceptable salts thereof, in a molar ratio of about 10:about 90, or about 50:about 50, or about 90:about 10. In further embodiments, the molar ratio of O-2 to O-3 is about 25 to about 55:about 45 to about 75. In other embodiments, the molar ratio of O-2 to O-3 is about 40:about 60. In further embodiments, the molar ratio of O-2 to O-3 is about 25:about 75. In yet other embodiments, the molar ratio of O-2 to O-3 is about 30:about 70. In still further embodiments, the molar ratio of O-2 to O-3 is about 35:about 65.

In yet other embodiments, the pharmaceutical composition contains

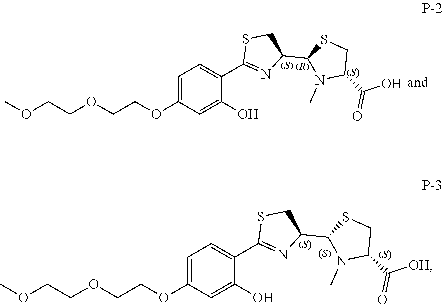

or pharmaceutically acceptable salts thereof, in a molar ratio of about 10:about 90, or about 50:about 50, or about 90:about 10. In further embodiments, the molar ratio of P-2 to P-3 is about 25 to about 55:about 45 to about 75. In other embodiments, the molar ratio of P-2 to P-3 is about 40:about 60. In further embodiments, the molar ratio of P-2 to P-3 is about 25:about 75. In yet other embodiments, the molar ratio of P-2 to P-3 is about 30:about 70. In still further embodiments, the molar ratio of P-2 to P-3 is about 35:about 65.

In still further embodiments, the pharmaceutical composition contains

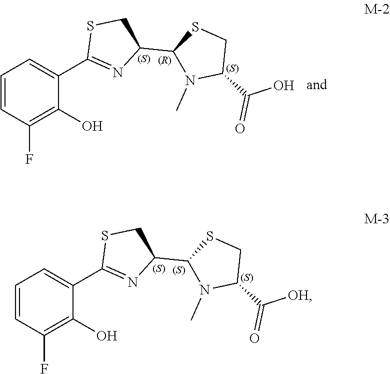

or pharmaceutically acceptable salts thereof, in a molar ratio of about 10:about 90, or about 50:about 50, or about 90:about 10. In further embodiments, the molar ratio of M-2 to M-3 is about 25 to about 55:about 45 to about 75. In other embodiments, the molar ratio of M-2 to M-3 is about 40:about 60. In further embodiments, the molar ratio of M-2 to M-3 is about 25:about 75. In yet other embodiments, the molar ratio of M-2 to M-3 is about 30:about 70. In still further embodiments, the molar ratio of M-2 to M-3 is about 35:about 65.

In still further embodiments, the pharmaceutical composition contains

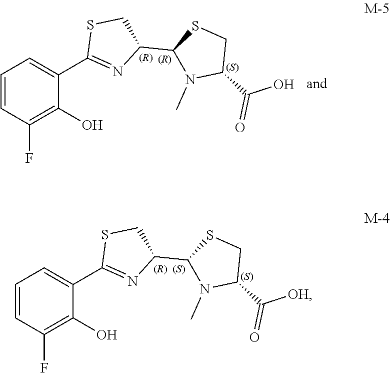

or pharmaceutically acceptable salts thereof, in a molar ratio of about 10:about 90, or about 50:about 50, or about 90:about 10. In further embodiments, the molar ratio of M-5 to M-4 is about 50:about 50.

In still further embodiments, the pharmaceutical composition contains

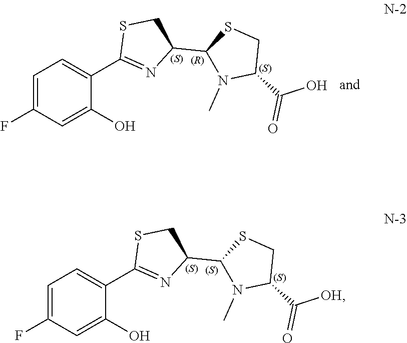

or pharmaceutically acceptable salts thereof, in a molar ratio of about 10:about 90, or about 50:about 50, or about 90:about 10. In further embodiments, the molar ratio of N-2 to N-3 is about 25 to about 55:about 45 to about 75. In other embodiments, the molar ratio of

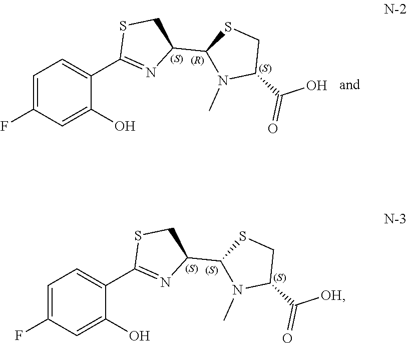

is about 40:about 60. In further embodiments, the molar ratio of N-2 to N-3 is about 25:about 75. In yet other embodiments, the molar ratio of N-2 to N-3 is about 30:about 70. In still further embodiments, the molar ratio of N-2 to N-3 is about 35:about 65.

In still further embodiments, the pharmaceutical composition contains

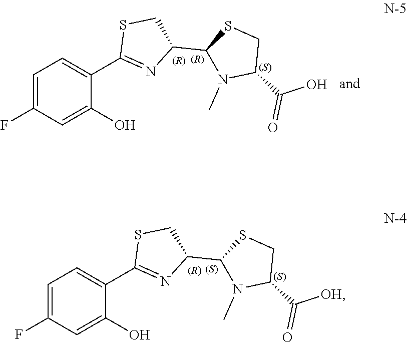

or pharmaceutically acceptable salts thereof, in a molar ratio of about 10:about 90, or about 50:about 50, or about 90:about 10. In further embodiments, the molar ratio of N-5 to N-4 is about 75:about 25.

In addition to the compounds described herein, the pharmaceutically acceptable compositions contain one or more pharmaceutically acceptable excipients. The pharmaceutically acceptable excipient is selected on the basis of the mode and route of administration. Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005); Handbook of Pharmaceutical Excipients, 6^{th} Edition, Rowe et al., Eds., Pharmaceutical Press (2009); and the USP/NF (United States Pharmacopeia and the National Formulary).

In some embodiments, the pharmaceutically acceptable excipient is one or more of an antioxidant, binder, buffer, coloring agent, diluent (e.g., solid or liquid), disintegrant, dispersing agent, dyestuff, filler, emulsifier, flavoring agent, lubricant, pH adjuster, pigment, preservative, stabilizer, solubilizing agent, solvent, suspending agents, sweetener, or wetting agent, or combination thereof.

Examples of suitable excipients include, without limitation, acacia, alginate, calcium phosphate, calcium carbonate, calcium silicate, carbopol gel, carboxymethyl cellulose, carnauba wax, cellulose, crospovidone, dextrose, diacetylated monoglycerides, ethylcellulose, gelatin, glyceryl monostearate 40-50, gum acacia, gum arabic, hydroxyethyl cellulose, hydroxypropyl cellulose, hydroxypropylmethyl cellulose, hypromellose phthalate, hypromellose, lactose, lecithin, magnesium stearate, kaolin, methacrylic acid copolymer type C, mannitol, methyl cellulose, methylhydroxybenzoate, microcrystalline cellulose, povidone, polyethylene glycol, polysorbate 80, polyvinylpyrrolidone, propylhydroxybenzoate, sodium carboxymethyl cellulose sodium hydroxide, sodium stearyl fumarate, sodium starch glycolate, starch, sorbitan monooleate sorbitol, sorbic acid, sucrose, talc, tragacanth, talc, triethyl citrate, titanium dioxide, yellow ferric oxide, talc, oil medium (e.g., peanut oil, liquid paraffin, mineral oil, olive oil, almond oil, glycerin, propylene glycol), or water,

When the excipient serves as a diluent, it can be a solid, semisolid, or liquid material (e.g., normal saline), which acts as a vehicle, carrier or medium for the active ingredient. As is known in the art, the type of diluent can vary depending upon the intended route of administration.

The pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are known in the art. See, e.g., Remington: The Science and Practice of Pharmacy, 21^{st} Ed., Gennaro, Ed., Lippencott Williams & Wilkins (2005), and Encyclopedia of Pharmaceutical Technology, eds. J. Swarbrick and J. C. Boylan, 1988-1999, Marcel Dekker, New York.

### Dosage Units

The disclosure also provides dosage units containing one or more compound or pharmaceutical composition disclosed herein. One skilled in the art would be able to select a dosage for use herein. In some embodiments, the dosage unit may be administered by any means known in the art. For example, the dosage unit may be a solid dosage form, liquid dosage form, or solid/liquid dosage form. In certain aspects, the dosage unit is a solid dosage form. In other aspects, the dosage form is a liquid dosage form.

The dosage unit may be formulated for the delivery that is most useful to the subject. In some embodiments, the dosage unit is for enteral or parenteral administration. Examples of enteral administration include, without limitation, oral, rectal, sublingual, or buccal. In certain embodiments, the dosage units are administered orally.

In other embodiments the dosage unit is for parenteral administration, i.e., a parenteral dosage unit. As used herein, the term “parenteral” refers to routes of administration aside from enteral administration. Examples of parenteral administration include, without limitation, buccal, epicutaneous, epidural, extra-amniotic, intra-arterial, intra-articular, intracardiac, intracavernous, intracerebral, intracerebroventricular, intradermal, intralesional, intramuscular, intraocular, intraosseous infusion, intraperitoneal, intrapulmonary, intrathecal, intrauterine, intravaginal, intravenous, intravesical, intravitreal, nasal, perivascular, subcutaneous, sublingual, transdermal, topical, transepithelial, or transmucosal. Parenteral administration may be by continuous infusion over a selected period of time. In certain embodiments, the dosage unit is administered intravenously, intraperitoneally, intramuscularly, or subcutaneously. In further embodiments, the dosage unit is administered orally, intravenously, intraperitoneally, intramuscularly, or subcutaneously. In further embodiments, the dosage unit is administered orally.

Parenteral dosage units are known in the art and include, without limitation, injectable solutions, inhalants, infusions, patches, and suppositories. In certain aspects, the parenteral dosage unit is an injectable solution. In other embodiments, the dosage unit is formulated for oral delivery, i.e., an oral dosage unit. In certain aspects, the oral dosage unit is a pill (e.g., tablet, caplet, capsule (e.g., soft gelatin, hard gelatin, gel capsule)), effervescent dosage form, elixir, film, liquid/solution (e.g., suspension, emulsion), lollipop, lozenge, paste, powder, sachet, or syrup. In further aspects, the oral dosage unit is a pill, tablet, capsule, syrup, liquid solution, powder, paste, patch, pump, or film. In yet other aspects, the oral dosage unit is a dry product for reconstitution with water or other suitable vehicle before use.

When the dosage form is a solid dosage form, an enteric coating can be applied or the solid dosage form may be scored. An enteric coating can be stable at low pH (e.g., in the stomach) and can dissolve at higher pH (e.g., in the small intestine).

The dosage form may also be a modified release dosage form. The term “modified release” as used herein refers to a dosage form that contains one or more release controlling excipient. The modified release dosage form may be an immediate-, delayed-, extended-, prolonged-, sustained-, pulsatile-, controlled-, extended, accelerated-, fast-, targeted-, programmed-release, or gastric retention dosage form. Suitable modified release dosage vehicles include, but are not limited to, hydrophilic or hydrophobic matrix devices, dosage forms containing water-soluble separating layer coatings, dosage forms containing enteric coatings, osmotic devices, multi-particulate devices, and combinations thereof. These dosage forms can be prepared according to known methods and techniques. See, e.g., Remington: The Science and Practice of Pharmacy, 21^{st} Ed., Gennaro, Ed., Lippencott Williams & Wilkins (2005); Modified-Release Drug Delivery Technology, Rathbone et al., Eds., Drugs and the Pharmaceutical Science, Marcel Dekker, Inc.: New York, N.Y., 2002; Vol. 126, which are incorporated herein by reference in their entireties.

Regardless of the type of dosage unit, it contains may contain a therapeutically effective amount of compound A, compound B, compound C, compound D, compound E, compound F, compound G, compound H, compound J, compound K, compound L, compound M, compound N, compound O, compound P, or compound Q, or a combination thereof.

One of skill in the art can determine a suitable amount of compound A, compound B, compound C, compound D, compound E, compound F, compound G, compound H, compound J, compound K, compound L, compound M, compound N, compound O, compound P, or compound Q to incorporate into the pharmaceutical compositions or dosage units of the disclosure. In certain embodiments, the pharmaceutical composition or dosage unit contains about 0.01 to about 1000 mg of one or more of compound A, compound B, compound C, compound D, compound E, compound F, compound G, compound H, compound J, compound K, compound L, compound M, compound N, compound O, compound P, or compound Q. In other embodiments, the pharmaceutical composition or dosage unit contains about 0.01, about 0.1, about 0.5, about 1, about 5, about 10, about 25, about 50, about 75, about 100, about 125, about 150, about 175, about 200, about 225, 250, about 275, about 300, about 350, about 400, about 450, about 500, about 550, about 600, about 650, about 700, about 750, about 800, about 850, about 900, about 950, or about 1000 mg of one or more of compound A, compound B, compound C, compound D, compound E, compound F, compound G, compound H, compound J, compound K, compound L, compound M, compound N, compound O, compound P, or compound Q. In further embodiments, the pharmaceutical composition or dosage unit contains about 0.01 to about 750, about 0.01 to about 500, about 0.01 to about 250, about 0.01 to about 100, about 0.01 to about 50, about 0.01 to about 25, about 0.01 to about 10, about 0.01 to about 5, about 0.01 to about 0.1, about 0.1 to about 1000, about 0.1 to about 750, about 0.1 to about 500, about 0.1 to about 250, about 0.1 to about 100, about 0.1 to about 50, about 0.1 to about 25, about 0.1 to about 10, about 0.1 to about 5, about 0.1 to about 1, about 1 to about 1000, about 1 to about 750, about 1 to about 500, about 1 to about 250, about 1 to about 100, about 1 to about 50, about 1 to about 25, about 1 to about 10, about 1 to about 5, about 5 to about 1000, about 5 to about 750, about 5 to about 500, about 5 to about 250, about 5 to about 100, about 5 to about 50, about 5 to about 25, about 5 to about 10, about 10 to about 1000, about 10 to about 750, about 10 to about 500, about 10 to about 250, about 10 to about 100, about 10 to about 50, about 10 to about 25, about 25 to about 1000, about 25 to about 750, about 25 to about 500, about 25 to about 250, about 25 to about 100, about 25 to about 50, about 50 to about 1000, about 50 to about 750, about 50 to about 500, about 50 to about 250, about 50 to about 100, about 100 to about 1000, about 100 to about 750, about 100 to about 500, about 100 to about 250, about 250 to about 1000, about 250 to about 750, about 250 to about 500, about 500 to about 1000, about 500 to about 750, or about 750 to about 100 mg.

### Treatment Methods

The compounds, pharmaceutical compositions, and dosage units discussed herein may be used in methods to modulate an inflammatory bowel disease marker. In some embodiments, methods for modulating an inflammatory bowel disease marker are provided and include administering one or more compound disclosed herein to the subject in need thereof. In other embodiments, methods for modulating an inflammatory bowel disease marker are provided and include administering one or more pharmaceutical composition disclosed herein to the subject in need thereof. In further embodiments, methods for modulating an inflammatory bowel disease marker are provided and include administering one or more dosage form disclosed herein to the subject in need thereof.

The term “modulate” or variations thereof, as used herein, refers to an observable change in the level of an inflammatory bowel disease marker in a subject, as measured using techniques and methods known in the art for the measurement of the inflammatory bowel disease. Modulating the inflammatory bowel disease marker level in a subject may result in a change of at least about 1% relative to prior to the marker level at the time of administration of the compounds, pharmaceutical compositions, or dosage forms. In some embodiments, the modulation results in a change of least about 5, about 10, about 15, about 20, about 25, about 30, about 35, about 40, about 45, about 50, about 55, about 60, about 65, about 70, about 75, about 80, about 85, about 90, about 95 or at least about 98% of the inflammatory bowel disease marker or more relative to prior to administration of the pharmaceutical composition. In other embodiments, modulating is increasing the level of an inflammatory bowel disease marker in a subject. In further embodiments, modulating includes decreasing the level of the inflammatory bowel disease marker level in the subject.

The term “inflammatory bowel disease marker” as used herein refers to a diagnostic marker that recognizes that a subject has inflammatory bowel disease. The marker may be a clinical marker (e.g., symptom), a diagnostic marker (e.g., test/scan), or a biochemical indicator (e.g., biomarker). The methods provided herein result in the decrease of at least one inflammatory bowel disease marker. In some embodiments, the methods result in a beneficial change (e.g., reduction or increase) of a clinical marker, diagnostic marker, biochemical indicator, or a combination thereof.

In certain embodiments, the inflammatory bowel disease marker is a diagnostic marker. Examples of diagnostic markers include results obtained from laboratory tests (e.g., blood tests, stool studies), endoscopic procedures (e.g., colonoscopy, flexible sigmoidoscopy, upper endoscopy, capsule endoscopy, or balloon-assisted enteroscopy), or imaging procedures (e.g., X-rays, computerized tomography scan, or magnetic resonance imaging). In certain aspects, the methods result in a positive change in a diagnostic marker. In certain embodiments, the methods result in a positive change in a laboratory test such as a blood test (showing anemia or infection) or stool study (showing blood or organisms). For example, the methods may result in a reduction or absence of anemia, an infection, blood in stool, or organisms in stool. In other aspects, the methods result in a positive change in an endoscopic procedure such as a colonoscopy, flexible sigmoidoscopy, upper endoscopy, capsule endoscopy, or balloon-assisted enteroscopy. For example, the methods result in a reduction in inflammation, tumors, lesions, polyps, fistula, or a perforated colon, as diagnosed by a colonoscopy, flexible sigmoidoscopy, upper endoscopy, capsule endoscopy, or balloon-assisted enteroscopy. Also, e.g., the methods result in a reduction in inflammation, tumors, lesions, polyps, fistula, or a perforated colon, as diagnosed by X-rays, computerized tomography scan, or magnetic resonance imaging. In certain embodiments, the methods result in a reduction of inflammation or ulcers in the intestinal lining of the subject, as observed by colonoscopy.

In other embodiments, the inflammatory bowel disease marker is a clinical marker. Examples of clinical markers of inflammatory bowel disease include, without limitation, ulcers (e.g., intestinal lining), inflammation (e.g., intestinal lining), diarrhea, fatigue, abdominal pain, cramping, blood in stool, reduced appetite, or unintended weight loss, or combinations thereof.

The methods may further result in a positive change in a biochemical indicator, such as a biomarker. Examples of biomarkers include, without limitation, tumor necrosis factor (TNF)-α (e.g., serum), interleukin (IL)-6 (e.g., serum), IL-1β (e.g., serum), nuclear factor κB (NF-κB, e.g., intestinal lining tissue), IL-8 (e.g., intestinal lining tissue), IL-23, IL-12 (e.g., intestinal lining tissue), IL-17A, M2-PK pyruvate kinase (e.g., feces), osteoprotegerin (e.g., intestinal lining tissue, serum, or feces), myeloperoxidase (MPO), high mobility group box-1 (HMGB1, e.g., feces), chitinase 3-like-1 (CHI3L1, e.g., serum or feces), human beta defensin 2 (H1BD2), matrix metalloproteinase (e.g., MMP2 or MMP9, e.g., serum or urine), calprotectin (e.g., feces), lactoferrin, peripheral antineutrophil cytoplasmic antibodies (pANCA, e.g., serum such as in subjects suffering from ulcerative colitis), anti-Saccharomyces cerevisiae antibodies (ASCA, e.g., serum such as in subjects suffering from Crohn's disease), eotaxin-1, monocyte chemoattractant protein-1 (MCP-1, e.g., intestinal lining tissue), macrophage inflammatory proteins (such as MIP-1 and MIP-2, e.g., intestinal lining tissue), regulated upon activation, normal T cell expressed and presumably secreted (RANTES, e.g., intestinal lining tissue), chemokine (C-X-C motif) ligand (CXCL-1, e.g., intestinal lining tissue), cysteine-cysteine motif chemokine ligand 20 (CCL20, e.g., intestinal lining tissue), granulocyte colony-stimulating factor (GCSF, e.g., intestinal lining tissue), granulocyte-macrophage colony-stimulating factor (GM-CSF, e.g., intestinal lining tissue), C-reactive protein (CRP), fibrinogen, cluster of differentiation (such as CD3, CD69, CD80, CD86), C4, sialic acid, C1 activation enzyme, C3a, C5, bradykinin, plasminogen activator, serum amyloid A protein (SAA), prostaglandin E₂ (PGE2), D2, F2/leukotriene A4 (LTA4), B4, C4, D4, cationic protein, oxygen derived free radicals, nitrogen derived free radicals, vasoactive amine (such as histamine or serotonin), integrin/adhesion molecules (such as a/B integrins, Ig superfamily, selectins, cadherins), erythrocyte sedimentation rate, procalcitonin, white blood cell count, immunoglobulin (such as IgA, IgG, or IgM), MHC, or interferon (IFN)-γ (e.g., intestinal lining tissue), or combinations thereof. In some aspects, the biomarker is TNF-α, IL-1β, IL-6, IL-8, IL-23, IL-12, or IL-17A. In further aspects, the biomarker is NF-κB, MCP-1, CCL20, or IFN-γ levels, or a combination thereof. In other aspects, the biomarker is M2-PK, osteoprotegerin, MPO, HMGB1, CHI3L1, HBD2, MMP, p-ANCA, or ASCA, or a combination thereof. In yet further aspects, the biomarker is SAA or eotaxin-1. In further aspects, the methods described herein may be used to reduce TNF-α serum levels, IL-6 serum levels, or IL-1β serum levels, or combinations thereof. In yet other aspects, the methods may be used to reduce NF-κB levels, IL-8 levels, MCP-1 levels, CCL20 levels, or IFNγ levels in the subject. In still further aspects, the methods are useful to reduce NF-κB levels in intestinal lining tissue, IL-8 levels in intestinal lining tissue, MCP-1 levels in intestinal lining tissue, CCL20 levels in intestinal lining tissue, or IFNγ levels in intestinal lining tissue in the subject. In other aspects, the methods described herein may be used to reduce intestinal lining inflammation and/or ulcers in the intestinal lining, as observed by colonoscopy. In further aspects, the methods described herein are useful to reduce NF-κB, MCP-1, CCL20, or IFN-γ levels in the subject. In still other aspects, the methods described herein are useful to reduce TNF-α, IL-1β, IL-6, IL-8, IL-23, IL-12, or IL-17A levels in the subject. In yet further aspects, the methods described herein may be used to reduce M2-PK pyruvate kinase, osteoprotegerin, MPO, HMGB1, CHI3L1, HBD2, MMP, calprotectin, lactoferrin, pANCA, or ASCA levels in the subject. In other aspects, the methods result in a reduction in the activity or level of TNF-α, IL-1β, IL-6, IL-8, IL-23, IL-12, or IL-17A, or a combination thereof, in the subject. In further aspects, the methods result in a reduction in the activity or level of NF-κB, MCP-1, CCL20, or IFN-7, or a combination thereof, in the subject. In yet other aspects, the methods result in a reduction in the activity or level of M2-PK, osteoprotegerin, MPO, HMGB1, CHI3L1, HBD2, MMP, calprotectin, lactoferrin, p-ANCA, or ASCA, or a combination thereof, in the subject. In still further aspects, the methods result in a reduction in the activity or level of one or both of SAA or eotaxin-1 in the subject.

Thus, the compounds disclosed herein may be used to treat inflammatory bowel disease. The term “inflammatory bowel disease” or “IBD” as used herein refers to a disorder that involves chronic inflammation of the digestive tract. IBD includes ulcerative colitis and Crohn's disease. In some embodiments, the IBD is ulcerative colitis. In other embodiments, the IBD is Crohn's disease. In further embodiments, the IBD includes ulcers in the intestinal lining. In yet other embodiments, the IBD includes inflammation of the intestinal lining. The subject may have inflammatory bowel disease or may at risk of developing inflammatory bowel disease. In some embodiments, the patient has or has been diagnosed with inflammatory bowel disease. In other embodiments, the patient is a risk of developing inflammatory bowel disease.

The term “at risk for developing inflammatory bowel disease” as used herein refers to a subject who has a genetic predisposition to developing inflammatory bowel disease. In some embodiments, subject have genetic mutations that cause inflammatory bowel disease. In other embodiments, a subject may have one or more genetic variations that may result in inflammatory bowel disease diagnosis.

Accordingly, the disclosure provides methods of treating inflammatory bowel disease in a subject in need thereof by administering one or more compound, pharmaceutical composition, or dosage unit to the subject in need thereof. In some embodiments, the methods include administering one or more compound disclosed herein to the subject in need thereof. In other embodiments, the methods include administering one or more pharmaceutical compositions disclosed herein to the subject in need thereof. In further embodiments, the methods include administering one or more dosage units to the subject in need thereof.

The methods including administering a therapeutically effective amount of one or more of compound A, compound B, compound C, compound D, compound E, compound F, compound G, compound H, compound J, compound K, compound L, compound M, compound N, compound O, compound P, or compound Q. In some embodiments, the therapeutically effective amount is about 0.01 to about 1000 mg/day. In other embodiments, the therapeutically effective amount is about 0.01 to about 750, about 0.01 to about 500, about 0.01 to about 250, about 0.01 to about 100, about 0.01 to about 50, about 0.01 to about 25, about 0.01 to about 10, about 0.01 to about 5, about 0.01 to about 0.1, about 0.1 to about 1000, about 0.1 to about 750, about 0.1 to about 500, about 0.1 to about 250, about 0.1 to about 100, about 0.1 to about 50, about 0.1 to about 25, about 0.1 to about 10, about 0.1 to about 5, about 0.1 to about 1, about 1 to about 1000, about 1 to about 750, about 1 to about 500, about 1 to about 250, about 1 to about 100, about 1 to about 50, about 1 to about 25, about 1 to about 10, about 1 to about 5, about 5 to about 1000, about 5 to about 750, about 5 to about 500, about 5 to about 250, about 5 to about 100, about 5 to about 50, about 5 to about 25, about 5 to about 10, about 10 to about 1000, about 10 to about 750, about 10 to about 500, about 10 to about 250, about 10 to about 100, about 10 to about 50, about 10 to about 25, about 25 to about 1000, about 25 to about 750, about 25 to about 500, about 25 to about 250, about 25 to about 100, about 25 to about 50, about 50 to about 1000, about 50 to about 750, about 50 to about 500, about 50 to about 250, about 50 to about 100, about 100 to about 1000, about 100 to about 750, about 100 to about 500, about 100 to about 250, about 250 to about 1000, about 250 to about 750, about 250 to about 500, about 500 to about 1000, about 500 to about 750, or about 750 to about 100 mg/day.

The methods described herein include administering compound A, compound B, compound C, compound D, compound E, compound F, compound G, compound H, compound J, compound K, compound L, compound M, compound N, compound O, compound P, or compound Q (optionally in a pharmaceutical composition) with an additional agent known to be therapeutically useful. In some embodiments, the additional agent is useful for treating inflammatory bowel disease. In other embodiments, the additional agent is anti-inflammatory drugs, immune system suppressors, biologics, antibiotics, anti-diarrheal medications, pain relievers, or supplements. Examples of anti-inflammatory drugs include, without limitation, corticosteroids and/or aminosalicylates, (e.g., mesalamine, balsalazide, or olsalazine). Examples of immune system suppressors include, without limitation, azathioprine, mercaptopurine, and methotrexate. Examples of biologics include, without limitation, infliximab, adalimumab, golimumab, certolizumab, vedolizumab, and ustekinumab. Examples of antibiotics include, without limitation, ciprofloxacin and metronidazole. Examples of anti-diarrheal medications include fiber supplement (e.g., psyllium powder or methylcellulose) or loperamide. An examples of a pain reliever is acetaminophen.

The methods may also include administering a therapeutically effective amount of one or more of compound A, compound B, compound C, compound D, compound E, compound F, compound G, compound H, compound J, compound K, compound L, compound M, compound N, compound O, compound P, or compound Q. In some embodiments, the therapeutically effective amount is about 0.01 to about 1000 mg/day. In other embodiments, the therapeutically effective amount is about 0.01 to about 750, about 0.01 to about 500, about 0.01 to about 250, about 0.01 to about 100, about 0.01 to about 50, about 0.01 to about 25, about 0.01 to about 10, about 0.01 to about 5, about 0.01 to about 0.1, about 0.1 to about 1000, about 0.1 to about 750, about 0.1 to about 500, about 0.1 to about 250, about 0.1 to about 100, about 0.1 to about 50, about 0.1 to about 25, about 0.1 to about 10, about 0.1 to about 5, about 0.1 to about 1, about 1 to about 1000, about 1 to about 750, about 1 to about 500, about 1 to about 250, about 1 to about 100, about 1 to about 50, about 1 to about 25, about 1 to about 10, about 1 to about 5, about 5 to about 1000, about 5 to about 750, about 5 to about 500, about 5 to about 250, about 5 to about 100, about 5 to about 50, about 5 to about 25, about 5 to about 10, about 10 to about 1000, about 10 to about 750, about 10 to about 500, about 10 to about 250, about 10 to about 100, about 10 to about 50, about 10 to about 25, about 25 to about 1000, about 25 to about 750, about 25 to about 500, about 25 to about 250, about 25 to about 100, about 25 to about 50, about 50 to about 1000, about 50 to about 750, about 50 to about 500, about 50 to about 250, about 50 to about 100, about 100 to about 1000, about 100 to about 750, about 100 to about 500, about 100 to about 250, about 250 to about 1000, about 250 to about 750, about 250 to about 500, about 500 to about 1000, about 500 to about 750, or about 750 to about 100 mg/day.

The methods described herein may further include administering compound A, compound B, compound C, compound D, compound E, compound F, compound G, compound H, compound J, compound K, compound L, compound M, compound N, compound O, compound P, or compound Q (optionally in a pharmaceutical composition) with an additional agent known to be therapeutically useful. In some embodiments, the additional agent is useful for treating inflammatory bowel disease. In other embodiments, the additional agent is anti-inflammatory drugs, immune system suppressors, biologics, antibiotics, anti-diarrheal medications, pain relievers, or supplements. Examples of anti-inflammatory drugs include, without limitation, corticosteroids and/or aminosalicylates, (e.g., mesalamine, balsalazide, or olsalazine). Examples of immune system suppressors include, without limitation, azathioprine, mercaptopurine, and methotrexate. Examples of biologics include, without limitation, infliximab, adalimumab, golimumab, certolizumab, vedolizumab, and ustekinumab. Examples of antibiotics include, without limitation, ciprofloxacin and metronidazole. Examples of anti-diarrheal medications include fiber supplement (e.g., psyllium powder or methylcellulose) or loperamide. An example of a pain reliever is acetaminophen.

In certain embodiments, the additional agent may be a therapy, i.e., non-pharmacological. For example, the subject may require nutritional support, i.e., a special diet, or be administered food enterally (e.g., feeding tube) or parenterally (e.g., injection of nutrients).

### Aspects A

Aspect A1. A compound that is compound A or compound G:

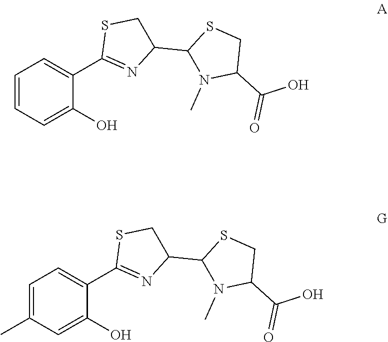 [SMILES: CN1C(C(=O)O)CSC1C1CSC(c2ccccc2O)=N1 ; Cc1ccc(C2=NC(C3SCC(C(=O)O)N3C)CS2)c(O)c1]

or a pharmaceutically acceptable salt thereof

Aspect A2. The compound of Aspects A1, that is compound A, or a pharmaceutically acceptable salt thereof.

Aspect A3. The compound of Aspect A1 or A2, wherein the compound is

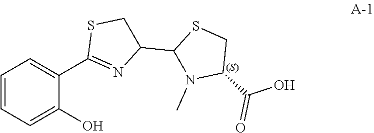

or a pharmaceutically acceptable salt thereof.

Aspect A4. The compound of any one of Aspects A1-A3, wherein the compound is

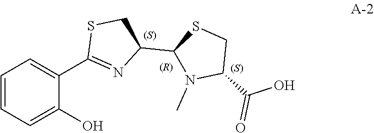

or a pharmaceutically acceptable salt thereof.

Aspect A5. The compound of any one of Aspects A1-A3, wherein the compound is

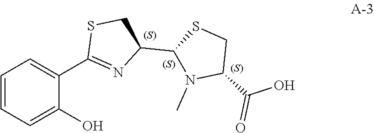

or a pharmaceutically acceptable salt thereof.

Aspect A6. The compound of any one of Aspects A1-A3, wherein the compound is

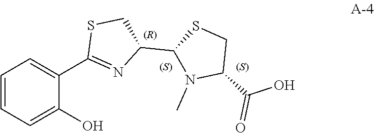

or a pharmaceutically acceptable salt thereof.

Aspect A7. The compound of any one of Aspects A1-A3, wherein the compound is

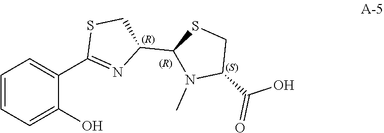

or a pharmaceutically acceptable salt thereof.

Aspect A8. The compound of Aspect A1, that is compound G, or a pharmaceutically acceptable salt thereof.

Aspect A9. The compound of Aspect A1 or A38, that is

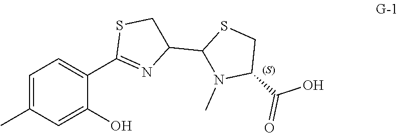

or a pharmaceutically acceptable salt thereof.

Aspect A10. The compound of any one of Aspects A1, A8, or A9 that is

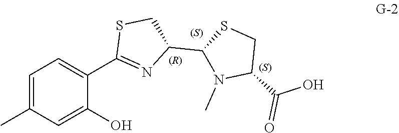

or a pharmaceutically acceptable salt thereof.

Aspect A11. The compound of any one of Aspects A1, A8, or A9 that is

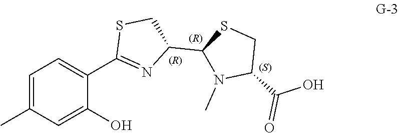

or a pharmaceutically acceptable salt thereof.

Aspect A12. The compound of any one of Aspects A1, A8, or A9 that is

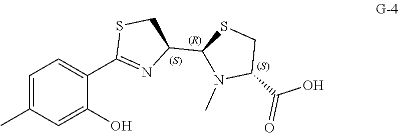

or a pharmaceutically acceptable salt thereof.

Aspect A13. The compound of any one of Aspects A1, A8, or A9 that is

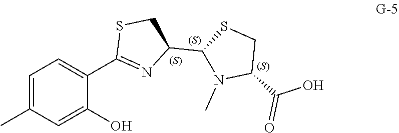

or a pharmaceutically acceptable salt thereof.

Aspect A14. A pharmaceutical composition comprising a therapeutically effective amount of one or more of the compound of any one of Aspects A1-A13 and a pharmaceutically acceptable excipient.

Aspect A15. The pharmaceutical composition of Aspect A14, comprising:

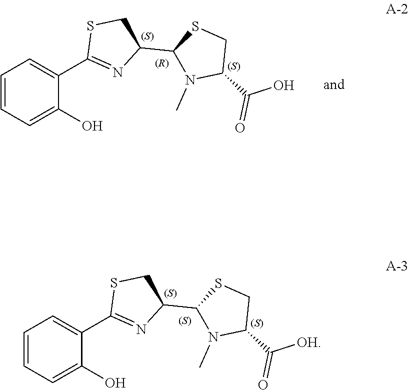

Aspect A16. The pharmaceutical composition of Aspect A15, wherein the molar ratio of

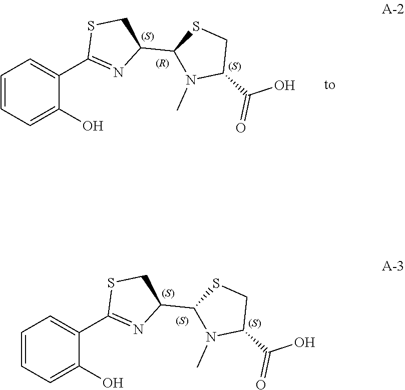

is about 40 to about 60.

Aspect A17. The pharmaceutical composition of Aspect A44, comprising

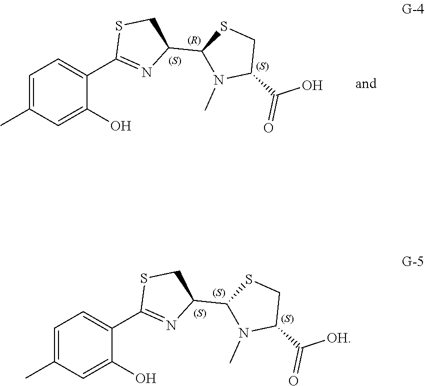

Aspect A18. The pharmaceutical composition of Aspect A48, wherein the molar ratio of

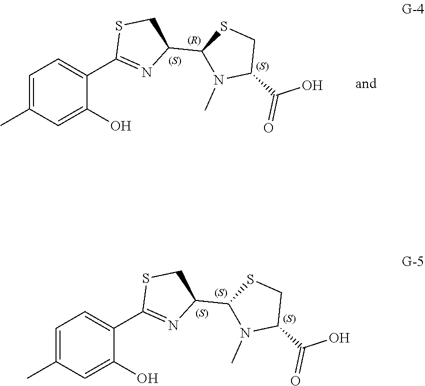

is about 40 to about 60.

Aspect A19. A dosage unit comprising compound of any one of Aspects A1-A13 or the pharmaceutical composition of any one of Aspects A14-A18.

Aspect A20. The dosage of unit of Aspect A19, that is an oral dosage unit such as a pill, tablet, capsule, syrup, liquid solution, powder, paste, or film or a parenteral dosage unit such as an injectable solution.

Aspect A21. The dosage unit of Aspect A19 or A20, comprising about 0.01 to about 1000 mg of the compound.

Aspect A22. The dosage unit of any one of Aspects A19-A21 that is a solid dosage form.

Aspect A23. The dosage unit of any one of Aspects A19-A21 that is a liquid dosage form.

Aspect A24. The method of any one of Aspects A24-A26, wherein the composition is administered orally, intravenously, intraperitoneally, intramuscularly, or subcutaneously.

Aspect A25. The method of Aspect A27, wherein the composition is administered orally.

Aspect A26. The method of any one of Aspects A24-A28, wherein bradykinesia frequency, bradykinesia intensity, resting tremor, muscle rigidity, or a combination thereof is reduced in the subject.

Aspect A27. The method of any one of Aspects A24-A29, wherein the subject's score on MDS-UPDRS scale is improved after administration of the pharmaceutical composition or the dosage unit.

### Aspects B

Aspect B1. A compound that is compound A or compound G:

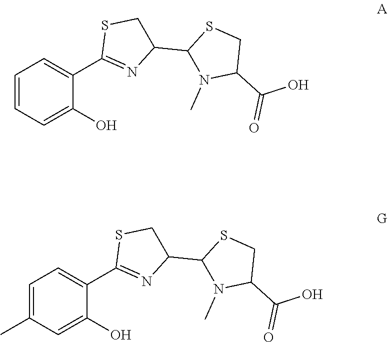 [SMILES: CN1C(C(=O)O)CSC1C1CSC(c2ccccc2O)=N1 ; Cc1ccc(C2=NC(C3SCC(C(=O)O)N3C)CS2)c(O)c1]

or a pharmaceutically acceptable salt thereof

Aspect B2. The compound of Aspect B1, that is compound A, or a pharmaceutically acceptable salt thereof.

Aspect B3. The compound of Aspect B1 or B2, wherein the compound is

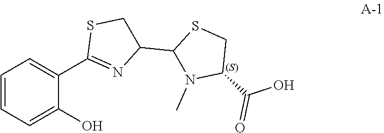

or a pharmaceutically acceptable salt thereof.

Aspect B4. The compound of any one of Aspects B1-B3, wherein the compound is

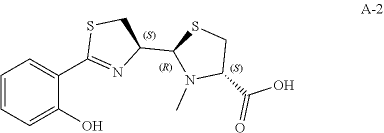

or a pharmaceutically acceptable salt thereof.

Aspect B5. The compound of any one of Aspects B1-B3, wherein the compound is

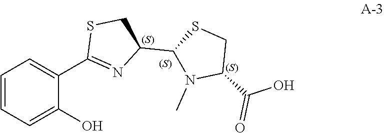

or a pharmaceutically acceptable salt thereof.

Aspect B6. The compound of any one of Aspects B1-B3, wherein the compound is

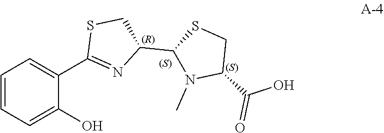

or a pharmaceutically acceptable salt thereof.

Aspect B7. The compound of any one of Aspects B1-B3, wherein the compound is

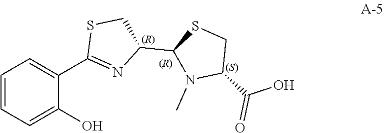

or a pharmaceutically acceptable salt thereof.

Aspect B8. The compound of Aspect B1, that is compound G, or a pharmaceutically acceptable salt thereof.

Aspect B9. The compound of Aspect B1 or B8, that is

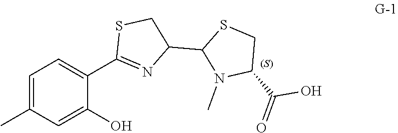

or a pharmaceutically acceptable salt thereof

Aspect B10. The compound of any one of Aspects B1, B8, or B9 that is

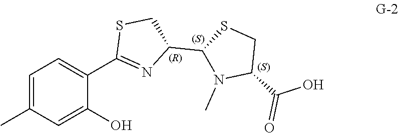

or a pharmaceutically acceptable salt thereof.

Aspect B11. The compound of any one of Aspects B1, B8, or B9 that is

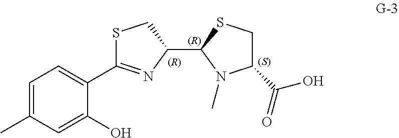

or a pharmaceutically acceptable salt thereof.

Aspect B12. The compound of any one of Aspects B1, B8, or B9 that is

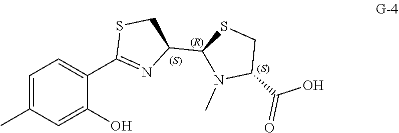

or a pharmaceutically acceptable salt thereof.

Aspect B13. The compound of any one of Aspects B1, B8, or B9 that is

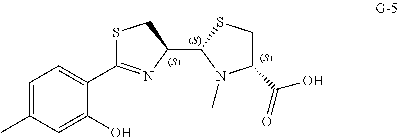

or a pharmaceutically acceptable salt thereof.

Aspect B14. A pharmaceutical composition comprising a therapeutically effective amount of one or more compound of any one of Aspects B1-B13 and a pharmaceutically acceptable excipient.

Aspect B15. The pharmaceutical composition of Aspect B14, comprising:

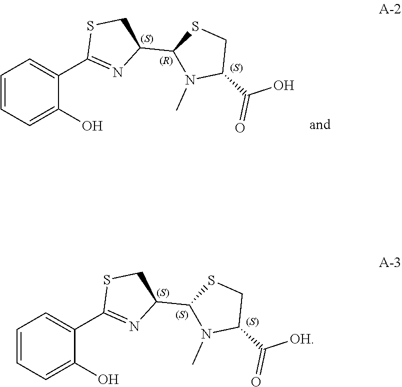

Aspect B16. The pharmaceutical composition of Aspect B15, wherein the molar ratio of

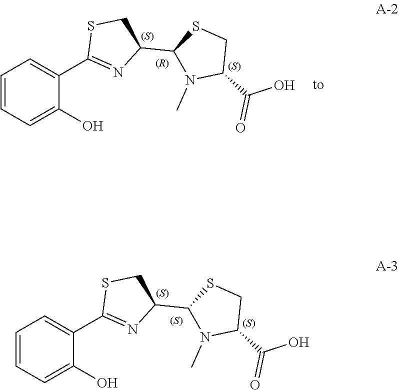

is about 40 to about 60.

Aspect B17. The pharmaceutical composition of Aspect B14, comprising

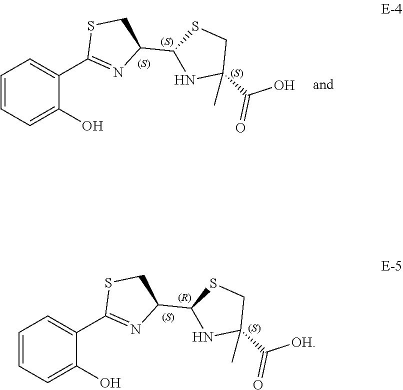

Aspect B18. The pharmaceutical composition of Aspect B14, comprising

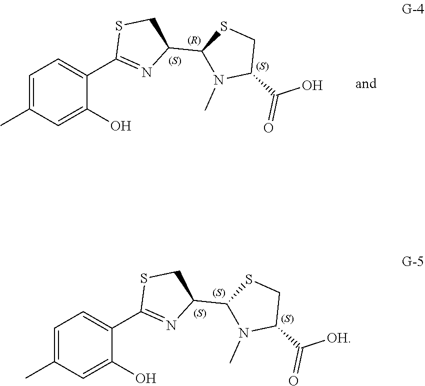

Aspect B19. The pharmaceutical composition of Aspects B18, wherein the molar ratio of

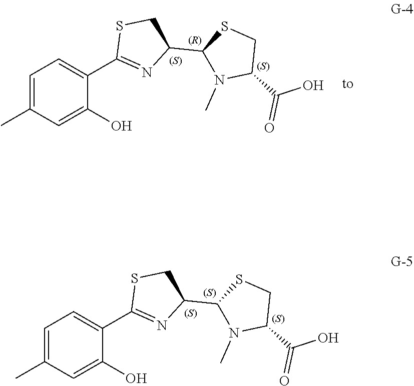

is about 40 to about 60.

Aspect B20. A dosage unit comprising compound of any one of Aspects B1-B13 or the pharmaceutical composition of any one of Aspects B14-B19.

Aspect B21. The dosage of unit of Aspects B20, that is an oral dosage unit such as a pill, tablet, capsule, syrup, liquid solution, powder, paste, or film or a parenteral dosage unit such as an injectable solution.

Aspect B22. The dosage unit of Aspects B20 or B21, comprising about 0.01 to about 1000 mg of the compound.

Aspect B23. The dosage unit of any one of Aspects B20-B22 that is a solid dosage form.

Aspect B24. The dosage unit of any one of Aspects B20-B22 that is a liquid dosage form.

Aspect B25. A method of modulating an inflammatory bowel disease (IBD) marker in a subject in need thereof, comprising administering the compound of any one of Aspects B1-B13, the pharmaceutical composition of any one of Aspects B14-B19, or the dosage form of any one of Aspects B20-B24 to the subject in need thereof.

Aspect B26. A method of treating an inflammatory bowel disease (IBD) in a subject in need thereof, comprising administering to the subject the compound of any one of Aspects B1-B13, the pharmaceutical composition of any one of Aspects B14-B19, or the dosage form of any one of Aspects B20-B24 to the subject in need thereof.

Aspect B27. The method of Aspects B25 or B26, wherein the subject has or is at risk of developing IBD.

Aspect B28. The method of any one of Aspects B25-B27, wherein the pharmaceutical composition or dosage unit is administered orally, intravenously, intraperitoneally, intramuscularly, or subcutaneously.

Aspect B29. The method of Aspect B28, wherein the pharmaceutical composition or dosage form is administered orally.

Aspect B30. The method of any one of Aspects B25-B29, wherein the IBD is ulcerative colitis.

Aspect B31. The method of any one of Aspects B25-B29, wherein the IBD is Crohn's disease.

Aspect B32. The method of any one of Aspects B25-B31, wherein inflammation or ulcers in the intestinal lining of the subject are reduced, as observed by colonoscopy.

Aspect B33. The method of any one of Aspects B25-B32, wherein tumor necrosis factor alpha (TNF-α), interleukin-1β (IL-1β), interleukin-6 (IL-6), interleukin-8 (IL-8), interleukin-23 (IL-23), interleukin 12 (IL-12), or interleukin-17A (IL-17A) levels, or a combination thereof, in the subject are reduced.

Aspect B34. The method of any one of Aspects B25-B33, wherein nuclear factor kappa B (NF-κB), monocyte chemoattractant protein-1 (MCP-1), cysteine-cysteine motif chemokine ligand 20 (CCL20), or interferon-γ (IFN-γ) levels, or a combination thereof, in the subject are reduced.

Aspect B35. The method of any one of Aspects B25-B34, wherein M2 pyruvate kinase (M2-PK), osteoprotegerin, myeloperoxidase (MPO), high mobility group box protein 1 (HMGB1), chitinase 3 like 1 (CHI3L1), human beta defensin 2 (HBD2), metalloproteinase (MMP), calprotectin, lactoferrin, peripheral antineutrophil cytoplasmic antibodies (p-ANCA), or anti-Saccharomyces cerevisiae antibodies (ASCA) levels, or a combination thereof, in the subject are reduced.

Aspect B36. The method of any one of Aspects B25-B35, wherein one or both serum amyloid A protein (SAA) or eotaxin-1 levels are reduced in the subject.

### Aspects C

Aspect C1. A compound that is compound A, compound B, compound C, compound D, compound E, compound F, or compound G:

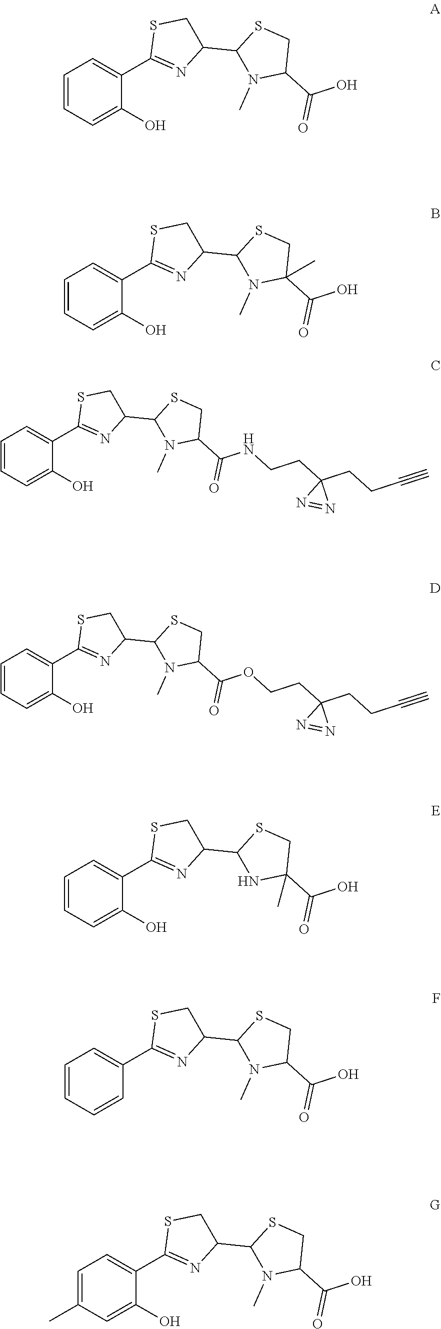

or a pharmaceutically acceptable salt thereof.

Aspect C2. The compound of Aspect C1, that is compound A, or a pharmaceutically acceptable salt thereof.

Aspect C3. The compound of Aspect C1 or C2, wherein the compound is

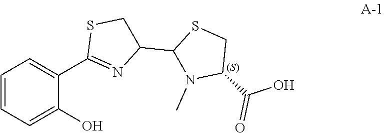

or a pharmaceutically acceptable salt thereof.

Aspect C4. The compound of any one of Aspects C₁-C₃, wherein the compound is

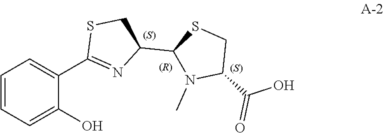

or a pharmaceutically acceptable salt thereof.

Aspect C5. The compound of any one of Aspects C₁-C₃, wherein the compound is

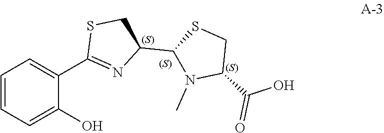

or a pharmaceutically acceptable salt thereof.

Aspect C6. The compound of any one of Aspects C₁-C₃, wherein the compound is

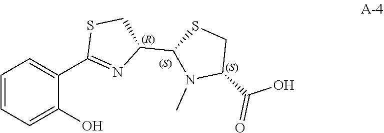

or a pharmaceutically acceptable salt thereof.

Aspect C7. The compound of any one of Aspects C₁-C₃, wherein the compound is

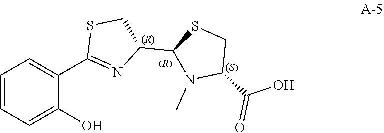

or a pharmaceutically acceptable salt thereof.

Aspect C8. The compound of Aspect C1, that is compound B, or a pharmaceutically acceptable salt thereof.

Aspect C9. The compound of Aspect C8, wherein the compound is

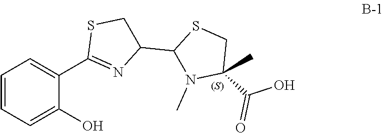

or a pharmaceutically acceptable salt thereof.

Aspect C10. The compound of any one of Aspects C1, C8, or C9, wherein the compound is

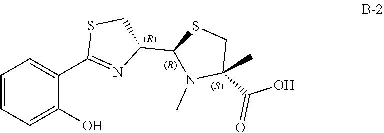

or a pharmaceutically acceptable salt thereof.

Aspect C11. The compound of any one of Aspects C1, C8, or C9, wherein the compound is

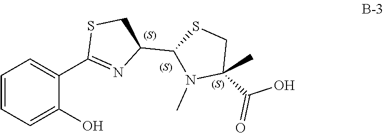

or a pharmaceutically acceptable salt thereof.

Aspect C12. The compound of any one of Aspects C1, C8, or C9, wherein the compound is

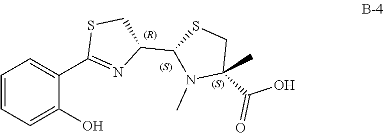

or a pharmaceutically acceptable salt thereof.

Aspect C13. The compound of any one of Aspects C1, C8, or C9, wherein the compound is

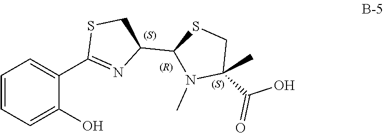

or a pharmaceutically acceptable salt thereof.

Aspect C14. The compound of Aspect C1, that is compound C, or a pharmaceutically acceptable salt thereof.

Aspect C15. The compound of Aspect C1 or C14, wherein the compound is:

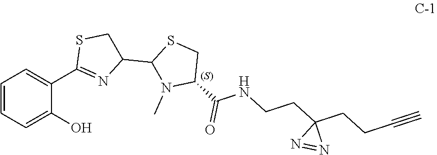

or a pharmaceutically acceptable salt thereof.

Aspect C16. The compound of any one of Aspects C1, C14, or 1C5, that is

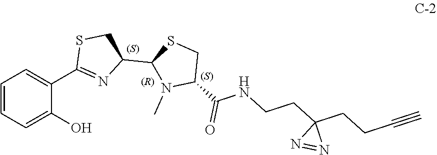

or a pharmaceutically acceptable salt thereof.

Aspect C17. The compound of any one of Aspects C1, C14, or C15, that is:

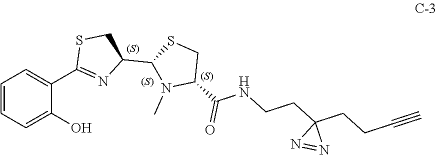

or a pharmaceutically acceptable salt thereof.

Aspect C18. The compound of any one of Aspects C1, C14, or C15, that is:

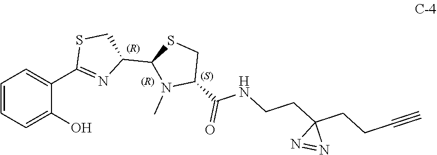

or a pharmaceutically acceptable salt thereof.

Aspect C19. The compound of any one of Aspects C1, C14, or C15, that is:

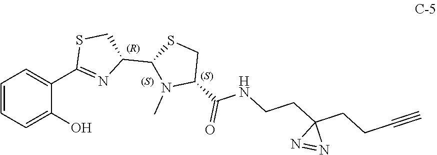

or a pharmaceutically acceptable salt thereof.

Aspect C20. The compound of Aspect C1, that is compound D, or a pharmaceutically acceptable salt thereof.

Aspect C21. The compound of Aspect C1 or C20, that is

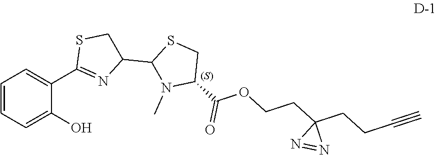

or a pharmaceutically acceptable salt thereof.

Aspect C22. The compound of any one of Aspects C1, C20, or C21, that is:

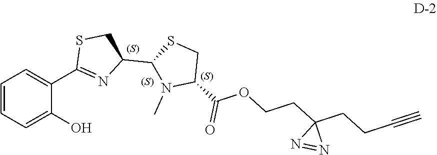

or a pharmaceutically acceptable salt thereof

Aspect C23. The compound of any one of Aspects C1, 20, or 21, that is:

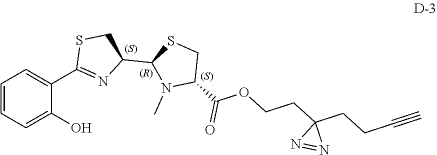

or a pharmaceutically acceptable salt thereof.

Aspect C24. The compound of any one of Aspects C1, C20, or C21, that is:

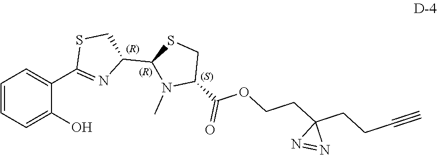

or a pharmaceutically acceptable salt thereof.

Aspect C25. The compound of any one of Aspects C1, C20, or C21, that is:

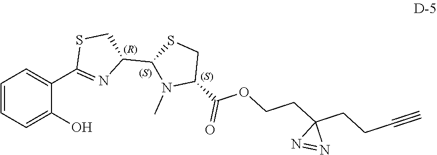

or a pharmaceutically acceptable salt thereof.

Aspect C26. The compound of Aspect C1, that is compound E, or a pharmaceutically acceptable salt thereof.

Aspect C27. The compound of Aspect C1 or C26, that is

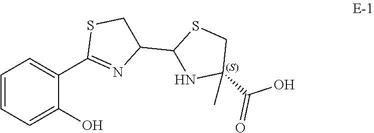

or a pharmaceutically acceptable salt thereof.

Aspect C28. The compound of any one of Aspects C1, C26, or C27 that is

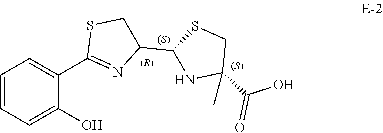

or a pharmaceutically acceptable salt thereof.

Aspect C29. The compound of any one of Aspects C1, C26, or C27 that is

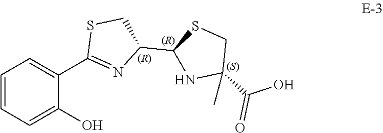

or a pharmaceutically acceptable salt thereof.

Aspect C30. The compound of any one of Aspects C1, C26, or C27 that is

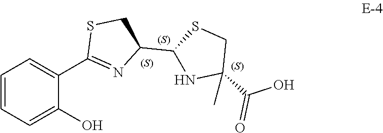

or a pharmaceutically acceptable salt thereof.

Aspect C31. The compound of any one of Aspects C1, C26, or C27 that is

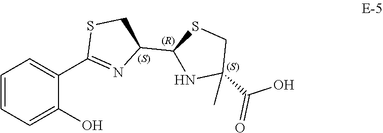

or a pharmaceutically acceptable salt thereof.

Aspect C32. The compound of Aspect C1, that is compound F, or a pharmaceutically acceptable salt thereof.

Aspect C33. The compound of Aspect C1 or C32, that is

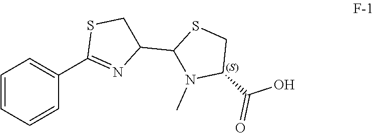

or a pharmaceutically acceptable salt thereof.

Aspect C34. The compound of any one of Aspects C1, C32, or C33 that is

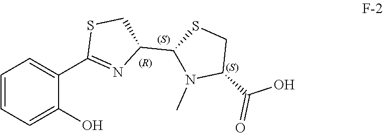

or a pharmaceutically acceptable salt thereof.

Aspect C35. The compound of any one of Aspects C1, C32, or C33 that is

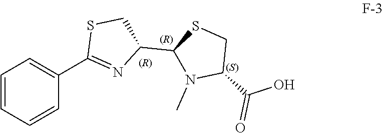

or a pharmaceutically acceptable salt thereof.

Aspect C36. The compound of any one of Aspects C1, C32, or C33 that is

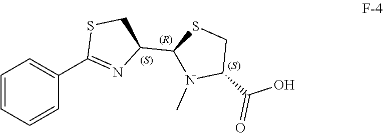

or a pharmaceutically acceptable salt thereof.

Aspect C37. The compound of any one of Aspects C1, C32, or C33 that is

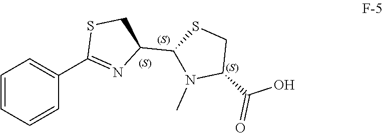

or a pharmaceutically acceptable salt thereof.

Aspect C38. The compound of Aspect C1, that is compound G, or a pharmaceutically acceptable salt thereof.

Aspect C39. The compound of Aspect C1 or C38, that is

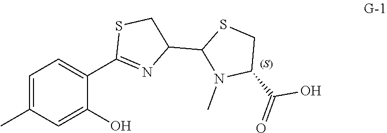

or a pharmaceutically acceptable salt thereof.

Aspect C40. The compound of any one of Aspects C1, C38, or C39 that is

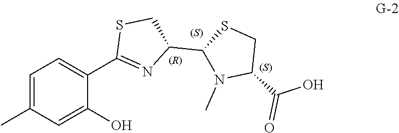

or a pharmaceutically acceptable salt thereof.

Aspect C41. The compound of any one of Aspects C1, C38, or C39 that is

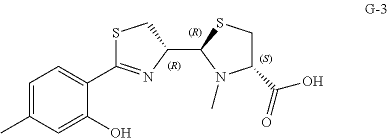

or a pharmaceutically acceptable salt thereof.

Aspect C42. The compound of any one of Aspects C1, C38, or C39 that is

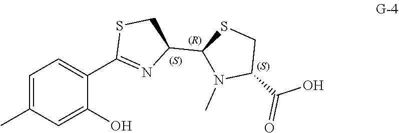

or a pharmaceutically acceptable salt thereof.

Aspect C43. The compound of any one of Aspects C1, C38, or C39 that is

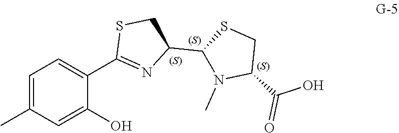

or a pharmaceutically acceptable salt thereof.

Aspect C44. A pharmaceutical composition comprising a therapeutically effective amount of one or more of the compound of any one of Aspects C1-C43 and a pharmaceutically acceptable excipient.

Aspect C45. The pharmaceutical composition of Aspect C44, comprising:

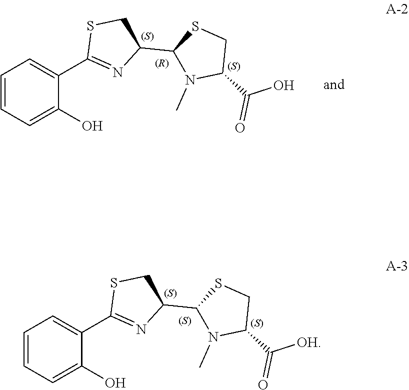

Aspect C46. The pharmaceutical composition of Aspect C45, wherein the molar ratio of

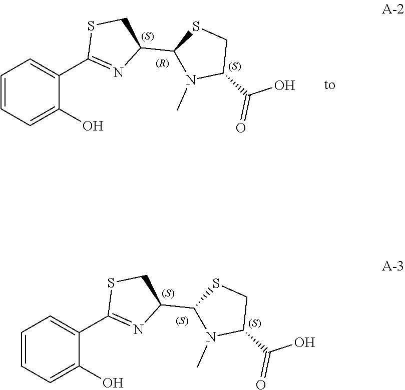

is about 40 to about 60.

Aspect C47. The pharmaceutical composition of Aspect C44, comprising

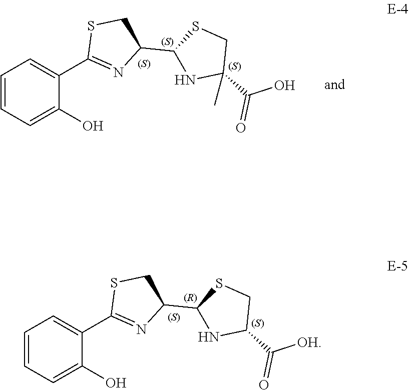

Aspect C48. The pharmaceutical composition of Aspect C44, comprising

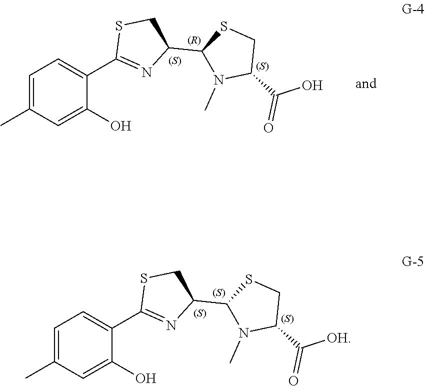

Aspect C49. The pharmaceutical composition of Aspect C48, wherein the molar ratio of

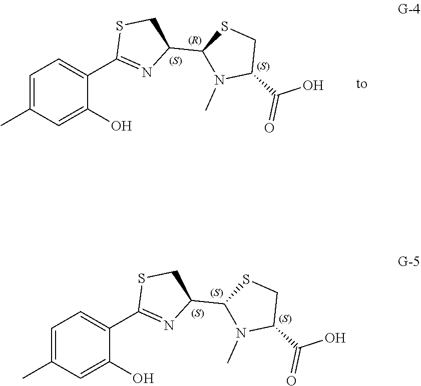

is about 40 to about 60.

Aspect C50. A dosage unit comprising compound of any one of Aspects C1-C43 or the pharmaceutical composition of any one of Aspects C44-C49.

Aspect C51. The dosage of unit of Aspect C50, that is an oral dosage unit such as a pill, tablet, capsule, syrup, liquid solution, powder, paste, or film or a parenteral dosage unit such as an injectable solution.

Aspect C52. The dosage unit of Aspect C50 or C51, comprising about 0.01 to about 1000 mg of the compound.

Aspect C53. The dosage unit of any one of Aspects C50-C52 that is a solid dosage form.

Aspect C54. The dosage unit of any one of Aspects C50-C52 that is a liquid dosage form.

Aspect C55. The method of any one of Aspects C55-C57, wherein the composition is administered orally, intravenously, intraperitoneally, intramuscularly, or subcutaneously.

Aspect C56. The method of Aspect C58, wherein the composition is administered orally.

Aspect C57. The method of any one of Aspects C55-C59, wherein bradykinesia frequency, bradykinesia intensity, resting tremor, muscle rigidity, or a combination thereof is reduced in the subject.

Aspect C58. The method of any one of Aspects C55-C60, wherein the subject's score on MDS-UPDRS scale is improved after administration of the pharmaceutical composition or the dosage unit.

### Aspects D

Aspect D1. A compound that is compound A, compound B, compound C, compound D, compound E, compound F, or compound G:

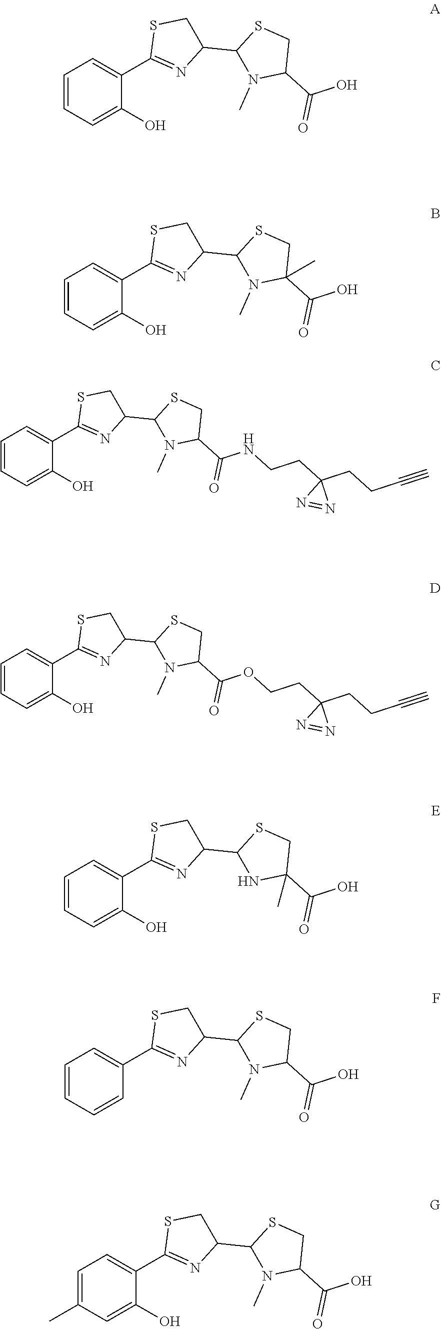

or a pharmaceutically acceptable salt thereof.

Aspect D2. The compound of Aspect D1, that is compound A, or a pharmaceutically acceptable salt thereof.

Aspect D3. The compound of Aspect D1 or D2, wherein the compound is

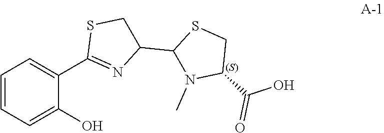

or a pharmaceutically acceptable salt thereof.

Aspect D4. The compound of any one of Aspects D1-D3, wherein the compound is

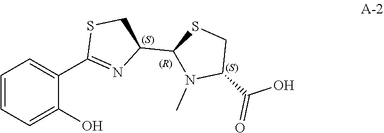

or a pharmaceutically acceptable salt thereof.

Aspect D5. The compound of any one of Aspects D1-D3, wherein the compound is

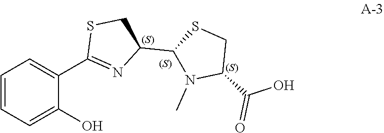

or a pharmaceutically acceptable salt thereof.

Aspect D6. The compound of any one of Aspects D1-D3, wherein the compound is

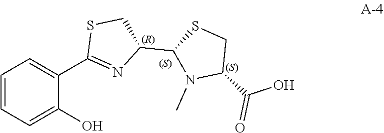

or a pharmaceutically acceptable salt thereof.

Aspect D7. The compound of any one of Aspects D1-D3, wherein the compound is

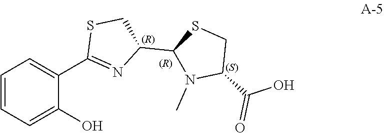

or a pharmaceutically acceptable salt thereof.

Aspect D8. The compound of Aspect D1, that is compound B, or a pharmaceutically acceptable salt thereof.

Aspect D9. The compound of Aspect D8, wherein the compound is

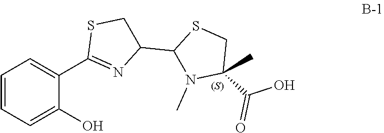

or a pharmaceutically acceptable salt thereof.

Aspect D10. The compound of any one of Aspects D1, D8, or D9, wherein the compound is

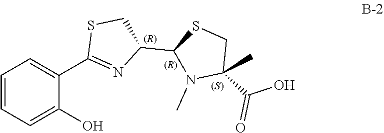

or a pharmaceutically acceptable salt thereof.

Aspect D11. The compound of any one of Aspects D1, D8, or D9, wherein the compound is

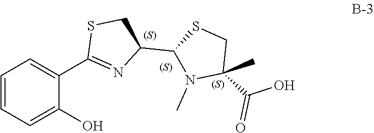

or a pharmaceutically acceptable salt thereof.

Aspect D12. The compound of any one of Aspects D1, D8, or D9, wherein the compound is

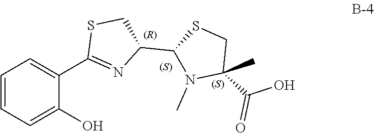

or a pharmaceutically acceptable salt thereof.

Aspect D13. The compound of any one of Aspects D1, D8, or D9, wherein the compound is

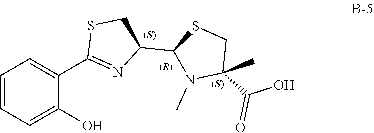

or a pharmaceutically acceptable salt thereof.

Aspect D14. The compound of Aspect 1, that is compound C, or a pharmaceutically acceptable salt thereof.

Aspect D15. The compound of Aspect D1 or D14, wherein the compound is:

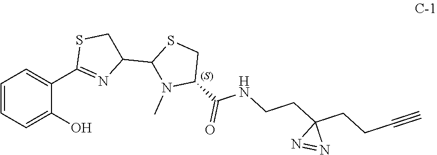

or a pharmaceutically acceptable salt thereof.

Aspect D16. The compound of any one of Aspects D1, D14, or D15, that is

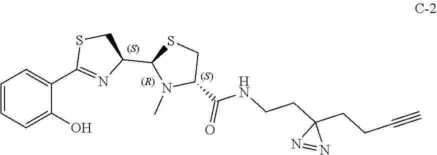

or a pharmaceutically acceptable salt thereof.

Aspect D17. The compound of any one of Aspects D1, D14, or D15, that is:

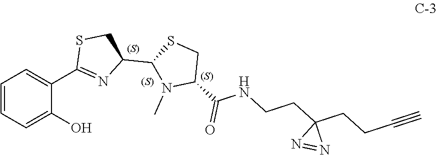

or a pharmaceutically acceptable salt thereof.

Aspect D18. The compound of any one of Aspects D1, D14, or D15, that is:

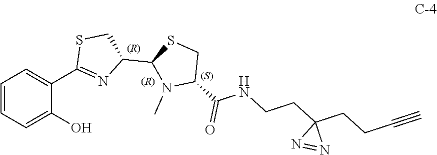

or a pharmaceutically acceptable salt thereof.

Aspect D19. The compound of any one of Aspects D1, D14, or D15, that is:

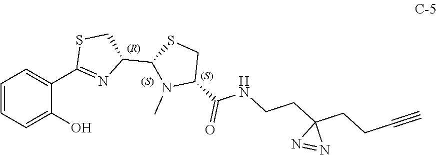

or a pharmaceutically acceptable salt thereof.

Aspect D20. The compound of Aspect D1, that is compound D, or a pharmaceutically acceptable salt thereof.

Aspect D21. The compound of Aspect D1 or D20, that is

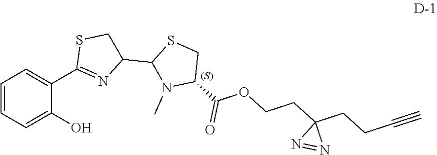

or a pharmaceutically acceptable salt thereof.

Aspect D22. The compound of any one of Aspects D1, D20, or D21, that is:

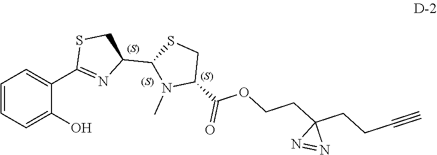

or a pharmaceutically acceptable salt thereof.

Aspect D23. The compound of any one of Aspects D1, D20, or D21, that is:

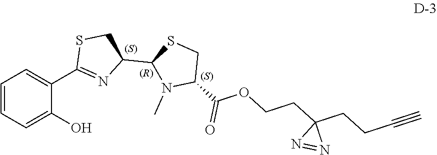

or a pharmaceutically acceptable salt thereof.

Aspect D24. The compound of any one of Aspects D1, D20, or D21, that is:

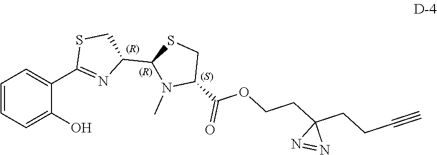

or a pharmaceutically acceptable salt thereof.

Aspect D25. The compound of any one of Aspects D1, D20, or D21, that is:

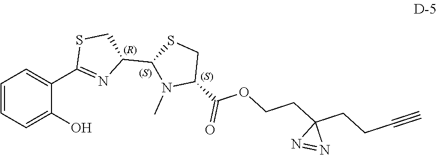

or a pharmaceutically acceptable salt thereof.

Aspect D26. The compound of Aspect D1, that is compound E, or a pharmaceutically acceptable salt thereof.

Aspect D27. The compound of Aspect D1 or D26, that is

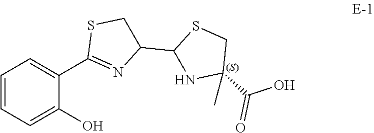

or a pharmaceutically acceptable salt thereof.

Aspect D28. The compound of any one of Aspects D1, D26, or D27 that is

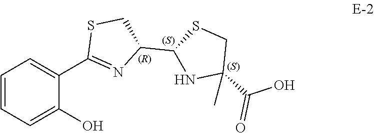

or a pharmaceutically acceptable salt thereof.

Aspect D29. The compound of any one of Aspects D1, D26, or D27 that is

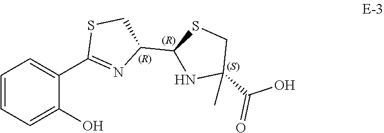

or a pharmaceutically acceptable salt thereof.

Aspect D30. The compound of any one of Aspects D1, D26, or D27 that is

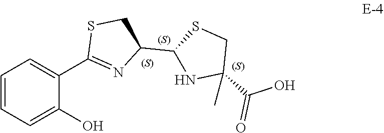

or a pharmaceutically acceptable salt thereof.

Aspect D31. The compound of any one of Aspects D1, D26, or D27 that is

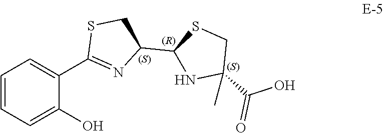

or a pharmaceutically acceptable salt thereof.

Aspect D32. The compound of Aspect D1, that is compound F, or a pharmaceutically acceptable salt thereof.

Aspect D33. The compound of Aspect D1 or D32, that is

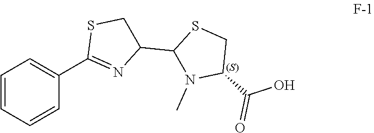

or a pharmaceutically acceptable salt thereof.

Aspect D34. The compound of any one of Aspects D1, D32, or D33 that is

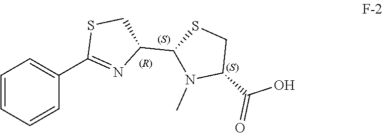

or a pharmaceutically acceptable salt thereof.

Aspect D35. The compound of any one of Aspects D1, D32, or D33 that is

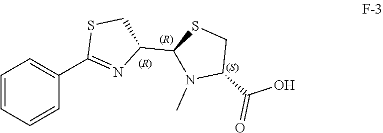

or a pharmaceutically acceptable salt thereof.

Aspect D36. The compound of any one of Aspects D1, D32, or D33 that is

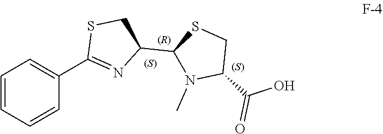

or a pharmaceutically acceptable salt thereof.

Aspect D37. The compound of any one of Aspects D1, D32, or D33 that is

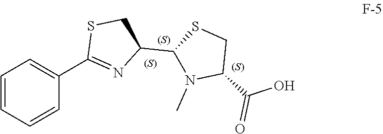

or a pharmaceutically acceptable salt thereof.

Aspect D38. The compound of Aspect D1, that is compound G, or a pharmaceutically acceptable salt thereof.

Aspect D39. The compound of Aspect D1 or D38, that is

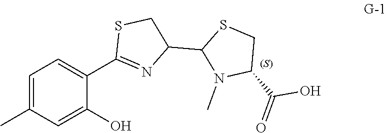

or a pharmaceutically acceptable salt thereof.

Aspect D40. The compound of any one of Aspects D1, D38, or D39 that is

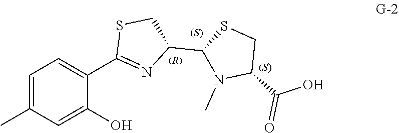

or a pharmaceutically acceptable salt thereof.

Aspect D41. The compound of any one of Aspects D1, D38, or D39 that is

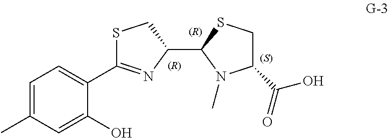

or a pharmaceutically acceptable salt thereof.

Aspect D42. The compound of any one of Aspects D1, D38, or D39 that is

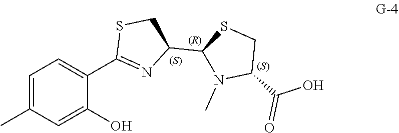

or a pharmaceutically acceptable salt thereof.

Aspect D43. The compound of any one of Aspects D1, D38, or D39 that is

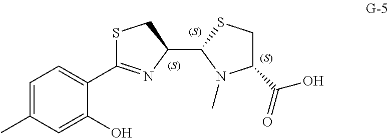

or a pharmaceutically acceptable salt thereof.

Aspect D44. A pharmaceutical composition comprising a therapeutically effective amount of one or more of the compound of any one of Aspects D1-D43 and a pharmaceutically acceptable excipient.

Aspect D45. The pharmaceutical composition of Aspect D44, comprising:

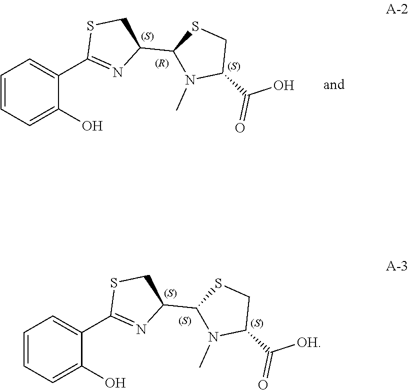

Aspect D46. The pharmaceutical composition of Aspect D45, wherein the molar ratio of

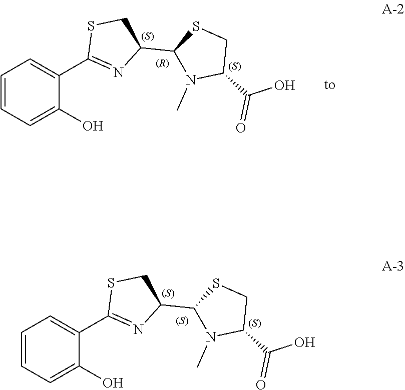

is about 40 to about 60.

Aspect D47. The pharmaceutical composition of Aspect D44, comprising

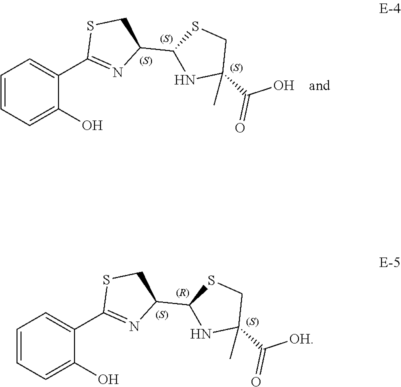

Aspect D48. The pharmaceutical composition of Aspect D44, comprising

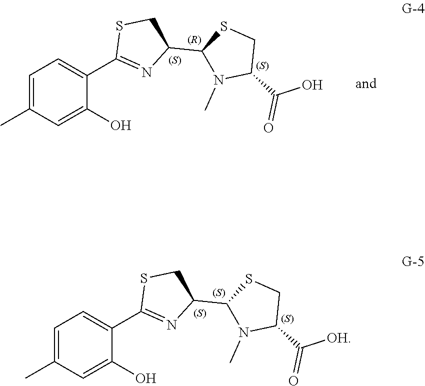

Aspect D49. The pharmaceutical composition of Aspect D48, wherein the molar ratio of

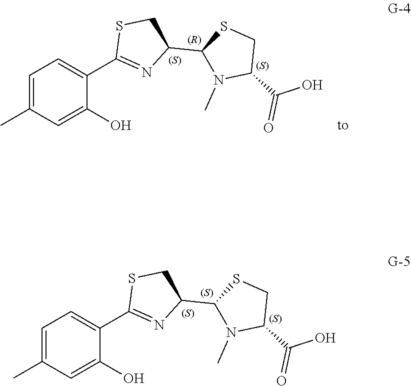

is about 40 to about 60.

Aspect D50. A dosage unit comprising compound of any one of Aspects D1-D43 or the pharmaceutical composition of any one of Aspects D44-D49.

Aspect D51. The dosage of unit of Aspect D50, that is an oral dosage unit such as a pill, tablet, capsule, syrup, liquid solution, powder, paste, or film or a parenteral dosage unit such as an injectable solution.

Aspect D52. The dosage unit of Aspect D50 or D51, comprising about 0.01 to about 1000 mg of the compound.

Aspect D53. The dosage unit of any one of Aspects D50-D52 that is a solid dosage form.

Aspect D54. The dosage unit of any one of Aspects D50-D52 that is a liquid dosage form.

Aspect D55. A method of modulating an inflammatory bowel disease (IBD) marker in a subject in need thereof, comprising administering the compound of any one of Aspects D1-D43, the pharmaceutical composition of any one of Aspects D44-4D9, or the dosage form of any one of Aspects D50-D54 to the subject in need thereof.

Aspect D56. A method of treating an inflammatory bowel disease (IBD) in a subject in need thereof, comprising administering to the subject the compound of any one of Aspects D1-D43, the pharmaceutical composition of any one of Aspects D44-D49, or the dosage form of any one of Aspects D50-D54 to the subject in need thereof.

Aspect D57. The method of Aspect D55 or D56, wherein the subject has or is at risk of developing IBD.

Aspect D58. The method of any one of Aspects D55-D57, wherein the pharmaceutical composition or dosage unit is administered orally, intravenously, intraperitoneally, intramuscularly, or subcutaneously.

Aspect D59. The method of Aspect D58, wherein the pharmaceutical composition or dosage form is administered orally.

Aspect D60. The method of any one of Aspects D55-D59, wherein the IBD is ulcerative colitis.

Aspect D61. The method of any one of Aspects D55-D59, wherein the IBD is Crohn's disease.

Aspect D62. The method of any one of Aspects D55-D61, wherein inflammation or ulcers in the intestinal lining of the subject are reduced, as observed by colonoscopy.

Aspect D63. The method of any one of Aspects D55-D62, wherein tumor necrosis factor alpha (TNF-α), interleukin-1β (IL-1β), interleukin-6 (IL-6), interleukin-8 (IL-8), interleukin-23 (IL-23), interleukin 12 (IL-12), or interleukin-17A (IL-17A) levels, or a combination thereof, in the subject are reduced.

Aspect D64. The method of any one of Aspects D55-D63, wherein nuclear factor kappa B (NF-κB), monocyte chemoattractant protein-1 (MCP-1), cysteine-cysteine motif chemokine ligand 20 (CCL20), or interferon-γ (IFN-γ) levels, or a combination thereof, in the subject are reduced.

Aspect D65. The method of any one of Aspects D55-D64, wherein M2 pyruvate kinase (M2-PK), osteoprotegerin, myeloperoxidase (MPO), high mobility group box protein 1 (HMGB1), chitinase 3 like 1 (CHI3L1), human beta defensin 2 (HBD2), metalloproteinase (MMP), calprotectin, lactoferrin, peripheral antineutrophil cytoplasmic antibodies (p-ANCA), or anti-Saccharomyces cerevisiae antibodies (ASCA) levels, or a combination thereof, in the subject are reduced.

Aspect D66. The method of any one of Aspects D55-D65, wherein one or both serum amyloid A protein (SAA) or eotaxin-1 levels are reduced in the subject.

The following examples are provided to illustrate some of the concepts described within this disclosure. While each Example is considered to provide specific individual embodiments of compositions, methods of preparation and use, none of the Examples should be considered to limit the more general embodiments described herein.

### EXAMPLES

### Example 1: Preparation of Compounds A-2 to A-5—(4S)-2-(2-(2-hydroxyphenyl)-4,5-dihydrothiazol-4-yl)-3-methylthiazolidine-4-carboxylic acid

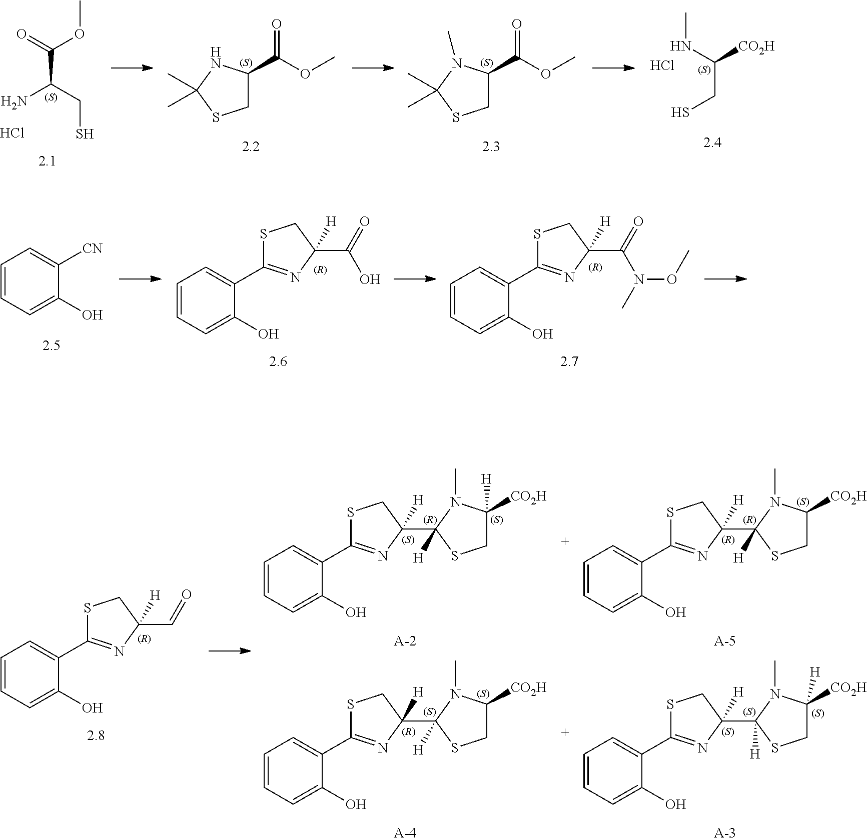

Synthesis of 2.2: A mixture of methyl (2S)-2-amino-3-sulfanyl-propanoate (2.1, 30 g, 174.78 mmol, 1 eq, HCl) in acetone (600 mL) was degassed and purged with N₂ for 3 times, and then the mixture was stirred at 60° C. for 12 h under N₂ atmosphere. The reaction mixture was concentrated under reduced pressure to remove acetone. The residue was diluted with ethyl acetate (200 mL), washed successively with water, 5% aqueous sodium bicarbonate solution (3×200 mL), and brine (100 mL), and then dried over anhydrous sodium sulfate, filtered and concentrated under reduced pressure to give a residue. The residue was purified by column chromatography (SiO₂, petroleum ether/ethyl acetate=0/1 to 3/1) to give 22.3 g of 2.2 (72%) as a colorless oil. (ES, m/z): [M+1]⁺ 176.1.

Synthesis of 2.3: To a solution of methyl (4S)-2,2-dimethylthiazolidine-4-carboxylate (2.2, 11 g, 62.8 mmol, 1 eq) and K₂CO₃ (13 g, 94.2 mmol, 1.5 eq) in DMF (50 mL) was added Me₂SO₄ (11.9 g, 94.2 mmol, 8.93 mL, 1.5 eq) dropwise with stirring at 0° C. The mixture was stirred at 15° C. for 12 h. The reaction mixture was quenched by the addition of water (10 mL) at 0° C. The residue was diluted with water (45 mL) and extracted with ethyl acetate (3×20 mL). The combined organic layers were washed with brine (30 mL), dried over anhydrous sodium sulfate, filtered and concentrated under reduced pressure to give a residue. The residue was purified by flash silica gel chromatography (ISCO®; 40 g SepaFlash® Silica Flash column, eluent of 0-5% ethyl acetate/petroleum ether gradient@60 mL/min) to give 6 g of 2.3 (50%) as a colorless oil. (ES, m/z): [M+1]⁺ 190.1.

Synthesis of 2.4: A mixture of methyl (4S)-2,2,3-trimethylthiazolidine-4-carboxylate (2.3, 4 g, 21.13 mmol, 1 eq) in HCl (6 M, 80 mL, 22.71 eq) was degassed and purged with Ar for 3 times, and then the mixture was stirred at 100° C. for 8 h under Ar atmosphere. The reaction mixture was concentrated under reduced pressure to give 3.8 g of 2.4 (crude, HCl) as a yellow solid. The crude product was used directly to the next step without further purification. (ES, m/z): [M+1]⁺ 136.0.

Synthesis of 2.6: To a solution of 2-hydroxybenzonitrile (2.5, 10 g, 83.95 mmol, 1 eq) in MeOH (150 mL) and phosphate buffer (150 mL) was added (2R)-2-amino-3-sulfanyl-propanoic acid (26.46 g, 167.90 mmol, 2 eq, HCl) at room temperature. The solution was adjusted to pH=6.4 by the addition of solid K₂CO₃ carefully and the reaction mixture was stirred at 60° C. for 12 h. The mixture was concentrated under reduced pressure, and the yellow crude residue was diluted with water (20 mL). The solution was adjusted to pH=2.0 by the addition of solid citric acid. After extraction with dichloromethane (30 mL×3), the organic layers were collected, dried over anhydrous sodium sulfate, filtered and concentrated under reduced pressure to give 2.6 (17 g, 84% yield) as a yellow solid. (ES, m/z): [M+1]⁺ 224.0.

Synthesis of 2.7: To a solution of (4R)-2-(2-hydroxyphenyl)-4,5-dihydrothiazole-4-carboxylic acid (2.6, 17 g, 72.34 mmol, 1 eq), N-methoxymethanamine (7.76 g, 79.58 mmol, 1.1 eq, HCl) and DECP (12.98 g, 79.58 mmol, 1.57 mL, 12.02 eq) in DMF (170 mL) was added DIPEA (14.02 g, 108.52 mmol, 18.9 mL, 1.5 eq) at 0° C. under N₂. The mixture was stirred for 30 min at 0° C., then another 2 h at 25° C. The reaction mixture was diluted with water (45 mL) and extracted with EtOAc (60 mL×3). The combined organic layers were washed with NaHCO₃ (30 mL×3), dried over Na₂SO₄, filtered and concentrated under reduced pressure to give a residue. The residue was purified by silica gel column chromatography (SiO₂, Petroleum ether:Ethyl acetate from 1:0 to 19:1) to give 2.7 (12.95 g, 96.48% yield) as alight yellow solid. (ES, m/z): [M+1]⁺ 267.1.

Synthesis of 2.8: To a solution of (R)-2-(2-hydroxyphenyl)-N-methoxy-N-methyl-4,5-dihydrothiazole-4-carboxamide (2.7, 12.00 g, 45.06 mmol, 1 eq) in THE (240 mL) was added LAH (2.57 g, 67.59 mmol, 1.5 eq) at −40° C. under N₂. The temperature was allowed to raise up to −30° C. in 0.5 h. The reaction mixture was then hydrolyzed by successive additions of aqueous solutions of saturated NH₄Cl (200 mL) and of a 1 M solution of KHSO₄ (200 mL). The mixture was vigorously stirred and allowed to warm up to the room temperature until two phases were formed. After extraction with dichloromethane (100 mL×3), the organic layers were combined, dried over anhydrous sodium sulfate and filtered before being evaporated under reduced pressure to give 2.8 (9.34 g, crude) as a yellow solid. The crude product was used to the next step without further purification. (ES, m/z): [M+1]⁺ 208.0.

Synthesis of A-2 to A-5: To a solution of (R)-2-(2-hydroxyphenyl)-4,5-dihydrothiazole-4-carbaldehyde (2.8, 9.34 g, 45.07 mmol, 1 eq) in a mixture of EtOH (300 mL) and H₂O (100 mL) was added (2S)-2-(methylamino)-3-sulfanyl-propanoic acid (2.4, 9.28 g, 54.08 mmol, 1.2 eq, HCl) and KOAc (30.96 g, 315.47 mmol, 7 eq) at 20° C. The suspension was gently stirred 12 h at 20° C. in the dark. Ethanol was removed under reduced pressure and the crude was diluted in water. The aqueous layer was acidified to pH=5 by the addition of solid citric acid, and then the solution was extracted with ethyl acetate (100 mL×3). The organic layers were combined, dried over anhydrous sodium sulfate, filtered and evaporated to dryness under reduced pressure. The residue was purified by prep-HPLC (column: Welch Xtimate C18 250*100 mm*10 μm; mobile phase: [water (NH₄HCO₃)-ACN]; B %: 5%-35%, 20 min) to give A-2 to A-5 (6.15 g, 42.06% yield), i.e., a mixture of four stereoisomers, as a light yellow solid. (ES, m/z): [M+1]⁺ 325.1.

### Example 2: Purification of Compounds A-2 to A-5—(2R,4S)-2-((S)-2-(2-hydroxyphenyl)-4,5-dihydrothiazol-4-yl)-3-methylthiazolidine-4-carboxylic acid (A-2); (2R,4S)-2-((R)-2-(2-hydroxyphenyl)-4,5-dihydrothiazol-4-yl)-3-methylthiazolidine-4-carboxylic acid (A-5); (2S,4S)-2-((R)-2-(2-hydroxyphenyl)-4,5-dihydrothiazol-4-yl)-3-methylthiazolidine-4-carboxylic acid (A-4); and (2S,4S)-2-((S)-2-(2-hydroxyphenyl)-4,5-dihydrothiazol-4-yl)-3-methylthiazolidine-4-carboxylic acid (A-3)

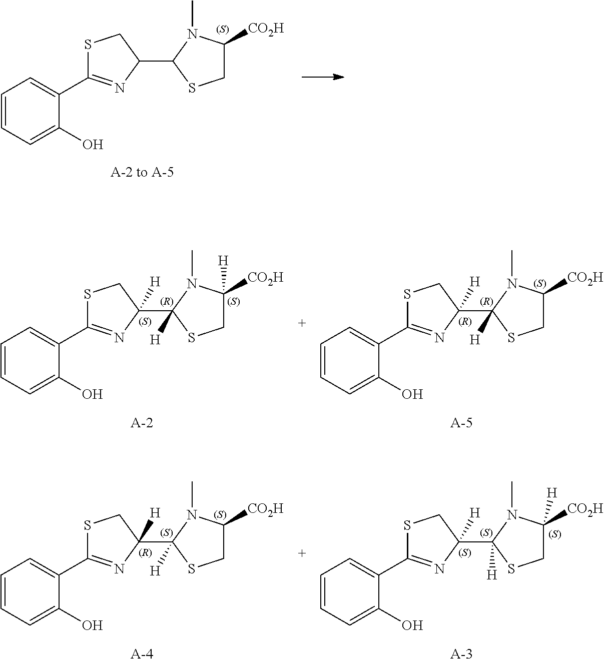

Two grams of the compound of Example 1 (A-2 to A-5) (mixture of 4 peaks) was purified by SFC separation twice to give 290 mg of A-2 (14.5% yield, de value is 87.56%, Peak 1), 80 mg of A-5 (4% yield, de value is 94.2%, Peak 2), 120 mg of A-4 (6% yield, de value is 96.22%, Peak 3) and 480 mg of A-3 (24% yield, de value is 98.38%, Peak 4) as light yellow solids. (ES, m/z): [M+1]⁺ 325.1.

Method 1: Instrument: Waters SFC80Q preparative SFC; Column: DAICEL CHIRALPAK AD (250 mm*30 mm, 10 μm); Mobile phase: A for CO₂ and B for MeOH (0.1% NH₃H₂O); Gradient: B %=30% isocratic elution mode; Flow rate: 57 g/min; Wavelength:220 nm; Column temperature: 40° C.; System back pressure: 100 bar.

Method 2: Instrument: Waters SFC80Q preparative SFC; Column: DAICEL CHIRALPAK IG (250 mm*30 mm, 10 μm); Mobile phase: A for CO₂ and B for MeOH (0.1% NH₃H₂O); Gradient: B %=46% isocratic elution mode; Flow rate: 75 g/min; Wavelength:220 nm; Column temperature: 40° C.; System back pressure: 100 bar.

A-2: ¹HNMR (400 MHz, acetone-d₆) δ=7.46-7.37 (m, 2H), 6.99-6.90 (m, 2H), 5.00 (q, J=8.3 Hz, 1H), 4.54 (d, J=8.5 Hz, 1H), 4.20 (t, J=6.6 Hz, 1H), 3.67 (dd, J=8.7, 11.3 Hz, 1H), 3.57-3.39 (m, 1H), 3.29-3.16 (m, 2H), 2.49 (s, 3H).

A-3: ¹HNMR (400 MHz, acetone-d₆) δ=7.46-7.38 (m, 2H), 6.97-6.91 (m, 2H), 5.22 (dt, J=5.4, 8.9 Hz, 1H), 4.63 (d, J=5.4 Hz, 1H), 3.71 (br t, J=7.4 Hz, 1H), 3.50 (d, J=8.9 Hz, 2H), 3.26-3.17 (m, 2H), 2.65 (s, 3H).

A-5: ¹HNMR (400 MHz, acetone-d6) δ=7.47-7.36 (m, 2H), 7.00-6.87 (m, 2H), 4.85 (br d, J=8.2 Hz, 1H), 4.34 (d, J=8.2 Hz, 1H), 3.99 (br s, 1H), 3.68-3.58 (m, 1H), 3.57-3.40 (m, 2H), 3.29 (br dd, J=7.0, 10.8 Hz, 1H), 2.63 (s, 3H).

A-4: ¹HNMR (400 MHz, acetone-d6) δ=7.46-7.37 (m, 2H), 6.98-6.89 (m, 2H), 5.29 (dt, J=4.7, 9.0 Hz, 1H), 5.07 (d, J=4.8 Hz, 1H), 4.25 (br d, J=4.9 Hz, 1H), 3.53-3.36 (m, 2H), 3.22 (dd, J=6.5, 10.4 Hz, 1H), 3.07 (br d, J=9.1 Hz, 1H), 2.71 (s, 3H).

### Example 3: Preparation of Compound G-2 to G-5: (2R,4S)-2-((S)-2-(2-hydroxy-4-methylphenyl)-4,5-dihydrothiazol-4-yl)-3-methylthiazolidine-4-carboxylic acid (G-4); (2S,4S)-2-((S)-2-(2-hydroxy-4-methylphenyl)-4,5-dihydrothiazol-4-yl)-3-methylthiazolidine-4-carboxylic acid (G-5); (2R,4S)-2-((R)-2-(2-hydroxy-4-methylphenyl)-4,5-dihydrothiazol-4-yl)-3-methylthiazolidine-4-carboxylic acid (G-3); (2S,4S)-2-((R)-2-(2-hydroxy-4-methylphenyl)-4,5-dihydrothiazol-4-yl)-3-methylthiazolidine-4-carboxylic acid (G-2)

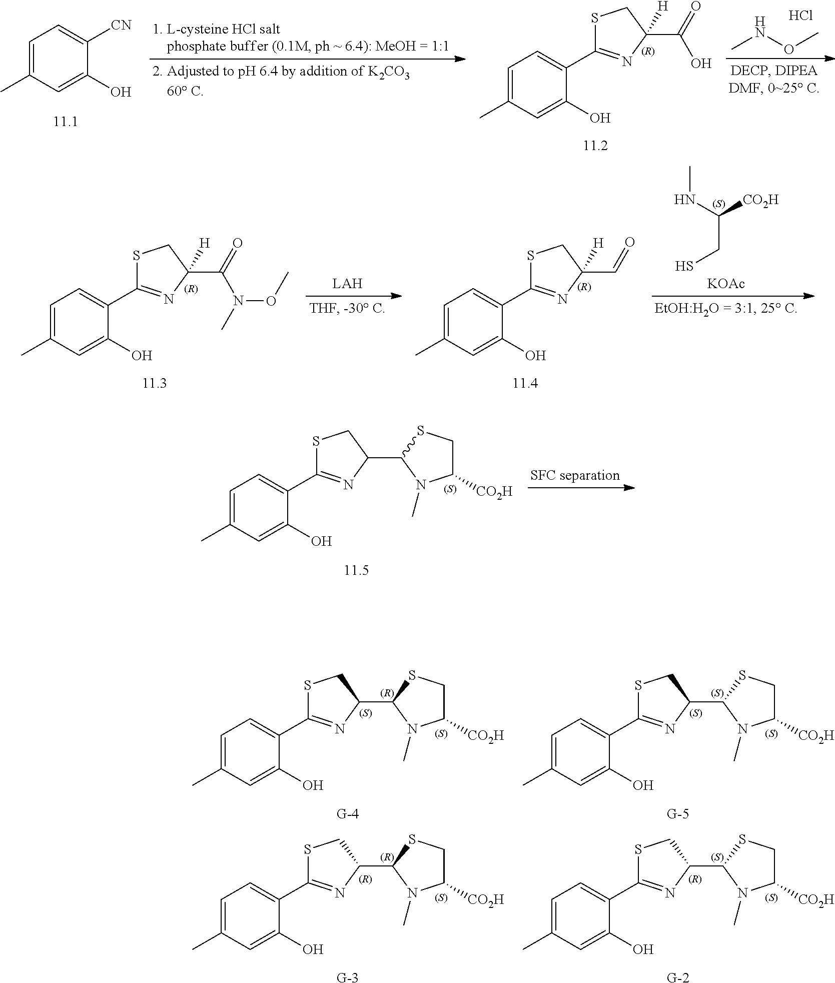

Synthesis of 11.2. To a solution of 2-hydroxy-4-methylbenzonitrile (11.1, 3 g, 22.53 mmol, 1 eq) in MeOH (30 mL) and phosphate buffer (150 mL) was added (2R)-2-amino-3-sulfanyl-propanoic acid (5.46 g, 45.06 mmol, 2 eq) and phosphate buffer (30 mL). The solution was adjusted to pH=6.4 by the addition of solid K₂CO₃ carefully and the reaction mixture was stirred at 60° C. for 12 h. The mixture was concentrated under reduced pressure, and the yellow crude residue was diluted with water (20 mL). The solution was adjusted to pH=2.0 by the addition of solid citric acid. After extraction with dichloromethane (3×20 mL), the organic layers were collected, dried over anhydrous sodium sulfate, filtered and concentrated under reduced pressure to give 5.35 g of 11.2 (98%, % purity, ee value is 100.0%) as a yellow solid. (ES, m/z): [M+1]⁺ 238.0.

Synthesis of 11.3. To a solution of (R)-2-(2-hydroxy-4-methylphenyl)-4,5-dihydrothiazole-4-carboxylic acid (11.2, 3 g, 12.64 mmol, 1 eq), N-methoxymethanamine (849.54 mg, 13.91 mmol, 1.1 eq, HCl) and DECP (2.39 g, 13.91 mmol, 2.21 mL, 95% purity, 1.1 eq) in DMF (5 mL) was added DIEA (2.45 g, 18.97 mmol, 3.30 mL, 1.5 eq) at 0° C. under N₂. The mixture was stirred for 30 min at 0° C., then another 2 h at 25° C. The reaction mixture was diluted with H₂O (10 mL) and extracted with EA (10 mL*3). The combined organic layers were washed with aq. NaHCO₃ and brine (10 mL), dried over Na₂SO₄, filtered and concentrated under reduced pressure to give a residue. The organic phase was washed with aq. NaHCO₃, then with brine, dried over anhydrous sodium sulfate, filtered and evaporated. The residue and the filter cake were purified by silica gel column chromatography (SiO₂, Petroleum ether:Ethyl acetate from 1:0 to 1:1) to give 2.05 g of 11.3 (57.84%, % ee value is 84.94%) as a light yellow solid. (ES, m/z): [M+1]⁺ 281.1.

Synthesis of 11.4. To a solution of (R)-2-(2-hydroxy-4-methylphenyl)-N-methoxy-N-methyl-4,5-dihydrothiazole-4-carboxamide (11.3, 1.8 g, 6.42 mmol, 1 eq) in THE (15 mL) was added LAH (365.54 mg, 9.63 mmol, 1.5 eq) at −40° C. under N₂. The temperature was allowed to rise up to −30° C. in 0.5 h. The reaction mixture was then hydrolyzed by successive additions of aqueous solutions of saturated NH₄Cl (30 mL) and of a 1 M solution of KHSO₄ (30 mL). The mixture was vigorously stirred and allowed to warm up to the room temperature until two phases were formed. After partition and extraction with ethyl acetate (2×20 mL), the organic layers were combined, dried over anhydrous sodium sulfate and filtered before being evaporated under reduced pressure to give 210 mg of 11.4 (crude) as a yellow solid. The crude product was used to the next step without further purification. (ES, m/z): [M+1]⁺ 222.2

Synthesis of 11.5. To a solution of (R)-2-(2-hydroxy-4-methylphenyl)-4,5-dihydrothiazole-4-carbaldehyde (11.4, 1.4 g, 6.33 mmol, 1 eq) in a mixture of EtOH (21 mL) and H₂O (7 mL) was added (2S)-2-(methylamino)-3-sulfanyl-propanoic acid (1.30 g, 7.59 mmol, 1.2 eq, HCl) and KOAc (4.35 g, 44.29 mmol, 7 eq) at 20° C. The suspension was gently stirred 12 h at 20° C. in the dark. Ethanol was removed under reduced pressure and the crude was diluted in water. The aqueous layer was acidified to pH=5 by the addition of solid citric acid, and then the solution was extracted with ethyl acetate (2×30 mL). The organic layers were combined, dried over anhydrous sodium sulfate, filtered and evaporated to dryness under reduced pressure. The residue was purified by prep-HPLC column: Welch Xtimate C18 250*70 mm #10 μm; mobile phase: [water (NH₄HCO₃)-ACN]; B %: 10%-40%, 20 min) to give 150 mg of 11.5 (25.69%) (a mixture of G-2, G-3, G-4, and G-5) as a light yellow solid. (ES, m/z): [M+1]⁺ 339.0.

Separation of G-2 to G-5. 300 mg of 11.5 (mixture of 4 peaks) was purified by twice SFC separation to give 70.91 mg of G-5 (23%, de value is 93%, Peak 1), 33.9 mg of G-4 (11%, de value is 99%, Peak 2), 28.91 mg of G-2 (9.6%, de value is 63%, Peak 3) and 67.94 mg of G-3 (22.6%, de value is 89%, Peak 4) as light yellow solids. (ES, m/z): [M+1]⁺ 339.0

Method 1: Instrument: Waters SFC 80Q preparative SFC; Column: DAICEL CHIRALPAK AD (250 mm*30 mm, 10 μm); mobile phase: A for CO₂ and B for IPA (0.1% NH₃·H₂O); Gradient: B from 38%-38% in 7 min

Method 2: Instrument: Waters SFC 80Q preparative SFC; Column: DAICEL CHIRALPAK IG (250 mm*30 mm, 10 μm); mobile phase: A for CO₂ and B for IPA (0.1% NH₃·H₂O); Gradient: B from 30%-30% in 15 min

G-4: ¹H NMR (400 MHz, acetone-d6) δ=7.29 (d, J=7.9 Hz, 1H), 6.81-6.70 (m, 2H), 4.98 (d, J=8.2 Hz, 1H), 4.53 (d, J=8.3 Hz, 1H), 4.25 (t, J=6.4 Hz, 1H), 3.64 (dd, J=8.6, 11.3 Hz, 1H), 3.44 (dd, J=7.9, 11.3 Hz, 1H), 3.23 (d, J=6.4 Hz, 2H), 2.50 (s, 3H), 2.31 (s, 3H)

G-5: ¹H NMR (400 MHz, acetone-d6) δ=7.30 (d, J=7.9 Hz, 1H), 6.85-6.64 (m, 2H), 5.25-5.11 (m, 1H), 4.61 (d, 1H), 3.76-3.65 (m, 1H), 3.47 (d, J=8.9 Hz, 1H), 3.20 (br d, J=7.0 Hz, 2H), 2.64 (s, 3H), 2.31 (s, 3H)

G-3: ¹H NMR (400 MHz, acetone-d6) δ=7.29 (d, J=7.9 Hz, 1H), 6.82-6.68 (m, 2H), 5.33-5.18 (m, 1H), 5.04 (d, J=4.8 Hz, 1H), 4.26 (dd, J=2.1, 6.5 Hz, 1H), 3.68-3.56 (m, 2H), 3.38-3.28 (m, 1H), 3.25-3.19 (m, 1H), 3.07 (s, 1H), 2.73-2.63 (m, 3H), 2.31 (s, 3H)

G-2: ¹H NMR (400 MHz, acetone-d6) δ=7.29 (d, J=8.0 Hz, 1H), 6.82-6.64 (m, 2H), 4.81 (d, J=8.2 Hz, 1H), 4.33 (d, J=8.3 Hz, 1H), 4.03 (dd, J=5.4, 6.7 Hz, 1H), 3.67-3.58 (m, 1H), 3.55-3.40 (m, 2H), 3.30 (dd, J=6.9, 11.0 Hz, 2H), 2.63 (s, 3H), 2.31 (s, 3H)

### Example 4: Preparation of (4S)-2-((S)-2-(2-hydroxy-4-methylphenyl)-4,5-dihydrothiazol-4-yl)-3-methylthiazolidine-4-carboxylic acid

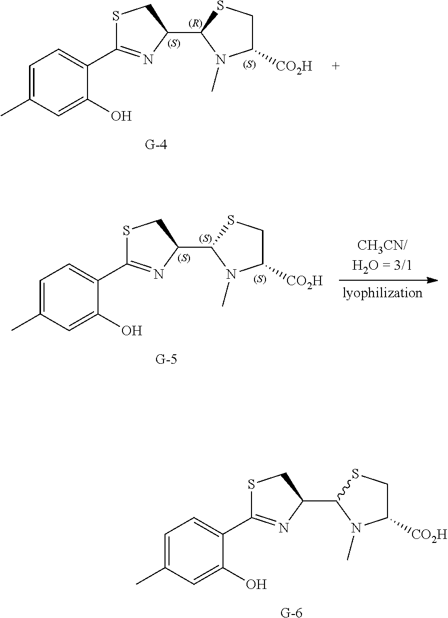

Synthesis of G-6. A solution of (2R,4S)-2-((S)-2-(2-hydroxy-4-methylphenyl)-4,5-dihydrothiazol-4-yl)-3-methylthiazolidine-4-carboxylic acid (G-4, 7.9 mg) and (2S,4S)-2-((S)-2-(2-hydroxy-4-methylphenyl)-4,5-dihydrothiazol-4-yl)-3-methylthiazolidine-4-carboxylic acid (G-5, 15.2 mg) was dissolved in ACN (3 mL) and H₂O (1 mL), and the mixture was under lyophilization to give 15.74 mg of G-6 (100%) as a light yellow solid. (ES, m/z): [M+1]⁺ 339.0.

G-6: 1H NMR (400 MHz, acetone) δ=7.30 (d, J=7.7 Hz, 1.74H), 6.81-6.71 (m, 3.48H), 5.25-5.12 (m, 1.02H), 5.07-4.92 (m, 0.78H), 4.61 (d, J=5.4 Hz, 0.98H), 4.54 (d, J=8.3 Hz, 0.74H), 4.25 (s, 0.80H), 3.72 (t, J=7.3 Hz, 1.18H), 3.64 (dd, J=8.8, 11.0 Hz, 0.93H), 3.52-3.42 (m, 3.14H), 3.26-3.18 (m, 3.92H), 2.65 (s, 3H), 2.52-2.47 (m, 2.72H), 2.31 (s, 5.85H)

### Example 5: Preparation of Compounds C-2 to C-5—(4S)—N-(2-(3-but-3-yn-1-yl)-3H-diazirin-3-yl)ethyl)-2-(2-(2-hydroxyphenyl)-4,5-dihydrothiazol-4-yl)-3-methyl-thiazolidine-4-carboxamide

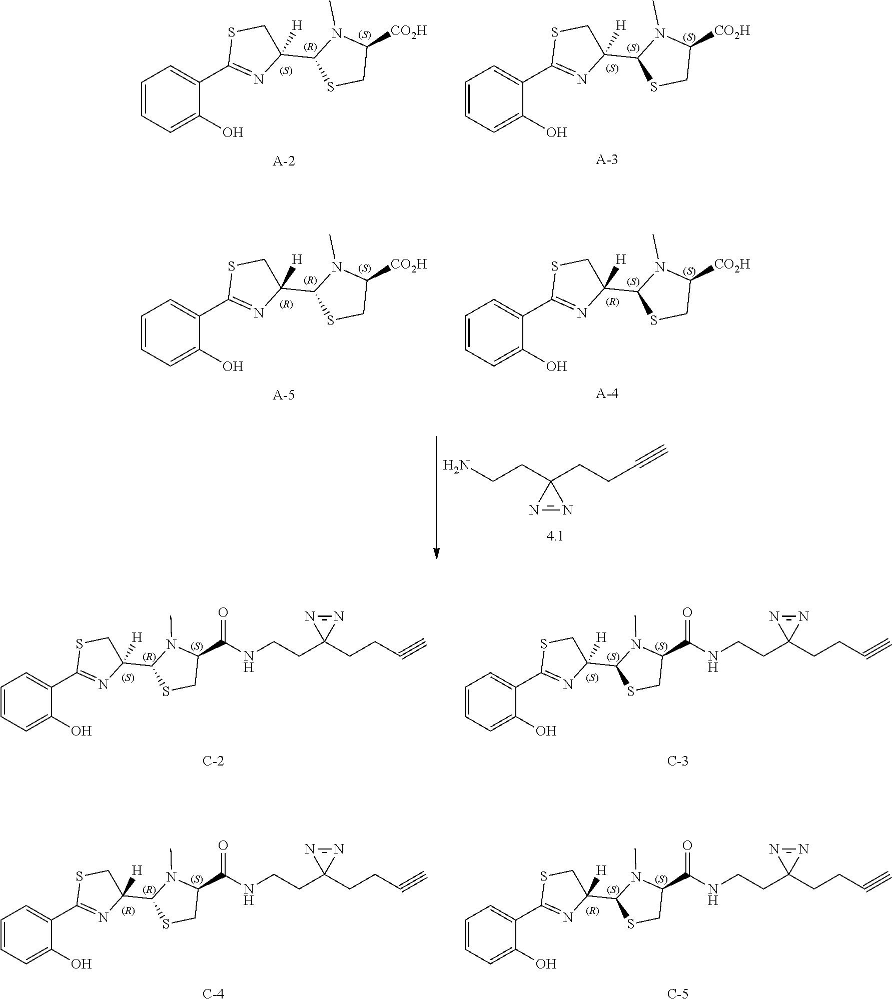

To a mixture of (4S)-2-(2-(2-hydroxyphenyl)-4,5-dihydrothiazol-4-yl)-3-methylthiazolidine-4-carboxylic acid (A-2-A-5, 40 mg, 123.30 μmol, 1 eq), 2-(3-but-3-ynyldiazirin-3-yl)ethanamine (4.1, 16.91 mg, 123.30 μmol, 1 eq) in DMF (2 mL) was added HATU (93.76 mg, 246.60 μmol, 2 eq) and DIEA (31.87 mg, 246.60 μmol, 42.95 μL, 2 eq) at 25° C. The mixture was degassed and purged with Ar for 3 times, and then stirred at 25° C. for 12 h under Ar atmosphere. The reaction mixture was filtered to give a solution. The solution was purified by prep-HPLC (neutral condition; column: Phenomenex Gemini-NX 80*40 mm*3 μm; mobile phase: [water (10 mM NH₄HCO₃)-ACN]; B %: 40%-70%, 8 min) to give 15 mg of C-2 to C-5 (27% yield), i.e., a mixture of four stereoisomers, as a black gum. (ES, m/z): [M+1]⁺ 444.1.

### Example 6: Preparation of Compound C-3—(2S,4S)—N-(2-(3-(but-3-yn-1-yl)-3H-diazirin-3-yl)ethyl)-2-((S)-2-(2-hydroxyphenyl)-4,5-dihydrothiazol-4-yl)-3-methylthiazolidine-4-carboxamide

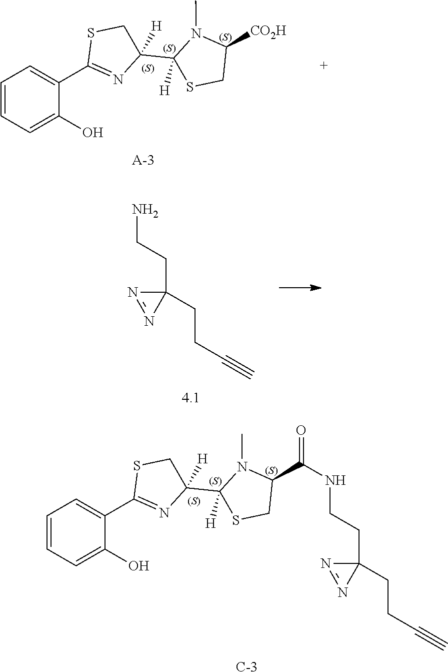

To a mixture of (4S)-2-[(4S)-2-(2-hydroxyphenyl)-4,5-dihydrothiazol-4-yl]-3-methyl-thiazolidine-4-carboxylic acid (A-3, 25 mg, 0.077 mmol, 1 eq), 2-(3-but-3-ynyldiazirin-3-yl)ethanamine (4.1, 10.57 mg, 0.077 mmol, 1 eq) in DMF (1 mL) was added HATU (58.60 mg, 0.154 mmol, 2 eq) and DIEA (19.92 mg, 0.154 mmol, 26.84 μL, 2 eq). The mixture was degassed and purged with Ar for 3 times, and then stirred at 25° C. for 12 h under Ar atmosphere. The reaction mixture was filtered to give a solution. The residue was purified by prep-HPLC (neutral condition; column: Waters Xbridge BEH C18 100*25 mm*5 μm; mobile phase: [water (10 mM NH₄HCO₃)-ACN]; B %: 35%-70%, 10 min) to afford 19 mg of C-3 (52.8%) obtained as a yellow oil. (ES, m/z): [M+1]⁺ 444.6. ¹H NMR (400 MHz, acetone) δ=12.49 (s, 1H), 7.57-7.48 (m, 1H), 7.47-7.35 (m, 2H), 7.03-6.86 (m, 2H), 5.13 (dt, J=6.1, 8.8 Hz, 1H), 4.62 (d, J=5.9 Hz, 1H), 3.68-3.55 (m, 2H), 3.53-3.43 (m, 1H), 3.22 (s, 2H), 3.12 (dq, J=3.7, 6.7 Hz, 2H), 2.65 (s, 3H), 2.39 (t, J=2.6 Hz, 1H), 2.03-1.99 (m, 2H), 1.63 (q, J=7.1 Hz, 4H)

### Example 7: Preparation of Compound C-2—(4S)—N-[2-(3-but-3-ynyldiazirin-3-yl)ethyl]-2-[(4S)-2-(2-hydroxyphenyl)-4,5-dihydrothiazol-4-yl]-3-methyl-thiazolidine-4-carboxamide

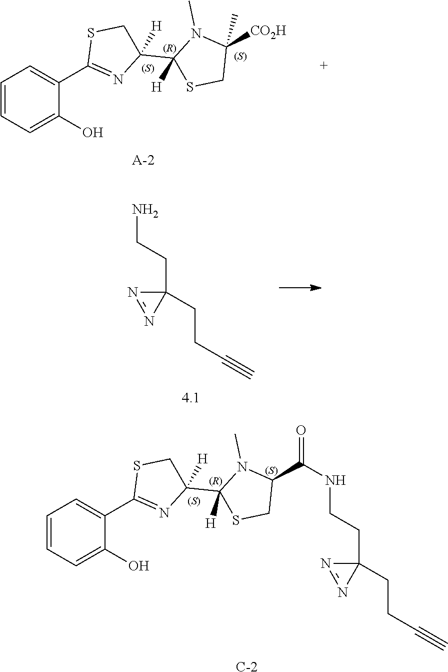

To a mixture of (4S)-2-[(4S)-2-(2-hydroxyphenyl)-4,5-dihydrothiazol-4-yl]-3-methyl-thiazolidine-4-carboxylic acid (A-2, 15 mg, 0.046 mmol, 1 eq), 2-(3-but-3-ynyldiazirin-3-yl)ethanamine (4.1, 6.34 mg, 0.046 mmol, 1 eq) in DMF (1 mL) was added HATU (35.16 mg, 0.092 mmol, 2 eq) and DIEA (11.95 mg, 0.092 μmol, 16.11 μL, 2 eq). The mixture was degassed and purged with Ar for 3 times, and then stirred at 25° C. for 12 h under Ar atmosphere. The reaction mixture was filtered to give a solution. The residue was purified by prep-HPLC (neutral condition; column: Waters Xbridge BEH C18 100*30 mm*10 μm; mobile phase: [water (10 mM NH₄HCO₃)-ACN]; B %: 45%-75%, 8 min) to give 13 mg of C-2 (62.7%) as a yellow oil. (ES, m/z): [M+1]⁺ 444.1. ¹H NMR (400 MHz, ACETONE-d₆) δ=7.65-7.53 (m, 1H), 7.52-7.31 (m, 2H), 6.96 (quin, J=7.5 Hz, 2H), 5.25-4.92 (m, 1H), 4.70-4.45 (m, 1H), 4.11 (dd, J=4.3, 7.2 Hz, 1H), 3.91-3.71 (m, 1H), 3.69-3.43 (m, 2H), 3.38-3.06 (m, 3H), 2.60-2.31 (m, 3H), 2.05-2.02 (m, 2H), 1.75-1.56 (m, 4H).

### Example 8: Preparation Compounds E-2 to E-5—(4S)-2-(2-(2-hydroxyphenyl)-4,5-dihydrothiazol-4-yl)-4-methyl-thiazolidine-4-carboxylic acid

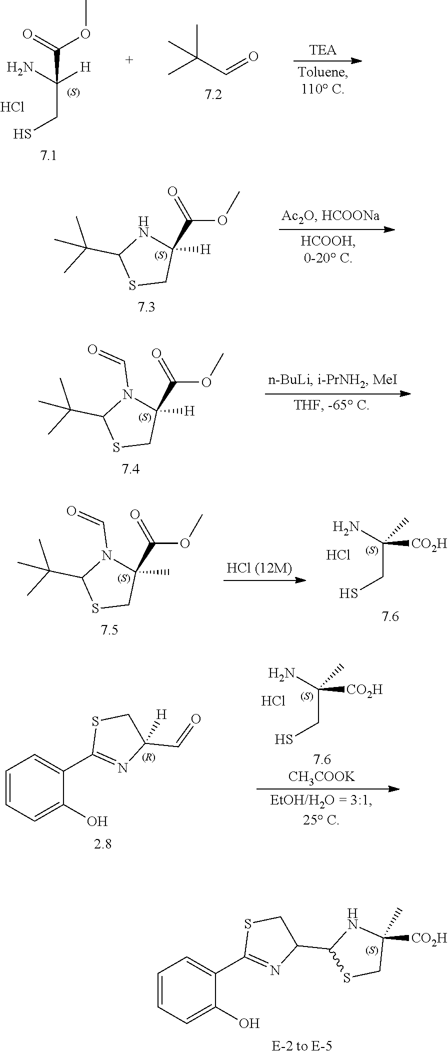

Synthesis of 7.3. To a solution of methyl (2S)-2-amino-3-sulfanyl-propanoate (7.1, 20 g, 116.52 mmol, 1 eq, HCl) in toluene (300 mL) was added 2,2-dimethylpropanal (7.2, 12.04 g, 139.82 mmol, 15.46 mL, 1.2 eq) in one portion, followed by the addition of TEA (17.69 g, 174.78 mmol, 24.33 mL, 1.5 eq), the mixture was heated to reflux (oil bath temperature: 110° C.) and remove water by Dean-Stark for 18 h. The mixture was cooled to room temperature, then filtered, the filter cake was washed with ethyl acetate (2×50 mL), and the filtrate was concentrated in vacuum to give a residue. The residue was diluted with water (50 mL) and extracted with ethyl acetate (3×50 mL), the organic phase was washed with brine (50 mL), dried over anhydrous sodium sulfate, filtered and concentrated in vacuum. The residue was purified by flash silica gel chromatography (ISCO®; 330 g SepaFlash® Silica Flash Column, Eluent of 0-30% Ethyl acetate/Petroleum ether gradient@150 mL/min) to give 20 g of 7.3 (84%) as a colorless oil. (ES, m/z): [M+1]⁺ 204.1.

Synthesis of 7.4. To a solution of methyl (4S)-2-tert-butylthiazolidine-4-carboxylate (7.4, 10 g, 49.19 mmol, 1 eq) in HCOOH (150 mL) was added Ac₂O (15.06 g, 147.56 mmol, 13.82 mL, 3 eq) and HCOONa (4.01 g, 59.03 mmol, 3.19 mL, 1.2 eq) under stirring at 0-5° C. The reaction mixture was warmed to 15° C. and stirred for 18 h. Solvent was removed under reduced pressure, the obtained oil was treated with a saturated solution of NaHCO₃ (100 mL) at 0° C., and ethyl acetate (100 mL) was added. The organic phase was washed with saturated solution of NaHCO₃ (100 mL), dried over anhydrous sodium sulfate, filtered, and the solvent was removed under reduced pressure. The residue combined with another batch was purified by flash silica gel chromatography (ISCO®; 330 g SepaFlash® Silica Flash Column, Eluent of 0˜50% Ethyl acetate/Petroleum ether gradient@150 mL/min) to give 22 g of 7.4 as a white solid. (ES, m/z): [M+1]⁺ 232.1.

Synthesis of 7.5. To the stirred solution of i-PrNH₂ (6.52 g, 110.24 mmol, 9.47 mL, 1.5 eq) in THE (150 mL) was added n-BuLi (2.5 M, 32.34 mL, 1.1 eq) dropwise at −65° C. under a nitrogen atmosphere. DMPU (37.68 g, 293.98 mmol, 35.55 mL, 4 eq) was added in dropwise at this temperature. After the addition, the reaction mixture was stirred at −65° C. for 1 h, the solution of methyl (4S)-2-tert-butyl-3-formyl-thiazolidine-4-carboxylate (7.4, 17 g, 73.49 mmol, 1 eq) in THE (100 mL) was added dropwise at −65° C. The reaction mixture was stirred for 1 h, then MeI (12.52 g, 88.19 mmol, 5.49 mL, 1.2 eq) was added dropwise. The reaction mixture was stirred at this temperature for another 1 h. After the reaction was finished, the reaction mixture was quenched with saturated NH₄Cl solution (20 mL) at 0-5° C. and extracted with ethyl acetate (3×100 mL), the organic phase was combined and washed with brine (50 mL), dried over anhydrous sodium sulfate, filtered and concentrated in vacuum. The crude was purified by flash silica gel chromatography (ISCO®; 330 g SepaFlash® Silica Flash Column, Eluent of 0˜25% Ethyl acetate/Petroleum ether gradient@100 mL/min) to give 12 g of 7.5 (65%) as a yellow solid. (ES, m/z): [M+1]⁺ 246.1.

Synthesis of 7.6. A solution of methyl (4S)-2-tert-butyl-3-formyl-4-methyl-thiazolidine-4-carboxylate (7.5, 1 g, 4.08 mmol, 1 eq) in HCl (10.04 g, 101.90 mmol, 9.84 mL, 37% purity, 25 eq) was degassed and purged with Ar for 3 times, then heated to 100° C. and stirred for 5 days. The reaction mixture was dried in vacuum to give 700 mg of 7.6 (crude) as a yellow solid. The crude product was used directly to next step without further purification.

Synthesis of E-2 to E-5. To a solution of (R)-2-(2-hydroxyphenyl)-4,5-dihydrothiazole-4-carbaldehyde (2.8, 200 mg, 965.02 mol, 1 eq) in a mixture of EtOH (24 mL) and H₂O (8 mL) was added (2S)-2-amino-2-methyl-3-sulfanyl-propanoic acid (7.6, 364.41 mg, 2.12 mmol, 2.2 eq, HCl) and KOAc (662.95 mg, 6.76 mmol, 7 eq) at 20° C. This suspension was gently stirred at 20° C. for 12 h in dark. Ethanol was removed under reduced pressure and the crude was diluted in water. The aqueous layer was acidified to pH=5 by the addition of solid citric acid before being extracted with ethyl acetate (2×30 mL). The organic layers were collected, dried over anhydrous sodium sulfate, filtered and evaporated to dryness under reduced pressure. The residue was purified by prep-HPLC (neutral condition; column: Phenomenex Gemini-NX 80*40 mm*3 μm; mobile phase: [water (10 mM NH₄HCO₃)-ACN]; B %: 10%-30%, 8 min) to give 150 mg of E-2, E-3, E-4, and E-5 (43% yield) as a mixture of four stereoisomers as a light yellow solid. (ES, m/z): [M+1]⁺ 325.1.

¹HNMR (400 MHz, ACETONE-d₆) δ=1.38 (s, 3H), 1.40 (s, 4H), 1.50 (s, 1H), 1.53 (s, 3H), 2.76-2.87 (m, 2H), 2.88-2.95 (m, 2H), 3.04 (d, J=10.13 Hz, 1H), 3.11 (d, J=10.25 Hz, 1H), 3.36-3.47 (m, 4H), 3.56 (s, 2H), 3.57-3.67 (m, 2H), 3.73 (dd, J=11.27, 8.76 Hz, 1H), 4.73 (d, J=7.27 Hz, 1H), 4.82 (d, J=7.63 Hz, 1H), 4.91 (q, J=7.91 Hz, 1H), 5.03-5.13 (m, 2H), 5.18 (td, J=9.03, 4.59 Hz, 1H), 6.89-6.99 (m, 7H), 7.37-7.47 (m, 7H).

### Example 9: Preparation of Compounds B-2 to B-5—(4S)-2-(2-(2-hydroxyphenyl)-4,5-dihydrothiazol-4-yl)-3,4-dimethylthiazolidine-4-carboxylic acid

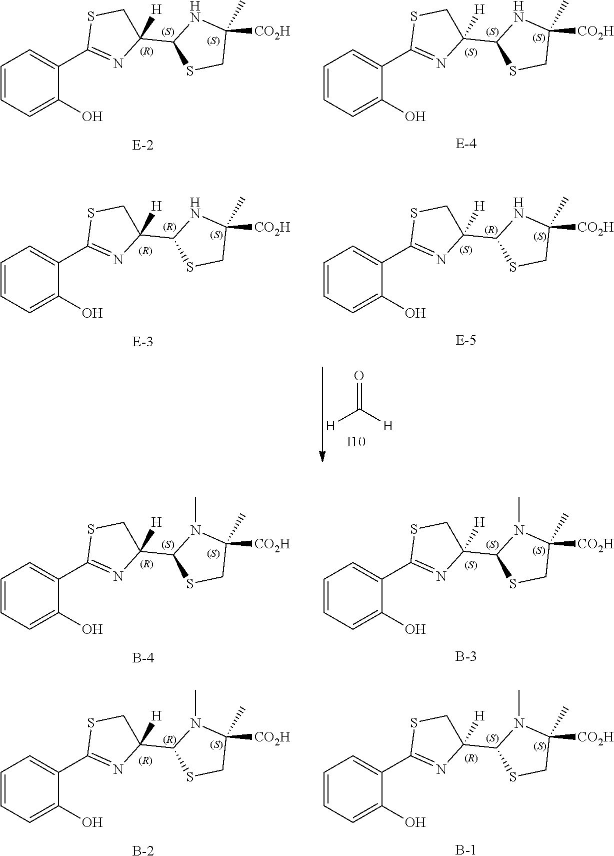

To a solution of (4S)-2-(2-(2-hydroxyphenyl)-4,5-dihydrothiazol-4-yl)-4-methylthiazolidine-4-carboxylic acid (E-2 to E-5, 40 mg, 123.30 μmol, 1 eq) and formaldehyde (I10, 40.02 mg, 493.19 μmol, 36.72 μL, 37% purity, 4 eq) in MeOH (5 mL) was added NaBH₃CN (30.99 mg, 493.19 μmol, 4 eq). The mixture was stirred at 15° C. for 16 h. The reaction mixture was concentrated under reduced pressure to give a residue. The residue was purified by prep-HPLC (neutral condition; column: Waters Xbridge BEH C18 100*30 mm*10 μm; mobile phase: [water (10 mM NH₄HCO₃)-ACN]; B %: 15%-45%, 8 min) to give 36 mg of B-2 to B-5 (83%), i.e., a mixture of four stereoisomers, as a white solid. (ES, m/z): [M+1]⁺ 339.1.

¹HNMR (400 MHz, CHLOROFORM-d) δ=1.46-1.62 (m, 3H), 2.52 (s, 1H), 2.58 (s, 1H), 2.61 (s, 1H), 2.65 (s, 1H), 2.76-2.95 (m, 1H), 3.14-3.66 (m, 3H), 4.38 (d, J=3.73 Hz, 1H), 4.45-4.54 (m, 1H), 4.69-4.80 (m, 1H), 4.99-5.14 (m, 1H), 5.16-5.29 (m, 1H), 6.85-6.94 (m, 1H), 6.96-7.04 (m, 1H), 7.32-7.45 (m, 2H).

### Example 10: Preparation of Compounds D2 to D5—2-(3-but-3-yn-1-yl)-3H-diazirin-3-yl)ethyl (4S)-2-(2-(2-hydroxyphenyl)-4,5-dihydrothiazol-4-yl)-3-methylthiazolidine-4-carboxylate

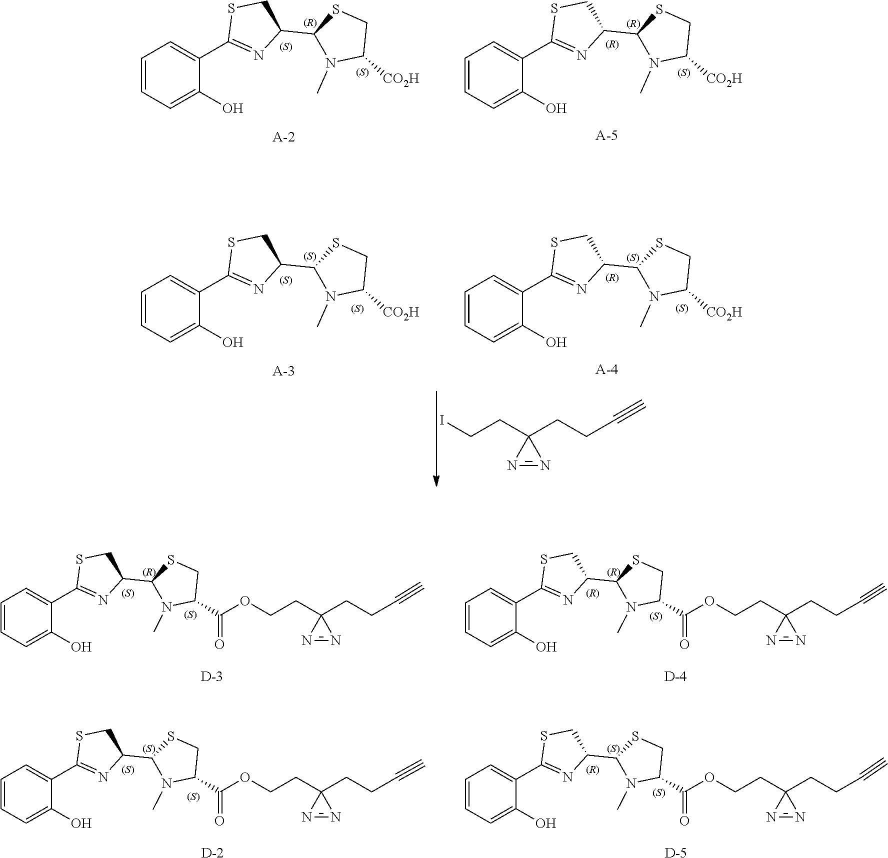

To a solution of (4S)-2-(2-(2-hydroxyphenyl)-4,5-dihydrothiazol-4-yl)-3-methylthiazolidine-4-carboxylic acid (A-2 to A-5, 45 mg, 138.71 μmol, 1 eq) in DMF (5 mL) was added 3-but-3-ynyl-3-(2-iodoethyl)diazirine (41.29 mg, 166.45 μmol, 1.2 eq), K₂CO₃ (23 mg, 166.45 μmol, 1.2 eq) under Ar. The mixture was stirred at 15° C. for 16 h under Ar. The reaction mixture was concentrated under reduced pressure to give a residue. The residue was purified by prep-HPLC (neutral condition, column: Waters Xbridge BEH C18 100*30 mm*10 μm; mobile phase: [water (10 mM NH₄HCO₃)-ACN]; B %: 50%-80%, 8 min) to give 18 mg of D-2 to D-5 (29%), i.e., a mixture of four stereoisomers, as a yellow gum. (ES, m/z): [M+1]⁺ 445.1. ¹HNMR (400 MHz, CHLOROFORM-d) δ=1.70 (br t, J=7.19 Hz, 2H), 1.77-1.88 (m, 2H), 1.97-2.09 (m, 3H), 2.54 (s, 1H), 2.62 (d, J=2.00 Hz, 2H), 2.71 (s, 1H), 3.07-3.13 (m, 1H), 3.15-3.34 (m, 2H), 3.38-3.59 (m, 2H), 3.68 (dd, J=9.07, 6.44 Hz, 1H), 3.84 (t, J=6.88 Hz, 1H), 4.00-4.14 (m, 2H), 4.17 (dd, J=6.13, 1.88 Hz, 1H), 4.27 (d, J=7.38 Hz, 1H), 4.54 (d, J=5.13 Hz, 1H), 4.61 (d, J=6.75 Hz, 1H), 4.85 (q, J=8.00 Hz, 1H), 4.90-5.01 (m, 1H), 5.04-5.19 (m, 1H), 6.88 (br t, J=7.50 Hz, 1H), 7.00 (br d, J=8.25 Hz, 1H), 7.31-7.45 (m, 2H), 12.13-12.78 (m, 1H).

### Example 11: Preparation of Compounds F-2 to F-5—(2R,4S)-3-methyl-2-((S)-2-phenyl-4,5-dihydrothiazol-4-yl)thiazolidine-4-carboxylic acid (F-4); (2R,4S)-3-methyl-2-((R)-2-phenyl-4,5-dihydrothiazol-4-yl)thiazolidine-4-carboxylic acid (F-3); (2S,4S)-3-methyl-2-((R)-2-phenyl-4,5-dihydrothiazol-4-yl)thiazolidine-4-carboxylic acid (F-2); (2S,4S)-3-methyl-2-((S)-2-phenyl-4,5-dihydrothiazol-4-yl)thiazolidine-4-carboxylic acid (F-5)

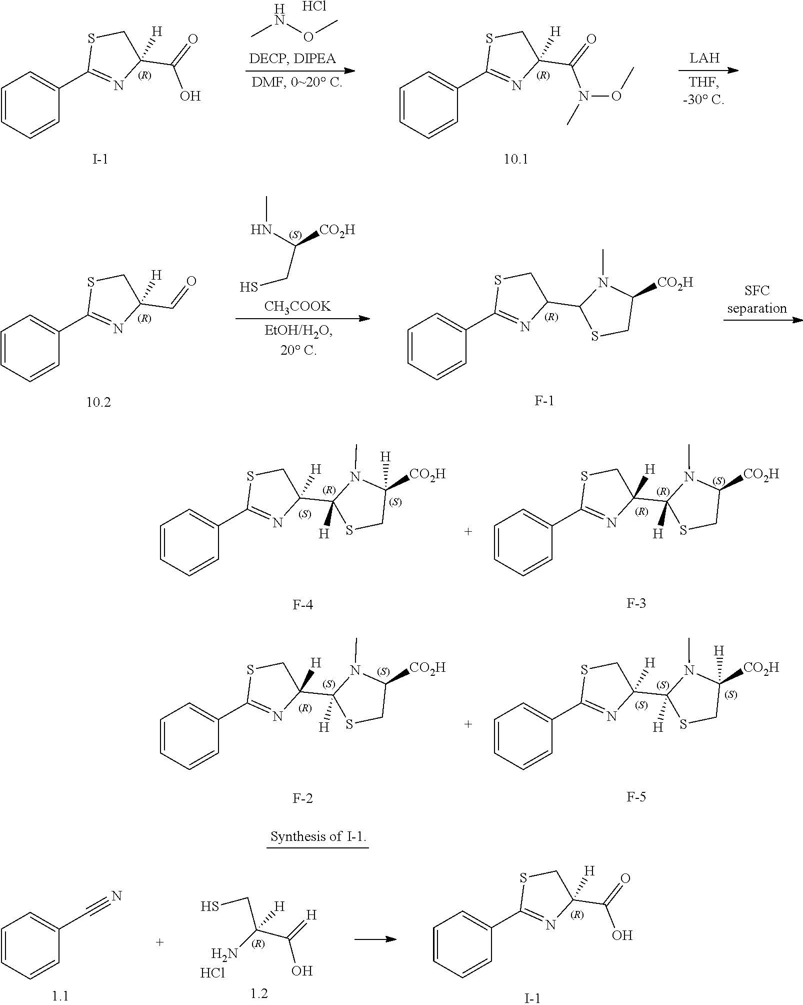

To a solution of benzonitrile (1.1, 1.0 g, 9.70 mmol, 990 μL, 1 eq) and (R)-2-amino-3-mercaptopropanoic acid hydrochloride (1.2, 2.29 g, 14.6 mmol, 1.5 eq) in MeOH (45.0 mL) and H₂O (17.5 mL) was added NaHCO₃ (2.04 g, 24.2 mmol, 943 L, 2.5 eq) and NaOH (2 mg, 48.5 mol, 0.005 eq) in one portion at 25° C. The mixture was stirred at 40° C. for 12 h. Then the mixture was concentrated in vacuum to remove most of the MeOH. The residue was extracted with ethyl acetate (2×20 mL), the aqueous phase was adjusted to pH around 3 with 2M HCl (aq.), then extracted with ethyl acetate (3×30 mL), the combined organic layers were washed with brine (20 mL), dried over anhydrous sodium sulfate and concentrated to give 1.5 g of I-1 (74%, % ee value is 100.0%) as a white solid. (ES, m/z): [M+1]⁺ 208.0.

Synthesis of 10.1. To a solution of (R)-2-phenyl-4,5-dihydrothiazole-4-carboxylic acid (900.00 mg, 4.34 mmol, 1 eq), N-methoxymethanamine (465.95 mg, 4.78 mmol, 1.1 eq, HCl salt) and DECP (820.17 mg, 4.78 mmol, 759.42 μL, 95% purity, 1.1 eq) in DMF (10 mL) was added DIPEA (1.12 g, 8.69 mmol, 1.51 mL, 2 eq) at 0° C. under N₂. The mixture was stirred for 30 min at 0° C., then the reaction mixture was stirred for 11.5 h at 25° C. After the reaction was finished, a 3% aqueous solution of citric acid (40 mL) was added drop-wise at room temperature, white solid was precipitated, and the slurry was then filtered. The filter cake was conserved and the filtrate was extracted twice with a mixture of toluene and ethyl acetate (1:1, v/v; 50 mL). The organic phase was washed with aq. NaHCO₃, then with brine, dried over anhydrous sodium sulfate, filtered and evaporated. The residue and the filter cake were purified by silica gel column chromatography (SiO₂, Petroleum ether:Ethyl acetate from 1:0 to 1:1) to give 900 mg of 10.1 (82%, ee value is 100.0%) as a light yellow solid. (ES, m/z): [M+1]⁺ 251.1.

Synthesis of 10.2. To a solution of (R)—N-methoxy-N-methyl-2-phenyl-4,5-dihydrothiazole-4-carboxamide (10.1, 700 mg, 2.80 mmol, 1 eq) in THE (20 mL) was added LAH (159.19 mg, 4.19 mmol, 1.5 eq) at −40° C. under N₂. The temperature was allowed to rise up to −30° C. in 0.5 h. The reaction mixture was then hydrolyzed by successive additions of aqueous solutions of saturated NH₄Cl (20 mL) and then a 1 M solution of KHSO₄ (10 mL). The mixture was vigorously stirred and allowed to warm up to the room temperature until two phases were formed. After partition and extraction with dichloromethane (2×20 mL), the organic layers were combined, dried over anhydrous sodium sulfate and filtered before being evaporated under reduced pressure to give 534 mg of 10.2 (crude) as a yellow solid. The crude product was used to the next step without further purification. (ES, m/z): [M+1]⁺ 192.0.

Synthesis of F-1. To a solution of (R)-2-phenyl-4,5-dihydrothiazole-4-carbaldehyde (10.2, 534 mg, 2.79 mmol, 1 eq) in a mixture of EtOH (60 mL) and H₂O (20 mL) was added (2S)-2-(methylamino)-3-sulfanyl-propanoic acid (575.12 mg, 3.35 mmol, 1.2 eq, HCl salt) and KOAc (1.92 g, 19.55 mmol, 7 eq) at 20° C. under Ar. The suspension was gently stirred 12 h at 20° C. in dark. Ethanol was removed under reduced pressure and the crude was diluted in water. The aqueous layer was acidified to pH=5 by the addition of solid citric acid, and then the solution was extracted with ethyl acetate (2×100 mL). The organic layers were combined, dried over anhydrous sodium sulfate, filtered and evaporated to dryness under reduced pressure. The residue was purified by prep-HPLC (neutral condition; neutral condition; column: Welch Xtimate C18 250*70 mm #10 μm; mobile phase: [water (10 mM NH₄HCO₃)-ACN]; B %: 15%-45%, 20 min) to give 300 mg mixture of F-1 (34%, 4 desired isomers and 2 aromatized isomers) as a light yellow solid. (ES, m/z): [M+1]⁺ 309.1.

Separation of F-2 to F-5. 500 mg of F-1 was purified by SFC separation twice to give 22.09 mg of F-4 (4%, de value is 99%, Peak 1), 57.16 mg of F-5 (11%, de value is 100%, Peak 2), 48.3 mg of F-3 (9%, de value is 95%, Peak 3) and 10.98 mg of F-2 (2%, de value is 100%, Peak 4) as light yellow solids. (ES, m/z): [M+1]⁺ 339.0.

### SFC Separation Method:

Method 1: Instrument: Waters SFC 80 Q preparative SFC; column: DAICEL CHIRALPAK IG (250 mm*30 mm, 10 μm); mobile phase: A for CO₂ and B for MeOH (0.1% NH₃·H₂O); Gradient: B from 40%-40% in 10 min.

Method 2: Instrument: Waters SFC 80 Q preparative SFC; column: DAICEL CHIRALPAK AS (250 mm*30 mm, 10 μm); mobile phase: A for CO₂ and B for MeOH (0.1% NH₃·H₂O); Gradient: B % from 35%-35% in 16 min.

F-4: ¹H NMR (400 MHz, acetone-d6) δ=7.85-7.79 (m, 2H), 7.55-7.43 (m, 3H), 4.92 (d, J=8.0 Hz, 1H), 4.57 (d, J=7.7 Hz, 1H), 4.30 (t, J=6.3 Hz, 1H), 3.66 (dd, J=8.7, 11.2 Hz, 1H), 3.46 (dd, J=8.6, 11.1 Hz, 1H), 3.25-3.18 (m, 3H), 2.48 (s, 3H)

F-5: ¹H NMR (400 MHz, acetone-d6) δ=7.81 (d, J=7.1 Hz, 2H), 7.58-7.38 (m, 3H), 5.01 (dt, J=5.8, 8.8 Hz, 1H), 4.56 (br d, J=5.5 Hz, 1H), 3.70 (t, J=7.5 Hz, 1H), 3.48 (br d, J=8.9 Hz, 2H), 3.26-3.17 (m, 2H), 2.63 (s, 3H)

F-3: ¹H NMR (400 MHz, acetone-d6) δ=7.95-7.69 (m, 2H), 7.65-7.33 (m, 3H), 5.01 (dt, J=5.4, 9.2 Hz, 1H), 4.49 (d, J=5.2 Hz, 1H), 3.89 (br t, J=6.4 Hz, 1H), 3.62 (dd, J=8.7, 11.1 Hz, 1H), 3.42-3.16 (m, 3H), 2.68 (s, 3H)

F-2: ¹H NMR (400 MHz, acetone-d6) δ=7.83-7.77 (m, 2H), 7.54-7.41 (m, 3H), 5.22-5.07 (m, 1H), 4.25 (dd, J=1.6, 6.5 Hz, 1H), 3.50-3.37 (m, 2H), 3.19 (dd, J=6.6, 10.4 Hz, 1H), 3.01 (br d, J=10.6 Hz, 2H), 2.72 (s, 3H)

### Example 12: (2R,4S)-2-((S)-2-(2-hydroxy-5-methylphenyl)-4,5-dihydrothiazol-4-yl)-3-methylthiazolidine-4-carboxylic acid and (2S,4S)-2-((S)-2-(2-hydroxy-5-methylphenyl)-4,5-dihydrothiazol-4-yl)-3-methylthiazolidine-4-carboxylic acid

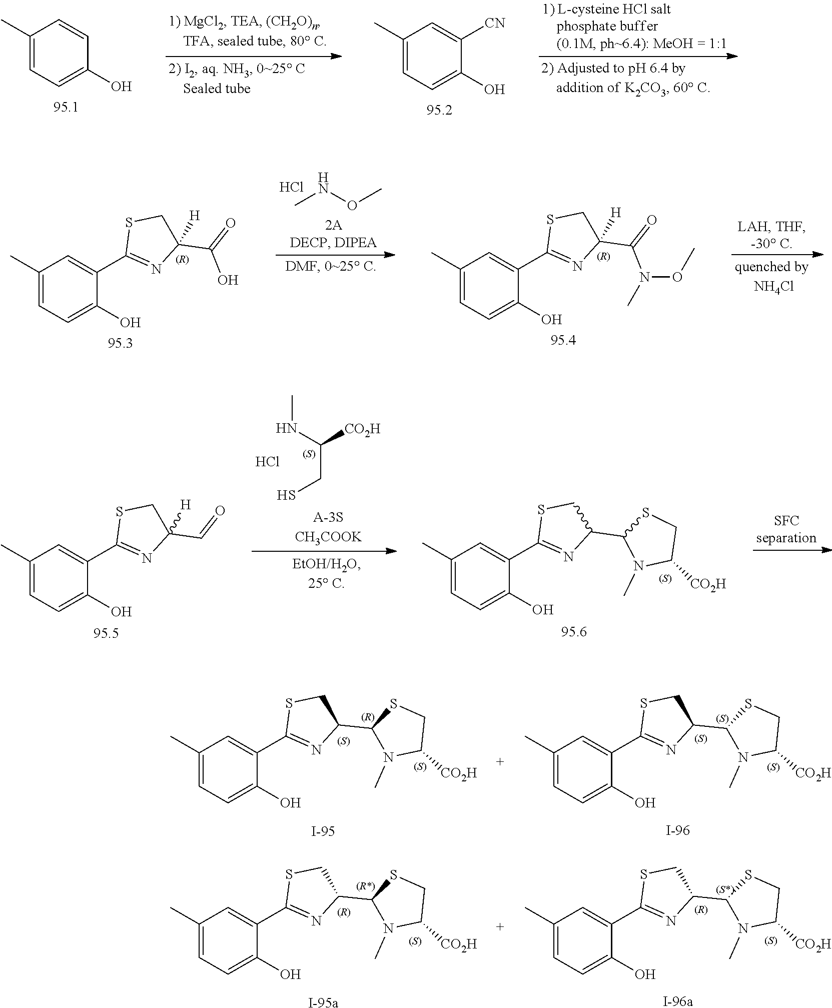

Synthesis of 95.2. To a mixture of p-cresol (95.1, 3 g, 27.74 mmol, 2.91 mL, 1 eq) in dry THF (70 mL) in a 100 mL of sealed tube was added anhydrous magnesium chloride (5.28 g, 55.48 mmol, 2.28 mL, 2 eq), TEA (8.42 g, 83.23 mmol, 11.58 mL, 3 eq), and paraformaldehyde (2.5 g, 83.23 mmol, 3 eq). The mixture was stirred for 16 h at 80° C. The reaction was used into the next step without further purification. NH₃·H₂O (254.83 g, 1.82 mol, 280.03 mL, 25% purity, 65.47 eq) and I₂ (8.46 g, 33.32 mmol, 6.71 mL, 1.2 eq) was added to the above mixture at 0° C., and the obtained mixture was stirred for 16 h at 25° C. The reaction mixture was quenched by sat. aq. Na₂SO₃ (40 mL). The obtained mixture was extracted with ethyl acetate (3×40 mL) and then the organic layer was combined and dried with Na₂SO₄. After that, the suspension was filtered, the filtrate was concentrated under reduced pressure. The residue was purified by flash silica gel chromatography (ISCO®; 40 g SepaFlash® Silica Flash Column, Eluent of 0-100% Ethyl acetate/Petroleum ether gradient @36 mL/min) to give 1.6 g of 95.2 (43%) as a yellow solid.

Synthesis of 95.3. To a solution of 2-hydroxy-5-methylbenzonitrile (95.2, 0.7 g, 5.26 mmol, 1 eq) in MeOH (10 mL) was added L-cysteine (1.66 g, 10.51 mmol, 2 eq, HCl salt) and phosphate buffer (10 mL) at 25° C. The solution was adjusted to pH=6.4 by the addition of solid K₂CO₃ carefully and the reaction mixture was stirred at 60° C. for 2 h. The mixture was concentrated under reduced pressure, and the yellow crude residue was diluted with water (20 mL). The solution was adjusted to pH=2.0 by the addition of solid citric acid. After extraction with dichloromethane (3×20 mL), the organic layers were collected, dried over anhydrous sodium sulfate, filtered and concentrated under reduced pressure to give 1.05 g of 95.3 (73%, ee value: 95.3%) as a yellow solid. (ES, m/z): [M+1]⁺ 238.1.

Synthesis of 95.4. To a solution of (R)-2-(2-hydroxy-5-methylphenyl)-4,5-dihydrothiazole-4-carboxylic acid (95.3, 1.05 g, 4.43 mmol, 1 eq), N,O-dimethylhydroxylamine (2A, 474.82 mg, 4.87 mmol, 1.1 eq, HCl) and DECP (793.99 mg, 4.87 mmol, 735.18 uL, 1.1 eq) in DMF (11 mL) was added DIPEA (857.90 mg, 6.64 mmol, 1.16 mL, 1.5 eq) at 0° C. under N₂. The mixture was stirred for 30 min at 0° C., then another 2 h at 25° C. The residue was poured into water (10 mL) and stirred for 1 min. The aqueous phase was extracted with ethyl acetate (3×10 mL). The combined organic phase was washed with aq·NaHCO₃, then with brine, dried with anhydrous Na₂SO₄, filtered and concentrated in vacuum. The residue was purified by column chromatography (SiO₂, Petroleum ether/Ethyl acetate=15/1 to 0/1) to give 1.14 g of 95.4 (58%, ee value: 97.1%) as a yellow solid. (ES, m/z): [M+1]⁺ 281.1.

Synthesis of 95.5. To a solution of (R)-2-(2-hydroxy-5-methylphenyl)-N-methoxy-N-methyl-4,5-dihydrothiazole-4-carboxamide (95.4, 1.2 g, 4.28 mmol, 1 eq) in THE (14.5 mL) was added LAH (243.69 mg, 6.42 mmol, 1.5 eq) at −30° C. under N₂. The temperature was allowed to rise up to −30° C. in 0.5 h. The reaction mixture was then hydrolyzed by successive additions of aqueous solutions of saturated NH₄Cl (20 mL) and 1 M solution of KHSO₄ (100 mL). The mixture was vigorously stirred and allowed to warm up to the room temperature until two phases were formed. After partition and extraction with ethyl acetate (3×50 mL), the organic layers were combined, dried over anhydrous sodium sulfate and filtered before being evaporated under reduced pressure to give 947 mg of 95.5 (crude) as a yellow oil. The crude product was used to the next step without further purification. (ES, m/z): [M+1]⁺ 240.2.

Synthesis of 95.6. To a solution of (R)-2-(2-hydroxy-5-methylphenyl)-4,5-dihydrothiazole-4-carbaldehyde (95.5, 947 mg, 4.28 mmol, 1 eq) in a mixture of EtOH (30 mL) and H₂O (10 mL) was added (2S)-2-(methylamino)-3-sulfanyl-propanoic acid (A-3S, 881.52 mg, 5.14 mmol, 1.2 eq, HCl) and KOAc (2.94 g, 29.96 mmol, 7 eq) at 25° C. The suspension was gently stirred 2.5 h at 25° C. in the dark. Ethanol was removed under reduced pressure and the crude was diluted in water. The aqueous layer was acidified to pH=5 by the addition of solid citric acid, and then the solution was extracted with ethyl acetate (2×30 mL). The organic layers were combined, dried over anhydrous sodium sulfate, filtered and evaporated to dryness under reduced pressure. The residue was purified by prep-HPLC (column: Welch Xtimate C18 250*70 mm #10 um; mobile phase: [water (NH₄HCO₃)-ACN]; B %: 5%-40%, 20 min) to give 320 mg of 95.6 (22%) as a yellow solid. (ES, m/z): [M+1]⁺ 339.2.

Synthesis of I-95, I-96, I-95a, and I-96a. 320 mg of 95.6 (mixture of 4 peaks) was purified by SFC separation to give 57.12 mg of I-95 (18%, de value: 84.9%, Peak 1), 55.04 mg of I-95a (17%, ee value: 91.6%, Peak 2), 18.52 mg of I-96a (6.0%, de value: 92.2%, Peak 3) and 78.97 mg of I-96 (24%, de value: 90.6%, Peak 4) as light yellow solids. (ES, m/z): [M+1]⁺ 339.1.

Method 1: Instrument: Waters SFC80 preparative SFC; Column: DAICEL CHIRALPAK IG (250 mm*30 mm, 10 μm); Mobile phase: A for CO₂ and B for MeOH (0.1% NH₃H₂O); Gradient: B %=50% isocratic elution mode; Flow rate: 80 g/min; Wavelength:220 nm; Column temperature: 40° C.; System back pressure: 100 bar

I-95: ¹H NMR (400 MHz, acetone-d6) δ=7.26-7.20 (m, 2H), 6.85 (d, J=8.9 Hz, 1H), 5.00 (q, J=8.3 Hz, 1H), 4.54 (d, J=8.3 Hz, 1H), 4.25 (t, J=6.4 Hz, 1H), 3.66 (dd, J=8.6, 11.3 Hz, 1H), 3.45 (dd, J=7.9, 11.3 Hz, 1H), 3.23 (d, J=6.6 Hz, 2H), 2.50 (s, 3H), 2.28 (s, 3H).

I-96: ¹H NMR (400 MHz, acetone-d6) δ=7.28-7.18 (m, 2H), 6.85 (d, J=8.9 Hz, 1H), 5.24-5.16 (m, 1H), 4.59 (br d, J=5.1 Hz, 1H), 3.70-3.60 (m, 1H), 3.48 (br d, J=9.0 Hz, 2H), 3.19 (br d, J=7.3 Hz, 2H), 2.63 (s, 3H), 2.28 (s, 3H).

I-95a: ¹H NMR (400 MHz, acetone-d6) δ=7.29-7.17 (m, 2H), 6.84 (d, J=9.0 Hz, 1H), 4.83 (br d, J=8.1 Hz, 1H), 4.33 (d, J=8.3 Hz, 1H), 4.01 (br t, J=5.8 Hz, 1H), 3.68-3.57 (m, 1H), 3.56-3.39 (m, 2H), 3.30 (dd, J=6.9, 11.0 Hz, 1H), 2.63 (s, 3H), 2.28 (s, 3H).

I-96a: ¹H NMR (400 MHz, acetone-d6) δ=7.28-7.14 (m, 2H), 6.92-6.76 (m, 1H), 5.26 (dt, J=4.6, 9.1 Hz, 1H), 5.05 (d, J=4.6 Hz, 1H), 4.25 (dd, J=1.7, 6.3 Hz, 1H), 3.48-3.33 (m, 2H), 3.21 (dd, J=6.5, 10.4 Hz, 1H), 3.12-2.96 (m, 1H), 2.70 (s, 3H), 2.27 (s, 3H).

### Example 13: (4S)-2-((S)-2-(2-hydroxy-5-methylphenyl)-4,5-dihydrothiazol-4-yl)-3-methylthiazolidine-4-carboxylic acid

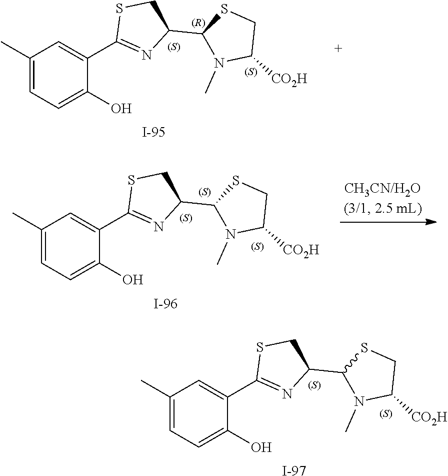

Synthesis of I-97. A solution of 50 mg of 1-95 and 75 mg of 1-96 in CH₃CN/H₂O (3/1, 2.5 mL) was shaken for 2 min to give a clear solution. The mixture was then lyophilized for 24 h to give 85.52 mg of I-97 (68%). (ES, m/z): [M+1]⁺ 339.1. ¹H NMR (400 MHz, acetone) δ=7.23 (br s, 2.93H), 6.85 (dd, J=2.7, 8.8 Hz, 1.52H), 5.24-5.14 (m, 0.86H), 5.04-4.96 (m, 0.61H), 4.62 (d, J=5.4 Hz, 0.87H), 4.55 (d, J=8.4 Hz, 0.68H), 4.25 (t, J=6.5 Hz, 0.71H), 3.75-3.60 (m, 1.99H), 3.55-3.40 (m, 3.27H), 3.27-3.18 (m, 4.05H), 2.65 (s, 3H), 2.50 (s, 2.12H), 2.28 (s, 5.16H).

### Example 14: (2R,4S)-2-((S)-2-(2-hydroxy-3-methylphenyl)-4,5-dihydrothiazol-4-yl)-3-methylthiazolidine-4-carboxylic acid and (2S,4S)-2-((S)-2-(2-hydroxy-3-methylphenyl)-4,5-dihydrothiazol-4-yl)-3-methylthiazolidine-4-carboxylic acid

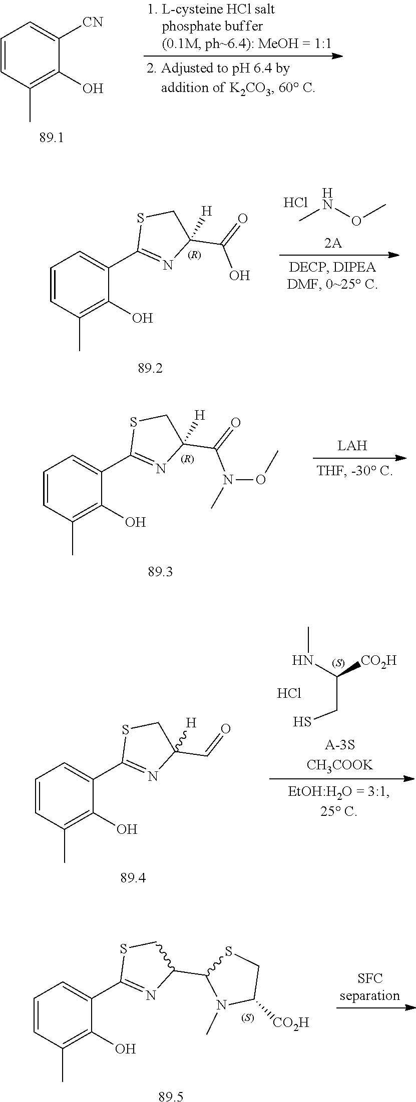

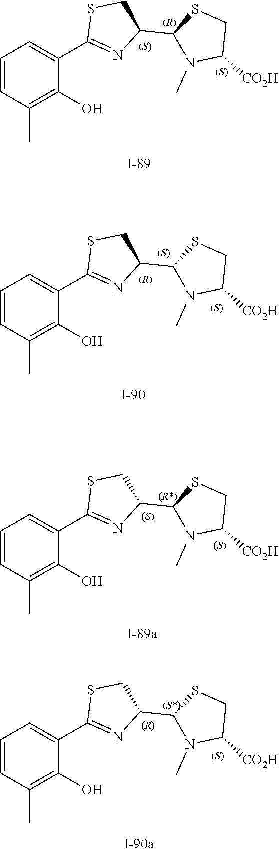

Synthesis of 89.2. To a solution of 2-hydroxy-3-methylbenzonitrile (89.1, 3 g, 22.53 mmol, 1 eq) in MeOH (30 mL) were added L-cysteine (7.10 g, 45.06 mmol, 2 eq, HCl salt) and phosphate buffer (30 mL) at room temperature. The solution was adjusted to pH=6.4 by the addition of solid K₂CO₃ carefully and the reaction mixture was stirred at 60° C. for 12 h. The mixture was concentrated under reduced pressure, and the yellow crude residue was diluted with water (500 mL). The solution was adjusted to pH=2.0 by the addition of solid citric acid. After extraction with ethyl acetate (3×500 mL), the organic layers were collected, dried over anhydrous sodium sulfate, filtered and concentrated under reduced pressure to give 5.06 g of 89.2 (94%, ee value: 93.4%) as a yellow solid. (ES, m/z): [M+1]⁺ 238.1.

Synthesis of 89.3. To a solution of (R)-2-(2-hydroxy-3-methylphenyl)-4,5-dihydrothiazole-4-carboxylic acid (89.2, 3 g, 12.64 mmol, 1 eq), N,O-dimethylhydroxylamine (2A, 1.36 g, 13.91 mmol, 1.1 eq, HCl) and DECP (2.27 g, 13.91 mmol, 2.10 mL, 1.1 eq) in DMF (30 mL) was added DIPEA (2.45 g, 18.97 mmol, 3.30 mL, 1.5 eq) dropwise at 0° C. under N₂. The mixture was stirred for 30 min at 0° C., then another 2 h at 25° C. The mixture was poured into water (30 mL) and extracted with ethyl acetate (3×30 mL). The organic phase was washed with aq. NaHCO₃, then washed with brine, dried over anhydrous sodium sulfate, filtered and evaporated. The residue and the filter cake were purified by silica gel column chromatography (SiO₂, Petroleum ether:Ethyl acetate from 1:0 to 1:1) to give 2.5 g of 89.3 (67%, ee value: 91.0%) as a light yellow solid. (ES, m/z): [M+1]⁺ 281.1.

Synthesis of 89.4. To a solution of (R)-2-(2-hydroxy-3-methylphenyl)-N-methoxy-N-methyl-4,5-dihydrothiazole-4-carboxamide (89.3, 2.2 g, 7.85 mmol, 1 eq) in THE (22 mL) was added LAH (446.72 mg, 11.77 mmol, 1.5 eq) at −40° C. under N₂. The temperature was allowed to rise up to −30° C. and stirred for 0.5 h. The reaction mixture was then hydrolyzed by successive additions of aqueous solutions of saturated NH₄Cl (25 mL) and of a 1 M solution of KHSO₄ (25 mL). The mixture was vigorously stirred and allowed to warm up to the room temperature until two phases were formed. After partition and extraction with ethyl acetate (2×200 mL), the organic layers were combined, dried over anhydrous sodium sulfate and filtered before being evaporated under reduced pressure to give 1.74 g of 89.4 (crude) as a yellow solid. The crude product was used to the next step without further purification. (ES, m/z): [M+1]⁺ 222.1.

Synthesis of 89.5. To a solution of 2-(2-hydroxy-3-methylphenyl)-4,5-dihydrothiazole-4-carbaldehyde (89.4, 1.74 g, 7.86 mmol, 1 eq) in a mixture of EtOH (51 mL) and H₂O (17 mL) was added (S)-3-mercapto-2-(methylamino)propanoic acid (A-3S, 1.62 g, 9.44 mmol, 1.2 eq, HCl) and KOAc (5.40 g, 55.04 mmol, 7 eq) at 25° C. under Ar. The suspension was gently stirred 16 h at 25° C. in the dark. Ethanol was removed under reduced pressure and the crude was diluted in water. The aqueous layer was acidified to pH=5 by the addition of solid citric acid, and then the solution was extracted with ethyl acetate (2×200 mL). The organic layers were combined, dried over anhydrous sodium sulfate, filtered and evaporated to dryness under reduced pressure. The residue was purified by prep-HPLC (column: Welch Xtimate C18 250*70 mm #10 um; mobile phase: [water (NH₄HCO₃)-ACN]; B %: 15%-40%, 20 min) to give 1 g of 89.5 (37%) as a light yellow solid. (ES, m/z): [M+1]⁺ 339.2.

Synthesis of I-89, I-90, I-89a, and I-90a. 500 mg of 89.5 (mixture of 4 peaks) was purified by twice SFC separation (method 1 followed by method 2) and 1 time prep-HPLC purification to give 2 mg of I-89a (0.4%, de value: 41.8%, Peak 1), 10 mg of I-90a (2.0%, de value: 94.1%, Peak 2), 38 mg of I-90 (7.6%, de value: 94.4%, Peak 3) and 14 mg of I-89 (2.8%, de value: 97.4%, Peak 4) as light yellow solids.

### SFC Separation Methods:

Method 1 (First time SFC separation): Instrument: Waters SFC80Q preparative SFC; Column: DAICEL CHIRALPAK ID (250 mm*30 mm, 10 μm); Mobile phase: A for CO₂ and B for MeOH (0.1% NH₃H₂O); Gradient: B %=56% isocratic elution mode; Flow rate: 72 g/min; Wavelength: 220 nm; Column temperature: 40° C.; System back pressure: 100 bar.

Method 2 (Second time SFC separation): Instrument: Waters SFC80Q preparative SFC; Column: REGIS (s,s) WHELK-01 (250 mm*30 mm, 10 μm); Mobile phase: A for CO₂ and B for MeOH (0.1% IPAm); Gradient: B %=35% isocratic elution mode; Flow rate: 62 g/min; Wavelength:220 nm; Column temperature: 40° C.; System back pressure: 100 bar.

### Prep-HPLC Purification:

Column: Phenomenex C18 80*40 mm*3 um; mobile phase: [water (NH₄HCO₃)-ACN]; B %: 10%-40%, 8 min.

I-90: ¹H NMR (400 MHz, acetone-d6) δ=7.28 (d, J=7.5 Hz, 2H), 6.83 (t, J=7.6 Hz, 1H), 5.22 (dt, J=5.6, 8.9 Hz, 1H), 4.63 (d, J=5.5 Hz, 1H), 3.69 (t, J=7.5 Hz, 1H), 3.49 (d, J=8.8 Hz, 2H), 3.23-3.18 (m, 2H), 2.65 (s, 3H), 2.22 (s, 3H).

I-89: ¹H NMR (400 MHz, acetone-d6) δ=7.30-7.26 (m, 2H), 6.82 (t, J=7.6 Hz, 1H), 5.01 (q, J=8.5 Hz, 1H), 4.55 (d, J=8.6 Hz, 1H), 4.23 (t, J=6.5 Hz, 1H), 3.66 (dd, J=8.7, 11.3 Hz, 1H), 3.51-3.43 (m, 1H), 3.28-3.19 (m, 2H), 2.50 (s, 3H), 2.23 (s, 3H).

I-89a: ¹H NMR (400 MHz, acetone-d6) δ=7.28 (d, J=7.6 Hz, 2H), 6.82 (t, J=7.4 Hz, 1H), 5.29 (dt, J=4.8, 9.3 Hz, 1H), 5.07 (d, J=4.6 Hz, 1H), 4.27 (dd, J=2.3, 6.6 Hz, 1H), 3.50-3.40 (m, 2H), 3.27-3.20 (m, 1H), 3.10-3.05 (m, 1H), 2.72 (s, 3H), 2.22 (s, 3H).

I-90a: ¹H NMR (400 MHz, acetone-d6) δ=7.28 (d, J=7.6 Hz, 2H), 6.82 (t, J=7.6 Hz, 1H), 4.84 (q, J=8.3 Hz, 1H), 4.35 (d, J=8.5 Hz, 1H), 4.02 (t, J=5.9 Hz, 1H), 3.68-3.61 (m, 1H), 3.57-3.43 (m, 2H), 3.31 (dd, J=6.9, 10.9 Hz, 1H), 2.64 (s, 3H), 2.22 (s, 3H).

### Example 15: (4S)-2-((S)-2-(2-hydroxy-3-methylphenyl)-4,5-dihydrothiazol-4-yl)-3-methylthiazolidine-4-carboxylic acid

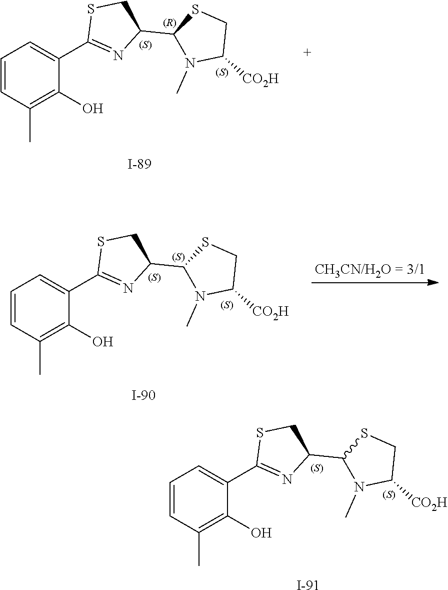

Synthesis of I-91. A solution of (2R,4S)-2-((S)-2-(2-hydroxy-3-methylphenyl)-4,5-dihydrothiazol-4-yl)-3-methylthiazolidine-4-carboxylic acid (I-89, 13.25 mg) and (2S,4S)-2-((S)-2-(2-hydroxy-3-methylphenyl)-4,5-dihydrothiazol-4-yl)-3-methylthiazolidine-4-carboxylic acid (I-90, 19.87 mg) in ACN/H₂O (3/1, 2 mL) was shaken for 2 min to give a clear solution. The clear solution was then lyophilized for 48 h to give 27.10 mg of I-91 (82%) as a light yellow solid. (ES, m/z): [M+1]⁺ 339.1.

¹H NMR (400 MHz, acetone-d6) δ=7.28 (d, J=7.7 Hz, 3.12H), 6.83 (t, J=7.6 Hz, 1.60H), 5.21 (dt, J=5.5, 8.9 Hz, 0.95H), 5.02 (q, J=8.3 Hz, 0.64H), 4.64 (d, J=5.5 Hz, 0.93H), 4.56 (d, J=8.4 Hz, 0.64H), 4.26 (t, J=6.5 Hz, 0.66H), 3.78-3.60 (m, 2.01H), 3.52-3.41 (m, 3.40H), 3.29-3.17 (m, 4.07H), 2.66 (s, 3.00H), 2.51 (s, 2.00H), 2.23 (d, J=2.3 Hz, 4.93H).

### Example 16: (2R,4S)-2-((S)-2-(5-fluoro-2-hydroxyphenyl)-4,5-dihydrothiazol-4-yl)-3-methylthiazolidine-4-carboxylic acid and (2S,4S)-2-((S)-2-(5-fluoro-2-hydroxyphenyl)-4,5-dihydrothiazol-4-yl)-3-methylthiazolidine-4-carboxylic acid

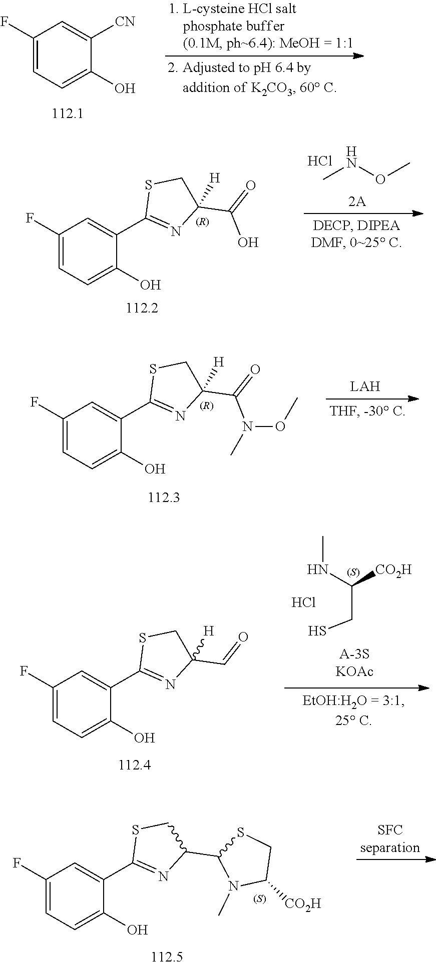

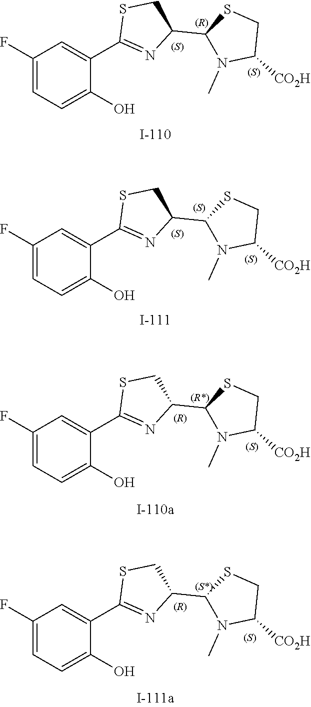

Synthesis of 112.2. To a mixture of 5-fluoro-2-hydroxybenzonitrile (112.1, 4.5 g, 32.82 mmol, 1 eq) in MeOH (45 mL) was added L-cysteine (10.35 g, 65.64 mmol, 2 eq, HCl salt) and phosphate buffer (45 mL) at room temperature. The solution was adjusted to pH=6.4 by the addition of solid K₂CO₃ carefully and the reaction mixture was stirred at 60° C. for 12 h. The mixture was concentrated under reduced pressure, and the yellow crude residue was diluted with water (40 mL). The solution was adjusted to pH=2.0 by the addition of solid citric acid. After extraction with ethyl acetate (3×60 mL). The combined organic layers were dried over Na₂SO₄, filtered and concentrated under reduced pressure to give a residue to give 7.47 g of 112.2 (94%, ee value: 89.0%) as a yellow solid. (ES, m/z): [M+1]⁺ 242.0.

Synthesis of 112.3. To a solution of (R)-2-(5-fluoro-2-hydroxyphenyl)-4,5-dihydrothiazole-4-carboxylic acid (112.2, 3 g, 11.81 mmol, 95% purity, 1 eq), N-methoxymethanamine (1.27 g, 13.00 mmol, 1.1 eq, HCl) and DECP (2.12 g, 13.00 mmol, 1.96 mL, 1.1 eq) in DMF (30 mL) was added DIEA (2.29 g, 17.72 mmol, 3.09 mL, 1.5 eq) at 0° C. under N₂. The mixture was stirred for 30 min at 0° C., then another 2 h at 25° C. The reaction mixture was diluted with water (45 mL) and extracted with ethyl acetate (60 mL×3). The combined organic layers were washed with NaHCO₃ (30 mL*3), dried over Na₂SO₄, filtered and concentrated under reduced pressure to give a residue to give 1.74 g of 112.3 (49%, ee value: 79.0%) as a light yellow solid. (ES, m/z): [M+1]⁺ 285.1.

Synthesis of 112.4. To a solution of (R)-2-(5-fluoro-2-hydroxyphenyl)-N-methoxy-N-methyl-4,5-dihydrothiazole-4-carboxamide (112.3, 2 g, 7.03 mmol, 1 eq) in THE (20 mL) was added LAH (400.45 mg, 10.55 mmol, 1.5 eq) at −40° C. under N₂. The temperature was allowed to rise up to −30° C. in 0.5 h. The reaction mixture was then hydrolyzed by successive additions of aqueous solutions of saturated NH₄Cl (42 mL) and of a 1 M solution of KHSO₄ (42 mL). The mixture was vigorously stirred and allowed to warm up to the room temperature until two phases were formed. After partition and extraction with ethyl acetate (3×30 mL), the organic layers were collected, dried over Na₂SO₄ and filtered before being evaporated under reduced pressure to give 1.58 g of 112.4 (crude) as a yellow oil. The crude product was used to the next step without further purification. (ES, m/z): [M+1]⁺ 226.1.

Synthesis of I-112.5. To a solution of (R)-2-(5-fluoro-2-hydroxyphenyl)-4,5-dihydrothiazole-4-carbaldehyde (112.4, 1.58 g, 7.01 mmol, 1 eq) in a mixture of EtOH (45 mL) and H₂O (15 mL) was added (2S)-2-(methylamino)-3-sulfanyl-propanoic acid (A-3S, 1.14 g, 8.42 mmol, 1.2 eq, HCl) and KOAc (4.82 g, 49.10 mmol, 7 eq). This suspension was gently stirred 12 h at 25° C. in the dark. Ethanol was removed under reduced pressure and the crude was diluted in water. The aqueous layer was acidified to pH=5 by the addition of solid citric acid, and then the solution was extracted with ethyl acetate (3×80 mL). The organic layers were collected, dried over Na₂SO₄, filtered and evaporated to dryness under reduced pressure. The residue was purified by prep-HPLC (column: Welch Xtimate C18 250*70 mm #10 um; mobile phase: [water (NH₄HCO₃)-ACN]; B %: 10%-40%, 20 min) to give 690 mg of I-112.5 (29%) as a light yellow solid. I-110 and I-111 were not recovered in the prep-HPLC purification. (ES, m/z): [M+1]⁺ 343.1.

Synthesis of I-110 I-111. 600 mg of I-112.5 (mixture of 2 peaks) was purified by twice SFC separation (same separation method) to give 160 mg of I-110 (27%, de value: 98.4%, Peak 1) and 224 mg of I-111 (37%, de value: 99.4%, Peak 2) as light yellow solids. (ES, m/z): [M+1]+ 343.1.

Method 1: Instrument: Waters SFC80 preparative SFC; Column: DAICEL CHIRALPAK IG (250 mm*30 mm, 10 μm); Mobile phase: A for CO₂ and B for EtOH (0.1% NH₃H₂O); Gradient: B %=50% isocratic elution mode; Flow rate: 80 g/min; Wavelength:220 nm; Column temperature: 40° C.; System back pressure: 100 bar.

I-110: ¹H NMR (400 MHz, acetone-d6) δ=7.29-7.12 (m, 2H), 6.98 (dd, J=4.6, 9.0 Hz, 1H), 5.05 (q, J=8.2 Hz, 1H), 4.56 (d, J=8.2 Hz, 1H), 4.25 (t, J=6.4 Hz, 1H), 3.71 (dd, J=8.7, 11.2 Hz, 1H), 3.51 (dd, J=8.0, 11.3 Hz, 1H), 3.28-3.19 (m, 2H), 2.51 (s, 3H).

I-111: ¹H NMR (400 MHz, acetone-d6) δ=7.26-7.12 (m, 2H), 6.97 (dd, J=4.6, 9.0 Hz, 1H), 5.26 (dt, J=5.2, 9.0 Hz, 1H), 4.61 (d, J=5.1 Hz, 1H), 3.67-3.50 (m, 3H), 3.19 (d, J=7.6 Hz, 2H), 2.63 (s, 3H).

### Example 17: (4S)-2-((S)-2-(5-fluoro-2-hydroxyphenyl)-4,5-dihydrothiazol-4-yl)-3-methylthiazolidine-4-carboxylic acid

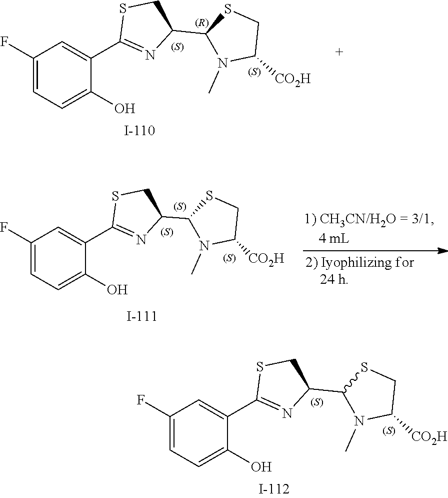

Synthesis of I-112. A solution of 148.88 mg of 1-110 and 223.32 mg of I-111 in ACN/H₂O (3/1, 4 mL) was shaken for 2 min to give a clear solution. The solution was then lyophilized for 24 h to give 337.12 mg of I-112 (91%). (ES, m/z): [M+1]=343.1.

¹H NMR (400 MHz, acetone-d6) δ=7.26-7.13 (m, 3.10H), 6.98 (ddd, J=3.1, 4.6, 9.0 Hz, 1.55H), 5.24 (dt, J=5.5, 8.9 Hz, 0.99H), 5.05 (q, J=8.3 Hz, 0.57H), 4.64 (d, J=5.4 Hz, 0.98H), 4.57 (d, J=8.3 Hz, 0.55H), 4.26 (t, J=6.5 Hz, 0.56H), 3.77-3.66 (m, 1.68H), 3.58-3.48 (m, 2.68H), 3.28-3.18 (m, 3.26H), 2.65 (s, 3.00H), 2.51 (s, 1.67H).

### Example 18: (2R,4S)-2-((S)-2-(2-fluoro-6-hydroxyphenyl)-4,5-dihydrothiazol-4-yl)-3-methylthiazolidine-4-carboxylic acid and (2S,4S)-2-((S)-2-(2-fluoro-6-hydroxyphenyl)-4,5-dihydrothiazol-4-yl)-3-methylthiazolidine-4-carboxylic acid

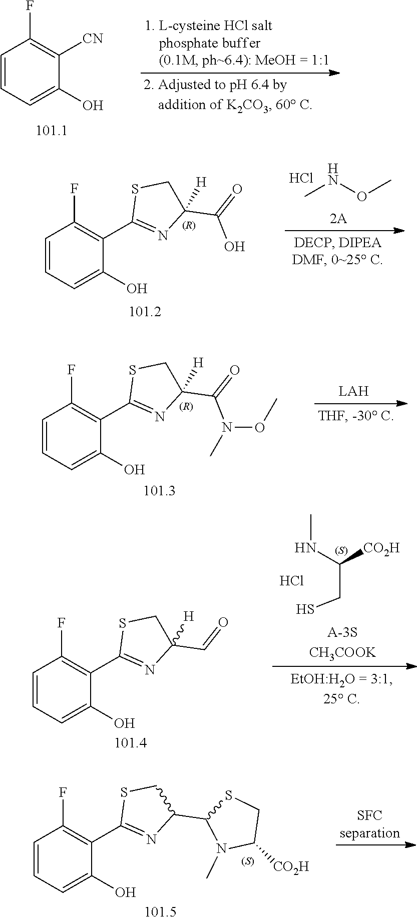

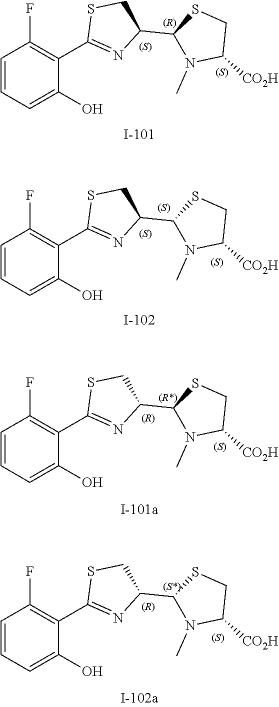

Synthesis of 101.2. To a solution of 2-fluoro-6-hydroxybenzonitrile (101.1, 3.3 g, 24.07 mmol, 1 eq) in MeOH (33 mL) were added L-cysteine (37.94 g, 240.68 mmol, 10 eq, HCl salt) and phosphate buffer (33 mL) at room temperature. The solution was adjusted to pH=6.4 by the addition of solid K₂CO₃ carefully and the reaction mixture was stirred at 60° C. for 12 h. The mixture was concentrated under reduced pressure, and the yellow crude residue was diluted with water (500 mL). The solution was adjusted to pH=2.0 by the addition of solid citric acid. After extraction with ethyl acetate (3×500 mL), the organic layers were collected, dried over anhydrous sodium sulfate, filtered and concentrated under reduced pressure to give 4.10 g of 101.2 (70%, ee value: 80.0%) as a yellow solid. (ES, m/z): [M+1]⁺ 242.3.

Synthesis of 101.3. To a solution of (R)-2-(2-fluoro-6-hydroxyphenyl)-4,5-dihydrothiazole-4-carboxylic acid (101.2, 4.10 g, 17.00 mmol, 1 eq), N,O-dimethylhydroxylamine (2A, 1.82 g, 18.70 mmol, 1.1 eq, HCl) and DECP (3.05 g, 18.70 mmol, 2.82 mL, 1.1 eq) in DMF (30 mL) was added DIPEA (3.29 g, 25.49 mmol, 4.44 mL, 1.5 eq) at 0° C. under N₂. The mixture was stirred for 30 min at 0° C., then another 2 h at 25° C. The mixture was poured into water (30 mL) and extracted with ethyl acetate (3×30 mL). The organic phase was washed with aq. NaHCO₃, and brine, dried over anhydrous sodium sulfate, filtered and evaporated. The residue was purified by silica gel column chromatography (SiO₂, Petroleum ether:Ethyl acetate from 1:0 to 1:1) to give 4.58 g of 101.3 (94%, ee value: 56.0%) as a light yellow solid. (ES, m/z): [M+1]⁺ 285.3.

Synthesis of 101.4. To a solution of (R)-2-(2-fluoro-6-hydroxyphenyl)-N-methoxy-N-methyl-4,5-dihydrothiazole-4-carboxamide (101.3, 4 g, 14.07 mmol, 1 eq) in THE (40 mL) was added LAH (800.90 mg, 21.10 mmol, 1.5 eq) at −40° C. under N₂. The temperature was allowed to rise up to −30° C. and stirred for 0.5 h. The reaction mixture was then hydrolyzed by successive additions aqueous solutions of saturated NH₄Cl (50 mL) and 1 M solution of KHSO₄ (50 mL). The mixture was vigorously stirred and allowed to warm up to the room temperature until two phases were formed. After partition and extraction with ethyl acetate (2×200 mL), the organic layers were combined, dried over anhydrous sodium sulfate and filtered before being evaporated under reduced pressure to give 2.5 g of 101.4 (crude) as a yellow solid. The crude product was used to the next step without further purification. (ES, m/z): [M+1]⁺ 226.2.

Synthesis of 101.5. To a solution of 2-(2-fluoro-6-hydroxyphenyl)-4,5-dihydrothiazole-4-carbaldehyde (101.4, 2.5 g, 11.10 mmol, 1 eq) in a mixture of EtOH (75 mL) and H₂O (25 mL) was added (S)-3-mercapto-2-(methylamino)propanoic acid (A-3S, 1.80 g, 13.32 mmol, 1.2 eq, HCl) and KOAc (7.63 g, 77.70 mmol, 7 eq) at 25° C. The suspension was gently stirred 16 h at 25° C. in the dark. Ethanol was removed under reduced pressure and the crude was diluted in water. The aqueous layer was acidified to pH=5 by the addition of solid citric acid, and then the solution was extracted with ethyl acetate (2×200 mL). The organic layers were combined, dried over anhydrous sodium sulfate, filtered and evaporated to dryness under reduced pressure. The residue was purified by prep-HPLC (column: Welch Xtimate C18 250*70 mm/10 um; mobile phase: [water (NH₄HCO₃)-ACN]; B %: 15%-50%, 20 min) to give 695 mg of 101.5 (18%) as a light yellow solid. (ES, m/z): [M+1]⁺ 343.3.

Synthesis of I-101, I-102, I-101a, I-102a. 500 mg of 101.5 (mixture of 4 peaks) was purified by three times SFC separation (Method 1 followed by Method 2 and 3) to give 80 mg of I-101a (16%, devalue: 85.0%, Peak 1), 47 mg of I-102a (9.4%, devalue: 98.0%, Peak 2), 79 mg of I-102 (16%, de value: 95.6%, Peak 3) and 50 mg of I-101 (10%, de value: 99.3%, Peak 4) as light yellow solids.

Method 1: Instrument: Waters SFC80 preparative SFC; Column: DAICEL CHIRALPAK IG (250 mm*30 mm, 10 um); Mobile phase: A for CO₂ and B for MeOH (0.1% NH₃H₂O); Gradient: B %=60% isocratic elution mode; Flow rate: 80 g/min; Wavelength: 220 nm; Column temperature: 40° C.; System back pressure: 100 bar.

Method 2: Instrument: Waters SFC80Q preparative SFC; Column: REGIS (s,s) WHELK-01 (250 mm*30 mm, 10 um); Mobile phase: A for CO₂ and B for MeOH (0.1% NH₃H₂O); Gradient: B %=42% isocratic elution mode; Flow rate: 70 g/min; Wavelength: 220 nm; Column temperature: 40° C.; System back pressure: 100 bar.

Method 3: Instrument: Waters SFC80Q preparative SFC; Column: DAICEL CHIRALPAK IG (250 mm*30 mm, 10 um); Mobile phase: A for CO₂ and B for IPA (0.1% NH₃H₂O); Gradient: B %=44% isocratic elution mode; Flow rate: 72 g/min; Wavelength: 220 nm; Column temperature: 40° C.; System back pressure: 100 bar.

I-101a: ¹H NMR (400 MHz, acetone-d6) δ=7.44-7.35 (m, 1H), 6.79 (br d, J=8.4 Hz, 1H), 6.73-6.64 (m, 1H), 4.74 (q, J=8.4 Hz, 1H), 4.33 (br d, J=8.1 Hz, 1H), 4.03 (br t, J=5.5 Hz, 1H), 3.64-3.53 (m, 1H), 3.52-3.41 (m, 2H), 3.32 (br dd, J=7.0, 10.6 Hz, 1H), 2.64 (s, 3H).

I-102a: ¹H NMR (400 MHz, acetone-d6) δ=7.40 (q, J=7.4 Hz, 1H), 6.79 (br d, J=8.1 Hz, 1H), 6.70 (br t, J=9.8 Hz, 1H), 5.23-5.13 (m, 1H), 5.05 (br d, J=3.5 Hz, 1H), 4.25 (br d, J=5.5 Hz, 1H), 3.39 (quin, J=9.9 Hz, 2H), 3.27-3.18 (m, 1H), 3.07 (br d, J=10.4 Hz, 1H), 2.70 (s, 3H).

I-102: ¹H NMR (400 MHz, acetone-d6) δ=7.45-7.35 (m, 1H), 6.79 (br d, J=8.3 Hz, 1H), 6.70 (br dd, J=8.7, 11.1 Hz, 1H), 5.15-5.05 (m, 1H), 4.61 (d, J=5.5 Hz, 1H), 3.77-3.67 (m, 1H), 3.44 (br d, J=9.2 Hz, 2H), 3.22 (br d, J=7.3 Hz, 2H), 2.64 (s, 3H).

I-101: ¹H NMR (400 MHz, acetone-d6) δ=7.45-7.36 (m, 1H), 6.80 (br d, J=8.3 Hz, 1H), 6.69 (br dd, J=8.6, 11.1 Hz, 1H), 4.98-4.88 (m, 1H), 4.54 (br d, J=8.2 Hz, 1H), 4.23 (br t, J=5.9 Hz, 1H), 3.62 (br t, J=10.1 Hz, 1H), 3.40 (br s, 1H), 3.22 (br d, J=5.1 Hz, 2H), 2.51 (s, 3H).

### Example 19: (4S)-2-((S)-2-(2-fluoro-6-hydroxyphenyl)-4,5-dihydrothiazol-4-yl)-3-methylthiazolidine-4-carboxylic acid

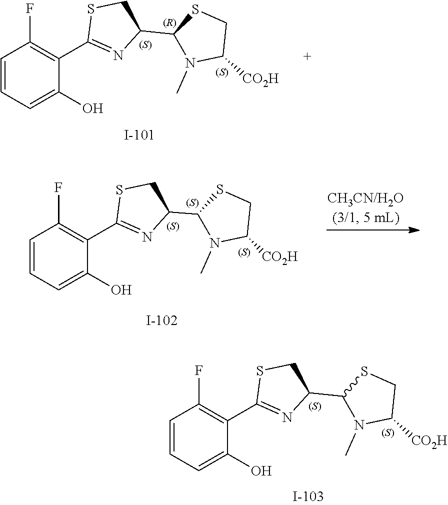

Synthesis of I-103. A solution of (2R,4S)-2-((S)-2-(2-fluoro-6-hydroxyphenyl)-4,5-dihydrothiazol-4-yl)-3-methylthiazolidine-4-carboxylic acid (I-101, 49.03 mg) and (2S,4S)-2-((S)-2-(2-fluoro-6-hydroxyphenyl)-4,5-dihydrothiazol-4-yl)-3-methylthiazolidine-4-carboxylic acid (I-102, 73.55 mg) in CH₃CN/H₂O (3/1, 5 mL) was shaken for 2 min to give a clear solution. The mixture was then lyophilized for 48 h to give 101.05 mg of I-103 (82%) as a light yellow solid. (ES, m/z): [M+1]⁺ 343.1.

¹H NMR (400 MHz, acetone-d6) δ=7.44-7.36 (m, 1.64H), 6.80 (td, J=1.2, 8.4 Hz, 1.62H), 6.70 (dd, J=8.3, 11.7 Hz, 1.65H), 5.16-5.05 (m, 0.98H), 4.93 (q, J=8.6 Hz, 0.66H), 4.61 (d, J=5.5 Hz, 0.96H), 4.54 (d, J=8.2 Hz, 0.66H), 4.25 (t, J=6.5 Hz, 0.68H), 3.73 (t, J=7.3 Hz, 0.99H), 3.62 (dd, J=9.4, 11.1 Hz, 0.72H), 3.48-3.36 (m, 2.81H), 3.23 (d, J=7.5 Hz, 3.33H), 2.65 (s, 3.00H), 2.52 (s, 1.90H).

### Example 20: (2R,4S)-2-((S)-2-(3-fluoro-2-hydroxyphenyl)-4,5-dihydrothiazol-4-yl)-3-methylthiazolidine-4-carboxylic acid and (2S,4S)-2-((S)-2-(3-fluoro-2-hydroxyphenyl)-4,5-dihydrothiazol-4-yl)-3-methylthiazolidine-4-carboxylic acid

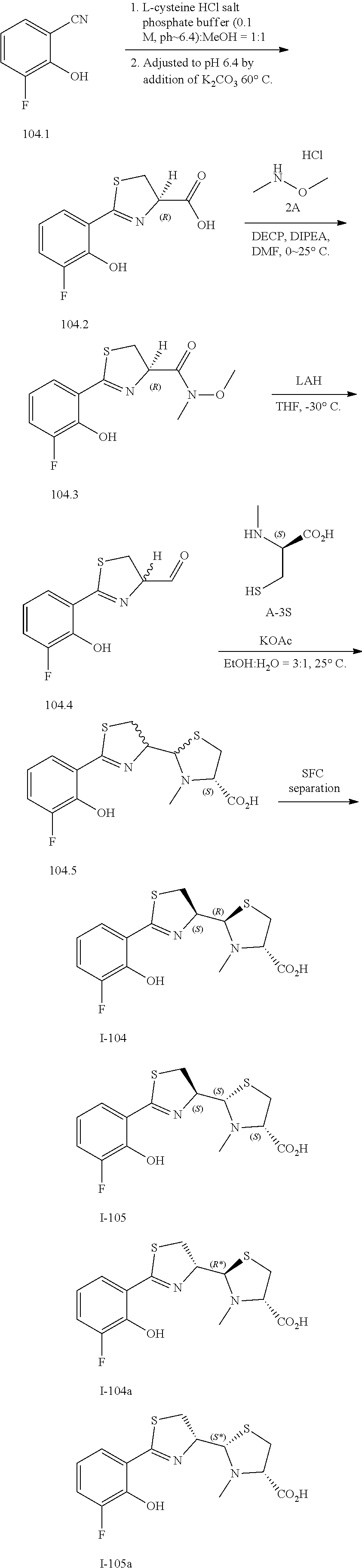

Synthesis of 104.2. To a solution of 3-fluoro-2-hydroxybenzonitrile (104.1, (4.7 g, 34.13 mmol, 1 eq) in MeOH (47 mL) was added L-cysteine (10.76 g, 68.27 mmol, 2 eq, HCl salt) and phosphate buffer (47 mL). The solution was adjusted to pH=6.4 by the addition of solid K₂CO₃ (5.19 g, 37.55 mmol, 1.1 eq) carefully and the reaction mixture was stirred at 60° C. for 12 h. The mixture was concentrated under reduced pressure, and the yellow crude residue was diluted with water (50 mL). The solution was adjusted to pH=2.0 by the addition of solid citric acid. After extraction with dichloromethane (3×50 mL), the organic layers were collected, dried over anhydrous sodium sulfate, filtered and concentrated under reduced pressure to give 6.18 g of 104.2 (75%, ee value: 100.0%) as a yellow solid. (ES, m/z): [M+1]⁺ 242.0.

Synthesis of 104.3. To a solution of (R)-2-(3-fluoro-2-hydroxyphenyl)-4,5-dihydrothiazole-4-carboxylic acid (104.2, 5 g, 20.73 mmol, 1 eq), N,O-dimethylhydroxylamine hydrochloride (2A, 2.22 g, 22.80 mmol, 1.1 eq, HCl) and diethoxyphosphorylformonitrile (3.91 g, 22.80 mmol, 3.62 mL, 95% purity, 1.1 eq) in DMF (50 mL) was added DIEA (4.02 g, 31.09 mmol, 5.42 mL, 1.5 eq) at 0° C. under N₂. The mixture was stirred for 30 min at 0° C., then another 2 h at 20° C. The reaction mixture was diluted with H₂O (50 mL) and extracted with ethyl acetate (50 mL*3). The combined organic layers were washed with aq. NaHCO₃ and brine (50 mL), dried over Na₂SO₄, filtered and concentrated under reduced pressure to give a residue. The residue was purified by silica gel column chromatography (SiO₂, Petroleum ether:Ethyl acetate from 1:0 to 1:1) to give 4.68 g of 104.3 (79%, ee value: 91.0%) as a light yellow solid. (ES, m/z): [M+1]⁺ 285.3.

Synthesis of 104.4. To a solution of (R)-2-(3-fluoro-2-hydroxyphenyl)-N-methoxy-N-methyl-4,5-dihydrothiazole-4-carboxamide (104.3, 3 g, 10.55 mmol, 1 eq) in THE (50 mL) was added LAH (600.67 mg, 15.83 mmol, 1.5 eq) at −40° C. under N₂. The temperature was allowed to rise up to −30° C. and stirred for 0.5 h. The reaction mixture was then hydrolyzed by successive additions of aqueous solutions of saturated NH₄Cl (30 mL) and 1 M solution of KHSO₄ (30 mL). The mixture was vigorously stirred and allowed to warm up to the room temperature until two phases were formed. After partition and extraction with ethyl acetate (2×50 mL), the organic layers were combined, dried over anhydrous sodium sulfate, filtered before being evaporated under reduced pressure to give 2.25 g of 104.4 (crude) as a yellow solid. The crude product was used to the next step without further purification. (ES, m/z): [M+1]⁺ 226.3

Synthesis of 104.5. To a solution of 2-(3-fluoro-2-hydroxyphenyl)-4,5-dihydrothiazole-4-carbaldehyde (104.4, 2.25 g, 9.99 mmol, 1 eq) in a mixture of EtOH (66 mL) and H₂O (22 mL) were successively added (S)-3-mercapto-2-(methylamino)propanoic acid (A-3S, 2.06 g, 11.99 mmol, 1.2 eq, HCl) and KOAc (6.86 g, 69.93 mmol, 7 eq) at 20° C. The suspension was gently stirred 16 h at 20° C. in the dark. Ethanol was removed under reduced pressure and the crude was diluted in water. The aqueous layer was acidified to pH=5 by the addition of solid citric acid, and then the solution was extracted with ethyl acetate (2×50 mL). The organic layers were combined, dried over anhydrous sodium sulfate, filtered and evaporated to dryness under reduced pressure. The residue was purified by prep-HPLC column: Welch Xtimate C18 250*70 mm #10 um; mobile phase: [water (NH₄HCO₃)-ACN]; B %: 15%-45%, 20 min) to give 485 mg of 104.5 (14%) as a light yellow solid. (ES, m/z): [M+1]⁺ 343.1.

Synthesis of I-104, I-105, I-104a, and I-105a. 523 mg of 104.5 (mixture of 4 peaks) was purified by twice SFC separation (Method 1 followed by Method 2) to give 55.4 mg of I-104a (11%, de value: 96.0%, Peak 1), 73.7 mg of I-105 (14%, de value: 96.0%, Peak 2), 27.1 mg of I-105a (5%, de value: 86.0%, Peak 3) and 105.11 mg of I-104 (20%, de value: 97.0%, Peak 4) as light yellow solids. (ES, m/z): [M+1]⁺ 343.1

Method 1: Instrument: Waters SFC 80 preparative SFC; Column: REGIS (s,s) WHELK-01 (250 mm*30 mm, 10 um); Mobile phase: A for CO₂ and B for MeOH (0.2% NH₃H₂O); Gradient: B %=45% isocratic elution mode; Flow rate: 60 g/min; Wavelength:220 nm; Column temperature: 40° C.; System back pressure: 100 bar.

Method 2: Instrument: Waters SFC 80 preparative SFC; Column: DAICEL CHIRALPAK IG (250 mm*30 mm, 10 um); Mobile phase: A for CO₂ and B for IPA (0.1% NH₃H₂O); Gradient: B %=35% isocratic elution mode; Flow rate: 65 g/min; Wavelength: 220 nm; Column temperature: 40° C.; System back pressure: 100 bar.

I-104a: ¹H NMR (400 MHz, acetone-d6) δ=7.34-7.18 (m, 2H), 6.90 (dt, J=4.8, 8.1 Hz, 1H), 4.87 (q, J=8.4 Hz, 1H), 4.36 (d, J=8.4 Hz, 1H), 4.02 (dd, J=5.4, 6.9 Hz, 1H), 3.73-3.61 (m, 1H), 3.58-3.51 (m, 1H), 3.44 (dd, J=5.4, 11.1 Hz, 1H), 3.31 (dd, J=6.9, 11.1 Hz, 1H), 2.63 (s, 3H)

I-105: ¹H NMR (400 MHz, acetone-d6) δ=7.36-7.23 (m, 2H), 6.97-6.86 (m, 1H), 5.24 (d, J=5.5 Hz, 1H), 4.64 (d, J=5.5 Hz, 1H), 3.74 (t, J=7.4 Hz, 1H), 3.54 (d, J=8.9 Hz, 2H), 3.27-3.19 (m, 2H), 2.65 (s, 3H)

I-105a: ¹H NMR (400 MHz, acetone-d6) δ=7.36-7.23 (m, 2H), 6.92 (br d, J=4.6 Hz, 1H), 5.33 (br d, J=4.6 Hz, 1H), 5.07 (d, J=4.8 Hz, 1H), 4.27 (dd, J=2.3, 6.5 Hz, 1H), 3.48 (dd, J=7.6, 9.1 Hz, 2H), 3.24 (dd, J=6.6, 10.4 Hz, 1H), 3.07 (dd, J=2.2, 10.4 Hz, 1H), 2.72 (s, 3H)

I-104: ¹H NMR (400 MHz, acetone-d6) δ=7.34-7.23 (m, 2H), 6.97-6.88 (m, 1H), 5.06 (q, J=8.3 Hz, 1H), 4.57 (d, J=8.1 Hz, 1H), 4.26 (t, J=6.4 Hz, 1H), 3.71 (dd, J=8.8, 11.3 Hz, 1H), 3.51 (dt, J=8.5, 11.3 Hz, 1H), 3.24 (d, J=6.4 Hz, 2H), 2.66-2.65 (m, 1H), 2.52 (s, 3H)

### Example 21: (4S)-2-((S)-2-(3-fluoro-2-hydroxyphenyl)-4,5-dihydrothiazol-4-yl)-3-methylthiazolidine-4-carboxylic acid

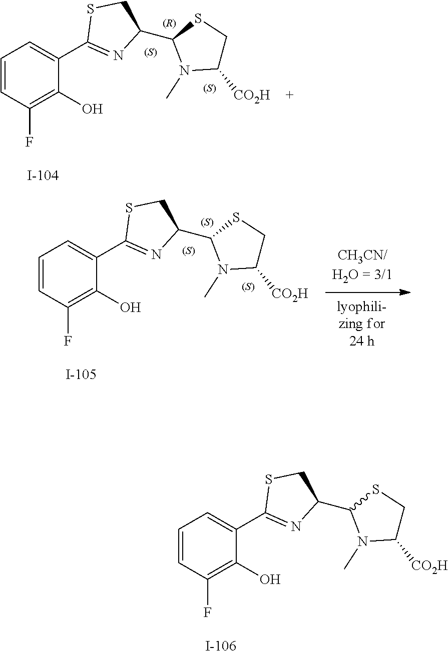

Synthesis of I-106. A solution of (2R,4S)-2-((S)-2-(3-fluoro-2-hydroxyphenyl)-4,5-dihydrothiazol-4-yl)-3-methylthiazolidine-4-carboxylic acid (I-104, 48.57 mg) and (2S,4S)-2-((S)-2-(3-fluoro-2-hydroxyphenyl)-4,5-dihydrothiazol-4-yl)-3-methylthiazolidine-4-carboxylic acid (I-105, 72.85 mg) in CH₃CN/H₂O (3/1, 4 mL) was shaken 2 min to give a clear solution. The mixture was then lyophilized for 24 h to give 94.65 mg of I-106 (78%) as a light yellow solid. (ES, m/z): [M+1]⁺ 343.1.

I-106: ¹H NMR (400 MHz, acetone-d6) δ=7.37-7.21 (m, 3H), 6.92 (dt, J=4.6, 8.0 Hz, 1.59H), 5.24 (dt, J=5.5, 8.9 Hz, 0.95H), 5.06 (d, J=8.5 Hz, 0.54H), 4.64 (d, J=5.6 Hz, 0.95H), 4.57 (d, J=8.1 Hz, 0.54H), 4.27 (t, J=6.4 Hz, 0.58H), 3.78-3.73 (m, 0.92H), 3.72-3.68 (m, 0.61H), 3.54 (d, J=8.9 Hz, 1.98H), 3.52-3.45 (m, 0.79H), 3.26-3.23 (m, 1.56H), 3.23-3.21 (m, 1.43H), 2.65 (s, 3.09H), 2.52 (s, 1.81H)

### Example 22: (4S)-2-((R)-2-(3-fluoro-2-hydroxyphenyl)-4,5-dihydrothiazol-4-yl)-3-methylthiazolidine-4-carboxylic acid

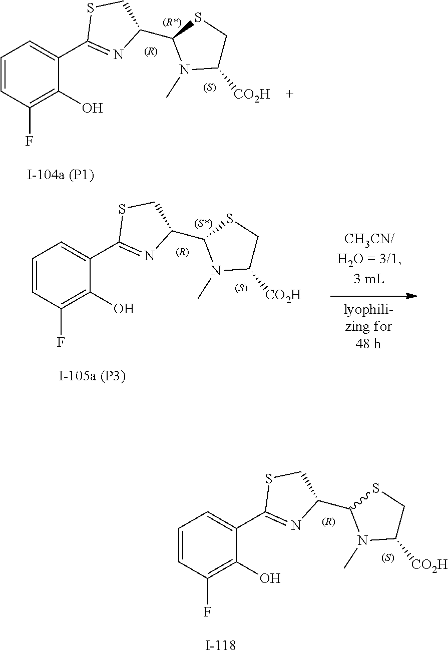

Synthesis of I-118. A solution of (2R,4S)-2-((R)-2-(3-fluoro-2-hydroxyphenyl)-4,5-dihydrothiazol-4-yl)-3-methylthiazolidine-4-carboxylic acid (I-104a, 51.88 mg) and (2S,4S)-2-((R)-2-(3-fluoro-2-hydroxyphenyl)-4,5-dihydrothiazol-4-yl)-3-methylthiazolidine-4-carboxylic acid (105a, 27.89 mg) in CH₃CN/H₂O (3/1, 3 mL) was shaken for 2 min to give a clear solution. The mixture was then lyophilized for 48 h to give 40.88 mg of I-118 (51%) as a light yellow solid. (ES, m/z): [M+1]⁺ 343.1.

I-118: ¹H NMR (400 MHz, acetone-d6) δ=7.51-7.23 (m, 2.69H), 6.94-6.87 (m, 1.28H), 5.33 (dt, J=4.8, 9.1 Hz, 0.44H), 5.06 (d, J=4.6 Hz, 0.39H), 4.87 (q, J=8.3 Hz, 0.90H), 4.37 (d, J=8.3 Hz, 0.84H), 4.27 (dd, J=2.4, 6.5 Hz, 0.41H), 4.02 (dd, J=5.4, 6.9 Hz, 0.89H), 3.70-3.64 (m, 1H), 3.60-3.52 (m, 1.19H), 3.51-3.42 (m, 1.90H), 3.32 (dd, J=6.9, 11.1 Hz, 1.06H), 3.23 (dd, J=6.5, 10.5 Hz, 0.71H), 3.07 (br dd, J=2.3, 10.5 Hz, 0.92H), 2.71 (s, 1.76H), 2.64 (s, 3.00H)

### Example 23: (2R,4S)-2-((S)-2-(4-fluoro-2-hydroxyphenyl)-4,5-dihydrothiazol-4-yl)-3-methylthiazolidine-4-carboxylic acid and (2S,4S)-2-((S)-2-(4-fluoro-2-hydroxyphenyl)-4,5-dihydrothiazol-4-yl)-3-methylthiazolidine-4-carboxylic acid

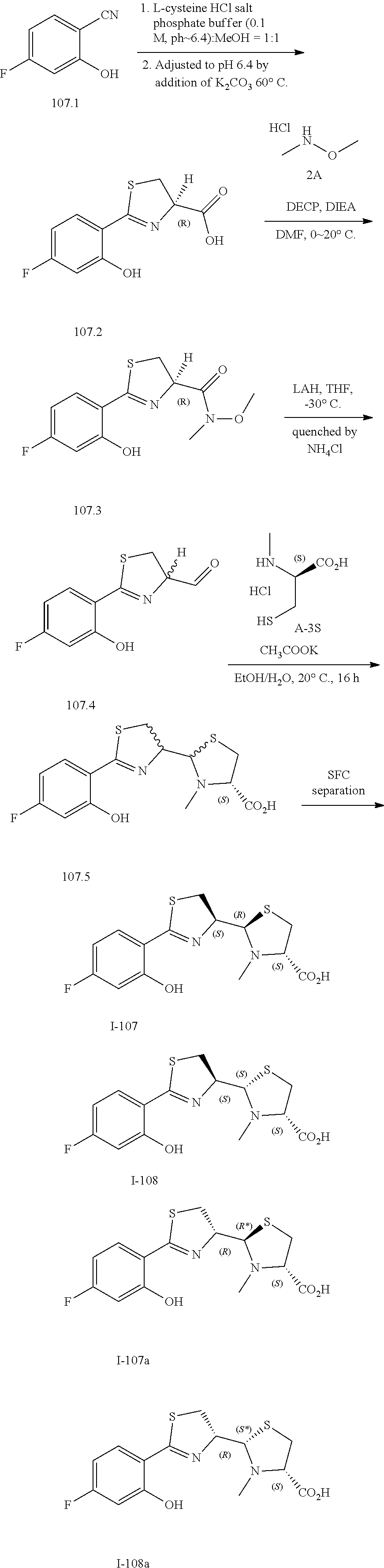

Synthesis of 107.2. A mixture of 4-fluoro-2-hydroxybenzonitrile (107.1, 5 g, 36.47 mmol, 1 eq) in phosphate buffer (50 mL) and MeOH (50 mL) was added L-cysteine (11.50 g, 72.93 mmol, 2 eq, HCl salt) at 25° C. The solution was adjusted to pH=6.4 by the addition of solid K₂CO₃ carefully and the reaction mixture was stirred at 60° C. for 12 h. The mixture was concentrated under reduced pressure, and the yellow crude residue was diluted with water (20 mL). The solution was adjusted to pH=2.0 by the addition of solid citric acid. After extraction with dichloromethane (3×20 mL), the organic layers were collected, dried over anhydrous sodium sulfate, filtered and concentrated under reduced pressure to give 7.4 g of 107.2 (84%, ee value: 100.0%) as a yellow solid. (ES, m/z): [M+1]⁺ 242.1.

Synthesis of 107.3. To a solution of (R)-2-(4-fluoro-2-hydroxyphenyl)-4,5-dihydrothiazole-4-carboxylic acid (107.2, 5.5 g, 22.80 mmol, 1 eq)), N,O-dimethylhydroxylamine (2A, 5.5 g, 22.80 mmol, 1 eq, HCl) and DECP (4.09 g, 25.08 mmol, 3.79 mL, 1.1 eq) in DMF (60 mL) was added DIPEA (4.42 g, 34.20 mmol, 5.96 mL, 1.5 eq) at 0° C. under N₂. The mixture was stirred for 30 min at 0° C., then another 2 h at 25° C. The mixture was poured into water (60 mL) and stirred for 1 min. The aqueous phase was extracted with ethyl acetate (3×60 mL). The combined organic phase was washed with aq·NaHCO₃, then with brine, dried with anhydrous Na₂SO₄, filtered and concentrated in vacuum. The residue was purified by column chromatography (SiO₂, Petroleum ether/Ethyl acetate=I/O to 1/1) to give 4 g of 107.3 (63%, ee value: 79.8%) as a white solid. (ES, m/z): [M+1]⁺ 285.1.

Synthesis of 107.4. To a solution of (R)-2-(4-fluoro-2-hydroxyphenyl)-N-methoxy-N-methyl-4,5-dihydrothiazole-4-carboxamide (107.3, 2 g, 7.03 mmol, 1 eq) in THE (20 mL) was added LAH (2 g, 7.03 mmol, 1 eq) at −30° C. under N₂. The mixture was stirred at −30° C. for 0.5 h. The reaction mixture was then hydrolyzed by successive additions of aqueous solutions of saturated NH₄Cl (40 mL) and 1 M solution of KHSO₄ (40 mL). The mixture was vigorously stirred and allowed to warm up to the room temperature until two phases were formed. After partition and extraction with ethyl acetate (2×200 mL), the organic layers were combined, dried over anhydrous sodium sulfate and filtered before being evaporated under reduced pressure to give 1.63 g of 107.4 (crude) as a yellow solid. The crude product was used to the next step without further purification.

Synthesis of 107.5. To a solution of 2-(4-fluoro-2-hydroxyphenyl)-4,5-dihydrothiazole-4-carbaldehyde (107.4, 1.58 g, 7.01 mmol, 1 eq) in a mixture of EtOH (45 mL) and H₂O (15 mL) was added (2S)-2-(methylamino)-3-sulfanyl-propanoic acid (A-3S, 1.14 g, 8.42 mmol, 1.2 eq, HCl) and KOAc (4.82 g, 49.10 mmol, 7 eq) at 25° C. The suspension was gently stirred 16 h at 25° C. in the dark. Ethanol was removed under reduced pressure and the crude was diluted in water. The aqueous layer was acidified to pH=5 by the addition of solid citric acid, and then the solution was extracted with ethyl acetate (2×200 mL). The organic layers were combined, dried over anhydrous sodium sulfate, filtered and evaporated to dryness under reduced pressure. The residue was purified by prep-HPLC (column: Phenomenex C18 80*40 mm*3 um; mobile phase: [water (NH₄HCO₃)-ACN]; B %: 5%-30%, 8 min) to give 730 mg of 107.5 (30%) as a yellow solid. (ES, m/z): [M+1]⁺ 343.1.

Synthesis of I-107, I-108, I-107a, and I-108a. 0.5 g of 107.5 (mixture of 4 peaks) was purified by twice SFC separation (Method 1 followed by Method 2) to give 10.06 mg of I-107 (2.0%, devalue: 100.0%, Peak 1), 57.79 mg of I-107a (not clean, mixture of I-107 and I-107a), 12.07 mg of I-108a (not clean, mixture of I-108 and 108a) and 163.15 mg of I-108 (32%, de value: 93.2%, Peak 4) as light yellow solids. (ES, m/z): [M+1]⁺ 343.1.

### SFC Separation Methods:

Method 1: Instrument: Waters SFC80Q preparative SFC; Column: DAICEL CHIRALPAK AD (250 mm*30 mm, 10 um); Mobile phase: A for CO₂ and B for MeOH (0.1% NH₃H₂O); Gradient: B %=38% isocratic elution mode; Flow rate: 65 g/min; Wavelength: 220 nm; Column temperature: 40° C.; System back pressure: 100 bar.

Method 2: Instrument: Waters SFC80Q preparative SFC; Column: REGIS(s,s) WHELK-01 (250 mm*30 mm, 10 um); Mobile phase: A for CO₂ and B for EtOH (0.1% NH₃H₂O); Gradient: B %=38% isocratic elution mode; Flow rate: 65 g/min; Wavelength:220 nm; Column temperature: 40° C.; System back pressure: 100 bar.

I-107: ¹H NMR (400 MHz, acetone-d6) δ=7.56-7.43 (m, 1H), 6.80-6.62 (m, 2H), 5.00 (q, J=8.1 Hz, 1H), 4.54 (d, J=8.3 Hz, 1H), 4.23 (br t, J=6.4 Hz, 1H), 3.68 (dd, J=8.9, 11.0 Hz, 1H), 3.47 (br dd, J=7.9, 11.2 Hz, 1H), 3.31-3.10 (m, 2H), 2.50 (s, 3H).

I-108: ¹H NMR (400 MHz, acetone-d6) δ=7.49 (dd, J=6.5, 8.6 Hz, 1H), 6.79-6.65 (m, 2H), 5.26-5.12 (m, 1H), 4.62 (d, J=5.5 Hz, 1H), 3.72 (dd, J=6.8, 7.9 Hz, 1H), 3.51 (d, J=8.9 Hz, 2H), 3.29-3.15 (m, 2H), 2.64 (s, 3H).

### Example 24: (4S)-2-((S)-2-(4-fluoro-2-hydroxyphenyl)-4,5-dihydrothiazol-4-yl)-3-methylthiazolidine-4-carboxylic acid

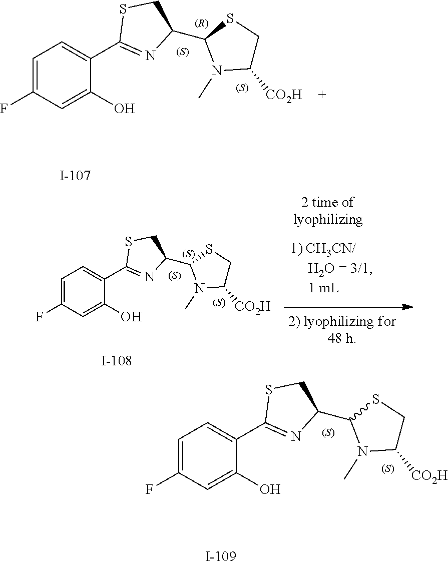

Synthesis of I-109. A solution of 10.06 mg of 1-107 and 15.15 mg of 1-108 in ACN/H₂O (3/1, 1 mL) was shaken for 2 min to give a clear solution. The solution was then lyophilized for 48 h to give 18.68 mg of I-109 (74%). (ES, m/z): [M+1]⁺ 343.1.

I-109: ¹H NMR (400 MHz, acetone-d6) δ=7.49 (dd, J=6.4, 8.6 Hz, 1.47H), 6.78-6.67 (m, 2.99H), 5.21 (dt, J=5.6, 8.8 Hz, 0.86H), 5.01 (q, J=8.3 Hz, 0.64H), 4.62 (d, J=5.5 Hz, 0.83H), 4.55 (d, J=8.3 Hz, 0.65H), 4.26 (t, J=6.5 Hz, 0.66H), 3.78-3.63 (m, 1.66H), 3.58-3.44 (m, 2.61H), 3.26-3.20 (m, 3.13H), 2.65 (s, 3.00H), 2.51 (s, 2.07H).

### Example 25: (2R,4S)-2-((S)-2-(3-hydroxypyridin-2-yl)-4,5-dihydrothiazol-4-yl)-3-methylthiazolidine-4-carboxylic acid and (2S,4S)-2-((S)-2-(3-hydroxypyridin-2-yl)-4,5-dihydrothiazol-4-yl)-3-methylthiazolidine-4-carboxylic acid

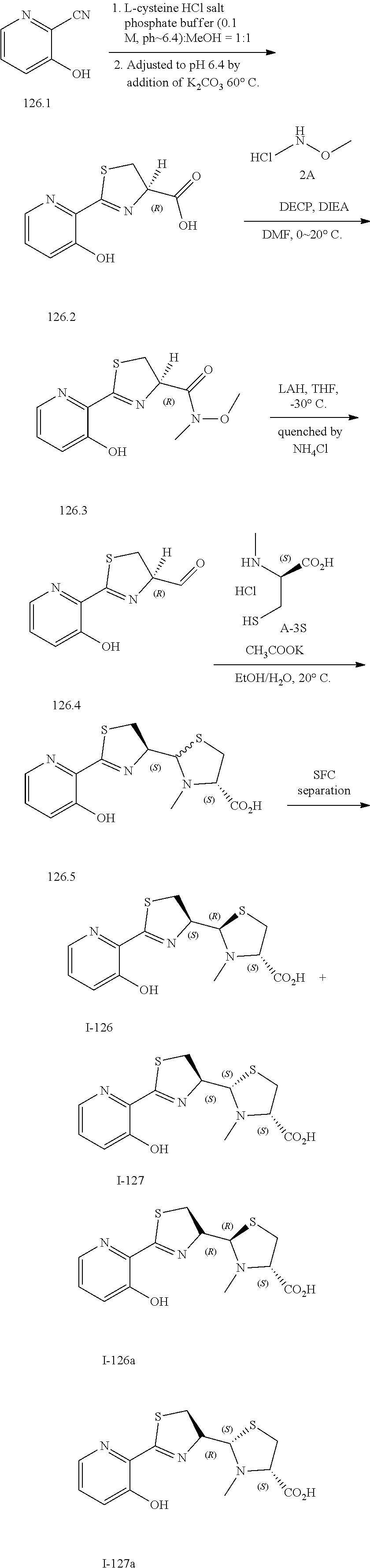

Synthesis of 126.2. To a mixture of 3-hydroxypicolinonitrile (126.1, 4.5 g, 37.47 mmol, 1 eq) in MeOH (45 mL) was added L-cysteine (11.81 g, 74.93 mmol, 2 eq, HCl salt) and phosphate buffer (45 mL). The solution was adjusted to pH=6.4 by addition of solid K₂CO₃ carefully and the reaction mixture was stirred at 60° C. for 12 hours. The mixture was concentrated under reduced pressure, and the yellow crude residue was diluted with water (200 mL). The solution was adjusted to pH=2.0 by the addition of solid citric acid. After extraction with ethyl acetate (3×200 mL), the organic layers were collected, dried over anhydrous sodium sulfate, filtered and concentrated under reduced pressure to give 7.46 g of 126.2 (85%, ee value: 85.6%) was obtained as a yellow solid. (ES, m/z): [M+1]⁺ 225.0.

Synthesis of 126.3. To a solution of (R)-2-(3-hydroxypyridin-2-yl)-4,5-dihydrothiazole-4-carboxylic acid (126.2, 7.46 g, 33.26 mmol, 1 eq), N,O-dimethylhydroxylamine hydrochloride (2A, 3.56 g, 33.6 mmol, 1.1 eq, HCl) and DECP (5.96 g, 33.6 mmol, 5.52 mL, 1.1 eq) in DMF (75 mL) was added DIEA (6.44 g, 49.9 mmol, 99.89 mL, 1.5 eq) at 0° C. under N₂. The mixture was stirred for 30 min at 0° C. and then stirred for 2 h at 20° C. The reaction mixture was diluted with water (100 mL) and extracted with ethyl acetate (3×100 mL). The combined organic phase was washed with aq·NaHCO₃ and brine, dried over anhydrous Na₂SO₄, filtered and concentrated to give 6.7 g of 126.3 (73%, ee value: 84.2%) as a yellow solid. (ES, m/z): [M+1]+ 268.1.

Synthesis of 126.4. To a solution of (R)-2-(3-hydroxypyridin-2-yl)-N-methoxy-N-methyl-4,5-dihydrothiazole-4-carboxamide (126.3, 5 g, 18.71 mmol, 1 eq) in THE (50 mL) was added LAH (1.06 g, 28.06 mmol, 1.5 eq) at −40° C. under N₂. The temperature was allowed to rise up to −30° C. and stirred for 0.5 h. The reaction mixture was then quenched by successive additions of aqueous solutions of saturated NH₄Cl (50 mL) and of a 1M solution of KHSO₄ (50 mL). The mixture was vigorously stirred and allowed to warm up to the room temperature until two phases were formed. After partition and extraction with ethyl acetate (3×200 mL), the organic layers were collected, dried over Na₂SO₄, filtered and concentrated under reduced pressure to give 2.99 g of 126.4 (76.6%, ee value: 84.2%). (ES, m/z): [M−1]⁻ 207.0.

Synthesis of 126.5. To a solution of (R)-2-(3-hydroxypyridin-2-yl)-4,5-dihydrothiazole-4-carbaldehyde (126.4, 2.78 g, 13.34 mmol, 1 eq) in a mixture of EtOH (30 mL) and H₂O (10 mL) were successively added (S)-3-mercapto-2-(methylamino)propanoic acid (A-3S, 2.75 g, 16.01 mmol, 1.2 eq, HCl) and KOAc (9.16 g, 93.38 mmol, 7 eq) at 25° C. This suspension was gently stirred 12 h at 20° C. in the dark. Ethanol was removed under reduced pressure and the crude was diluted in water. The aqueous layer was acidified to pH=5 by the addition of solid citric acid, and then the solution was extracted with ethyl acetate (2×100 mL). The organic layers were combined, dried over anhydrous sodium sulfate, filtered and concentrated to dryness under reduced pressure. The residue was purified by prep-HPLC (column: Phenomenex C18 80*40 mm*3 um; mobile phase: [water (NH₄HCO₃)-ACN]; B %: 1%-30%, 8 min) to give 1.21 g of 126.5 (27%) as a light yellow solid. (ES, m/z): [M+1]⁺ 326.1.

Synthesis of I-126, I-127, I-126a, I-127a. 1 g of 126.5 (mixture of 4 peaks) was purified by three times SFC separation (Method 1 followed by Method 2 and 3) to give 100 mg of I-127 (0.1%, de value: 100.0%, Peak 2), 75 mg of I-126 (0.075%, de value: 96%, Peak 1), 9 mg of I-127a (mixture of I-127 and 127a) and 12 mg of I-126a (0.012%, de value: 100%, Peak 4) as light yellow solids. (ES, m/z): [M+1]⁺ 326.0.

Method 1: Instrument: Waters SFC80 preparative SFC; Column: DAICEL CHIRALPAK IG (250 mm*30 mm, 10 um); Mobile phase: A for CO₂ and B for EtOH (0.1% NH₃H₂O); Gradient: B %=50% isocratic elution mode; Flow rate: 80 g/min; Wavelength:220 nm; Column temperature: 40° C.; System back pressure: 100 bar.

Method 2: Instrument: Waters SFC80 preparative SFC; Column: DAICEL CHIRALCEL OJ (250 mm*30 mm, 10 um); Mobile phase: A for CO₂ and B for EtOH (0.1% NH₃H₂O); Gradient: B %=28% isocratic elution mode; Flow rate: 56 g/min; Wavelength:220 nm; Column temperature: 40° C.; System back pressure: 100 bar.

Method 3: Instrument: Waters SFC80 preparative SFC; Column: DAICEL CHIRALPAK IH (250 mm*30 mm, 10 um); Mobile phase: A for CO₂ and B for IPA (0.1% NH₃H₂O); Gradient: B %=40% isocratic elution mode; Flow rate: 70 g/min; Wavelength:220 nm; Column temperature: 40° C.; System back pressure: 100 bar.

I-126: ¹H NMR (400 MHz, acetone) δ=8.17 (dd, J=1.6, 4.4 Hz, 1H), 7.44-7.35 (m, 2H), 4.94 (q, J=8.4 Hz, 1H), 4.38 (d, J=8.0 Hz, 1H), 4.03 (dd, J=5.2, 6.8 Hz, 1H), 3.56 (dd, J=1.2, 11.6 Hz, 1H), 3.49-3.41 (m, 2H), 3.32 (dd, J=6.8, 10.8 Hz, 1H), 2.66 (s, 3H).

I-127: ¹H NMR (400 MHz, acetone) δ=8.17 (dd, J=2.1, 4.4 Hz, 1H), 7.46-7.31 (m, 2H), 5.31-5.22 (m, 1H), 4.64 (d, J=6 Hz, 1H), 3.73 (t, J=7.6 Hz, 1H), 3.42 (d, J=9.2 Hz, 2H), 3.23 (d, J=7.6 Hz, 2H), 2.65 (s, 3H).

I-126a: ¹H NMR (400 MHz, acetone) δ=8.21-8.14 (m, 1.19H), 7.46-7.34 (m, 2.49H), 5.31-5.25 (m, 1H), 5.32 (s, 0.22H), 5.14-5.05 (m, 1.02H), 4.64 (d, J=5.6 Hz, 0.22H), 4.58 (d, J=8.4 Hz, 0.98H), 4.27 (t, J=6.4 Hz, 1.02H), 3.93-3.85 (m, 0.90H), 3.76-3.71 (m, 0.23H), 3.62-3.55 (m, 1H), 3.45-3.35 (m, 1.93H), 3.26-3.21 (m, 2.45H), 2.65 (s, 0.87H), 2.52 (s, 3H).

I-127a: ¹H NMR (400 MHz, acetone) δ=8.20-8.15 (m, 1H), 7.45-7.39 (m, 1H), 7.39-7.35 (m, 1H), 5.44-5.32 (m, 1H), 5.08 (d, J=4.4 Hz, 1H), 4.32-4.24 (m, 1H), 3.46-3.30 (m, 2H), 3.28-3.19 (m, 1H), 3.12-3.02 (m, 1H), 2.72 (s, 3H).

### Example 26: (4S)-2-((S)-2-(3-hydroxypyridin-2-yl)-4,5-dihydrothiazol-4-yl)-3-methylthiazolidine-4-carboxylic acid

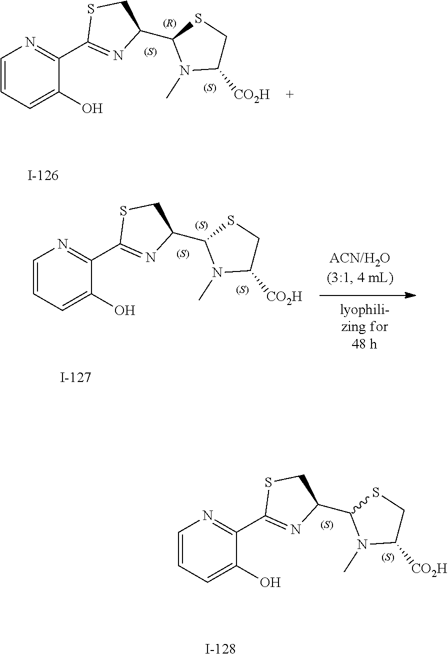

Synthesis of I-128. A solution of (2R,4S)-2-((S)-2-(3-hydroxypyridin-2-yl)-4,5-dihydrothiazol-4-yl)-3-methylthiazolidine-4-carboxylic acid (I-126, 60.28 mg) and (2S,4S)-2-((S)-2-(3-hydroxypyridin-2-yl)-4,5-dihydrothiazol-4-yl)-3-methylthiazolidine-4-carboxylic acid (I-127, 90.42 mg) was dissolved in ACN (3 mL) and H₂O (1 mL), and the mixture was lyophilized to give 125.32 mg of I-128 (83%) as a light yellow solid. (ES, m/z): [M+1]⁺ 326.1. ¹H NMR (400 MHz, acetone) δ=8.17 (br s, 2H), 7.47-7.34 (m, 4.07H), 5.37-5.25 (m, 1.20H), 4.99-4.89 (m, 0.81H), 4.64 (br d, J=5.2 Hz, 1.22H), 4.38 (br d, J=8.4 Hz, 0.81H), 4.06-3.98 (m, 0.81H), 3.72 (br t, J=7.2 Hz, 1.25H), 3.61-3.52 (m, 1.16H), 3.48-3.39 (m, 4.03H), 3.31 (br dd, J=7.2, 10.8 Hz, 1.09H), 3.22 (br d, J=7.2 Hz, 2.30H), 2.65 (s, 6H)

### Example 27: (2R,4S)-2-((S)-2-(2-hydroxy-4-(2-(2-methoxyethoxy)ethoxy) phenyl)-4,5-dihydrothiazol-4-yl)-3-methylthiazolidine-4-carboxylic acid and (2S,4S)-2-((S)-2-(2-hydroxy-4-(2-(2-methoxyethoxy)ethoxy) phenyl)-4,5-dihydrothiazol-4-yl)-3-methylthiazolidine-4-carboxylic acid

Synthesis of 123.2. To a solution of 2,4-dihydroxybenzoic acid (123.1, 50 g, 324.42 mmol, 1 eq), acetone (28.26 g, 486.63 mmol, 35.78 mL, 1.5 eq), DMAP (2.38 g, 19.47 mmol, 0.06 eq) in dimethoxyethane (200 mL) was added dropwise thionyl chloride (77.19 g, 648.85 mmol, 47.07 mL, 2 eq) at 0° C. for 1 h and then stirred at 25° C. for 7 h. The reaction mixture was quenched with a saturated solution of NaHCO₃ (400 mL) and extracted with ethyl acetate (2×100 mL). The organic phase was washed with a saturated aqueous NaCl solution (100 mL), dried over anhydrous Na₂SO₄ and concentrated to give a residue and the residue was triturated with dichloromethane to afford the product as an off-yellow solid. The residue was purified by column chromatography (SiO₂, Petroleum ether:Ethyl acetate=1:1 to 0:1) to give 12.45 g of 123.2 (crude) as a yellow solid. (ES, m/z): [M+1]⁺ 195.1.

Synthesis of 123.3. To a solution of 7-hydroxy-2,2-dimethyl-4H-benzo[d][1,3]dioxin-4-one (123.2, 11.45 g, 58.96 mmol, 1 eq) and 1-bromo-2-(2-methoxyethoxy)ethane (11.87 g, 64.86 mmol, 1.1 eq) in ACN (110 mL) was added K₂CO₃ (24.45 g, 176.89 mmol, 3 eq). The mixture was stirred at 90° C. for 5 h. The suspension was filtered through a pad of Celite and the filter cake was washed with ethyl acetate (3×50 mL). The combined filtrates were concentrated to dryness to give a residue. The residue was purified by column chromatography (SiO₂, Petroleum ether:Ethyl acetate=99:1 to 10:1) to give 14.05 g of 123.3 (80%) as a light yellow oil. (ES, m/z): [M+1]⁺ 297.1.

Synthesis of 123.4. A solution of 7-(2-(2-methoxyethoxy)ethoxy)-2,2-dimethyl-4H-benzo[d][1,3]dioxin-4-one (123.3, 5.75 g, 19.24 mmol, 1 eq) in NH₃/i-PrOH (10 M, 60 mL) was stirred for 0.5 h at 15° C. Then the mixture was heated to 80° C. and stirred for 12 hr. The reaction mixture was filtered and concentrated under reduced pressure to give a residue. The residue was purified by column chromatography (SiO₂, Petroleum ether:Ethyl acetate=3:1 to 1:1) to give 4.22 g of 123.4 (86%) as a light yellow oil. (ES, m/z): [M+1]⁺ 256.1.

Synthesis of 123.5. To a solution of 2-hydroxy-4-(2-(2-methoxyethoxy)ethoxy)benzamide (123.4, 4.22 g, 15.20 mmol, 1 eq) in THE (40 mL) was added burgess reagent (5.91 g, 22.80 mmol, 1.5 eq). The mixture was stirred at 60° C. for 12 h. The reaction mixture was diluted with H₂O (100 mL) and extracted with ethyl acetate (3×200 mL). The combined organic layers were washed with brine (200 mL), dried over anhydrous sodium sulfate, filtered and concentrated under reduced pressure to give a residue. The residue was purified by column chromatography (SiO₂, DCM:MeOH=30:1 to 10:1) to give 1.1 g of 123.5 (28%) as a yellow solid. (ES, m/z): [M−1]⁻ 236.1.

Synthesis of 123.6. To a solution of 2-hydroxy-4-(2-(2-methoxyethoxy)ethoxy)benzonitrile (123.5, 980 mg, 4.13 mmol, 1 eq) in MeOH (10 mL) was added L-cysteine (1.30 g, 8.26 mmol, 2 eq, HCl salt) and phosphate buffer (10 mL) at room temperature. The solution was adjusted to pH=6.4 by the addition of solid K₂CO₃ carefully and the reaction mixture was stirred at 60° C. for 12 h. The mixture was concentrated under reduced pressure, and the yellow crude residue was diluted with water (100 mL). The solution was adjusted to pH=2.0 by the addition of solid citric acid. After extraction with dichloromethane (3×100 mL), the organic layers were collected, dried over anhydrous sodium sulfate, filtered and concentrated under reduced pressure to give 1.35 g of 123.6 (96%, ee value: 100%) as a yellow solid. (ES, m/z): [M+1]⁺ 342.0.

Synthesis of 123.7. To a solution of (R)-2-(2-hydroxy-4-(2-(2-methoxyethoxy)ethoxy)phenyl)-4,5-dihydrothiazole-4-carboxylic acid (123.6, 1.85 g, 5.42 mmol, 1 eq), N,O-dimethylhydroxylamine hydrochloride (2A, 1.11 g, 11.38 mmol, 2.1 eq, HCl) and DECP (1.95 g, 11.38 mmol, 1.81 mL, 2.1 eq) in DMF (30 mL) was added DIEA (1.75 g, 13.55 mmol, 2.36 mL, 2.5 eq) at 0° C. under N₂. The mixture was stirred for 30 min at 0° C. and another 2 h at 20° C. The reaction mixture was diluted with H₂O (50 mL) and extracted with ethyl acetate (50 mL×3). The combined organic layers were washed with aq. NaHCO₃ and brine, dried over Na₂SO₄, filtered and concentrated under reduced pressure to give a residue. The residue was purified by silica gel column chromatography (SiO₂, Petroleum ether:Ethyl acetate from 1:0 to 1:1) to give 1.27 g of 123.7 (61%, ee value: 86.0%) as a light yellow solid. (ES, m/z): [M+1]⁺ 385.2.

Synthesis of 123.8. To a solution of (R)-2-(2-hydroxy-4-(2-(2-methoxyethoxy)ethoxy)phenyl)-N-methoxy-N-methyl-4,5-dihydrothiazole-4-carboxamide (123.7, 1.27 g, 2.73 mmol, 1 eq) in THE (20 mL) was added LAH (155.47 mg, 4.10 mmol, 1.5 eq) at −40° C. under N₂. The temperature was allowed to rise up to −30° C. and stirred for 0.5 h. The reaction mixture was then quenched by successive additions of aqueous solutions of saturated NH₄Cl (30 mL) and 1 M solution of KHSO₄ (30 mL). The mixture was vigorously stirred and allowed to warm up to the room temperature until two phases were formed. After partition and extraction with ethyl acetate (2×50 mL), the organic layers were combined, dried over anhydrous sodium sulfate, filtered and concentrated under reduced pressure to give 690 mg of 123.8 (crude) as a yellow solid. The crude product was used to the next step without further purification. (ES, m/z): [M+1]⁺ 326.1.

Synthesis of 123.9 To a solution of (R)-2-(2-hydroxy-4-(2-(2-methoxyethoxy)ethoxy)phenyl)-4,5-dihydrothiazole-4-carbaldehyde (123.8, 690 mg, 2.12 mmol, 1 eq) in a mixture of EtOH (21 mL) and H₂O (7 mL) was added (S)-3-mercapto-2-(methylamino)propanoic acid (A-3S, 436.79 mg, 2.54 mmol, 1.2 eq, HCl) and KOAc (1.46 g, 14.84 mmol, 7 eq) at 25° C. The suspension was gently stirred 12 h at 25° C. in the dark. Ethanol was removed under reduced pressure and the crude was diluted in water. The aqueous layer was acidified to pH=5 by the addition of solid citric acid, and then the solution was extracted with ethyl acetate (2×50 mL). The organic layers were combined, dried over anhydrous sodium sulfate, filtered and evaporated to dryness under reduced pressure. The residue was purified by prep-HPLC (column: Welch Xtimate C18 250*70 mm #10 um; mobile phase: [water (NH₄HCO₃)-ACN]; B %: 10%-35%, 20 min) to give 360 mg of 123.9 (38%) as a light yellow solid. (ES, m/z): [M+1]⁺ 443.1.

Synthesis of I-123, I-124, I-123a, I-124a 360 mg of 123.9 (mixture of 4 peaks) was purified by twice SFC separation (Method 1 followed by Method 2) to give 23.19 mg of I-123 (6%, de value: 100%, Peak 2), 46.82 mg of I-124 (13%, de value: 97%, Peak 1) and 20.11 mg of I-123a (6%, de value: 97%, Peak 3) and 28.19 mg of I-124a (8%, de value: 98%, Peak 4) as light yellow solids. (ES, m/z): [M+1]⁺ 443.1

Method 1 (First time SFC separation): Instrument: Waters SFC 80 Q preparative SFC; Column: DAICEL CHIRALPAK AD (250 mm*30 mm, 10 um); Mobile phase: A for CO₂ and B for EtOH (0.1% NH₃H₂O); Gradient: B %=44% isocratic elution mode; Flow rate: 72 g/min; Wavelength: 220 nm; Column temperature: 40° C.; System back pressure: 100 bar.

Method 2 (Second time SFC separation): Instrument: Waters SFC 150 AP preparative SFC; Column: DAICEL CHIRALPAK AD (250 mm*30 mm, 10 um) Mobile phase: A for CO₂ and B for MEOH (0.1% NH₃H₂O); Gradient: B %=50% isocratic elution mode; Flow rate: 70 g/min; Wavelength: 220 nm; Column temperature: 35 degrees centigrade; System back pressure: 120 bar.

I-123: ¹H NMR (400 MHz, acetone) δ=7.32 (d, J=8.4 Hz, 1H), 6.56-6.42 (m, 2H), 4.95 (q, J=8 Hz, 1H), 4.53 (d, J=9.6 Hz, 1H), 4.25 (br t, J=6.4 Hz, 1H), 4.18 (br t, J=4.8 Hz, 2H), 3.85-3.79 (t, J=4.8 Hz, 2H), 3.67-3.58 (m, 3H), 3.5 (t, J=4.8 Hz 2H), 3.47-3.41 (m, 1H), 3.29 (s, 3H), 3.23 (d, J=6 Hz, 2H), 2.50 (s, 3H)

I-124: ¹H NMR (400 MHz, acetone) δ=7.33 (d, J=8.4 Hz, 1H), 6.55-6.46 (m, 2H), 5.13 (dt, J=5.6, 8.8 Hz, 1H), 4.58 (d, J=5.6 Hz, 1H), 4.20-4.15 (br t, J=7.8 Hz, 2H), 3.82 (br dd, J=4.0, 5.6 Hz, 2H), 3.71 (br t, J=7.2 Hz, 1H), 3.64 (dd, J=4.8, 5.6 Hz, 2H), 3.50 (dd, J=4.0, 5.6 Hz, 2H), 3.45 (d, J=8.4 Hz, 2H), 3.29 (s, 3H), 3.26-3.17 (m, 2H), 2.64 (s, 3H)

I-123a: ¹H NMR (400 MHz, acetone) δ=7.32 (d, J=8.8 Hz, 1H), 6.56-6.46 (m, 2H), 4.78 (q, J=8.4 Hz, 1H), 4.32 (d, J=8.0 Hz, 1H), 4.17 (t, J=4.4 Hz, 2H), 4.02 (br t, J=6 Hz, 1H), 3.85-3.79 (m, 2H), 3.66-3.62 (m, 2H), 3.61-3.56 (m, 1H), 3.53-3.40 (m, 4H), 3.34-3.26 (m, 4H), 2.63 (s, 3H)

I-124a: ¹H NMR (400 MHz, acetone) δ=7.32 (d, J=8.4 Hz, 1H), 6.52 (dd, J=2.4, 8.8 Hz, 1H), 6.48 (d, J=2.4 Hz, 1H), 5.21 (m, 1H), 5.03 (br d, J=4.4 Hz, 1H), 4.26-4.23 (m, 1H), 4.17 (br t, J=4.4 Hz, 2H), 3.81 (br t, J=4.8 Hz, 2H), 3.64 (dd, J=4.8, 6.4 Hz, 2H), 3.50 (dd, J=3.6, 5.6 Hz, 2H), 3.40 (br t, J=7.2 Hz, 2H), 3.29 (s, 3H), 3.21 (dd, J=6.4, 10 Hz, 1H), 3.07 (br d, J=8.8 Hz, 1H), 2.69 (s, 3H)

### Example 28: (4S)-2-((S)-2-(2-hydroxy-4-(2-(2-methoxyethoxy)ethoxy)phenyl)-4,5-dihydrothiazol-4-yl)-3-methylthiazolidine-4-carboxylic acid

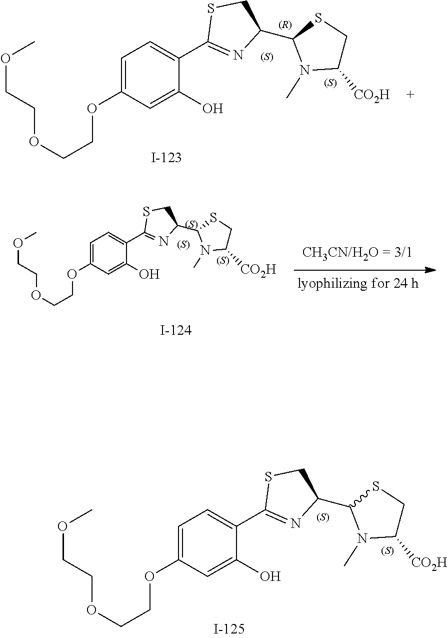

Synthesis of I-125. A solution of (2R,4S)-2-((S)-2-(2-hydroxy-4-(2-(2-methoxyethoxy)ethoxy)phenyl)-4,5-dihydrothiazol-4-yl)-3-methylthiazolidine-4-carboxylic acid (I-123, 23.17 mg) and (2S,4S)-2-((S)-2-(2-hydroxy-4-(2-(2-methoxyethoxy)ethoxy)phenyl)-4,5-dihydrothiazol-4-yl)-3-methylthiazolidine-4-carboxylic acid (I-124, 34.76 mg) in CH₃CN/H₂O (3/1, 4 mL) was shaken for 2 min to give a clear solution. The mixture was then lyophilized for 24 h to give 50.89 mg of I-125 (87%) as a light yellow solid. (ES, m/z): [M+1]⁺ 443.1

I-125. ¹H NMR (400 MHz, acetone) δ=7.32 (dd, J=2.4, 8.4 Hz, 1.51H), 6.54-6.47 (m, 3.19H), 5.18-5.07 (m, 1.15H), 4.94 (q, J=8.0 Hz, 0.73H), 4.58 (br d, J=5.6 Hz, 0.98H), 4.51 (br d, J=8.4 Hz, 0.64H), 4.23 (br t, J=6.4 Hz, 0.86H), 4.17 (br t, J=4.4 Hz, 3.66H), 3.81 (t, J=4.8 Hz, 3.82H), 3.71 (br t, J=7.6 Hz, 1.34H), 3.66-3.62 (m, 3.82H), 3.52-3.47 (m, 3.58H), 3.45 (br d, J=9.2 Hz, 2.31H), 3.29 (br s, 4.99H), 3.24-3.19 (m, 3.09H), 2.63 (s, 3H), 2.48 (s, 1.80H)

### Example 29: (4S)-3-methyl-2-((S)-2-phenyl-4,5-dihydrothiazol-4-yl)thiazolidine-4-carboxylic acid

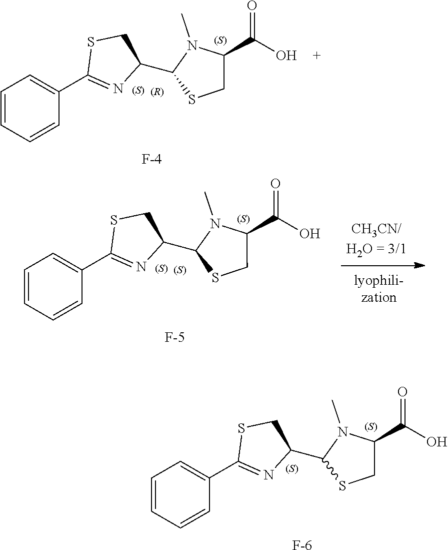

Synthesis of F-6 (2R,4S)-3-methyl-2-((S)-2-phenyl-4,5-dihydrothiazol-4-yl)thiazolidine-4-carboxylic acid (F-4, 7.01 mg) and (2S,4S)-3-methyl-2-((S)-2-phenyl-4,5-dihydrothiazol-4-yl)thiazolidine-4-carboxylic acid (F-5, 10.55 mg) were dissolved in ACN (3 mL) and H₂O (1 mL), and the mixture was under lyophilization. To give 10.37 mg of F-6 (100%) as a light yellow solid. (ES, m/z): [M+1]⁺ 339.0.

F-6: ¹H NMR (400 MHz, acetone-d6) δ=7.82 (d, J=8.1 Hz, 3H), 7.59-7.40 (m, 5H), 5.03 (br d, J=5.6 Hz, 1H), 4.93 (d, J=8.1 Hz, 0.71H), 4.66-4.51 (m, 1.72H), 4.36-4.22 (m, 0.72H), 3.73-3.68 (m, 1H), 3.66-3.60 (m, 0.60H), 3.50 (d, J=9.0 Hz, 2H), 3.47-3.41 (m, 0.63H), 3.25-3.18 (m, 3H), 2.65 (s, 3H), 2.49 (s, 2H)

### Example 30: (2S,4R)-2-((R)-2-(2-hydroxyphenyl)-4,5-dihydrothiazol-4-yl)-3-methylthiazolidine-4-carboxylic acid and (2R,4R)-2-((R)-2-(2-hydroxyphenyl)-4,5-dihydrothiazol-4-yl)-3-methylthiazolidine-4-carboxylic acid

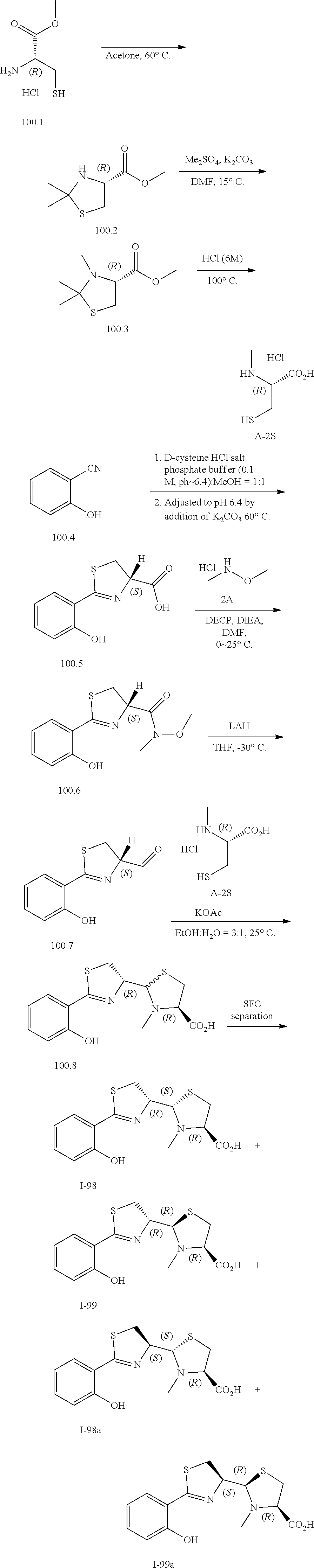

Synthesis of 100.2. A mixture of (R)-methyl 2-amino-3-mercaptopropanoate (5 g, 29.13 mmol, 1 eq, HCl) in acetone-d₆ (100 mL) was degassed and purged with N₂ for 3 times, and then the mixture was stirred at 60° C. for 16 hr under N₂ atmosphere. The reaction mixture was concentrated under reduced pressure to remove acetone-d6. The residue was diluted with ethyl acetate (50 mL), washed successively with water, 5% aqueous sodium bicarbonate solution (3×50 mL), and brine (25 mL), and then dried over anhydrous sodium sulfate, filtered and concentrated under reduced pressure to give a residue. The residue was purified by column chromatography (SiO₂, Petroleum ether/Ethyl acetate=0/1 to 15.6/1) to give 4.6 g of 100.2 (90.11%) as a colorless oil. (ES, m/z): [M+1]⁺ 176.0.

Synthesis of 100.3. To a solution of (R)-methyl 2,2-dimethylthiazolidine-4-carboxylate (100.2, 3.08 g, 17.58 mmol, 1 eq) and K₂CO₃ (3.64 g, 26.36 mmol, 1.5 eq) in DMF (30 mL) was added Me₂SO₄ (6.65 g, 52.73 mmol, 5.00 mL, 3 eq) dropwise with stirring at 0° C. The mixture was stirred at 15° C. for 5 h. The reaction mixture was quenched by the addition of water (20 mL) at 0° C. The residue was diluted with water (10 mL) and extracted with ethyl acetate (3×30 mL). The combined organic layers were washed with brine (30 mL). The combined organic layers were dried over Na₂SO₄, filtered and concentrated under reduced pressure to give a residue. The residue was purified by column chromatography (SiO₂, Petroleum ether/Ethyl acetate=60/1) to give 1.5 g of 103.3 (46%) as a colorless oil. (ES, m/z): [M+1]⁺ 190.8.

Synthesis of A-2S. A mixture of (R)-methyl 2,2,3-trimethylthiazolidine-4-carboxylate (103.3, 1.5 g, 21.13 mmol, 1 eq) in HCl (6 M, 20 mL, 15.14 eq) was degassed and purged with Ar for 3 times, and then the mixture was stirred at 100° C. for 8 hr under Ar atmosphere. The reaction mixture was concentrated under reduced pressure to give 1.36 g of A-2S (crude, HCl) as a brown solid. The crude product was used directly to the next step without further purification. (ES, m/z): [M+1]⁺ 136.1.

Synthesis of 100.5. To a mixture of 2-hydroxybenzonitrile (100.4, 2 g, 16.79 mmol, 1 eq) in MeOH (20 mL) and phosphate buffer (20 mL) was added (2S)-2-amino-3-sulfanyl-propanoic acid (5.29 g, 33.58 mmol, 2 eq, HCl) at room temperature. The solution was adjusted to pH=6.4 by the addition of solid K₂CO₃ carefully and the reaction mixture was stirred at 60° C. for 12 h. The mixture was concentrated under reduced pressure, and the yellow crude residue was diluted with water (20 mL). The solution was adjusted to pH=2.0 by the addition of solid citric acid. After extraction with EA (3×30 mL), the combined organic layers were dried over Na₂SO₄, filtered and concentrated under reduced pressure to give a residue to give 3.7 g of 100.5 (96.24%, % ee value is 93.12%) as a yellow solid. (ES, m/z): [M+1]⁺ 223.9.

Synthesis of 100.6. To a solution of (S)-2-(2-hydroxyphenyl)-4,5-dihydrothiazole-4-carboxylic acid (100.5, 2.1 g, 8.94 mmol, 1 eq), N-methoxymethanamine (2A, 958.84 mg, 9.83 mmol, 1.1 eq, HCl) and DECP (1.60 g, 9.83 mmol, 1.48 mL, 1.1 eq) in DMF (20 mL) was added DIEA (1.73 g, 13.40 mmol, 2.33 mL, 1.5 eq) at 0° C. under N₂. The mixture was stirred for 30 min at 0° C., then another 2 h at 25° C. The reaction mixture was diluted with water 45 mL and extracted with EA 180 mL (60 mL*3). The combined organic layers were washed with NaHCO₃ 90 mL (30 mL*3), dried over Na₂SO₄, filtered and concentrated under reduced pressure to give a residue to give 2 g of 100.6 (81%, ee value is 83.62%) as a light yellow solid. (ES, m/z): [M+1]⁺ 267.0.

Synthesis of 100.7. To a solution of (S)-2-(2-hydroxyphenyl)-N-methoxy-N-methyl-4,5-dihydrothiazole-4-carboxamide (100.6, 2.1 g, 7.89 mmol, 1 eq) in THE (42 mL) was added LAH (448.93 mg, 11.83 mmol, 1.5 eq) at −40° C. under N₂. The temperature was allowed to rise up to −30° C. in 0.5 h. The reaction mixture was then hydrolyzed by successive additions of aqueous solutions of saturated NH₄Cl (42 mL) and a 1 M solution of KHSO₄ (42 mL). The mixture was vigorously stirred and allowed to warm up to the room temperature until two phases were formed. After partition and extraction with EA (3×30 mL), the organic layers were collected, dried over Na2SO4 and filtered before being evaporated under reduced pressure to give 1.27 g of 100.7 (crude) as a yellow oil. The crude product was used to the next step without further purification. (ES, m/z): [M+1]⁺ 208.1.

Synthesis of I-100.8. To a solution of (S)-2-(2-hydroxyphenyl)-4,5-dihydrothiazole-4-carbaldehyde (100.7, 1.3 g, 6.27 mmol, 1 eq) in a mixture of EtOH (60 mL) and H₂O (20 mL) was added (R)-3-mercapto-2-(methylamino)propanoic acid (A-2S, 1.29 g, 7.53 mmol, 1.2 eq, HCl) and KOAc (4.31 g, 43.91 mmol, 7 eq). This suspension was gently stirred 12 h at 20° C. in the dark. Ethanol was removed under reduced pressure and the crude was diluted in water. The aqueous layer was acidified to pH=5 by the addition of solid citric acid, and then the solution was extracted with ethyl acetate (3×100 mL). The organic layers were collected, dried over Na₂SO₄, filtered and evaporated to dryness under reduced pressure. The residue was purified by prep-HPLC (column: Welch Xtimate C18 250*70 mm #10 um; mobile phase: [water (NH₄HCO₃)-ACN]; B %: 10%-40%, 20 min) to give 540 mg of I-100.8 (26.54%) as a light yellow solid. (ES, m/z): [M+1]⁺ 325.1.

Synthesis of I-98, I-99, I-98a, and I-99a. 200 mg of I-100.8 (mixture of 4 peaks) was purified by twice SFC separation to give 40 mg of I-98 (20%, ee value is 77.21%, Peak 4), 50 mg of I-99 (25%, ee value is 84.92%, Peak 1), 23.67 mg of I-98a (11.83%, ee value is 86.04%, Peak 3) and 9.73 mg of I-99a (4.86%, ee value is 56.66%, Peak 2) as light yellow solids. (ES, m/z): [M+1]⁺ 325.1.

### SFC Separation Methods:

Method 1: Instrument: Waters SFC80Q preparative SFC; column: DAICEL CHIRALPAK AD (250 mm*30 mm, 10 um); mobile phase: [0.1% NH₃H₂O EtOH]; B %: 33%-33%, 9 min.

Method 2: Instrument: Waters SFC80Q preparative SFC; Column: DAICEL CHIRALPAK AD (250 mm*30 mm, 10 um); mobile phase: [0.1% NH₃H₂O IPA]; B %: 35%-35%, 9 min.

I-98: ¹H NMR (400 MHz, acetone-d6) δ=7.49-7.35 (m, 2H), 7.01-6.87 (m, 2H), 5.02 (d, J=8.3 Hz, 1H), 4.56 (d, J=8.3 Hz, 1H), 4.26 (t, J=6.5 Hz, 1H), 3.67 (dd, J=8.6, 11.3 Hz, 1H), 3.47 (dd, J=7.9, 11.4 Hz, 1H), 3.28-3.18 (m, 2H), 2.51 (s, 3H).

I-99: ¹H NMR (400 MHz, acetone-d6) δ=7.48-7.37 (m, 2H), 6.98-6.89 (m, 2H), 5.22 (d, J=5.5 Hz, 1H), 4.63 (d, J=5.4 Hz, 1H), 3.72 (dd, J=6.8, 8.0 Hz, 1H), 3.50 (d, J=8.9 Hz, 2H), 3.22 (dd, J=1.8, 7.4 Hz, 2H), 2.65 (s, 3H).

I-98a: ¹H NMR (400 MHz, acetone-d6) δ=7.48-7.35 (m, 2H), 6.99-6.87 (m, 2H), 4.85 (q, J=8.4 Hz, 1H), 4.35 (d, J=8.3 Hz, 1H), 4.03 (t, J=6.1 Hz, 1H), 3.68-3.61 (m, 1H), 3.54 (dd, J=8.2, 11.4 Hz, 1H), 3.45 (dd, J=5.3, 11.0 Hz, 1H), 2.64 (s, 3H).

I-99a: ¹H NMR (400 MHz, acetone-d6) δ=7.48-7.35 (m, 2H), 6.99-6.88 (m, 2H), 5.30 (dt, J=4.7, 9.0 Hz, 1H), 5.07 (d, J=4.8 Hz, 1H), 4.27 (dd, J=2.3, 6.6 Hz, 1H), 3.52-3.38 (m, 2H), 3.23 (dd, J=6.5, 10.5 Hz, 1H), 3.07 (dd, J=2.1, 10.5 Hz, 1H), 2.72 (s, 3H).

### Example 31: (4R)-2-((R)-2-(2-hydroxyphenyl)-4,5-dihydrothiazol-4-yl)-3-methylthiazolidine-4-carboxylic acid

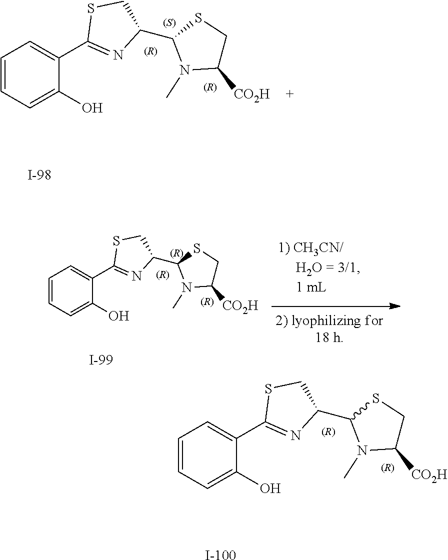

Synthesis of I-100. A solution 20 mg of 1-98 and 30 mg of 1-99 in CH₃CN/H₂O (3/1, 4 mL) was shaken for 2 min to give a clear solution. The solution was then lyophilized for 48 h to give 42.56 mg of I-100 (100%, 59.5+40.49% purity, stereochemical ratio: 58.48: 41.38). (ES, m/z): [M+1]+ 339.1.

¹H NMR (400 MHz, acetone-d6) δ=7.48-7.37 (m, 3H), 6.94 (br dd, J=4.1, 7.9 Hz, 3H), 5.22 (dt, J=5.4, 8.9 Hz, 1H), 5.03 (q, J=8.3 Hz, 0.61H), 4.64 (d, J=5.5 Hz, 1H), 4.56 (d, J=8.4 Hz, 0.54H), 4.27 (t, J=6.5 Hz, 0.65H), 3.77-3.63 (m, 2H), 3.54-3.43 (m, 2.83H), 3.28-3.17 (m, 3H), 2.65 (s, 3H), 2.51 (s, 1.62H).

### Example 32: (4R)—N-(2-(3-(but-3-yn-1-yl)-3H-diazirin-3-yl)ethyl)-2-((R)-2-(2-hydroxy phenyl)-4,5-dihydrothiazol-4-yl)-3-methylthiazolidine-4-carboxamide

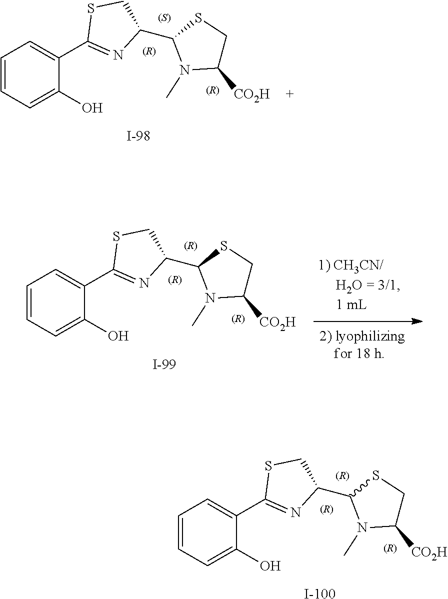

Synthesis of I-138. To a solution of (4R)-2-((R)-2-(2-hydroxyphenyl)-4,5-dihydrothiazol-4-yl)-3-methylthiazolidine-4-carboxylic acid (I-100, 20 mg, 61.65 μmol, 1 eq) and 2-(3-(but-3-yn-1-yl)-3H-diazirin-3-yl)ethanamine (16.91 mg, 123.30 mol, 2 eq) in DMF (1.5 mL) was added EDCI (17.73 mg, 92.47 mol, 1.5 eq), DIEA (39.84 mg, 308.24 mol, 53.69 uL, 5 eq) and HOBt (12.50 mg, 92.47 mol, 1.5 eq). The mixture was stirred at 15° C. for 16 hr. The mixture was purified by prep-HPLC (column: Waters Xbridge BEH C18 100*30 mm*10 um; mobile phase: [water (NH₄HCO₃)-ACN]; B %: 45%-70%, 8 min) to give 17.96 mg of I-138 (64%, stereochemical ratio: 61:38) as a yellow gum. (ES, m/z): [M+1]⁺ 444.0. ¹H NMR (400 MHz, chloroform-d) 6=12.67 (br s, 0.93H), 12.17 (s, 0.61H), 7.46-7.30 (m, 4.22H), 7.03-6.98 (m, 1.55H), 6.94-6.84 (m, 2.24H), 5.03 (q, J=8.6 Hz, 0.62H), 4.86 (q, J=8.4 Hz, 1H), 4.48 (d, J=8.8 Hz, 0.59H), 4.29 (d, J=8.4 Hz, 1H), 3.89 (dd, J=3.6, 8 Hz, 0.83H), 3.69 (t, J=7.6 Hz, 1H), 3.64-3.48 (m, 2.97H), 3.39-3.31 (m, 1.98H), 3.30-3.22 (m, 1.81H), 3.22-3.15 (m, 1H), 3.15-3.05 (m, 1.85H), 3.02 (dd, J=7.2, 13.8 Hz, 0.58H), 2.65 (s, 3H), 2.52 (s, 1.75H), 2.03-1.95 (m, 4.73H), 1.75 (m, 1.29H), 1.71-1.60 (m, 6.70H).

### Example 33: (4S)-2-((S)-2-(2-hydroxyphenyl)-4,5-dihydrothiazol-4-yl)-3-methylthiazolidine-4-carboxylic acid

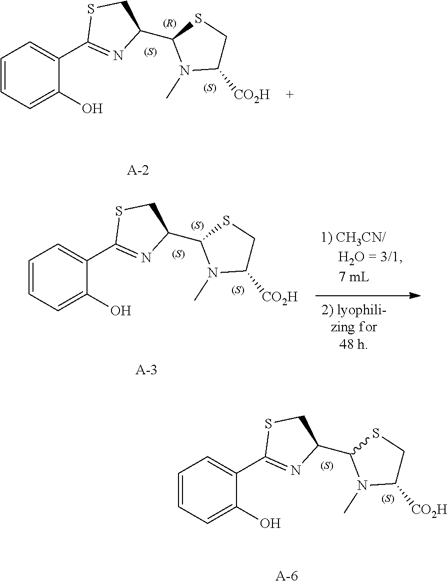

Synthesis of A-6. A solution of (2R,4S)-2-((S)-2-(2-hydroxyphenyl)-4,5-dihydrothiazol-4-yl)-3-methylthiazolidine-4-carboxylic acid (A-2, 307.01 mg) and (2S,4S)-2-((S)-2-(2-hydroxyphenyl)-4,5-dihydrothiazol-4-yl)-3-methylthiazolidine-4-carboxylic acid (A-3, 460.51 mg) in CH₃CN/H₂O (3/1, 7 mL) was shaken for 2 min to give a clear solution. The solution was then lyophilized for 48 h to give 732.43 mg of I-121 (95%). (ES, m/z): [M+1]+ 325.1.

¹H NMR (400 MHz, acetone-d6) δ=7.46-7.38 (m, 2.94H), 7.01-6.88 (m, 3.05H), 5.22 (dt, J=5.4, 8.9 Hz, 0.99H), 5.06 (m, 0.52H), 4.63 (d, J=5.4 Hz, 0.99H), 4.60-4.51 (m, 0.56H), 4.24 (t, J=6.5 Hz, 0.52H), 3.74-3.64 (m, 1.52H), 3.55-3.44 (m, 2.56H), 3.28-3.17 (m, 3.09H), 2.65 (s, 3.00H), 2.50 (s, 1.53H).

### Example 34: (4S)-2-((R)-2-(2-hydroxyphenyl)-4,5-dihydrothiazol-4-yl)-3-methylthiazolidine-4-carboxylic acid

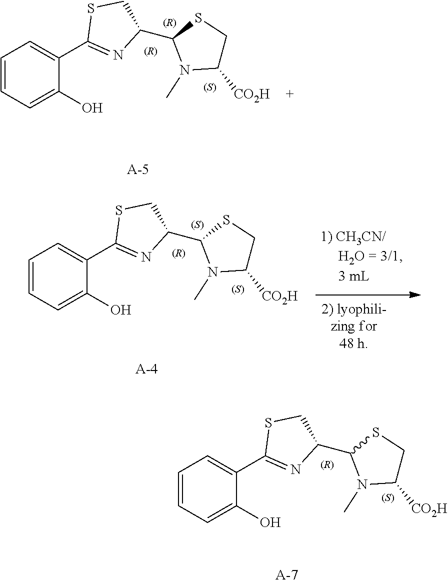

Synthesis of A-7. A solution of (2R,4S)-2-((R)-2-(2-hydroxyphenyl)-4,5-dihydrothiazol-4-yl)-3-methylthiazolidine-4-carboxylic acid (A-5, 46.41 mg) and (2S,4S)-2-((R)-2-(2-hydroxyphenyl)-4,5-dihydrothiazol-4-yl)-3-methylthiazolidine-4-carboxylic acid (A-4, 15.47 mg) in CH₃CN/H₂O (3/1, 3 mL) was shaken for 2 min to give a clear solution. The solution was then lyophilized for 48 h to give 50.36 mg of A-7 (81%). (ES, m/z): [M+1]+ 325.1.

¹H NMR (400 MHz, acetone-d6) δ=7.50-7.33 (m, 2.76H), 7.02-6.86 (m, 2.78H), 5.28 (dt, J=4.8, 9.1 Hz, 0.35H), 5.06 (br d, J=4.1 Hz, 0.31H), 4.92-4.80 (m, 1.02H), 4.34 (br d, J=8.1 Hz, 1.06H), 4.25 (br d, J=5.0 Hz, 0.38H), 4.00 (br s, 1.07H), 3.69-3.59 (m, 1.22H), 3.58-3.48 (m, 1.17H), 3.48-3.38 (m, 1.75H), 3.35-3.26 (m, 1.07H), 3.22 (br dd, J=6.4, 10.3 Hz, 0.41H), 3.06 (br d, J=11.0 Hz, 0.37H), 2.71 (s, 1.05H), 2.63 (s, 3.00H).

### Example 35: Biological Assays

### A. Dectin-1 Assay:

Compounds described herein are added to mammalian cells for bioactivity assessment. The HEK-Blue Dectin-1b cells line (InVivoGen) releases a reporter protein (secreted embryonic alkaline phosphatase, SEAP) to the culture supernatant in response to Dectin-1 activation. HEK-Blue reporter cells were grown in DMEM culture media with 10% decomplemented fetal calf serum (InvivoGen, 1% Pen/Strep antibiotic (InvivoGen), 0.2% Normocin antibiotic (InvivoGen). On the day prior to the assay, cells were seeded in a 96 flat-bottom well plate at 50,000 cells/well and further incubated for 20 hours. On the day of the assay cells were incubated with 1 μM, 10 μM or 30 μM of the compounds described herein or with 1 μM of the SYKi IV Dectin-1b Inhibitor (Millipore) or with DMSO (vehicle control) for 6 hours, followed by the addition of beta-glucan peptide (BGP, a Dectin-1 ligand at 30 μg/mL) and incubation for 16-18 hours. After incubation was concluded, the cultures supernatants were collected and Dectin-1 related activity was assessed by measurement of the SEAP-reporter protein presence, using Quanti-Blue assay (Invivogen) and these values were adjusted by the Cell Viability levels that was assessed using the Cell-Titer-Glo2 Reagent (Promega).

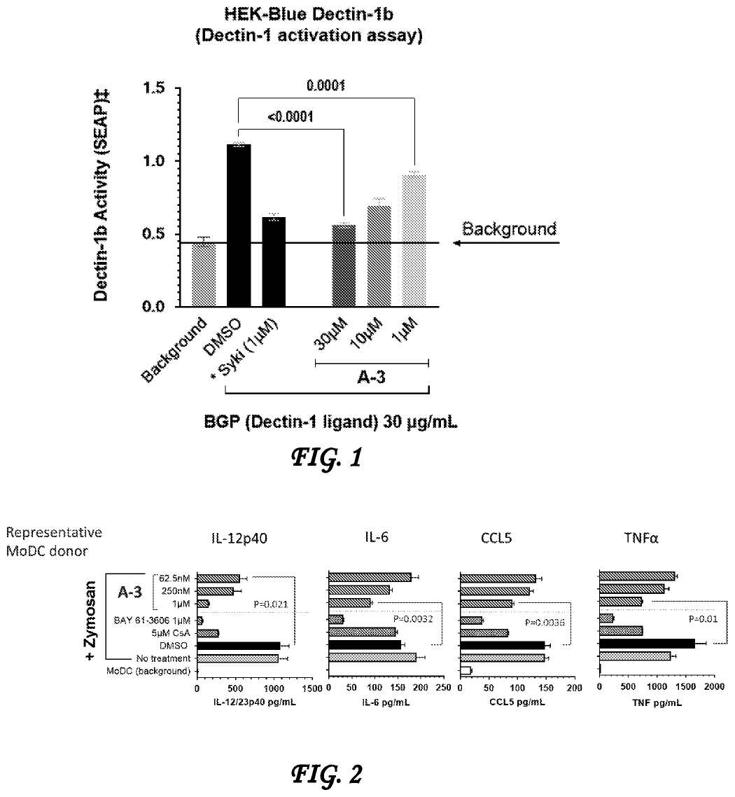

Results represent cell number adjusted SEAP levels in a triplicate setting for each condition and cell numbers and viability corrections reflected less than 5% corrections. See, e.g., FIG. 1 for compound A-3.

| Compound | Activity |
|---|---|
| A-1 | <30 μM |
| E-1 | <30 μM |
| C-1 | <30 μM |
| B-1 | <30 μM |
| D-1 | <30 μM |
| A-2 | <30 μM |
| A-3 | <30 μM |
| C-2 | <30 μM |
| C-3 | <30 μM |
| F-2 | <30 μM |
| G-4 + G-5 | <30 μM |
| A-2 + A-3 | <30 μM |

Compounds A1 and B1 show inhibition of BGP-induced Dectin-1b reporter gene activity in a concentration-dependent manner over a range from 1 μM to 30 μM, with maximal inhibition at 30 μM, the maximum concentration tested. Compound E1 is less active in this HEK-Dectin-1b assay at these concentrations. See, FIG. 4.

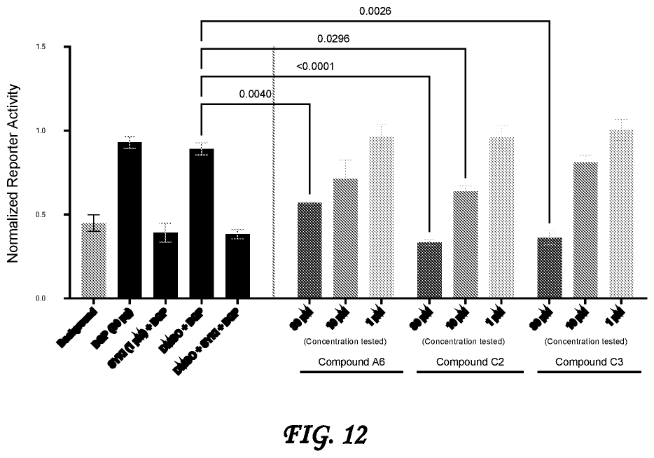

Compounds A6, C2 and C3 demonstrate dose-dependent inhibition of BGP-induced Dectin-1b signaling in BEK cells over a range from 1 μM to 30 μM, with maximal inhibition at 30 μM, the maximum concentration tested. See, FIG. 12.

### B. Monocyte Derived DC (MoDC) Assays Protocols

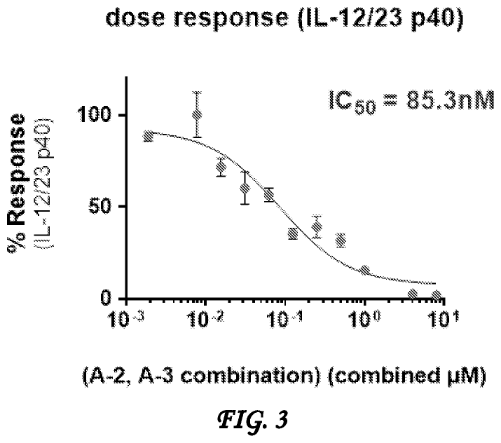

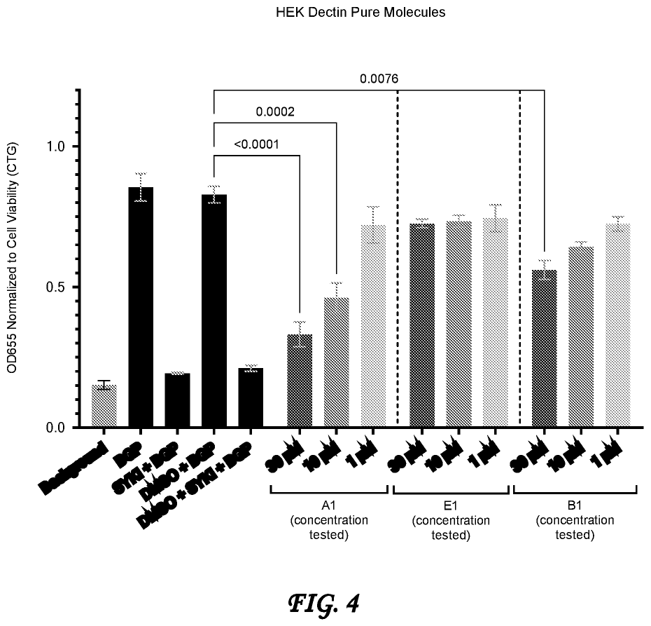

Monocyte derived DC (MoDC) were differentiated from healthy human donor derived CD14+ monocytes. Commercially available freshly prepared Leukopaks were obtained from healthy blood donor and kept refrigerated on ice before spin centrifuged and the pelleted cells are rinsed and resuspended with MACS buffer (Miltenyi Biotec). Next, CD14+ monocytes were purified using MultiMACS™ Cell24 Separator-Plus instrumentation and reagents according to the manufacturer (Miltenyi Biotec) instructions. The resulting purified CD14⁺ cell fraction was washed, enumerated, and subsequently cryopreserved for later use. At the experiment first day cryopreserved CD14′ cells were thawed, rinsed with culture media, and then resuspended in culture media principally composed of RPMI (Invitrogen) supplemented with 10% v/v heat inactivated FBS. For generation of MoDC, these CD14⁺ cells were enumerated and seeded into 75 cm² tissue culture flasks in 30 mL of culture media supplemented with GM-CSF (40-100 ng/mL) and IL-4 (25 ng/mL). Every other day, half of the culture media was replaced with fresh media (containing the same cytokine supplementation) and after 6-7 days of culture, fully differentiated and matured MoDC were obtained as confirmed by conventional Immuno-phenotype markers (levels of expression or absence of surface CD80, CD83, CD86, CD209, HLA-DR, CD14 & CD64) that were verified by flow-cytometry. Fully differentiated (mature) MoDCs, were seeded into 96 flat-well plates and incubated with a various concentration of the compound described herein, or with control solution (DMSO), or with the pharmacologic inhibitors Cyclosporin-A (CsA) and BAY 61-3606 (which is a known inhibitor of Syk kinase) for 6 hours. Next, the MoDC culture well were added with Dectin-1(CLEC7a1)-ligand (Zymosan at 5 μg/mL), or the Toll-like receptor agonist R848, and further incubated for 18-20 hours. After incubation concluded, culture supernatants were collected, and the levels of inflammatory cytokines and chemokines was measured in a triplicate setting for each condition by Luminex® multiplex assay (FIG. 2) or by ELISA (FIG. 3).

| Compound | Activity |
|---|---|
| B-1 | <30 μM |

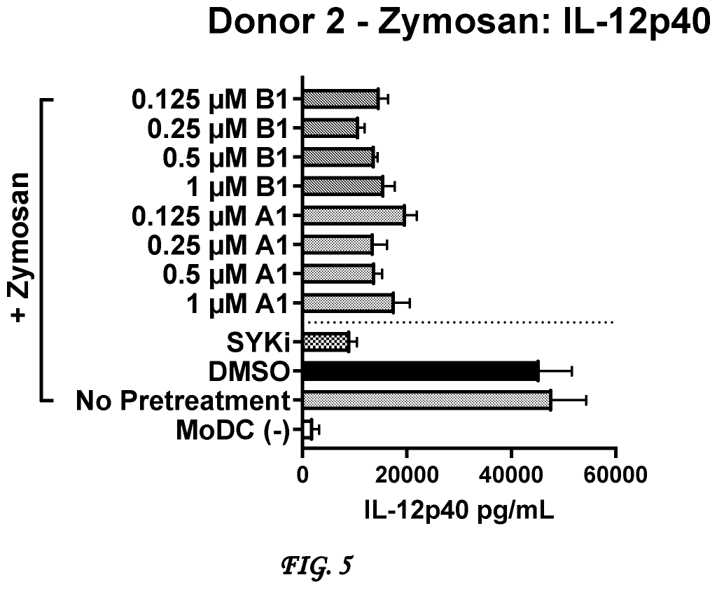

Compounds A1 and B1 show potent inhibition of Zymosan-induced production of the pro-inflammatory cytokine IL-12p40 in monocyte-derived dendritic cells from healthy human donor 2. See, FIG. 5.

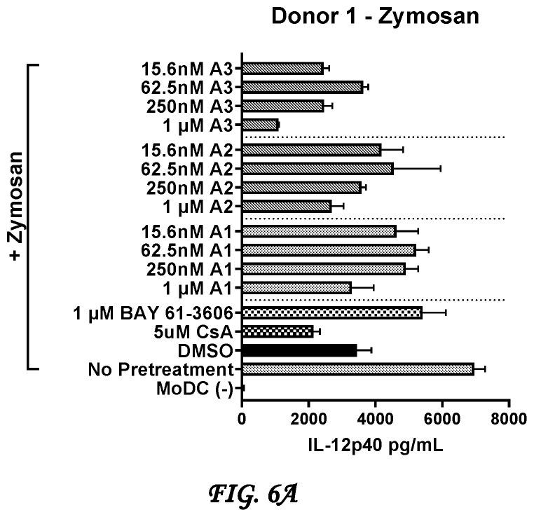

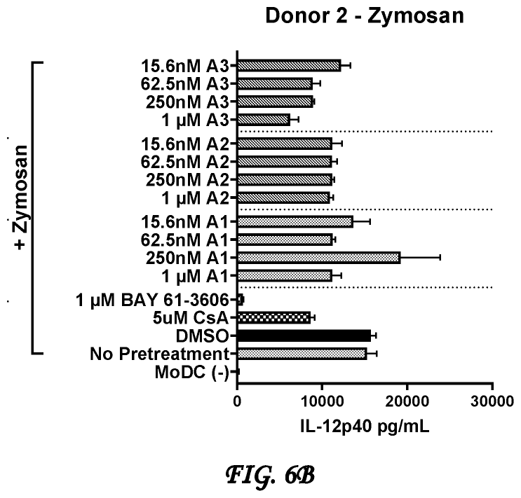

Compounds A1, A2 and A3 show inhibition of Zymosan-induced production of the pro-inflammatory cytokine IL-12p40 in monocyte-derived dendritic cells from healthy human donors 1, 2, and 3. Compounds were tested at 15.5 nM to 1 μM final concentration, with maximal inhibition at the highest concentration, 1 μM. See, FIGS. 6A-6C.

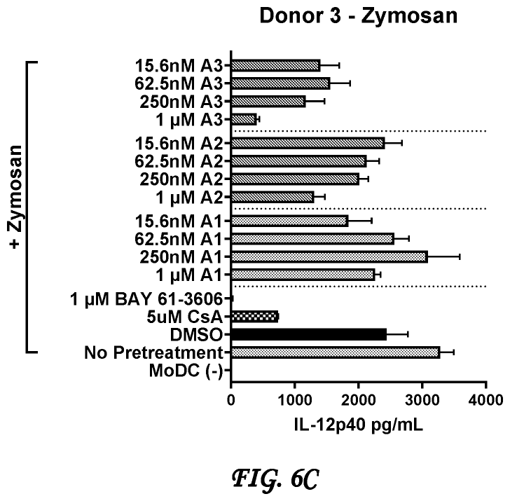

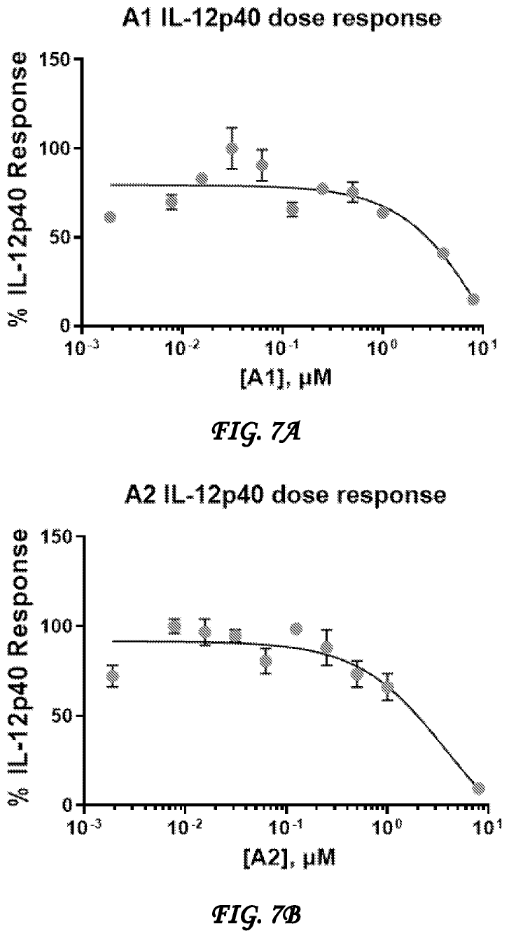

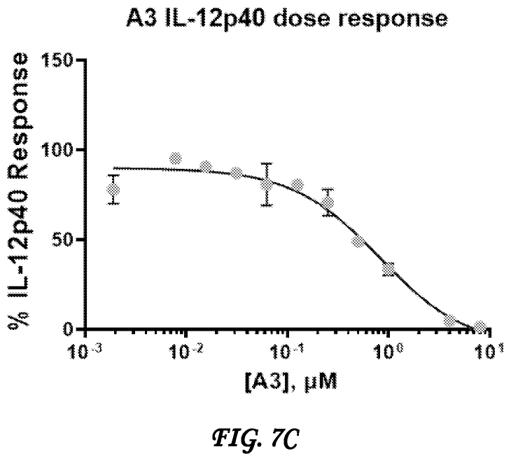

Compounds A1, A2 and A3 show dose-dependent inhibition of Zymosan-induced production of the pro-inflammatory cytokine IL-12p40 in monocyte-derived dendritic cells from healthy human donor 1. See, FIGS. 7A-7C.

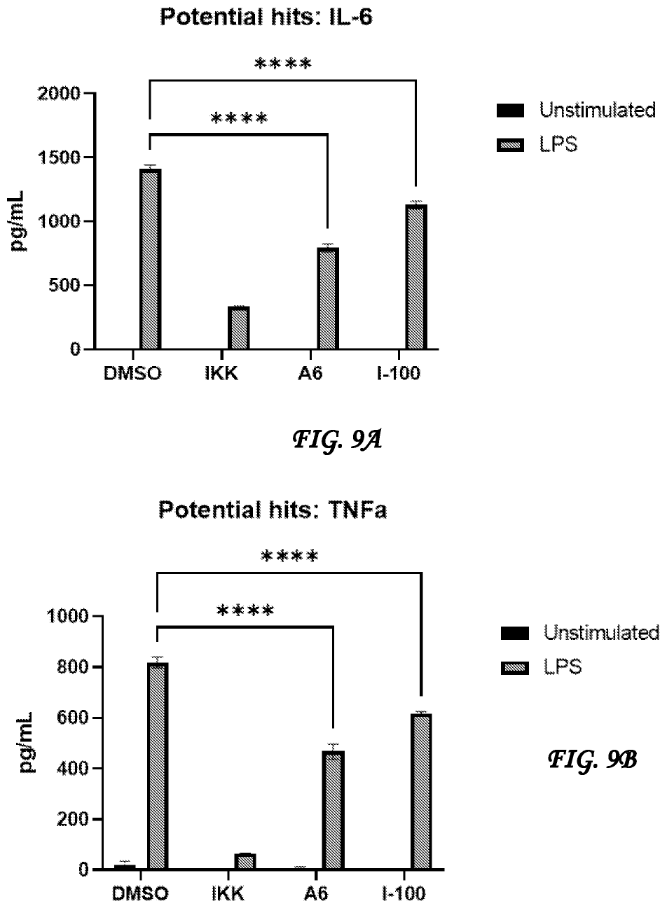

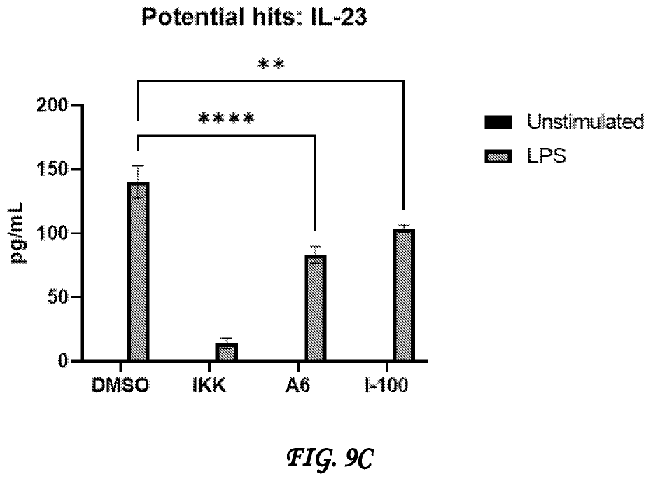

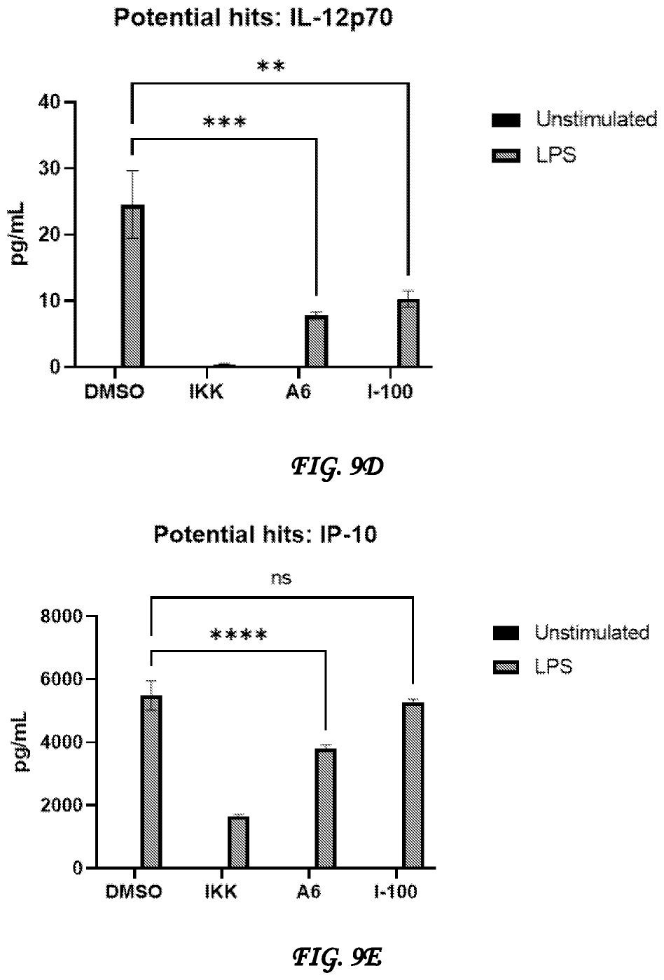

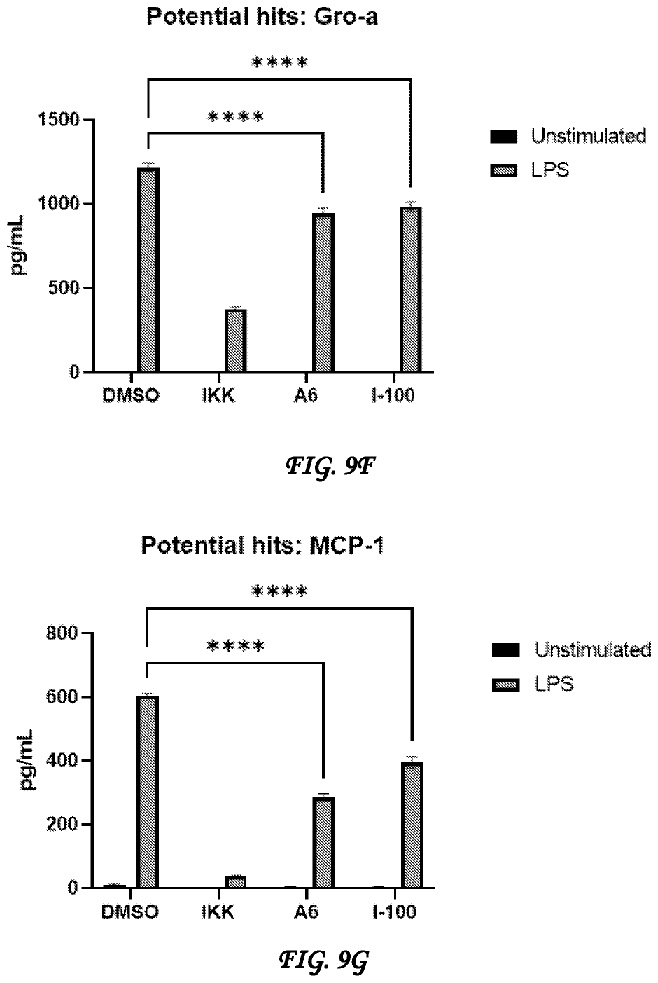

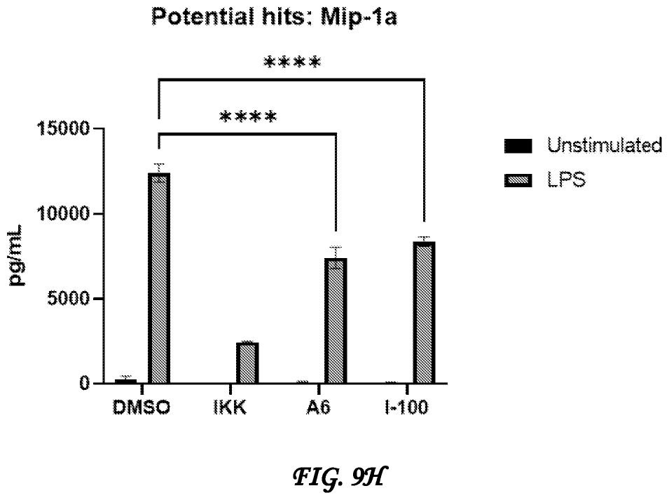

Compounds A6 and I-100 inhibit several pro-inflammatory cytokines in LPS-stimulated MoDC from healthy human donor 14. A6 causes a statistically significant decrease in IL-6 (FIG. 9A), TNFα (FIG. 9B), IL-23 (FIG. 9C), IL-12p70 (FIG. 9D), IP-10 (FIG. 9E), Gro-α (FIG. 9F), MCP-1 (FIG. 9G) and Mip-1α (FIG. 9H). I-100 causes a statistically significant decrease in IL-6, TNFα, IL-23, IL-12p70, Gro-α, MCP-1 and Mip-1α. See, FIGS. 9A-9H.

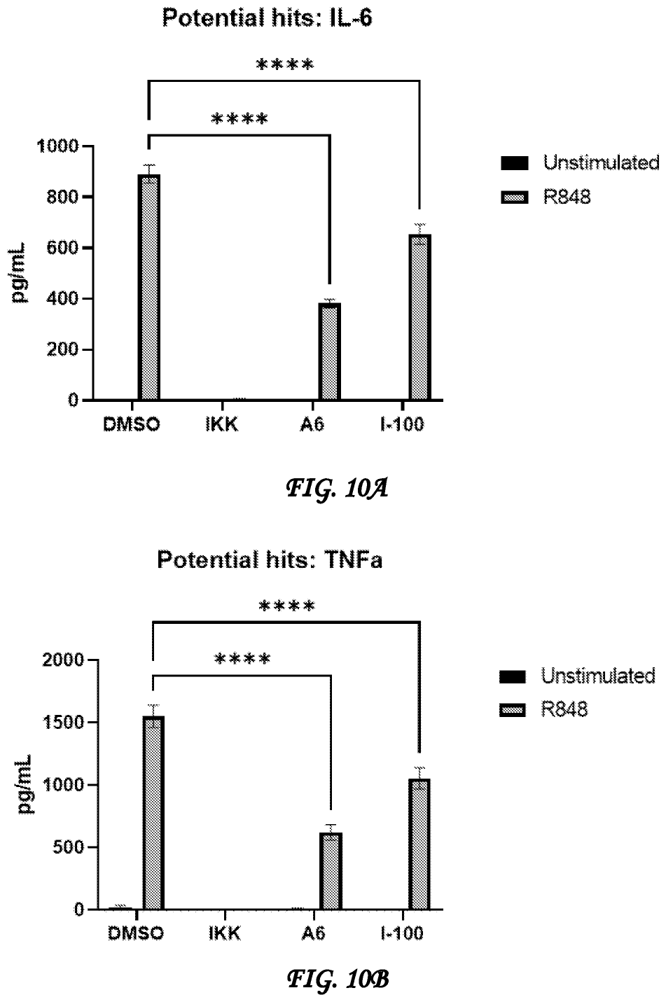

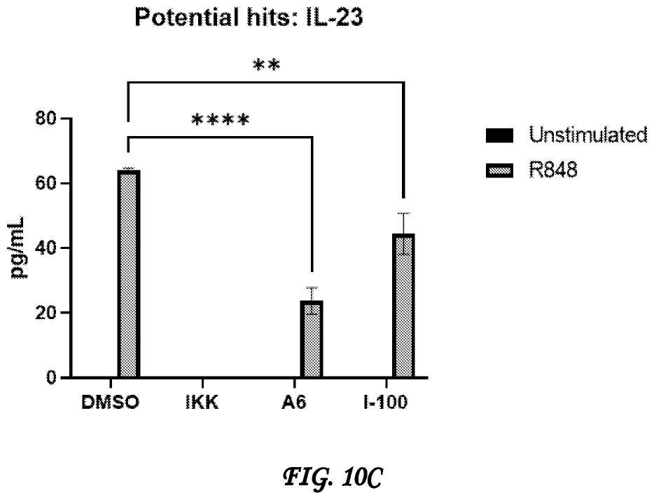

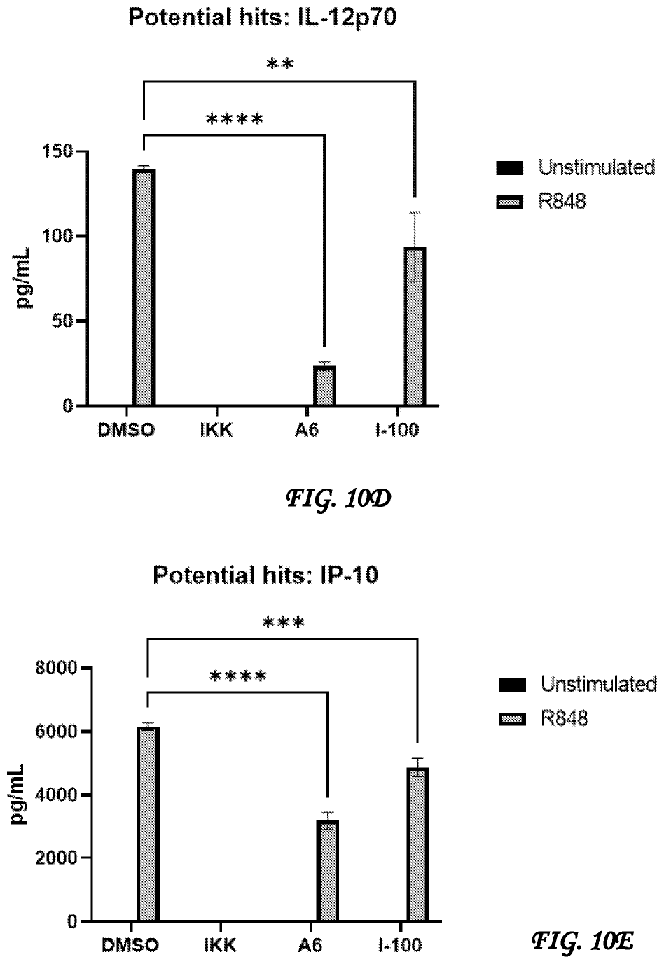

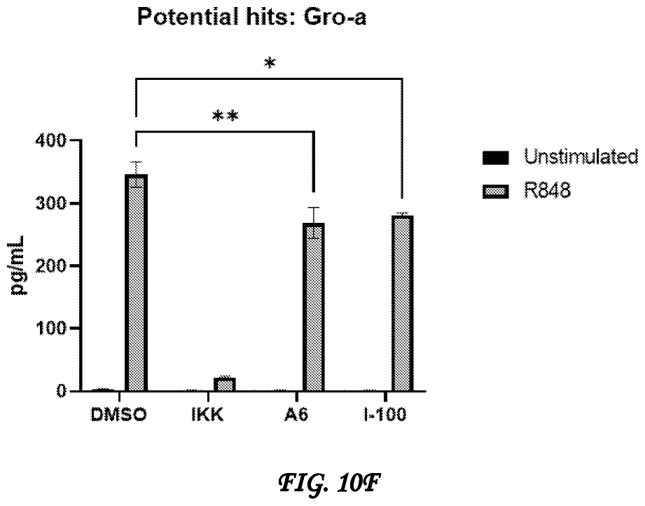

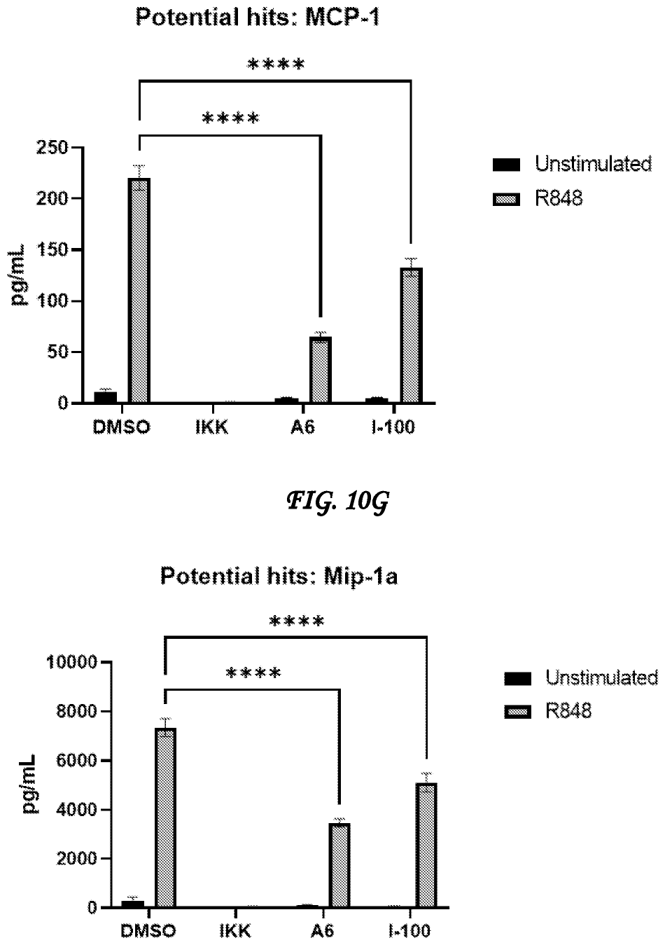

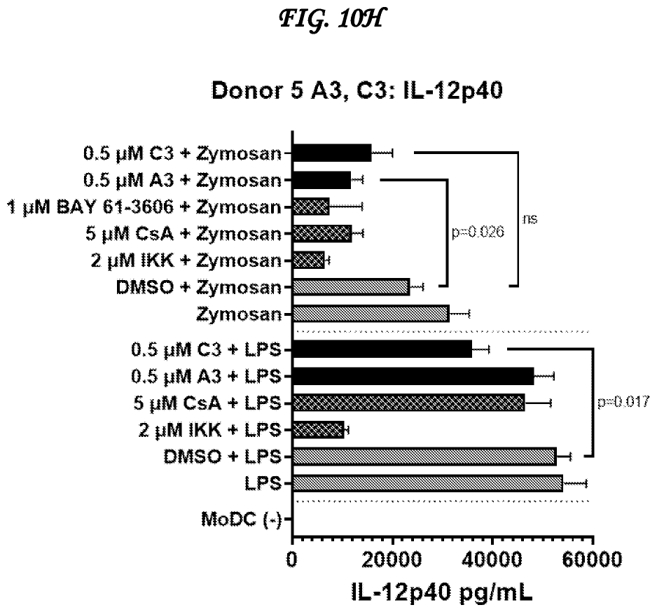

Compounds A6 and I-100 inhibit several pro-inflammatory cytokines in R848-stimulated MoDC from healthy human donor 14. A6 causes a statistically significant decrease in IL-6 (FIG. 10A), TNFα (FIG. 10B), IL-23 (FIG. 10C), IL-12p70 (FIG. 10D), IP-10 (FIG. 10E), Gro-α (FIG. 10F), MCP-1 (FIG. 10G) and Mip-1α (FIG. 10H). I-100 causes a statistically significant decrease in IL-6, TNFα, IL-23, IL-12p70, IP-10, Gro-α, MCP-1 and Mip-1α. See, FIGS. 10A-10H.

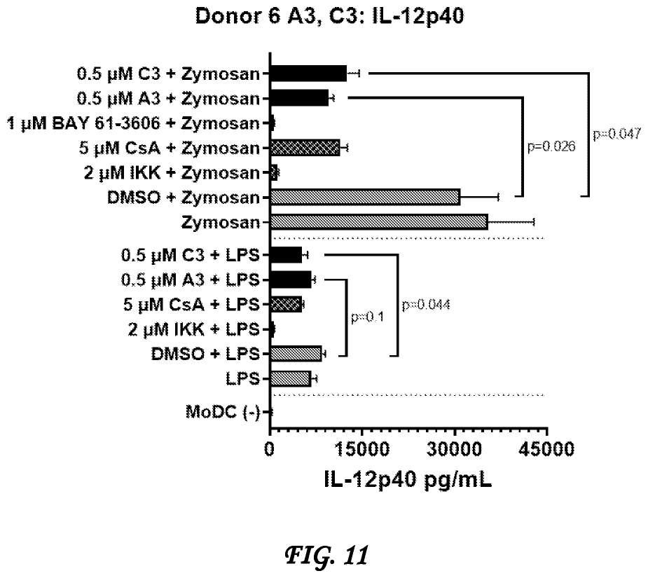

Compounds A3 and C3 inhibit zymosan-induced IL-12p40 in primary human MoDC from healthy donors 5 and 6 when tested at 0.5 μM final concentration. Compound C3 inhibits zymosan-induced IL-12p40 in primary human monocyte-derived dendritic cells from healthy donors 5 (FIG. 11A) and 6 (FIG. 11B) when tested at 0.5 μM final concentration, while compound A3 does not achieve statistically significant inhibition at 0.5 μM final concentration.

### C.A.IRF Assay:

Compounds described herein are added to mammalian cells for bioactivity assessment. The THP-1 dual reporter cell line (InVivoGen) secretes a reporter protein (Lucia luciferase or LUC) in response to activation by the pro-inflammatory bacterial component lipopolysaccharide (LPS). THP-1 dual reporter cells were grown in RPMI 1640 culture medium with 2 mM L-glutamine, 25 mM HEPES, 10% heat-inactivated fetal bovine serum, 100 g/ml Normocin antibiotic (InvivoGen), Pen-Strep antibiotic (100 U/ml-100 g/ml, InvivoGen). On the day of the assay, cells were seeded in a 96 flat-bottom well plate at 50,000 cells/well and incubated for 2 hours. Cells were then incubated with 1 μM or 10 μM of the compounds described herein or with 1 μM of KK-16 (Selleck), a potent inhibitor of IκB kinases (IKKs) or with DMSO (vehicle control) for 2 hours, followed by the addition of LPS at 25 ng/mL and incubation for 24 hours. After incubation was concluded, the cultures supernatants were collected and IRF related activity was assessed by measurement of the Lucia luciferase, using Quanti-Luc assay (InvivoGen) and these values were adjusted by the Cell Viability levels that was assessed using the Cell-Titer-Glo2 Reagent (Promega).

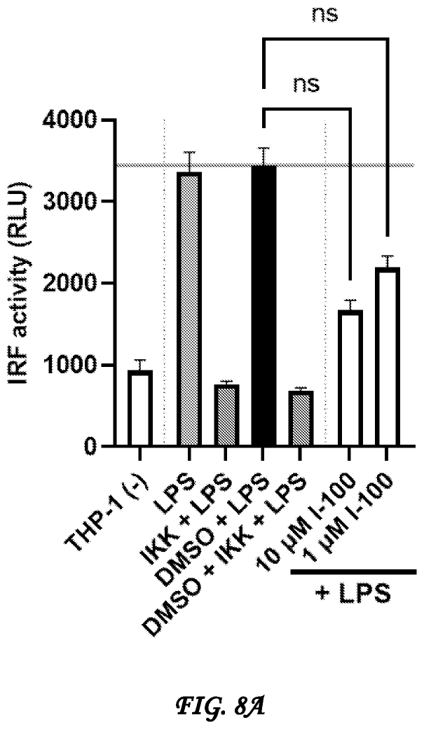

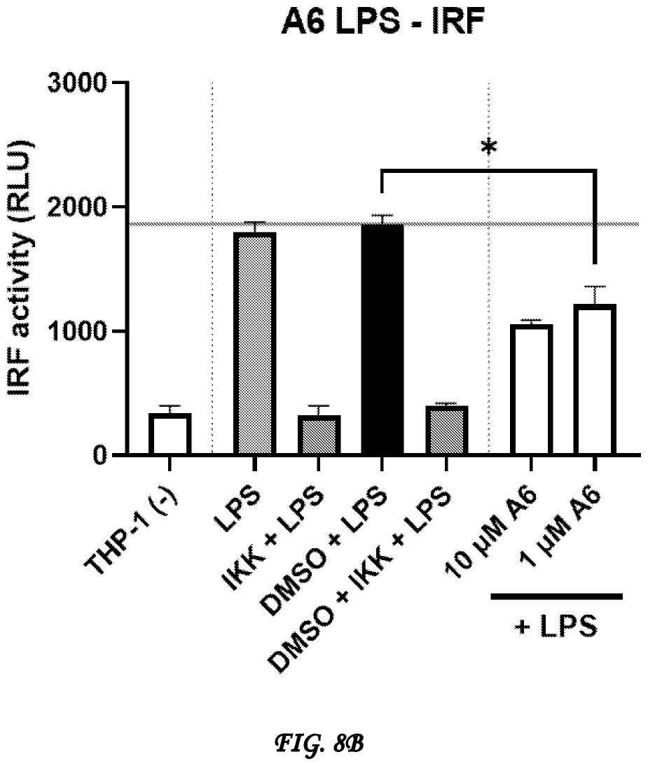

Compound I-100 shows inhibition of LPS-induced IRF reporter activity. This inhibition does not meet statistical significance when tested at 1 and 10 μM. See, FIG. 8A. Compound A6 shows inhibition of LPS-induced IRF reporter activity in a cellular assay.

This inhibition meets statistical significance when tested at 1 μM. See, FIG. 8B.

### Example 36: Compound Interconversion

This examples provide the kinetics of compounds A-2 and A-3. More specifically, A-6 is a mixture of compounds A-2 and A-3 and interconvert in solutions.

In this example, A-6 was combined with 12 nM of Na₃PO₄, DCl, and D₂O at a pH of 7-7.5. This solution was then monitored using 1H-NMR at 37° C.

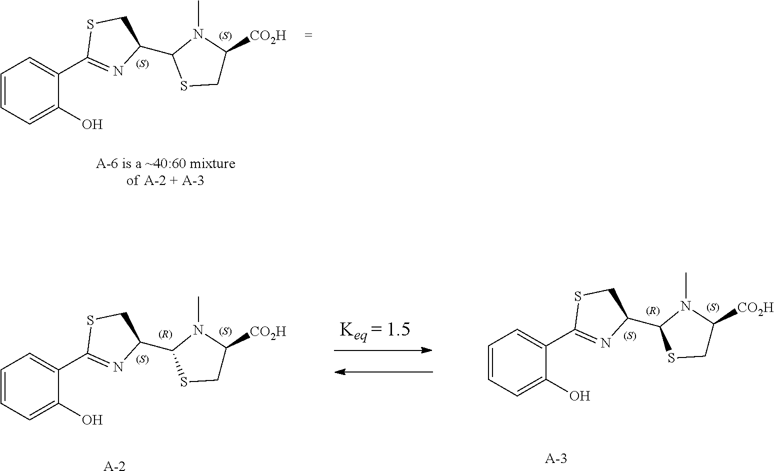

Interconversion between compounds A-2 and A-3 was seen and monitored for 5 days. See, Table 1.

TABLE 1

| Compound | <5 min | 20 min | ~1 hour | ~3 hours | ~7 hours | ~24 hours | 5 days |
|---|---|---|---|---|---|---|---|
| A-2→A-3 | 10.3/1 | 1.2/1 | 1/1 | 1/1.4 | 1/1.5 | 1/1.46 | 1/1.48 |
| A-3→A-2 | 40/1 | 11/1 | 5/1 | 2.3/1 | 1.4/1 | 1.47/1 | 1.46/1 |

These data show that conversion of A-2 to A-3 nearly reaches the 1/about 1.5 equilibrium in about 3 hours at 37° C. in the neutral aqueous buffer. Conversion of A-3 to A2 nearly reaches the about 1.5/1 equilibrium in about 7 hours.

It is to be understood that while the invention has been described in conjunction with the preferred specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains. In addition to the embodiments described herein, the present disclosure contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

The disclosures of each patent, patent application, and publication cited or described in this document are hereby incorporated herein by reference, each in its entirety, for all purposes.

1. A compound of Formula I: 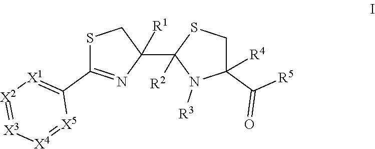
wherein:
R¹, R², R³, and R⁴ are each, independently, H or optionally substituted C₁₋₆alkyl;
R⁵ is OH, optionally substituted C₁₋₆alkoxy, —N(R^{A})(optionally substituted C₁₋₆alkyl), or —X(R^{A})—R¹⁵—CH(X⁶)(X⁷)R¹¹R¹²;
R¹⁵ is —C₁₋₆alk-;
R^{A} is H or optionally substituted C₁₋₆alkyl;
X is O or NH;
X¹ is CR⁶ or N;
X² is CR⁷ or N;
X³ is CR⁸ or N;
X⁴ is CR⁹ or N;
X⁵ is CR¹⁰ or N;
X⁶ and X⁷ are each, independently, H or optionally substituted C₁₋₆alkyl, or both are N and join to form a diazirinyl ring;
R⁶, R⁷, R⁸, R⁹, and R¹⁰ are each, independently, H, OH, halo, optionally substituted C₁₋₆alkyl, optionally substituted C₁₋₆alkoxy, or —(OC₁₋₆alk)ₙ-C₁₋₆alkyl;
R¹¹ and R¹² are each, independently, optionally substituted C₁₋₆alkyl, optionally substituted C₂₋₆alkenyl, optionally substituted C₂₋₆alkynyl, optionally substituted C₁₋₆alkoxy, OH, NH₂, or N(R¹³)(R¹⁴);
R¹³ and R¹⁴ are each, independently, H or C₁₋₆alkyl;
n is 2-8; and
provided that only one of X¹—X⁵ is N;
or a pharmaceutically acceptable salt thereof.

2. The compound of claim 1, wherein R¹, R², R³, and R⁴ are each, independently, H.

3. The compound of claim 1, wherein R¹, R², R³, and R⁴ are each, independently, optionally substituted C₁₋₆alkyl, such as methyl, ethyl, propyl, butyl, pentyl, or hexyl, or such as methyl; or such as R¹ is methyl; or such as R² is methyl; or
such as R³ is methyl; or such as R⁴ is methyl; or such as R¹ is H; or such as R² is H; or
such as R³ is H; or such as R⁴ is H.

4. The compound of claim 1, wherein R⁵, R⁶, R⁷, R⁸, R⁹, R¹⁰, R¹¹ and/or R¹² are each, independently, OH.

5. The compound of claim 1, wherein R⁵ is optionally substituted C₁₋₆alkoxy, such as optionally substituted C₅₋₆alkyl, or such as optionally substituted C₅alkyl, or such as optionally substituted C6alkyl.

6. The compound of claim 1, wherein R⁵ is —N(R^{A})(optionally substituted C₁₋₆alkyl), such as —N(R^{A})(optionally substituted C₅₋₆alkyl), or such as —N(R^{A})(optionally substituted C₅alkyl), or such as —N(R^{A})(optionally substituted C₆alkyl).

7. The compound of claim 1, wherein R⁵ is —X(R^{A})—R¹³—CH(X⁶)(X⁷)R¹¹R¹².

8. The compound of claim 1, wherein R¹⁸ is —CH₂—, —CH₂CH₂—, —CH₂CH₂CH₂—, or —CH₂CH₂CH₂CH₂—.

9. The compound of claim 1, wherein R^{A} is H or optionally substituted C₁₋₆alkyl.

10. The compound of claim 1, wherein X is O.

11. The compound of claim 1, wherein X is NH and X¹, X², X³, X⁴, and/or X⁵ are each, independently N.

12. The compound of claim 1, wherein X¹ is CR⁶.

13. The compound of claim 1, wherein X², X³, X⁴, and/or X⁵ are each, independently, CR⁷ or N.

14. The compound of claim 1, wherein X⁶ and X⁷ are each, independently, H, or such as X⁶ is H; or such as X⁷ is H.

15. The compound of claim 1, wherein X⁶, X⁷, R⁶, R⁷, R⁸, R⁹, R¹⁰, R¹¹ and/or R¹² are each, independently, optionally substituted C₁₋₆alkyl, such as methyl, ethyl, propyl, butyl, pentyl, or hexyl, or such as methyl.

16. The compound of claim 1, wherein X⁶ and X⁷ are N and join to form a diazirinyl ring.

17. The compound of claim 1, wherein R⁶, R⁷, R⁸, R⁹, R¹⁰, R¹³ and/or R¹⁴ are, independently, H.

18. The compound of claim 1, wherein R⁶, R⁷, R⁸, R⁹, and R¹⁰ are each, independently, halo.

19. The compound of claim 1, wherein R⁶, R⁷, R⁸, R⁹, and R¹⁰ are each, independently, optionally substituted C₁₋₆alkoxy.

20. The compound of claim 1, wherein R⁶, R⁷, R⁸, R⁹, and R¹⁰ are each, independently, —(OC₁₋₆alk)ₙ-C₁₋₆alkyl.

21. The compound of claim 1, wherein R¹¹ and R¹² are each, independently, optionally substituted C₂₋₆alkenyl, such as ethenyl, propenyl, butenyl, pentenyl, or hexenyl.

22. The compound of claim 1, wherein R¹¹ and R¹² are each, independently, optionally substituted C₂₋₆alkynyl, such as ethynyl, propynyl, butynyl, pentynyl, or hexynyl.

23. The compound of claim 1, wherein R¹¹ and R¹² are each, independently, optionally substituted C₁₋₆alkoxy, such as methoxy, ethoxy, propoxy, butoxy, pentoxy, or hexoxy.

24. The compound of claim 1, wherein R¹¹ and R¹² are each, independently, NH₂ or N(R¹³)(R¹⁴), such as NHCH₃, or such as N(CH₃)₂.

25. The compound of claim 1, wherein R¹³ and R¹⁴ are each, independently, C₁₋₆alkyl, such as methyl, ethyl, propyl, butyl, pentyl, or hexyl.

26. The compound of claim 1, wherein n is 3-6, or 2, or 3, or 4, or 5, or 6, or 7, or 8.

27. The compound of claim 1, that is 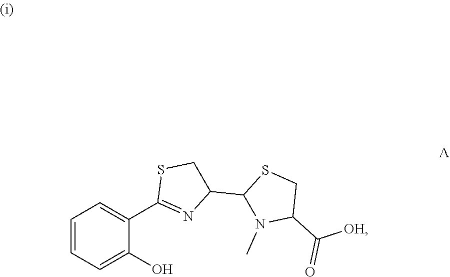 [SMILES: CN1C(C(=O)O)CSC1C1CSC(c2ccccc2O)=N1]
or a pharmaceutically acceptable salt thereof, such as 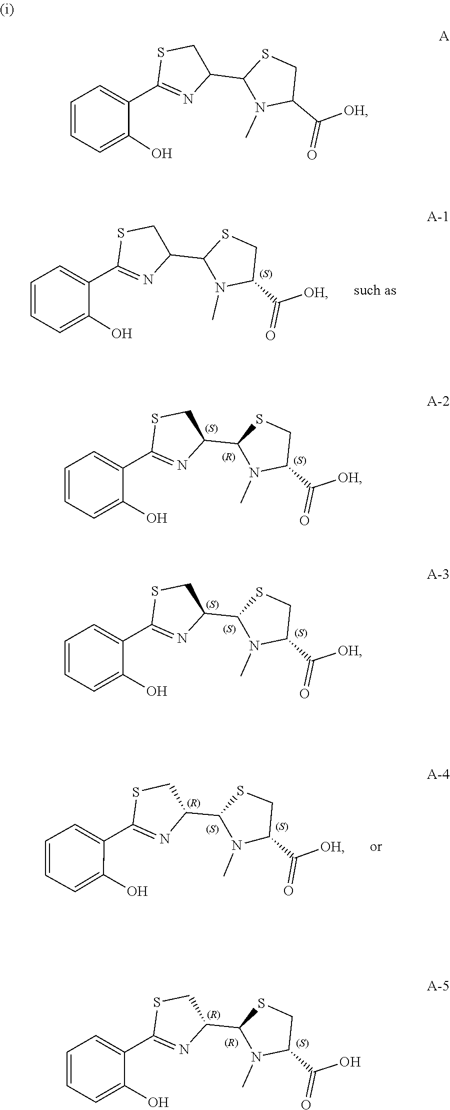
or a pharmaceutically acceptable salt thereof; 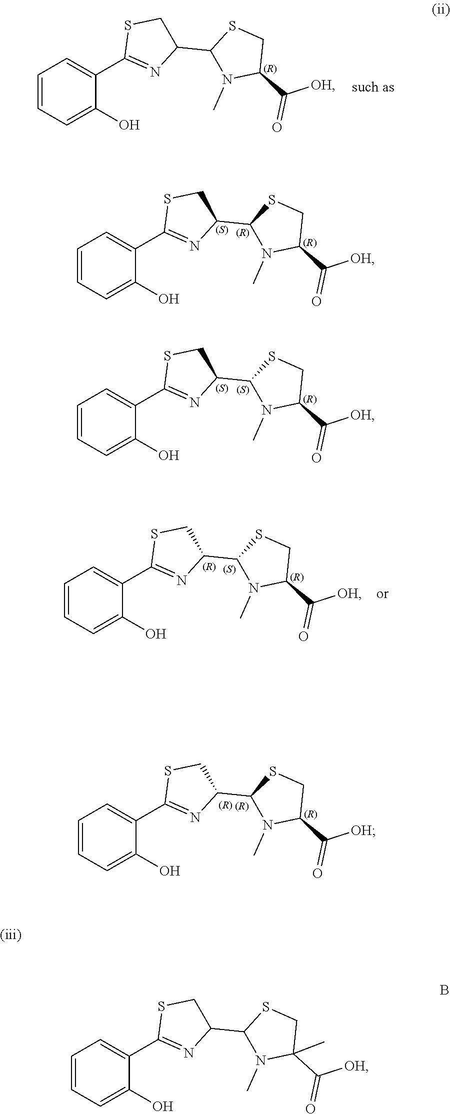
or a pharmaceutically acceptable salt thereof, such as 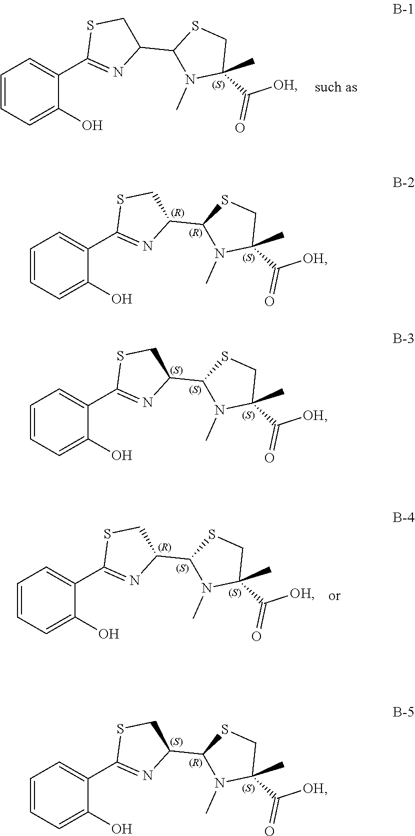
or a pharmaceutically acceptable salt thereof; 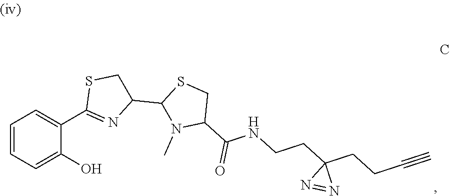 [SMILES: C#CCCC1(CCNC(=O)C2CSC(C3CSC(c4ccccc4O)=N3)N2C)N=N1]
or a pharmaceutically acceptable salt thereof, such as 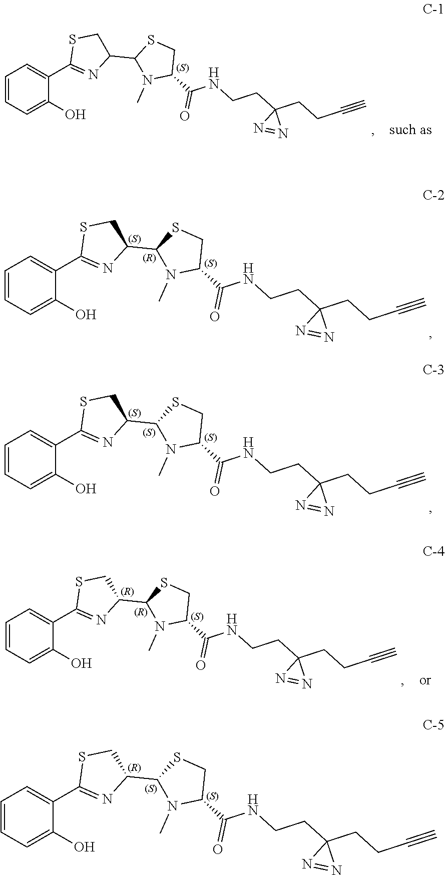
or a pharmaceutically acceptable salt thereof; 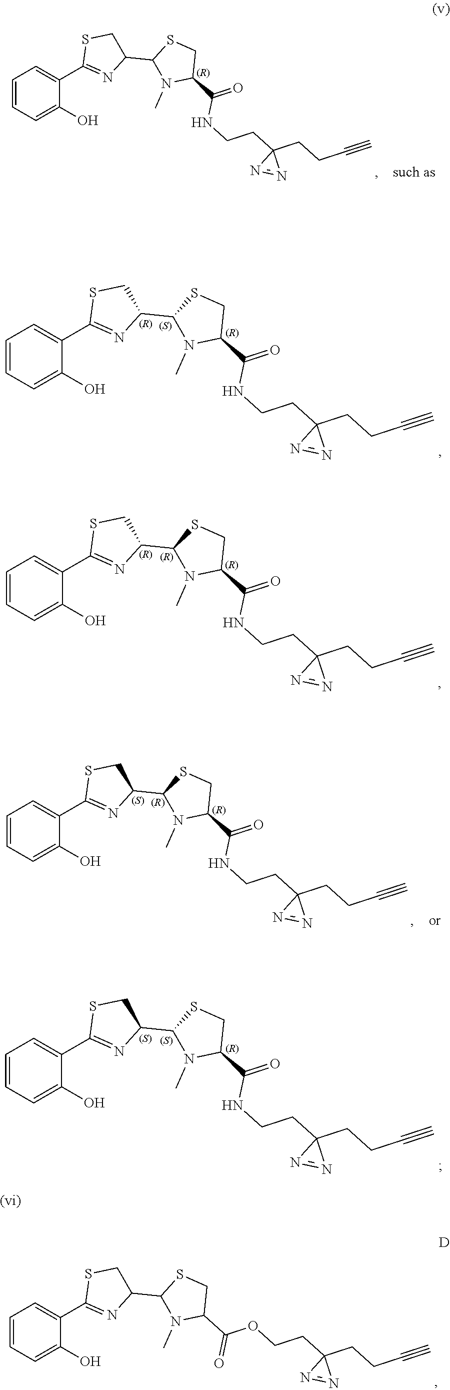
or a pharmaceutically acceptable salt thereof, such as 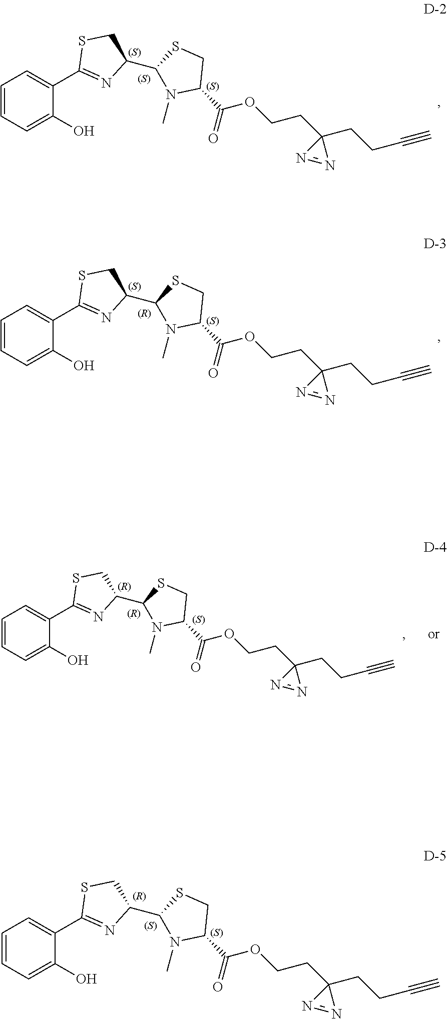
or a pharmaceutically acceptable salt thereof; 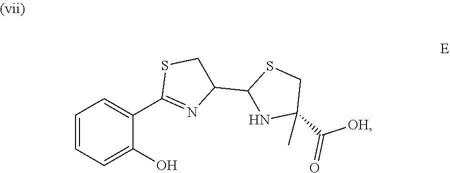 [SMILES: C[C@]1(C(=O)O)CSC(C2CSC(c3ccccc3O)=N2)N1]
or a pharmaceutically acceptable salt thereof, such as 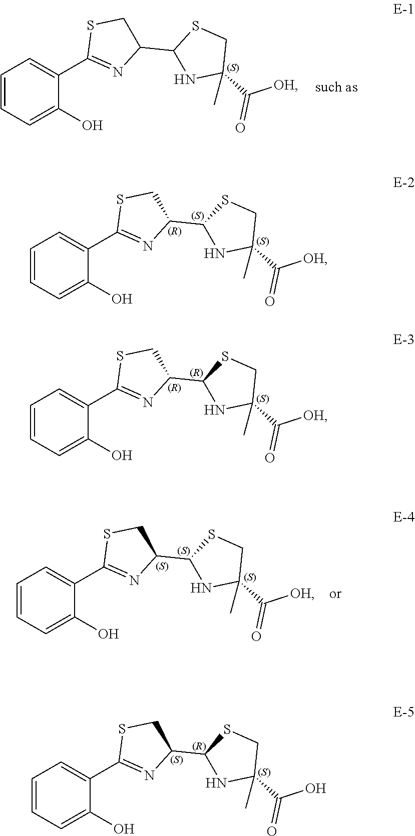
or a pharmaceutically acceptable salt thereof, 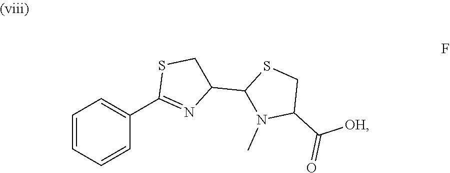 [SMILES: CN1C(C(=O)O)CSC1C1CSC(c2ccccc2)=N1]
or a pharmaceutically acceptable salt thereof, such as 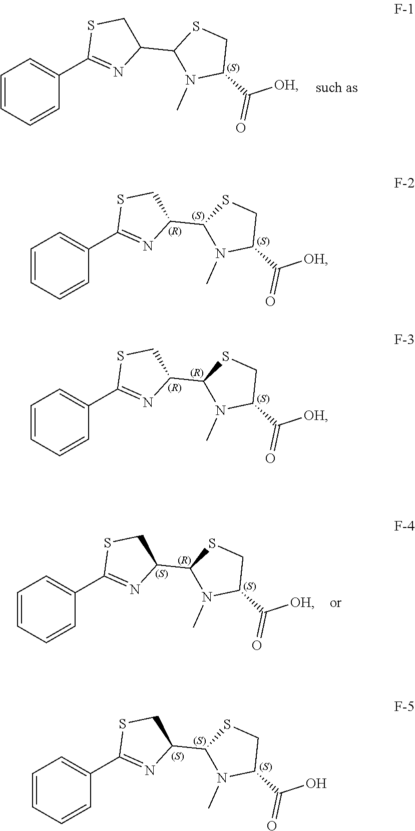
or a pharmaceutically acceptable salt thereof, 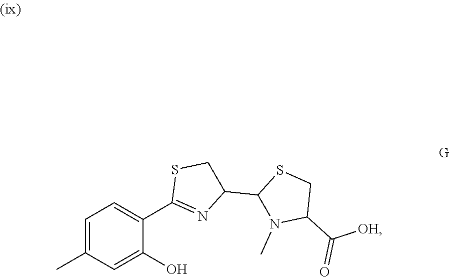 [SMILES: Cc1ccc(C2=NC(C3SCC(C(=O)O)N3C)CS2)c(O)c1]
or a pharmaceutically acceptable salt thereof, such as 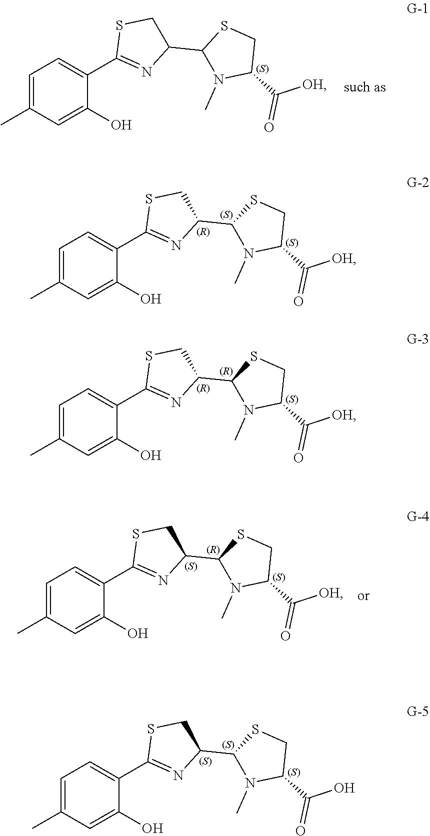
or a pharmaceutically acceptable salt thereof, 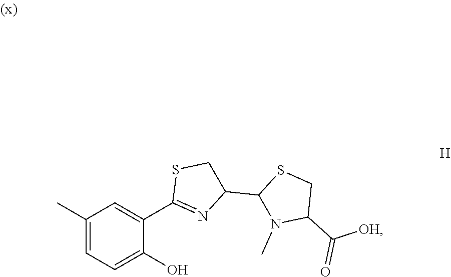 [SMILES: Cc1ccc(O)c(C2=NC(C3SCC(C(=O)O)N3C)CS2)c1]
or a pharmaceutically acceptable salt thereof, such as 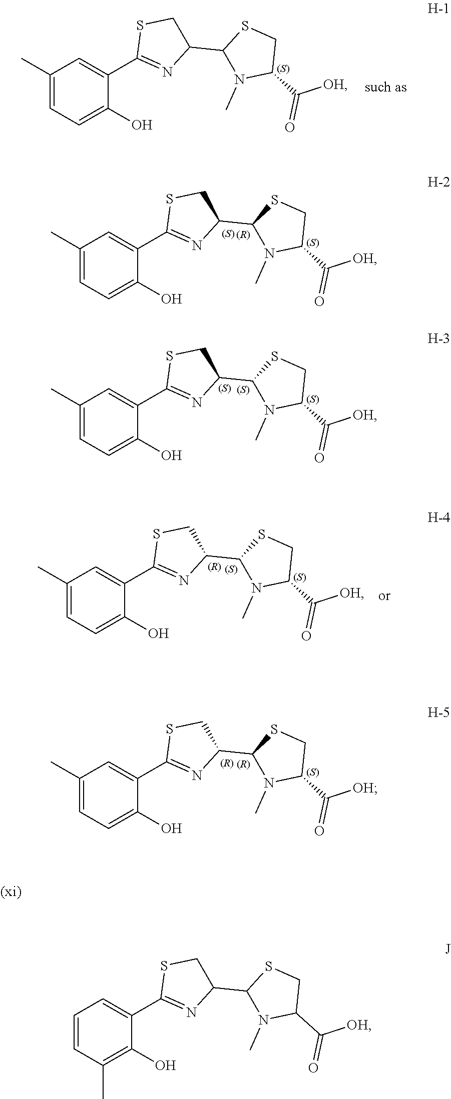
or a pharmaceutically acceptable salt thereof, such as 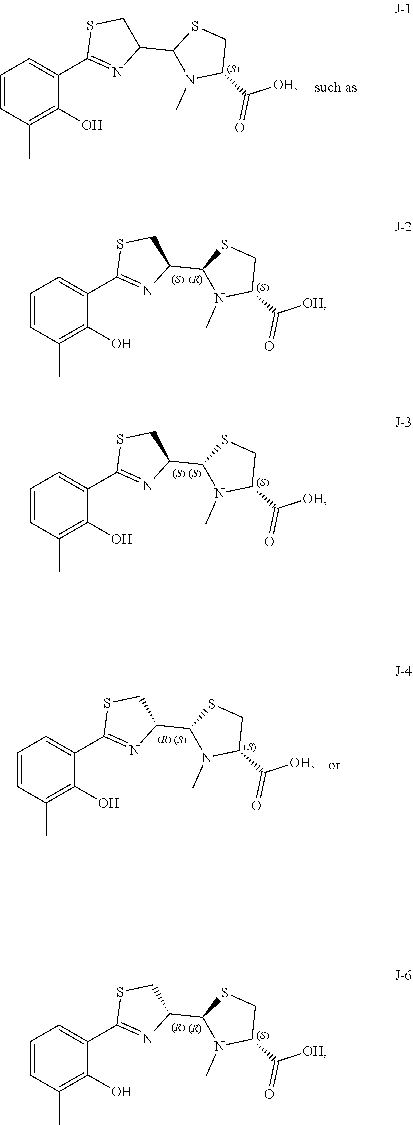
or a pharmaceutically acceptable salt thereof, 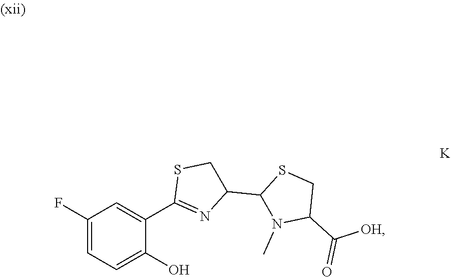 [SMILES: CN1C(C(=O)O)CSC1C1CSC(c2cc(F)ccc2O)=N1]
or a pharmaceutically acceptable salt thereof, such as 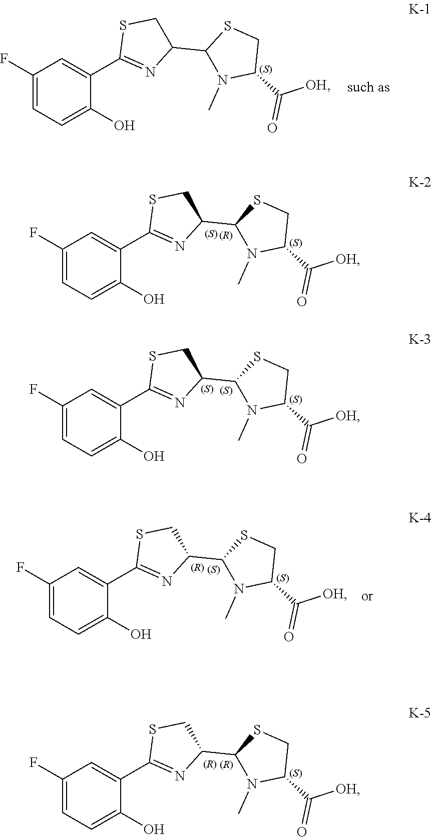
or a pharmaceutically acceptable salt thereof; 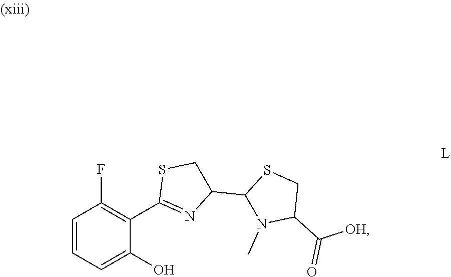 [SMILES: CN1C(C(=O)O)CSC1C1CSC(c2c(O)cccc2F)=N1]
or a pharmaceutically acceptable salt thereof, such as 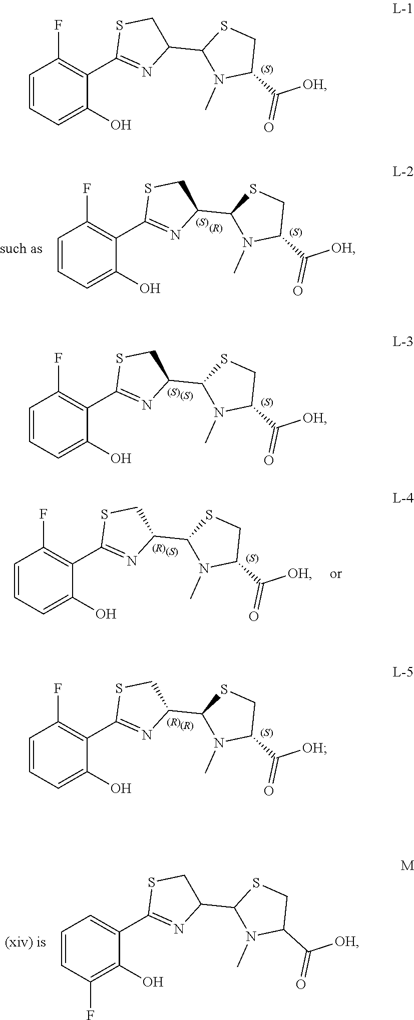
or a pharmaceutically acceptable salt thereof; such as 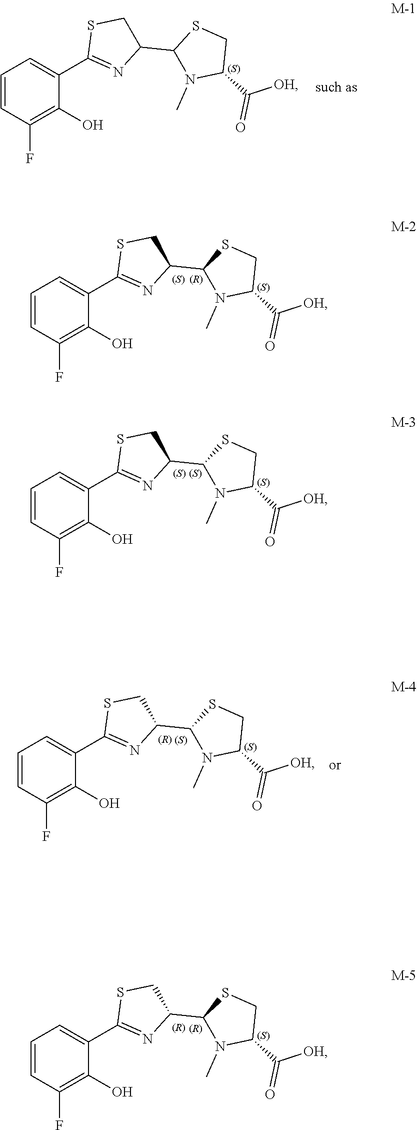
or a pharmaceutically acceptable salt thereof, 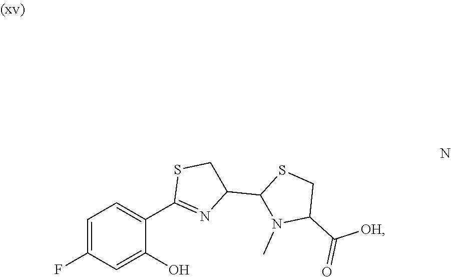 [SMILES: CN1C(C(=O)O)CSC1C1CSC(c2ccc(F)cc2O)=N1]
or a pharmaceutically acceptable salt thereof, such as 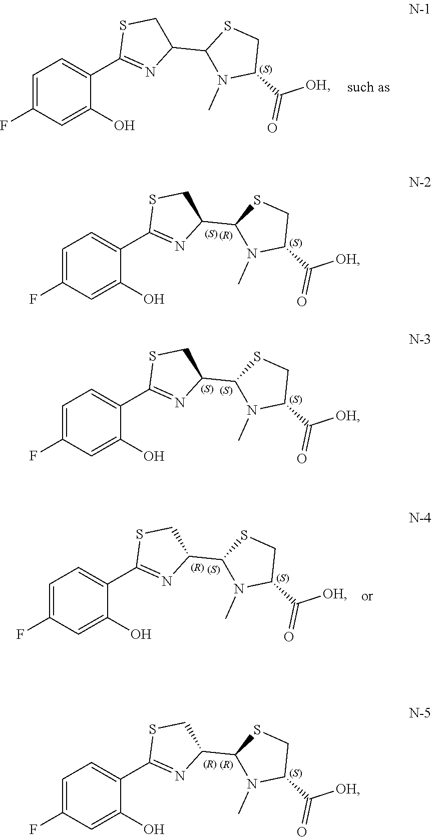
or a pharmaceutically acceptable salt thereof; 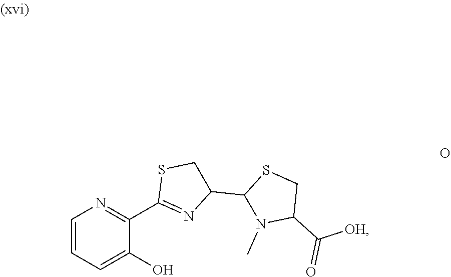 [SMILES: CN1C(C(=O)O)CSC1C1CSC(c2ncccc2O)=N1]
or a pharmaceutically acceptable salt thereof, such as 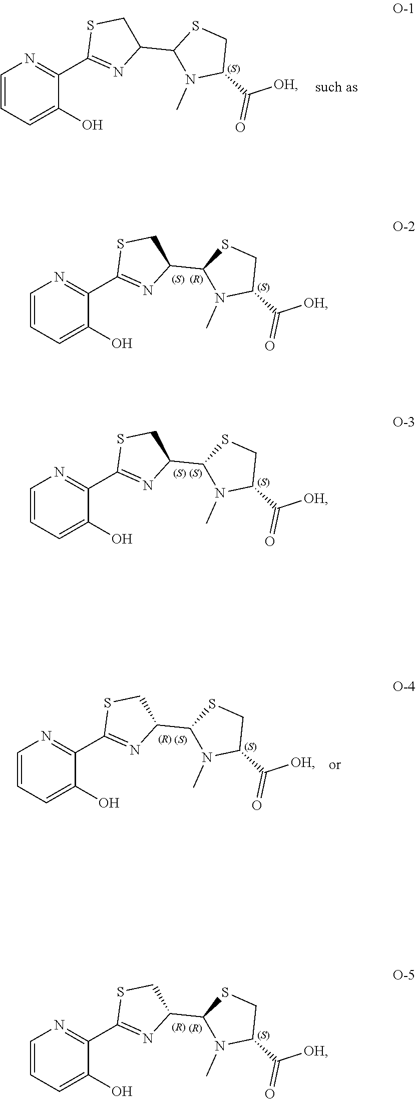
or a pharmaceutically acceptable salt thereof; or 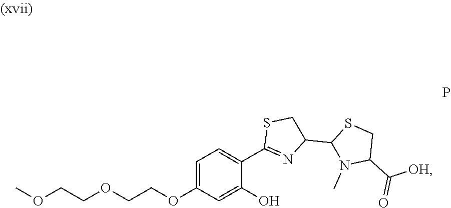 [SMILES: COCCOCCOc1ccc(C2=NC(C3SCC(C(=O)O)N3C)CS2)c(O)c1]
or a pharmaceutically acceptable salt thereof, such as 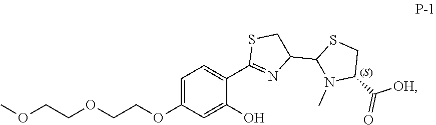
such as 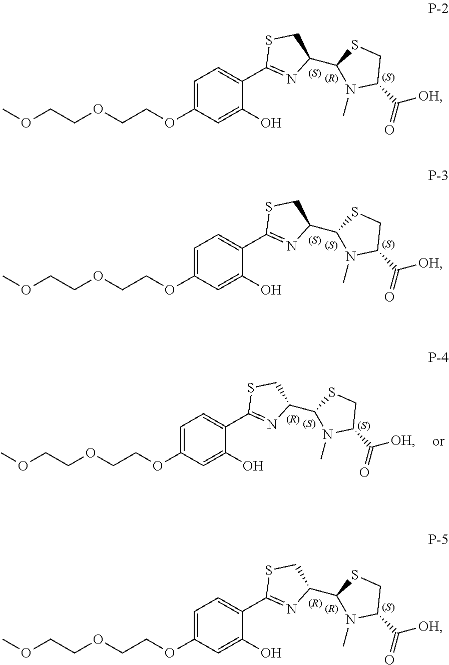
or a pharmaceutically acceptable salt thereof.

28. The compound of claim 1, wherein the compound is of Formula II, III, IV, or V:

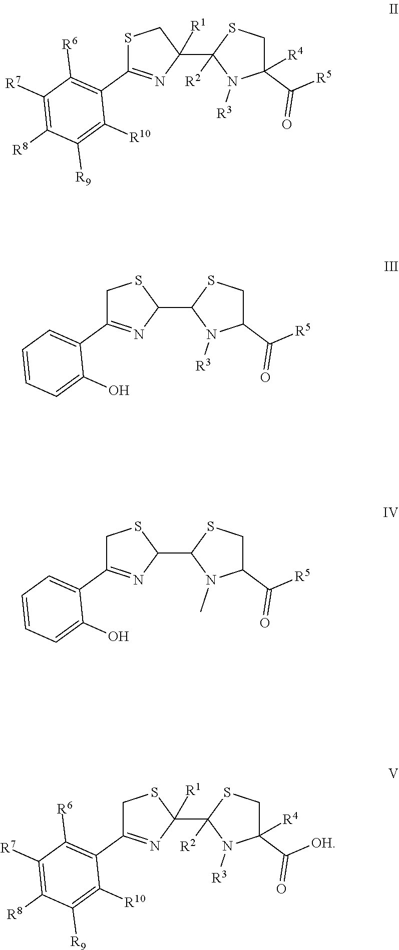

29. A pharmaceutical composition comprising a therapeutically effective amount of one or more of the compound of claim 1 and a pharmaceutically acceptable excipient.

30. The pharmaceutical composition of claim 29, comprising two diastereomers of the compound, wherein the ratio of a first diastereomer to a second diastereomer is about 10:about 90, or about 25:about 75, or about 40:about 60, or about 50:about 50, or about 60:about 40, or about 75:about 25, or about 90:about 10.

31. A dosage unit comprising compound of claim 1, optionally about 0.01 to about 1000 mg of the compound.

32. A method of modulating an inflammatory bowel disease (IBD) marker or treating an inflammatory bowel disease (IBD) in a subject in need thereof, comprising administering the compound of claim 1 to the subject in need thereof.

33. The method of claim 32, wherein the subject has or is at risk of developing IBD.

34. The method of claim 32, wherein the IBD is ulcerative colitis or Crohn's disease.

35. The method of claim 32, wherein:
tumor necrosis factor alpha (TNF-α), interleukin-1β (IL-1β0), interleukin-6 (IL-6), interleukin-8 (IL-8), interleukin-23 (IL-23), interleukin 12 (IL-12), or interleukin-17A (IL-17A) levels, or a combination thereof, in the subject are reduced; and/or
nuclear factor kappa B (NF-κB), monocyte chemoattractant protein-1 (MCP-1), cysteine-cysteine motif chemokine ligand 20 (CCL20), or interferon-7 (IFN-7) levels, or a combination thereof, in the subject are reduced; and/or
M2 pyruvate kinase (M2-PK), osteoprotegerin, myeloperoxidase (MPO), high mobility group box protein 1 (HMGB1), chitinase 3 like 1 (CHI3L1), human beta defensin 2 (HBD2), metalloproteinase (MMP), calprotectin, lactoferrin, peripheral antineutrophil cytoplasmic antibodies (p-ANCA), or anti-Saccharomyces cerevisiae antibodies (ASCA) levels, or a combination thereof, in the subject are reduced; and/or
inflammation or ulcers in the intestinal lining of the subject are reduced, as observed by colonoscopy; and/or
one or both serum amyloid A protein (SAA) or eotaxin-1 levels are reduced in the subject.

36-90. (canceled)
